SEQUENCE LISTING (848 sequences)

SEQ ID NO: 1 - RNA, 12 nt, Streptococcus pyogenes
  source 1..12: mol_type = other RNA
gttttagagcta

SEQ ID NO: 2 - RNA, 25 nt, Campylobacter jejuni
  source 1..25: mol_type = other RNA
gttttagtccctttttaaatttctt

SEQ ID NO: 3 - RNA, 13 nt, Campylobacter jejuni
  source 1..13: mol_type = other RNA
gttttagtccctt

SEQ ID NO: 4 - skipped

SEQ ID NO: 5 - RNA, 14 nt, Streptococcus pyogenes
  source 1..14: mol_type = other RNA
tagcaagttaaaat

SEQ ID NO: 6 - RNA, 25 nt, Campylobacter jejuni
  source 1..25: mol_type = other RNA
aagaaatttaaaaagggactaaaat

SEQ ID NO: 7 - RNA, 13 nt, Campylobacter jejuni
  source 1..13: mol_type = other RNA
aagggactaaaat

SEQ ID NO: 8 - RNA, 11 nt, unidentified
  misc_feature 1..11: note = Parcubacteria bacterium
  source 1..11: mol_type = other RNA
aaatttctact

SEQ ID NO: 9 - RNA, 12 nt, Streptococcus pyogenes
  source 1..12: mol_type = other RNA
aaggctagtccg

SEQ ID NO: 10 - RNA, 11 nt, Campylobacter jejuni
  source 1..11: mol_type = other RNA
aaagagtttgc

SEQ ID NO: 11 - RNA, 34 nt, Streptococcus pyogenes
  source 1..34: mol_type = other RNA
ttatcaacttgaaaaagtggcaccgagtcggtgc

SEQ ID NO: 12 - RNA, 38 nt, Campylobacter jejuni
  source 1..38: mol_type = other RNA
gggactctgcggggttacaatcccctaaaaccgctttt

SEQ ID NO: 13 - RNA, 22 nt, Streptococcus thermophilus
  source 1..22: mol_type = other RNA
gttttagagctgtgttgtttcg

SEQ ID NO: 14 - RNA, 24 nt, Streptococcus thermophilus
  source 1..24: mol_type = other RNA
cgaaacaacacagcgagttaaaat

SEQ ID NO: 15 - RNA, 13 nt, Streptococcus thermophilus
  source 1..13: mol_type = other RNA
aaggcttagtccg

SEQ ID NO: 16 - RNA, 38 nt, Streptococcus thermophilus
  source 1..38: mol_type = other RNA
tactcaacttgaaaaggtggcaccgattcggtgttttt

SEQ ID NO: 17 - DNA, 20 bp, synthetic construct
  misc_feature 1..20: note = Target sequence
  source 1..20: mol_type = other DNA
atcattggcagactagttcg

SEQ ID NO: 18 - DNA, 20 bp, synthetic construct
  misc_feature 1..20: note = Target sequence
  source 1..20: mol_type = other DNA
cgaactagtctgccaatgat

SEQ ID NO: 19 - DNA, 20 bp, Homo sapiens
  source 1..20: mol_type = genomic DNA
tcctatcacattggaataca

SEQ ID NO: 20 - DNA, 20 bp, Homo sapiens
  source 1..20: mol_type = genomic DNA
ggttacagttcctatcacat

SEQ ID NO: 21 - DNA, 20 bp, Homo sapiens
  source 1..20: mol_type = genomic DNA
gccatgtattccaatgtgat

SEQ ID NO: 22 - DNA, 20 bp, Homo sapiens
  source 1..20: mol_type = genomic DNA
gtgataggaactgtaacctc

SEQ ID NO: 23 - DNA, 20 bp, Homo sapiens
  source 1..20: mol_type = genomic DNA
cccctcttacctttttccag

SEQ ID NO: 24 - DNA, 20 bp, Homo sapiens
  source 1..20: mol_type = genomic DNA
gaactgtaacctctggaaaa

SEQ ID NO: 25 - DNA, 20 bp, Homo sapiens
  source 1..20: mol_type = genomic DNA
ccagaagccaatgagcagca

SEQ ID NO: 26 - DNA, 20 bp, Homo sapiens
  source 1..20: mol_type = genomic DNA
cttttgtccttgctgctcat

SEQ ID NO: 27 - DNA, 20 bp, Homo sapiens
  source 1..20: mol_type = genomic DNA
ccttgctgctcattggcttc

SEQ ID NO: 28 - DNA, 20 bp, Homo sapiens
  source 1..20: mol_type = genomic DNA
cttgctgctcattggcttct

SEQ ID NO: 29 - DNA, 20 bp, Homo sapiens
  source 1..20: mol_type = genomic DNA
tgggactgcgtgacctgtca

SEQ ID NO: 30 - DNA, 20 bp, Homo sapiens
  source 1..20: mol_type = genomic DNA
gggactgcgtgacctgtcac

SEQ ID NO: 31 - DNA, 20 bp, Homo sapiens
  source 1..20: mol_type = genomic DNA
gtccacagggctcccgtgac

SEQ ID NO: 32 - DNA, 20 bp, Homo sapiens
  source 1..20: mol_type = genomic DNA
gacctgtcacgggagccctg

SEQ ID NO: 33 - DNA, 20 bp, Homo sapiens
  source 1..20: mol_type = genomic DNA
tggctgtgcagatgtccaca

SEQ ID NO: 34 - DNA, 20 bp, Homo sapiens
  source 1..20: mol_type = genomic DNA
ttggctgtgcagatgtccac

SEQ ID NO: 35 - DNA, 20 bp, Homo sapiens
  source 1..20: mol_type = genomic DNA
acatctgcacagccaagccg

SEQ ID NO: 36 - DNA, 20 bp, Homo sapiens
  source 1..20: mol_type = genomic DNA
catctgcacagccaagccgc

SEQ ID NO: 37 - DNA, 20 bp, Homo sapiens
  source 1..20: mol_type = genomic DNA
catgggaatgtcccgcggct

SEQ ID NO: 38 - DNA, 20 bp, Homo sapiens
  source 1..20: mol_type = genomic DNA
ggattcatgggaatgtcccg

SEQ ID NO: 39 - DNA, 20 bp, Homo sapiens
  source 1..20: mol_type = genomic DNA
taaatgcacatgggattcat

SEQ ID NO: 40 - DNA, 20 bp, Homo sapiens
  source 1..20: mol_type = genomic DNA
gtaaatgcacatgggattca

SEQ ID NO: 41 - DNA, 20 bp, Homo sapiens
  source 1..20: mol_type = genomic DNA
ggggagcggtaaatgcacat

SEQ ID NO: 42 - DNA, 20 bp, Homo sapiens
  source 1..20: mol_type = genomic DNA
cggggagcggtaaatgcaca

SEQ ID NO: 43 - DNA, 20 bp, Homo sapiens
  source 1..20: mol_type = genomic DNA
catgtgcatttaccgctccc

SEQ ID NO: 44 - DNA, 20 bp, Homo sapiens
  source 1..20: mol_type = genomic DNA
ttgccttcttctccggggag

SEQ ID NO: 45 - DNA, 20 bp, Homo sapiens
  source 1..20: mol_type = genomic DNA
ctcagttgccttcttctccg

SEQ ID NO: 46 - DNA, 20 bp, Homo sapiens
  source 1..20: mol_type = genomic DNA
cctcagttgccttcttctcc

SEQ ID NO: 47 - DNA, 20 bp, Homo sapiens
  source 1..20: mol_type = genomic DNA
tcctcagttgccttcttctc

SEQ ID NO: 48 - DNA, 20 bp, Homo sapiens
  source 1..20: mol_type = genomic DNA
ttaccgctccccggagaaga

SEQ ID NO: 49 - DNA, 20 bp, Homo sapiens
  source 1..20: mol_type = genomic DNA
cccggagaagaaggcaactg

SEQ ID NO: 50 - DNA, 20 bp, Homo sapiens
  source 1..20: mol_type = genomic DNA
gaagaaggcaactgaggatg

SEQ ID NO: 51 - DNA, 20 bp, Homo sapiens
  source 1..20: mol_type = genomic DNA
aagaaggcaactgaggatga

SEQ ID NO: 52 - DNA, 20 bp, Homo sapiens
  source 1..20: mol_type = genomic DNA
gggctcagaacagaagatcc

SEQ ID NO: 53 - DNA, 20 bp, Homo sapiens
  source 1..20: mol_type = genomic DNA
ctcagaacagaagatcccgg

SEQ ID NO: 54 - DNA, 20 bp, Homo sapiens
  source 1..20: mol_type = genomic DNA
cacgccggttggtggcctcc

SEQ ID NO: 55 - DNA, 20 bp, Homo sapiens
  source 1..20: mol_type = genomic DNA
acacgccggttggtggcctc

SEQ ID NO: 56 - DNA, 20 bp, Homo sapiens
  source 1..20: mol_type = genomic DNA
agatcccggaggccaccaac

SEQ ID NO: 57 - DNA, 20 bp, Homo sapiens
  source 1..20: mol_type = genomic DNA
ttcccagacacgccggttgg

SEQ ID NO: 58 - DNA, 20 bp, Homo sapiens
  source 1..20: mol_type = genomic DNA
cagttcccagacacgccggt

SEQ ID NO: 59 - DNA, 20 bp, Homo sapiens
  source 1..20: mol_type = genomic DNA
tggacagttcccagacacgc

SEQ ID NO: 60 - DNA, 20 bp, Homo sapiens
  source 1..20: mol_type = genomic DNA
aggccaccaaccggcgtgtc

SEQ ID NO: 61 - DNA, 20 bp, Homo sapiens
  source 1..20: mol_type = genomic DNA
ggccaccaaccggcgtgtct

SEQ ID NO: 62 - DNA, 20 bp, Homo sapiens
  source 1..20: mol_type = genomic DNA
gcgtgtctgggaactgtcca

SEQ ID NO: 63 - DNA, 20 bp, Homo sapiens
  source 1..20: mol_type = genomic DNA
agcaaagcgggaattggcct

SEQ ID NO: 64 - DNA, 20 bp, Homo sapiens
  source 1..20: mol_type = genomic DNA
agtggtagcaaagcgggaat

SEQ ID NO: 65 - DNA, 20 bp, Homo sapiens
  source 1..20: mol_type = genomic DNA
atagaaagtggtagcaaagc

SEQ ID NO: 66 - DNA, 20 bp, Homo sapiens
  source 1..20: mol_type = genomic DNA
gatagaaagtggtagcaaag

SEQ ID NO: 67 - DNA, 20 bp, Homo sapiens
  source 1..20: mol_type = genomic DNA
tgccaggtgctgatagaaag

SEQ ID NO: 68 - DNA, 20 bp, Homo sapiens
  source 1..20: mol_type = genomic DNA
taccactttctatcagcacc

SEQ ID NO: 69 - DNA, 20 bp, Homo sapiens
  source 1..20: mol_type = genomic DNA
tgtcattcttggaatctgcc

SEQ ID NO: 70 - DNA, 20 bp, Homo sapiens
  source 1..20: mol_type = genomic DNA
aatgttatcattgtcattct

SEQ ID NO: 71 - DNA, 20 bp, Homo sapiens
  source 1..20: mol_type = genomic DNA
tggagatactcaggggtgac

SEQ ID NO: 72 - DNA, 20 bp, Homo sapiens
  source 1..20: mol_type = genomic DNA
aaagccgtggagatactcag

SEQ ID NO: 73 - DNA, 20 bp, Homo sapiens
  source 1..20: mol_type = genomic DNA
aaaagccgtggagatactca

SEQ ID NO: 74 - DNA, 20 bp, Homo sapiens
  source 1..20: mol_type = genomic DNA
caaaagccgtggagatactc

SEQ ID NO: 75 - DNA, 20 bp, Homo sapiens
  source 1..20: mol_type = genomic DNA
gtcacccctgagtatctcca

SEQ ID NO: 76 - DNA, 20 bp, Homo sapiens
  source 1..20: mol_type = genomic DNA
cttggtcatagcaaaagccg

SEQ ID NO: 77 - DNA, 20 bp, Homo sapiens
  source 1..20: mol_type = genomic DNA
ggcttttgctatgaccaagc

SEQ ID NO: 78 - DNA, 20 bp, Homo sapiens
  source 1..20: mol_type = genomic DNA
gcttttgctatgaccaagct

SEQ ID NO: 79 - DNA, 20 bp, Homo sapiens
  source 1..20: mol_type = genomic DNA
gtcattacaggcacccagct

SEQ ID NO: 80 - DNA, 20 bp, Homo sapiens
  source 1..20: mol_type = genomic DNA
ttgctggagggtgtcattac

SEQ ID NO: 81 - DNA, 20 bp, Homo sapiens
  source 1..20: mol_type = genomic DNA
tacctccatcagttgctgga

SEQ ID NO: 82 - DNA, 20 bp, Homo sapiens
  source 1..20: mol_type = genomic DNA
gtacctccatcagttgctgg

SEQ ID NO: 83 - DNA, 20 bp, Homo sapiens
  source 1..20: mol_type = genomic DNA
gtcgtacctccatcagttgc

SEQ ID NO: 84 - DNA, 20 bp, Homo sapiens
  source 1..20: mol_type = genomic DNA
tgacaccctccagcaactga

SEQ ID NO: 85 - DNA, 20 bp, Homo sapiens
  source 1..20: mol_type = genomic DNA
caccctccagcaactgatgg

SEQ ID NO: 86 - DNA, 20 bp, Homo sapiens
  source 1..20: mol_type = genomic DNA
atcagatgttttctcagata

SEQ ID NO: 87 - DNA, 20 bp, Homo sapiens
  source 1..20: mol_type = genomic DNA
tcagtttggcaaagaagaag

SEQ ID NO: 88 - DNA, 20 bp, Homo sapiens
  source 1..20: mol_type = genomic DNA
atagagtcggcagttcagtt

SEQ ID NO: 89 - DNA, 20 bp, Homo sapiens
  source 1..20: mol_type = genomic DNA
tgttggcttttcgatagagt

SEQ ID NO: 90 - DNA, 20 bp, Homo sapiens
  source 1..20: mol_type = genomic DNA
tactaacttggaggatttgt

SEQ ID NO: 91 - DNA, 20 bp, Homo sapiens
  source 1..20: mol_type = genomic DNA
attggctgatactaacttgg

SEQ ID NO: 92 - DNA, 20 bp, Homo sapiens
  source 1..20: mol_type = genomic DNA
gcgattggctgatactaact

SEQ ID NO: 93 - DNA, 20 bp, Homo sapiens
  source 1..20: mol_type = genomic DNA
tttgtctccaaaaaggcgat

SEQ ID NO: 94 - DNA, 20 bp, Homo sapiens
  source 1..20: mol_type = genomic DNA
gtatcagccaatcgcctttt

SEQ ID NO: 95 - DNA, 20 bp, Homo sapiens
  source 1..20: mol_type = genomic DNA
taagggatttgtctccaaaa

SEQ ID NO: 96 - DNA, 20 bp, Homo sapiens
  source 1..20: mol_type = genomic DNA
gtaggtctcattgaaggtaa

SEQ ID NO: 97 - DNA, 20 bp, Homo sapiens
  source 1..20: mol_type = genomic DNA
ggtaggtctcattgaaggta

SEQ ID NO: 98 - DNA, 20 bp, Homo sapiens
  source 1..20: mol_type = genomic DNA
gtcctggtaggtctcattga

SEQ ID NO: 99 - DNA, 20 bp, Homo sapiens
  source 1..20: mol_type = genomic DNA
taccttcaatgagacctacc

SEQ ID NO: 100 - DNA, 20 bp, Homo sapiens
  source 1..20: mol_type = genomic DNA
caactcactgatgtcctggt

SEQ ID NO: 101 - DNA, 20 bp, Homo sapiens
  source 1..20: mol_type = genomic DNA
ataccaactcactgatgtcc

SEQ ID NO: 102 - DNA, 20 bp, Homo sapiens
  source 1..20: mol_type = genomic DNA
ctaccaggacatcagtgagt

SEQ ID NO: 103 - DNA, 20 bp, Homo sapiens
  source 1..20: mol_type = genomic DNA
gacatcagtgagttggtata

SEQ ID NO: 104 - DNA, 20 bp, Homo sapiens
  source 1..20: mol_type = genomic DNA
gaagtccaggggctggagct

SEQ ID NO: 105 - DNA, 20 bp, Homo sapiens
  source 1..20: mol_type = genomic DNA
tggagccaagctccagcccc

SEQ ID NO: 106 - DNA, 20 bp, Homo sapiens
  source 1..20: mol_type = genomic DNA
tcaccttgaagtccaggggc

SEQ ID NO: 107 - DNA, 20 bp, Homo sapiens
  source 1..20: mol_type = genomic DNA
caactcaccttgaagtccag

SEQ ID NO: 108 - DNA, 20 bp, Homo sapiens
  source 1..20: mol_type = genomic DNA
gcaactcaccttgaagtcca

SEQ ID NO: 109 - DNA, 20 bp, Homo sapiens
  source 1..20: mol_type = genomic DNA
tgcaactcaccttgaagtcc

SEQ ID NO: 110 - DNA, 20 bp, Homo sapiens
  source 1..20: mol_type = genomic DNA
gctccagcccctggacttca

SEQ ID NO: 111 - DNA, 20 bp, Homo sapiens
  source 1..20: mol_type = genomic DNA
gattgctctgcattttcctg

SEQ ID NO: 112 - DNA, 20 bp, Homo sapiens
  source 1..20: mol_type = genomic DNA
aaatgcagagcaatccagag

SEQ ID NO: 113 - DNA, 20 bp, Homo sapiens
  source 1..20: mol_type = genomic DNA
ccatttgttgatggccgctc

SEQ ID NO: 114 - DNA, 20 bp, Homo sapiens
  source 1..20: mol_type = genomic DNA
attggacacccatttgttga

SEQ ID NO: 115 - DNA, 20 bp, Homo sapiens
  source 1..20: mol_type = genomic DNA
ccagagcggccatcaacaaa

SEQ ID NO: 116 - DNA, 20 bp, Homo sapiens
  source 1..20: mol_type = genomic DNA
cagagcggccatcaacaaat

SEQ ID NO: 117 - DNA, 20 bp, Homo sapiens
  source 1..20: mol_type = genomic DNA
gattcggccttcggtcttat

SEQ ID NO: 118 - DNA, 20 bp, Homo sapiens
  source 1..20: mol_type = genomic DNA
tgggtgtccaataagaccga

SEQ ID NO: 119 - DNA, 20 bp, Homo sapiens
  source 1..20: mol_type = genomic DNA
gacatcggtgattcggcctt

SEQ ID NO: 120 - DNA, 20 bp, Homo sapiens
  source 1..20: mol_type = genomic DNA
agggaatgacatcggtgatt

SEQ ID NO: 121 - DNA, 20 bp, Homo sapiens
  source 1..20: mol_type = genomic DNA
ggcttccgagggaatgacat

SEQ ID NO: 122 - DNA, 20 bp, Homo sapiens
  source 1..20: mol_type = genomic DNA
aatcaccgatgtcattccct

SEQ ID NO: 123 - DNA, 20 bp, Homo sapiens
  source 1..20: mol_type = genomic DNA
agctcattgatggcttccga

SEQ ID NO: 124 - DNA, 20 bp, Homo sapiens
  source 1..20: mol_type = genomic DNA
gagctcattgatggcttccg

SEQ ID NO: 125 - DNA, 20 bp, Homo sapiens
  source 1..20: mol_type = genomic DNA
cagaacagtgagctcattga

SEQ ID NO: 126 - DNA, 20 bp, Homo sapiens
  source 1..20: mol_type = genomic DNA
catcaatgagctcactgttc

SEQ ID NO: 127 - DNA, 20 bp, Homo sapiens
  source 1..20: mol_type = genomic DNA
tgagctcactgttctggtgc

SEQ ID NO: 128 - DNA, 20 bp, Homo sapiens
  source 1..20: mol_type = genomic DNA
tctgagtaccttgaagtaaa

SEQ ID NO: 129 - DNA, 20 bp, Homo sapiens
  source 1..20: mol_type = genomic DNA
ggttaacaccatttacttca

SEQ ID NO: 130 - DNA, 20 bp, Homo sapiens
  source 1..20: mol_type = genomic DNA
actttgacttccacaggccc

SEQ ID NO: 131 - DNA, 20 bp, Homo sapiens
  source 1..20: mol_type = genomic DNA
tgattctcttccagggcctg

SEQ ID NO: 132 - DNA, 20 bp, Homo sapiens
  source 1..20: mol_type = genomic DNA
ggctgaactttgacttccac

SEQ ID NO: 133 - DNA, 20 bp, Homo sapiens
  source 1..20: mol_type = genomic DNA
gttccttccttgtgttctca

SEQ ID NO: 134 - DNA, 20 bp, Homo sapiens
  source 1..20: mol_type = genomic DNA
agttccttccttgtgttctc

SEQ ID NO: 135 - DNA, 20 bp, Homo sapiens
  source 1..20: mol_type = genomic DNA
agttcagccctgagaacaca

SEQ ID NO: 136 - DNA, 20 bp, Homo sapiens
  source 1..20: mol_type = genomic DNA
cagccctgagaacacaagga

SEQ ID NO: 137 - DNA, 20 bp, Homo sapiens
  source 1..20: mol_type = genomic DNA
aaggaaggaactgttctaca

SEQ ID NO: 138 - DNA, 20 bp, Homo sapiens
  source 1..20: mol_type = genomic DNA
gaactgttctacaaggctga

SEQ ID NO: 139 - DNA, 20 bp, Homo sapiens
  source 1..20: mol_type = genomic DNA
ttcagcatctatgatgtacc

SEQ ID NO: 140 - DNA, 20 bp, Homo sapiens
  source 1..20: mol_type = genomic DNA
gcatctatgatgtaccagga

SEQ ID NO: 141 - DNA, 20 bp, Homo sapiens
  source 1..20: mol_type = genomic DNA
gataacggaacttgccttcc

SEQ ID NO: 142 - DNA, 20 bp, Homo sapiens
  source 1..20: mol_type = genomic DNA
aggaaggcaagttccgttat

SEQ ID NO: 143 - DNA, 20 bp, Homo sapiens
  source 1..20: mol_type = genomic DNA
cttcagccacgcgccgataa

SEQ ID NO: 144 - DNA, 20 bp, Homo sapiens
  source 1..20: mol_type = genomic DNA
caagttccgttatcggcgcg

SEQ ID NO: 145 - DNA, 20 bp, Homo sapiens
  source 1..20: mol_type = genomic DNA
cgttatcggcgcgtggctga

SEQ ID NO: 146 - DNA, 20 bp, Homo sapiens
  source 1..20: mol_type = genomic DNA
gcgcgtggctgaaggcaccc

SEQ ID NO: 147 - DNA, 20 bp, Homo sapiens
  source 1..20: mol_type = genomic DNA
gaagggcaactcaagcacct

SEQ ID NO: 148 - DNA, 20 bp, Homo sapiens
  source 1..20: mol_type = genomic DNA
tgaagggcaactcaagcacc

SEQ ID NO: 149 - DNA, 20 bp, Homo sapiens
  source 1..20: mol_type = genomic DNA
gtgcttgagttgcccttcaa

SEQ ID NO: 150 - DNA, 20 bp, Homo sapiens
  source 1..20: mol_type = genomic DNA
gtgatgtcatcacctttgaa

SEQ ID NO: 151 - DNA, 20 bp, Homo sapiens
  source 1..20: mol_type = genomic DNA
ggtgatgtcatcacctttga

SEQ ID NO: 152 - DNA, 20 bp, Homo sapiens
  source 1..20: mol_type = genomic DNA
caaaggtgatgacatcacca

SEQ ID NO: 153 - DNA, 20 bp, Homo sapiens
  source 1..20: mol_type = genomic DNA
cttgggcaagatgaggacca

SEQ ID NO: 154 - DNA, 20 bp, Homo sapiens
  source 1..20: mol_type = genomic DNA
tctcaggcttgggcaagatg

SEQ ID NO: 155 - DNA, 20 bp, Homo sapiens
  source 1..20: mol_type = genomic DNA
gccaggctcttctcaggctt

SEQ ID NO: 156 - DNA, 20 bp, Homo sapiens
  source 1..20: mol_type = genomic DNA
ggccaggctcttctcaggct

SEQ ID NO: 157 - DNA, 20 bp, Homo sapiens
  source 1..20: mol_type = genomic DNA
accttggccaggctcttctc

SEQ ID NO: 158 - DNA, 20 bp, Homo sapiens
  source 1..20: mol_type = genomic DNA
gcccaagcctgagaagagcc

SEQ ID NO: 159 - DNA, 20 bp, Homo sapiens
  source 1..20: mol_type = genomic DNA
gcctgagaagagcctggcca

SEQ ID NO: 160 - DNA, 20 bp, Homo sapiens
  source 1..20: mol_type = genomic DNA
gttccttctctaccttggcc

SEQ ID NO: 161 - DNA, 20 bp, Homo sapiens
  source 1..20: mol_type = genomic DNA
ggtgagttccttctctacct

SEQ ID NO: 162 - DNA, 20 bp, Homo sapiens
  source 1..20: mol_type = genomic DNA
gagcctggccaaggtagaga

SEQ ID NO: 163 - DNA, 20 bp, Homo sapiens
  source 1..20: mol_type = genomic DNA
agagaaggaactcaccccag

SEQ ID NO: 164 - DNA, 20 bp, Homo sapiens
  source 1..20: mol_type = genomic DNA
ccactcttgcagcacctctg

SEQ ID NO: 165 - DNA, 20 bp, Homo sapiens
  source 1..20: mol_type = genomic DNA
gccactcttgcagcacctct

SEQ ID NO: 166 - DNA, 20 bp, Homo sapiens
  source 1..20: mol_type = genomic DNA
agccactcttgcagcacctc

SEQ ID NO: 167 - DNA, 20 bp, Homo sapiens
  source 1..20: mol_type = genomic DNA
ccccagaggtgctgcaagag

SEQ ID NO: 168 - DNA, 20 bp, Homo sapiens
  source 1..20: mol_type = genomic DNA
agaggtgctgcaagagtggc

SEQ ID NO: 169 - DNA, 20 bp, Homo sapiens
  source 1..20: mol_type = genomic DNA
gcaagagtggctggatgaat

SEQ ID NO: 170 - DNA, 20 bp, Homo sapiens
  source 1..20: mol_type = genomic DNA
agagtggctggatgaattgg

SEQ ID NO: 171 - DNA, 20 bp, Homo sapiens
  source 1..20: mol_type = genomic DNA
tgaattggaggagatgatgc

SEQ ID NO: 172 - DNA, 20 bp, Homo sapiens
  source 1..20: mol_type = genomic DNA
attggaggagatgatgctgg

SEQ ID NO: 173 - DNA, 20 bp, Homo sapiens
  source 1..20: mol_type = genomic DNA
caatgcggaagcggggcatg

SEQ ID NO: 174 - DNA, 20 bp, Homo sapiens
  source 1..20: mol_type = genomic DNA
ccgtcctcaatgcggaagcg

SEQ ID NO: 175 - DNA, 20 bp, Homo sapiens
  source 1..20: mol_type = genomic DNA
gccgtcctcaatgcggaagc

SEQ ID NO: 176 - DNA, 20 bp, Homo sapiens
  source 1..20: mol_type = genomic DNA
agccgtcctcaatgcggaag

SEQ ID NO: 177 - DNA, 20 bp, Homo sapiens
  source 1..20: mol_type = genomic DNA
catgccccgcttccgcattg

SEQ ID NO: 178 - DNA, 20 bp, Homo sapiens
  source 1..20: mol_type = genomic DNA
aactgaagccgtcctcaatg

SEQ ID NO: 179 - DNA, 20 bp, Homo sapiens
  source 1..20: mol_type = genomic DNA
ccccgcttccgcattgagga

SEQ ID NO: 180 - DNA, 20 bp, Homo sapiens
  source 1..20: mol_type = genomic DNA
tgaggacggcttcagtttga

SEQ ID NO: 181 - DNA, 20 bp, Homo sapiens
  source 1..20: mol_type = genomic DNA
gaaggagcagctgcaagaca

SEQ ID NO: 182 - DNA, 20 bp, Homo sapiens
  source 1..20: mol_type = genomic DNA
aaggagcagctgcaagacat

SEQ ID NO: 183 - DNA, 20 bp, Homo sapiens
  source 1..20: mol_type = genomic DNA
cagggctgaacagatcgaca

SEQ ID NO: 184 - DNA, 20 bp, Homo sapiens
  source 1..20: mol_type = genomic DNA
ctgggagtttggacttttca

SEQ ID NO: 185 - DNA, 20 bp, Homo sapiens
  source 1..20: mol_type = genomic DNA
cctgggagtttggacttttc

SEQ ID NO: 186 - DNA, 20 bp, Homo sapiens
  source 1..20: mol_type = genomic DNA
cctagacaaacctgggagtt

SEQ ID NO: 187 - DNA, 20 bp, Homo sapiens
  source 1..20: mol_type = genomic DNA
cctgaaaagtccaaactccc

SEQ ID NO: 188 - DNA, 20 bp, Homo sapiens
  source 1..20: mol_type = genomic DNA
cggccttctgcaacaatacc

SEQ ID NO: 189 - DNA, 20 bp, Homo sapiens
  source 1..20: mol_type = genomic DNA
cttccaggtattgttgcaga

SEQ ID NO: 190 - DNA, 20 bp, Homo sapiens
  source 1..20: mol_type = genomic DNA
ctgagacatagaggtcatct

SEQ ID NO: 191 - DNA, 20 bp, Homo sapiens
  source 1..20: mol_type = genomic DNA
ggaatgcatctgagacatag

SEQ ID NO: 192 - DNA, 20 bp, Homo sapiens
  source 1..20: mol_type = genomic DNA
tgtctcagatgcattccata

SEQ ID NO: 193 - DNA, 20 bp, Homo sapiens
  source 1..20: mol_type = genomic DNA
tcacctcaagaaatgcctta

SEQ ID NO: 194 - DNA, 20 bp, Homo sapiens
  source 1..20: mol_type = genomic DNA
attccataaggcatttcttg

SEQ ID NO: 195 - DNA, 20 bp, Homo sapiens
  source 1..20: mol_type = genomic DNA
gacctgcaggtaaatgaaga

SEQ ID NO: 196 - DNA, 20 bp, Homo sapiens
  source 1..20: mol_type = genomic DNA
acggccagcaatcacaacag

SEQ ID NO: 197 - DNA, 20 bp, Homo sapiens
  source 1..20: mol_type = genomic DNA
agtaccgctgttgtgattgc

SEQ ID NO: 198 - DNA, 20 bp, Homo sapiens
  source 1..20: mol_type = genomic DNA
ccctgttggggtttagcgaa

SEQ ID NO: 199 - DNA, 20 bp, Homo sapiens
  source 1..20: mol_type = genomic DNA
gccgttcgctaaaccccaac

SEQ ID NO: 200 - DNA, 20 bp, Homo sapiens
  source 1..20: mol_type = genomic DNA
ccgttcgctaaaccccaaca

SEQ ID NO: 201 - DNA, 20 bp, Homo sapiens
  source 1..20: mol_type = genomic DNA
ccttgaaagtcaccctgttg

SEQ ID NO: 202 - DNA, 20 bp, Homo sapiens
  source 1..20: mol_type = genomic DNA
gccttgaaagtcaccctgtt

SEQ ID NO: 203 - DNA, 20 bp, Homo sapiens
  source 1..20: mol_type = genomic DNA
ggccttgaaagtcaccctgt

SEQ ID NO: 204 - DNA, 20 bp, Homo sapiens
  source 1..20: mol_type = genomic DNA
ccccaacagggtgactttca

SEQ ID NO: 205 - DNA, 20 bp, Homo sapiens
  source 1..20: mol_type = genomic DNA
gggtgactttcaaggccaac

SEQ ID NO: 206 - DNA, 20 bp, Homo sapiens
  source 1..20: mol_type = genomic DNA
aaaaaccaggaaaggcctgt

SEQ ID NO: 207 - DNA, 20 bp, Homo sapiens
  source 1..20: mol_type = genomic DNA
caaggccaacaggcctttcc

SEQ ID NO: 208 - DNA, 20 bp, Homo sapiens
  source 1..20: mol_type = genomic DNA
tctcttataaaaaccaggaa

SEQ ID NO: 209 - DNA, 20 bp, Homo sapiens
  source 1..20: mol_type = genomic DNA
gaacttctcttataaaaacc

SEQ ID NO: 210 - DNA, 20 bp, Homo sapiens
  source 1..20: mol_type = genomic DNA
atgaagataatagtgttcag

SEQ ID NO: 211 - DNA, 20 bp, Homo sapiens
  source 1..20: mol_type = genomic DNA
tctgaacactattatcttca

SEQ ID NO: 212 - DNA, 20 bp, Homo sapiens
  source 1..20: mol_type = genomic DNA
ctgaacactattatcttcat

SEQ ID NO: 213 - DNA, 20 bp, Homo sapiens
  source 1..20: mol_type = genomic DNA
attttacttaacacaagggt

SEQ ID NO: 214 - DNA, 20 bp, Homo sapiens
  source 1..20: mol_type = genomic DNA
gaacattttacttaacacaa

SEQ ID NO: 215 - DNA, 20 bp, Homo sapiens
  source 1..20: mol_type = genomic DNA
agaacattttacttaacaca

SEQ ID NO: 216 - DNA, 20 bp, Homo sapiens
  source 1..20: mol_type = genomic DNA
ggttacagttcctatcacat

SEQ ID NO: 217 - DNA, 20 bp, Homo sapiens
  source 1..20: mol_type = genomic DNA
ccaagccgcgggacattccc

SEQ ID NO: 218 - DNA, 20 bp, Homo sapiens
  source 1..20: mol_type = genomic DNA
taaatgcacatgggattcat

SEQ ID NO: 219 - DNA, 20 bp, Homo sapiens
  source 1..20: mol_type = genomic DNA
cggggagcggtaaatgcaca

SEQ ID NO: 220 - DNA, 20 bp, Homo sapiens
  source 1..20: mol_type = genomic DNA
ccccggagaagaaggcaact

SEQ ID NO: 221 - DNA, 20 bp, Homo sapiens
  source 1..20: mol_type = genomic DNA
acacgccggttggtggcctc

SEQ ID NO: 222 - DNA, 20 bp, Homo sapiens
  source 1..20: mol_type = genomic DNA
atagaaagtggtagcaaagc

SEQ ID NO: 223 - DNA, 20 bp, Homo sapiens
  source 1..20: mol_type = genomic DNA
atcagcacctggcagattcc

SEQ ID NO: 224 - DNA, 20 bp, Homo sapiens
  source 1..20: mol_type = genomic DNA
aatgttatcattgtcattct

SEQ ID NO: 225 - DNA, 20 bp, Homo sapiens
  source 1..20: mol_type = genomic DNA
ataacattttcctgtcaccc

SEQ ID NO: 226 - DNA, 20 bp, Homo sapiens
  source 1..20: mol_type = genomic DNA
caaaagccgtggagatactc

SEQ ID NO: 227 - DNA, 20 bp, Homo sapiens
  source 1..20: mol_type = genomic DNA
cggcttttgctatgaccaag

SEQ ID NO: 228 - DNA, 20 bp, Homo sapiens
  source 1..20: mol_type = genomic DNA
cgtacctccatcagttgctg

SEQ ID NO: 229 - DNA, 20 bp, Homo sapiens
  source 1..20: mol_type = genomic DNA
ttcagtttggcaaagaagaa

SEQ ID NO: 230 - DNA, 20 bp, Homo sapiens
  source 1..20: mol_type = genomic DNA
aggatttgttggcttttcga

SEQ ID NO: 231 - DNA, 20 bp, Homo sapiens
  source 1..20: mol_type = genomic DNA
gattggctgatactaacttg

SEQ ID NO: 232 - DNA, 20 bp, Homo sapiens
  source 1..20: mol_type = genomic DNA
ggtaggtctcattgaaggta

SEQ ID NO: 233 - DNA, 20 bp, Homo sapiens
  source 1..20: mol_type = genomic DNA
tgagacctaccaggacatca

SEQ ID NO: 234 - DNA, 20 bp, Homo sapiens
  source 1..20: mol_type = genomic DNA
tccagcccctggacttcaag

SEQ ID NO: 235 - DNA, 20 bp, Homo sapiens
  source 1..20: mol_type = genomic DNA
cccatttgttgatggccgct

SEQ ID NO: 236 - DNA, 20 bp, Homo sapiens
  source 1..20: mol_type = genomic DNA
tccagagcggccatcaacaa

SEQ ID NO: 237 - DNA, 20 bp, Homo sapiens
  source 1..20: mol_type = genomic DNA
gtgtccaataagaccgaagg

SEQ ID NO: 238 - DNA, 20 bp, Homo sapiens
  source 1..20: mol_type = genomic DNA
agctcattgatggcttccga

SEQ ID NO: 239 - DNA, 20 bp, Homo sapiens
  source 1..20: mol_type = genomic DNA
actgttctacaaggctgatg

SEQ ID NO: 240 - DNA, 20 bp, Homo sapiens
  source 1..20: mol_type = genomic DNA
ggctgaaggcacccaggtgc

SEQ ID NO: 241 - DNA, 20 bp, Homo sapiens
  source 1..20: mol_type = genomic DNA
ttgaagggcaactcaagcac

SEQ ID NO: 242 - DNA, 20 bp, Homo sapiens
  source 1..20: mol_type = genomic DNA
actcttgcagcacctctggg

SEQ ID NO: 243 - DNA, 20 bp, Homo sapiens
  source 1..20: mol_type = genomic DNA
agccactcttgcagcacctc

SEQ ID NO: 244 - DNA, 20 bp, Homo sapiens
  source 1..20: mol_type = genomic DNA
actcaccccagaggtgctgc

SEQ ID NO: 245 - DNA, 20 bp, Homo sapiens
  source 1..20: mol_type = genomic DNA
cagaggtgctgcaagagtgg

SEQ ID NO: 246 - DNA, 20 bp, Homo sapiens
  source 1..20: mol_type = genomic DNA
ggtgctgcaagagtggctgg

SEQ ID NO: 247 - DNA, 20 bp, Homo sapiens
  source 1..20: mol_type = genomic DNA
tcacctcaagaaatgcctta

SEQ ID NO: 248 - DNA, 20 bp, Homo sapiens
  source 1..20: mol_type = genomic DNA
tccataaggcatttcttgag

SEQ ID NO: 249 - DNA, 20 bp, Homo sapiens
  source 1..20: mol_type = genomic DNA
ggccgttcgctaaaccccaa

SEQ ID NO: 250 - DNA, 20 bp, Homo sapiens
  source 1..20: mol_type = genomic DNA
ggccttgaaagtcaccctgt

SEQ ID NO: 251 - DNA, 20 bp, Homo sapiens
  source 1..20: mol_type = genomic DNA
aacactattatcttcatggg

SEQ ID NO: 252 - DNA, 20 bp, Homo sapiens
  source 1..20: mol_type = genomic DNA
aagaacattttacttaacac

SEQ ID NO: 253 - DNA, 22 bp, Homo sapiens
  source 1..22: mol_type = genomic DNA
gaggttacagttcctatcacat

SEQ ID NO: 254 - DNA, 22 bp, Homo sapiens
  source 1..22: mol_type = genomic DNA
ctgtcacgggagccctgtggac

SEQ ID NO: 255 - DNA, 22 bp, Homo sapiens
  source 1..22: mol_type = genomic DNA
gccttcttctccggggagcggt

SEQ ID NO: 256 - DNA, 22 bp, Homo sapiens
  source 1..22: mol_type = genomic DNA
gggaattggccttggacagttc

SEQ ID NO: 257 - DNA, 22 bp, Homo sapiens
  source 1..22: mol_type = genomic DNA
tcccgctttgctaccactttct

SEQ ID NO: 258 - DNA, 22 bp, Homo sapiens
  source 1..22: mol_type = genomic DNA
gcttggtcatagcaaaagccgt

SEQ ID NO: 259 - DNA, 22 bp, Homo sapiens
  source 1..22: mol_type = genomic DNA
tatgaccaagctgggtgcctgt

SEQ ID NO: 260 - DNA, 22 bp, Homo sapiens
  source 1..22: mol_type = genomic DNA
tcagttgctggagggtgtcatt

SEQ ID NO: 261 - DNA, 22 bp, Homo sapiens
  source 1..22: mol_type = genomic DNA
atgacaccctccagcaactgat

SEQ ID NO: 262 - DNA, 22 bp, Homo sapiens
  source 1..22: mol_type = genomic DNA
ttgacttctataggtatttaag

SEQ ID NO: 263 - DNA, 22 bp, Homo sapiens
  source 1..22: mol_type = genomic DNA
tttctcagatatggtgtcaaac

SEQ ID NO: 264 - DNA, 22 bp, Homo sapiens
  source 1..22: mol_type = genomic DNA
tttgtctccaaaaaggcgattg

SEQ ID NO: 265 - DNA, 22 bp, Homo sapiens
  source 1..22: mol_type = genomic DNA
gtccaggggctggagcttggct

SEQ ID NO: 266 - DNA, 22 bp, Homo sapiens
  source 1..22: mol_type = genomic DNA
ggtgattcggccttcggtctta

SEQ ID NO: 267 - DNA, 22 bp, Homo sapiens
  source 1..22: mol_type = genomic DNA
tgagctcactgttctggtgctg

SEQ ID NO: 268 - DNA, 22 bp, Homo sapiens
  source 1..22: mol_type = genomic DNA
taccttgaagtaaatggtgtta

SEQ ID NO: 269 - DNA, 22 bp, Homo sapiens
  source 1..22: mol_type = genomic DNA
tgctggttaacaccatttactt

SEQ ID NO: 270 - DNA, 22 bp, Homo sapiens
  source 1..22: mol_type = genomic DNA
gtggaagtcaaagttcagccct

SEQ ID NO: 271 - DNA, 22 bp, Homo sapiens
  source 1..22: mol_type = genomic DNA
ggagagtcgtgttcagcatcta

SEQ ID NO: 272 - DNA, 22 bp, Homo sapiens
  source 1..22: mol_type = genomic DNA
ccttcctggtacatcatagatg

SEQ ID NO: 273 - DNA, 22 bp, Homo sapiens
  source 1..22: mol_type = genomic DNA
cgccgataacggaacttgcctt

SEQ ID NO: 274 - DNA, 22 bp, Homo sapiens
  source 1..22: mol_type = genomic DNA
gttccgttatcggcgcgtggct

SEQ ID NO: 275 - DNA, 22 bp, Homo sapiens
  source 1..22: mol_type = genomic DNA
gtcatcacctttgaagggcaac

SEQ ID NO: 276 - DNA, 22 bp, Homo sapiens
  source 1..22: mol_type = genomic DNA
ctccaattcatccagccactct

SEQ ID NO: 277 - DNA, 22 bp, Homo sapiens
  source 1..22: mol_type = genomic DNA
agaggtcatctcggccttctgc

SEQ ID NO: 278 - DNA, 22 bp, Homo sapiens
  source 1..22: mol_type = genomic DNA
attccataaggcatttcttgag

SEQ ID NO: 279 - DNA, 22 bp, Homo sapiens
  source 1..22: mol_type = genomic DNA
tgaagaaggcagtgaagcagct

SEQ ID NO: 280 - DNA, 22 bp, Homo sapiens
  source 1..22: mol_type = genomic DNA
gcgaacggccagcaatcacaac

SEQ ID NO: 281 - DNA, 22 bp, Homo sapiens
  source 1..22: mol_type = genomic DNA
ggtttttataagagaagttcct

SEQ ID NO: 282 - DNA, 22 bp, Homo sapiens
  source 1..22: mol_type = genomic DNA
tgcaaagaataagaacatttta

SEQ ID NO: 283 - DNA, 20 bp, Homo sapiens
  source 1..20: mol_type = genomic DNA
tgaagaaagtacatgcactt

SEQ ID NO: 284 - DNA, 20 bp, Homo sapiens
  source 1..20: mol_type = genomic DNA
gaagaaagtacatgcacttt

SEQ ID NO: 285 - DNA, 20 bp, Homo sapiens
  source 1..20: mol_type = genomic DNA
caggggcaagattaagcagc

SEQ ID NO: 286 - DNA, 20 bp, Homo sapiens
  source 1..20: mol_type = genomic DNA
atcagcattaagaggggcag

SEQ ID NO: 287 - DNA, 20 bp, Homo sapiens
  source 1..20: mol_type = genomic DNA
aatcagcattaagaggggca

SEQ ID NO: 288 - DNA, 20 bp, Homo sapiens
  source 1..20: mol_type = genomic DNA
gaatcagcattaagaggggc

SEQ ID NO: 289 - DNA, 20 bp, Homo sapiens
  source 1..20: mol_type = genomic DNA
ctcagaatcagcattaagag

SEQ ID NO: 290 - DNA, 20 bp, Homo sapiens
  source 1..20: mol_type = genomic DNA
cctcagaatcagcattaaga

SEQ ID NO: 291 - DNA, 20 bp, Homo sapiens
  source 1..20: mol_type = genomic DNA
tcctcagaatcagcattaag

SEQ ID NO: 292 - DNA, 20 bp, Homo sapiens
  source 1..20: mol_type = genomic DNA
ccctcttaatgctgattctg

SEQ ID NO: 293 - DNA, 20 bp, Homo sapiens
  source 1..20: mol_type = genomic DNA
gaagaacacacaattatcac

SEQ ID NO: 294 - DNA, 20 bp, Homo sapiens
  source 1..20: mol_type = genomic DNA
ttatttttactttatagata

SEQ ID NO: 295 - DNA, 20 bp, Homo sapiens
  source 1..20: mol_type = genomic DNA
gaatgcataagtttcagtgg

SEQ ID NO: 296 - DNA, 20 bp, Homo sapiens
  source 1..20: mol_type = genomic DNA
aatgaatgcataagtttcag

SEQ ID NO: 297 - DNA, 20 bp, Homo sapiens
  source 1..20: mol_type = genomic DNA
gcattcattttgtgcattca

SEQ ID NO: 298 - DNA, 20 bp, Homo sapiens
  source 1..20: mol_type = genomic DNA
ttcattttgtgcattcaagg

SEQ ID NO: 299 - DNA, 20 bp, Homo sapiens
  source 1..20: mol_type = genomic DNA
tgtgcattcaaggcggatga

SEQ ID NO: 300 - DNA, 20 bp, Homo sapiens
  source 1..20: mol_type = genomic DNA
ttttcatgattgctttacat

SEQ ID NO: 301 - DNA, 20 bp, Homo sapiens
  source 1..20: mol_type = genomic DNA
cttttcatgattgctttaca

SEQ ID NO: 302 - DNA, 20 bp, Homo sapiens
  source 1..20: mol_type = genomic DNA
cagtgcgaagaatttatata

SEQ ID NO: 303 - DNA, 20 bp, Homo sapiens
  source 1..20: mol_type = genomic DNA
agtgcgaagaatttatatat

SEQ ID NO: 304 - DNA, 20 bp, Homo sapiens
  source 1..20: mol_type = genomic DNA
gtgcgaagaatttatatatg

SEQ ID NO: 305 - DNA, 20 bp, Homo sapiens
  source 1..20: mol_type = genomic DNA
tgcgaagaatttatatatgg

SEQ ID NO: 306 - DNA, 20 bp, Homo sapiens
  source 1..20: mol_type = genomic DNA
tttatatatgggggatgtga

SEQ ID NO: 307 - DNA, 20 bp, Homo sapiens
  source 1..20: mol_type = genomic DNA
tcagaatcgatttgaaagtc

SEQ ID NO: 308 - DNA, 20 bp, Homo sapiens
  source 1..20: mol_type = genomic DNA
tgcaaaaaaatgtgtacaag

SEQ ID NO: 309 - DNA, 20 bp, Homo sapiens
  source 1..20: mol_type = genomic DNA
aaaaaatgtgtacaagaggt

SEQ ID NO: 310 - DNA, 20 bp, Homo sapiens
  source 1..20: mol_type = genomic DNA
tgattacagataatgcaaac

SEQ ID NO: 311 - DNA, 20 bp, Homo sapiens
  source 1..20: mol_type = genomic DNA
ataaagacaacattgcaaca

SEQ ID NO: 312 - DNA, 20 bp, Homo sapiens
  source 1..20: mol_type = genomic DNA
tcttccaaaaagcagaaatc

SEQ ID NO: 313 - DNA, 20 bp, Homo sapiens
  source 1..20: mol_type = genomic DNA
aaagccagatttctgctttt

SEQ ID NO: 314 - DNA, 20 bp, Homo sapiens
  source 1..20: mol_type = genomic DNA
tgctttttggaagaagatcc

SEQ ID NO: 315 - DNA, 20 bp, Homo sapiens
  source 1..20: mol_type = genomic DNA
atataacctcgacatattcc

SEQ ID NO: 316 - DNA, 20 bp, Homo sapiens
  source 1..20: mol_type = genomic DNA
gaagatcctggaatatgtcg

SEQ ID NO: 317 - DNA, 20 bp, Homo sapiens
  source 1..20: mol_type = genomic DNA
tatgtcgaggttatattacc

SEQ ID NO: 318 - DNA, 20 bp, Homo sapiens
  source 1..20: mol_type = genomic DNA
ctgattgttataaaaatacc

SEQ ID NO: 319 - DNA, 20 bp, Homo sapiens
  source 1..20: mol_type = genomic DNA
cagtgtgaacgtttcaagta

SEQ ID NO: 320 - DNA, 20 bp, Homo sapiens
  source 1..20: mol_type = genomic DNA
tgtgaacgtttcaagtatgg

SEQ ID NO: 321 - DNA, 20 bp, Homo sapiens
  source 1..20: mol_type = genomic DNA
tttcaagtatggtggatgcc

SEQ ID NO: 322 - DNA, 20 bp, Homo sapiens
  source 1..20: mol_type = genomic DNA
ttcaagtatggtggatgcct

SEQ ID NO: 323 - DNA, 20 bp, Homo sapiens
  source 1..20: mol_type = genomic DNA
caaaattgttcatattgccc

SEQ ID NO: 324 - DNA, 20 bp, Homo sapiens
  source 1..20: mol_type = genomic DNA
tatgaacaattttgagacac

SEQ ID NO: 325 - DNA, 20 bp, Homo sapiens
  source 1..20: mol_type = genomic DNA
tgcaagaacatttgtgaaga

SEQ ID NO: 326 - DNA, 20 bp, Homo sapiens
  source 1..20: mol_type = genomic DNA
ctgtatttttttccagcgaa

SEQ ID NO: 327 - DNA, 20 bp, Homo sapiens
  source 1..20: mol_type = genomic DNA
tccacctggaaaccattcgc

SEQ ID NO: 328 - DNA, 20 bp, Homo sapiens
  source 1..20: mol_type = genomic DNA
ttttccagcgaatggtttcc

SEQ ID NO: 329 - DNA, 20 bp, Homo sapiens
  source 1..20: mol_type = genomic DNA
tccagcgaatggtttccagg

SEQ ID NO: 330 - DNA, 20 bp, Homo sapiens
  source 1..20: mol_type = genomic DNA
gggttccataattatccacc

SEQ ID NO: 331 - DNA, 20 bp, Homo sapiens
  source 1..20: mol_type = genomic DNA
ggtttccaggtggataatta

SEQ ID NO: 332 - DNA, 20 bp, Homo sapiens
  source 1..20: mol_type = genomic DNA
gttattcacagcattgagct

SEQ ID NO: 333 - DNA, 20 bp, Homo sapiens
  source 1..20: mol_type = genomic DNA
agttattcacagcattgagc

SEQ ID NO: 334 - DNA, 20 bp, Homo sapiens
  source 1..20: mol_type = genomic DNA
cttggttgattgcggagtca

SEQ ID NO: 335 - DNA, 20 bp, Homo sapiens
  source 1..20: mol_type = genomic DNA
ccttggttgattgcggagtc

SEQ ID NO: 336 - DNA, 20 bp, Homo sapiens
  source 1..20: mol_type = genomic DNA
ctgggaaccttggttgattg

SEQ ID NO: 337 - DNA, 20 bp, Homo sapiens
  source 1..20: mol_type = genomic DNA
cctgactccgcaatcaacca

SEQ ID NO: 338 - DNA, 20 bp, Homo sapiens
  source 1..20: mol_type = genomic DNA
accaaaaaggctgggaacct

SEQ ID NO: 339 - DNA, 20 bp, Homo sapiens
  source 1..20: mol_type = genomic DNA
agattcttaccaaaaaggct

SEQ ID NO: 340 - DNA, 20 bp, Homo sapiens
  source 1..20: mol_type = genomic DNA
aagattcttaccaaaaaggc

SEQ ID NO: 341 - DNA, 20 bp, Homo sapiens
  source 1..20: mol_type = genomic DNA
accaaggttcccagcctttt

SEQ ID NO: 342 - DNA, 20 bp, Homo sapiens
  source 1..20: mol_type = genomic DNA
ccacaagattcttaccaaaa

SEQ ID NO: 343 - DNA, 20 bp, Homo sapiens
  source 1..20: mol_type = genomic DNA
cctttttggtaagaatcttg

SEQ ID NO: 344 - DNA, 20 bp, Homo sapiens
  source 1..20: mol_type = genomic DNA
gtgaaattctaaaaacaatc

SEQ ID NO: 345 - DNA, 20 bp, Homo sapiens
  source 1..20: mol_type = genomic DNA
tgattgtttttagaatttca

SEQ ID NO: 346 - DNA, 20 bp, Homo sapiens
  source 1..20: mol_type = genomic DNA
tagaatttcacggtccctca

SEQ ID NO: 347 - DNA, 20 bp, Homo sapiens
  source 1..20: mol_type = genomic DNA
gctggagtgagacaccatga

SEQ ID NO: 348 - DNA, 20 bp, Homo sapiens
  source 1..20: mol_type = genomic DNA
tgctggagtgagacaccatg

SEQ ID NO: 349 - DNA, 20 bp, Homo sapiens
  source 1..20: mol_type = genomic DNA
cgacacaatcctctgtctgc

SEQ ID NO: 350 - DNA, 20 bp, Homo sapiens
  source 1..20: mol_type = genomic DNA
tgtctcactccagcagacag

SEQ ID NO: 351 - DNA, 20 bp, Homo sapiens
  source 1..20: mol_type = genomic DNA
gtagtagaatctgttctcat

SEQ ID NO: 352 - DNA, 20 bp, Homo sapiens
  source 1..20: mol_type = genomic DNA
ttctactacaattcagtcat

SEQ ID NO: 353 - DNA, 20 bp, Homo sapiens
  source 1..20: mol_type = genomic DNA
tctactacaattcagtcatt

SEQ ID NO: 354 - DNA, 20 bp, Homo sapiens
  source 1..20: mol_type = genomic DNA
atccactgtacttaaatggg

SEQ ID NO: 355 - DNA, 20 bp, Homo sapiens
  source 1..20: mol_type = genomic DNA
cacatccactgtacttaaat

SEQ ID NO: 356 - DNA, 20 bp, Homo sapiens
  source 1..20: mol_type = genomic DNA
ccacatccactgtacttaaa

SEQ ID NO: 357 - DNA, 20 bp, Homo sapiens
  source 1..20: mol_type = genomic DNA
tgccgcccatttaagtacag

SEQ ID NO: 358 - DNA, 20 bp, Homo sapiens
  source 1..20: mol_type = genomic DNA
ccatttaagtacagtggatg

SEQ ID NO: 359 - DNA, 20 bp, Homo sapiens
  source 1..20: mol_type = genomic DNA
catttaagtacagtggatgt

SEQ ID NO: 360 - DNA, 20 bp, Homo sapiens
  source 1..20: mol_type = genomic DNA
atttaagtacagtggatgtg

SEQ ID NO: 361 - DNA, 20 bp, Homo sapiens
  source 1..20: mol_type = genomic DNA
tttaagtacagtggatgtgg

SEQ ID NO: 362 - DNA, 20 bp, Homo sapiens
  source 1..20: mol_type = genomic DNA
tgccctcagacattcttgtt

SEQ ID NO: 363 - DNA, 20 bp, Homo sapiens
  source 1..20: mol_type = genomic DNA
cttccaaacaagaatgtctg

SEQ ID NO: 364 - DNA, 20 bp, Homo sapiens
  source 1..20: mol_type = genomic DNA
ttccaaacaagaatgtctga

SEQ ID NO: 365 - DNA, 20 bp, Homo sapiens
  source 1..20: mol_type = genomic DNA
tgtctgagggcatgtaaaaa

SEQ ID NO: 366 - DNA, 20 bp, Homo sapiens
  source 1..20: mol_type = genomic DNA
atgaaacctataagaggaag

SEQ ID NO: 367 - DNA, 20 bp, Homo sapiens
  source 1..20: mol_type = genomic DNA
ctttggatgaaacctataag

SEQ ID NO: 368 - DNA, 20 bp, Homo sapiens
  source 1..20: mol_type = genomic DNA
ggcctccttttgatattctt

SEQ ID NO: 369 - DNA, 20 bp, Homo sapiens
  source 1..20: mol_type = genomic DNA
ttcatccaaagaatatcaaa

SEQ ID NO: 370 - DNA, 20 bp, Homo sapiens
  source 1..20: mol_type = genomic DNA
atccaaagaatatcaaaagg

SEQ ID NO: 371 - DNA, 20 bp, Homo sapiens
  source 1..20: mol_type = genomic DNA
tttttcttttggttttaatt

SEQ ID NO: 372 - DNA, 20 bp, Homo sapiens
  source 1..20: mol_type = genomic DNA
ctgcttctttctttttcttt

SEQ ID NO: 373 - DNA, 20 bp, Homo sapiens
  source 1..20: mol_type = genomic DNA
tgtgtgttcttcatcttcct

SEQ ID NO: 374 - DNA, 20 bp, Homo sapiens
  source 1..20: mol_type = genomic DNA
ttatttttactttatagata

SEQ ID NO: 375 - DNA, 20 bp, Homo sapiens
  source 1..20: mol_type = genomic DNA
atccgccttgaatgcacaaa

SEQ ID NO: 376 - DNA, 20 bp, Homo sapiens
  source 1..20: mol_type = genomic DNA
attcattttgtgcattcaag

SEQ ID NO: 377 - DNA, 20 bp, Homo sapiens
  source 1..20: mol_type = genomic DNA
tacatgggccatcatccgcc

SEQ ID NO: 378 - DNA, 20 bp, Homo sapiens
  source 1..20: mol_type = genomic DNA
tatataaattcttcgcactg

SEQ ID NO: 379 - DNA, 20 bp, Homo sapiens
  source 1..20: mol_type = genomic DNA
tattttcactcgacagtgcg

SEQ ID NO: 380 - DNA, 20 bp, Homo sapiens
  source 1..20: mol_type = genomic DNA
gtgcgaagaatttatatatg

SEQ ID NO: 381 - DNA, 20 bp, Homo sapiens
  source 1..20: mol_type = genomic DNA
atgggggatgtgaaggaaat

SEQ ID NO: 382 - DNA, 20 bp, Homo sapiens
  source 1..20: mol_type = genomic DNA
gaatcgatttgaaagtctgg

SEQ ID NO: 383 - DNA, 20 bp, Homo sapiens
  source 1..20: mol_type = genomic DNA
ttgattacagataatgcaaa

SEQ ID NO: 384 - DNA, 20 bp, Homo sapiens
  source 1..20: mol_type = genomic DNA
tgctttttggaagaagatcc

SEQ ID NO: 385 - DNA, 20 bp, Homo sapiens
  source 1..20: mol_type = genomic DNA
aatataacctcgacatattc

SEQ ID NO: 386 - DNA, 20 bp, Homo sapiens
  source 1..20: mol_type = genomic DNA
gtgtgaacgtttcaagtatg

SEQ ID NO: 387 - DNA, 20 bp, Homo sapiens
  source 1..20: mol_type = genomic DNA
gaacaattttgagacactgg

SEQ ID NO: 388 - DNA, 20 bp, Homo sapiens
  source 1..20: mol_type = genomic DNA
ttccagcgaatggtttccag

SEQ ID NO: 389 - DNA, 20 bp, Homo sapiens
  source 1..20: mol_type = genomic DNA
gagttattcacagcattgag

SEQ ID NO: 390 - DNA, 20 bp, Homo sapiens
  source 1..20: mol_type = genomic DNA
atggaacccagctcaatgct

SEQ ID NO: 391 - DNA, 20 bp, Homo sapiens
  source 1..20: mol_type = genomic DNA
cttggttgattgcggagtca

SEQ ID NO: 392 - DNA, 20 bp, Homo sapiens
  source 1..20: mol_type = genomic DNA
ctgggaaccttggttgattg

SEQ ID NO: 393 - DNA, 20 bp, Homo sapiens
  source 1..20: mol_type = genomic DNA
aggttcccagcctttttggt

SEQ ID NO: 394 - DNA, 20 bp, Homo sapiens
  source 1..20: mol_type = genomic DNA
cgacacaatcctctgtctgc

SEQ ID NO: 395 - DNA, 20 bp, Homo sapiens
  source 1..20: mol_type = genomic DNA
gtgtctcactccagcagaca

SEQ ID NO: 396 - DNA, 20 bp, Homo sapiens
  source 1..20: mol_type = genomic DNA
tcccaatgactgaattgtag

SEQ ID NO: 397 - DNA, 20 bp, Homo sapiens
  source 1..20: mol_type = genomic DNA
tgggcggcatttcccaatga

SEQ ID NO: 398 - DNA, 20 bp, Homo sapiens
  source 1..20: mol_type = genomic DNA
atgccgcccatttaagtaca

SEQ ID NO: 399 - DNA, 20 bp, Homo sapiens
  source 1..20: mol_type = genomic DNA
aaacaattttacttccaaac

SEQ ID NO: 400 - DNA, 20 bp, Homo sapiens
  source 1..20: mol_type = genomic DNA
cctcttataggtttcatcca

SEQ ID NO: 401 - DNA, 20 bp, Homo sapiens
  source 1..20: mol_type = genomic DNA
aggcctccttttgatattct

SEQ ID NO: 402 - DNA, 20 bp, Homo sapiens
  source 1..20: mol_type = genomic DNA
gaaatttttgttaaaaatat

SEQ ID NO: 403 - DNA, 22 bp, Homo sapiens
  source 1..22: mol_type = genomic DNA
tgggttctgtatttcagagatg

SEQ ID NO: 404 - DNA, 22 bp, Homo sapiens
  source 1..22: mol_type = genomic DNA
tcttcattgtgtaaatcatctc

SEQ ID NO: 405 - DNA, 22 bp, Homo sapiens
  source 1..22: mol_type = genomic DNA
cagagatgatttacacaatgaa

SEQ ID NO: 406 - DNA, 22 bp, Homo sapiens
  source 1..22: mol_type = genomic DNA
tgatttacacaatgaagaaagt

SEQ ID NO: 407 - DNA, 22 bp, Homo sapiens
  source 1..22: mol_type = genomic DNA
caggcatacagaagcccaaagt

SEQ ID NO: 408 - DNA, 22 bp, Homo sapiens
  source 1..22: mol_type = genomic DNA
ggcaggggcaagattaagcagc

SEQ ID NO: 409 - DNA, 22 bp, Homo sapiens
  source 1..22: mol_type = genomic DNA
aatgctgattctgaggaagatg

SEQ ID NO: 410 - DNA, 22 bp, Homo sapiens
  source 1..22: mol_type = genomic DNA
tgctgattctgaggaagatgaa

SEQ ID NO: 411 - DNA, 22 bp, Homo sapiens
  source 1..22: mol_type = genomic DNA
tacatgggccatcatccgcctt

SEQ ID NO: 412 - DNA, 22 bp, Homo sapiens
  source 1..22: mol_type = genomic DNA
tcacatcccccatatataaatt

SEQ ID NO: 413 - DNA, 22 bp, Homo sapiens
  source 1..22: mol_type = genomic DNA
aagtctggaagagtgcaaaaaa

SEQ ID NO: 414 - DNA, 22 bp, Homo sapiens
  source 1..22: mol_type = genomic DNA
aacctacctcttgtacacattt

SEQ ID NO: 415 - DNA, 22 bp, Homo sapiens
  source 1..22: mol_type = genomic DNA
agggttcccagaaacctacctc

SEQ ID NO: 416 - DNA, 22 bp, Homo sapiens
  source 1..22: mol_type = genomic DNA
cactgttttgtctgattgttat

SEQ ID NO: 417 - DNA, 22 bp, Homo sapiens
  source 1..22: mol_type = genomic DNA
aggcatccaccatacttgaaac

SEQ ID NO: 418 - DNA, 22 bp, Homo sapiens
  source 1..22: mol_type = genomic DNA
ttgttcatattgcccaggcatc

SEQ ID NO: 419 - DNA, 22 bp, Homo sapiens
  source 1..22: mol_type = genomic DNA
gcctgggcaatatgaacaattt

SEQ ID NO: 420 - DNA, 22 bp, Homo sapiens
  source 1..22: mol_type = genomic DNA
cacaatcctctgtctgctggag

SEQ ID NO: 421 - DNA, 22 bp, Homo sapiens
  source 1..22: mol_type = genomic DNA
tagtagaatctgttctcattgg

SEQ ID NO: 422 - DNA, 22 bp, Homo sapiens
  source 1..22: mol_type = genomic DNA
aattgtagtagaatctgttctc

SEQ ID NO: 423 - DNA, 22 bp, Homo sapiens
  source 1..22: mol_type = genomic DNA
gtcattgggaaatgccgcccat

SEQ ID NO: 424 - DNA, 22 bp, Homo sapiens
  source 1..22: mol_type = genomic DNA
ttgttttcatttcccccacatc

SEQ ID NO: 425 - RNA, 20 nt, synthetic construct
  misc_feature 1..20: note = Guide sequence
  source 1..20: mol_type = other RNA
tcctatcacattggaataca

SEQ ID NO: 426 - RNA, 20 nt, synthetic construct
  misc_feature 1..20: note = Guide sequence
  source 1..20: mol_type = other RNA
ggttacagttcctatcacat

SEQ ID NO: 427 - RNA, 20 nt, synthetic construct
  misc_feature 1..20: note = Guide sequence
  source 1..20: mol_type = other RNA
gccatgtattccaatgtgat

SEQ ID NO: 428 - RNA, 20 nt, synthetic construct
  misc_feature 1..20: note = Guide sequence
  source 1..20: mol_type = other RNA
gtgataggaactgtaacctc

SEQ ID NO: 429 - RNA, 20 nt, synthetic construct
  misc_feature 1..20: note = Guide sequence
  source 1..20: mol_type = other RNA
cccctcttacctttttccag

SEQ ID NO: 430 - RNA, 20 nt, synthetic construct
  misc_feature 1..20: note = Guide sequence
  source 1..20: mol_type = other RNA
gaactgtaacctctggaaaa

SEQ ID NO: 431 - RNA, 20 nt, synthetic construct
  misc_feature 1..20: note = Guide sequence
  source 1..20: mol_type = other RNA
ccagaagccaatgagcagca

SEQ ID NO: 432 - RNA, 20 nt, synthetic construct
  misc_feature 1..20: note = Guide sequence
  source 1..20: mol_type = other RNA
cttttgtccttgctgctcat

SEQ ID NO: 433 - RNA, 20 nt, synthetic construct
  misc_feature 1..20: note = Guide sequence
  source 1..20: mol_type = other RNA
ccttgctgctcattggcttc

SEQ ID NO: 434 - RNA, 20 nt, synthetic construct
  misc_feature 1..20: note = Guide sequence
  source 1..20: mol_type = other RNA
cttgctgctcattggcttct

SEQ ID NO: 435 - RNA, 20 nt, synthetic construct
  misc_feature 1..20: note = Guide sequence
  source 1..20: mol_type = other RNA
tgggactgcgtgacctgtca

SEQ ID NO: 436 - RNA, 20 nt, synthetic construct
  misc_feature 1..20: note = Guide sequence
  source 1..20: mol_type = other RNA
gggactgcgtgacctgtcac

SEQ ID NO: 437 - RNA, 20 nt, synthetic construct
  misc_feature 1..20: note = Guide sequence
  source 1..20: mol_type = other RNA
gtccacagggctcccgtgac

SEQ ID NO: 438 - RNA, 20 nt, synthetic construct
  misc_feature 1..20: note = Guide sequence
  source 1..20: mol_type = other RNA
gacctgtcacgggagccctg

SEQ ID NO: 439 - RNA, 20 nt, synthetic construct
  misc_feature 1..20: note = Guide sequence
  source 1..20: mol_type = other RNA
tggctgtgcagatgtccaca

SEQ ID NO: 440 - RNA, 20 nt, synthetic construct
  misc_feature 1..20: note = Guide sequence
  source 1..20: mol_type = other RNA
ttggctgtgcagatgtccac

SEQ ID NO: 441 - RNA, 20 nt, synthetic construct
  misc_feature 1..20: note = Guide sequence
  source 1..20: mol_type = other RNA
acatctgcacagccaagccg

SEQ ID NO: 442 - RNA, 20 nt, synthetic construct
  misc_feature 1..20: note = Guide sequence
  source 1..20: mol_type = other RNA
catctgcacagccaagccgc

SEQ ID NO: 443 - RNA, 20 nt, synthetic construct
  misc_feature 1..20: note = Guide sequence
  source 1..20: mol_type = other RNA
catgggaatgtcccgcggct

SEQ ID NO: 444 - RNA, 20 nt, synthetic construct
  misc_feature 1..20: note = Guide sequence
  source 1..20: mol_type = other RNA
ggattcatgggaatgtcccg

SEQ ID NO: 445 - RNA, 20 nt, synthetic construct
  misc_feature 1..20: note = Guide sequence
  source 1..20: mol_type = other RNA
taaatgcacatgggattcat

SEQ ID NO: 446 - RNA, 20 nt, synthetic construct
  misc_feature 1..20: note = Guide sequence
  source 1..20: mol_type = other RNA
gtaaatgcacatgggattca

SEQ ID NO: 447 - RNA, 20 nt, synthetic construct
  misc_feature 1..20: note = Guide sequence
  source 1..20: mol_type = other RNA
ggggagcggtaaatgcacat

SEQ ID NO: 448 - RNA, 20 nt, synthetic construct
  misc_feature 1..20: note = Guide sequence
  source 1..20: mol_type = other RNA
cggggagcggtaaatgcaca

SEQ ID NO: 449 - RNA, 20 nt, synthetic construct
  misc_feature 1..20: note = Guide sequence
  source 1..20: mol_type = other RNA
catgtgcatttaccgctccc

SEQ ID NO: 450 - RNA, 20 nt, synthetic construct
  misc_feature 1..20: note = Guide sequence
  source 1..20: mol_type = other RNA
ttgccttcttctccggggag

SEQ ID NO: 451 - RNA, 20 nt, synthetic construct
  misc_feature 1..20: note = Guide sequence
  source 1..20: mol_type = other RNA
ctcagttgccttcttctccg

SEQ ID NO: 452 - RNA, 20 nt, synthetic construct
  misc_feature 1..20: note = Guide sequence
  source 1..20: mol_type = other RNA
cctcagttgccttcttctcc

SEQ ID NO: 453 - RNA, 20 nt, synthetic construct
  misc_feature 1..20: note = Guide sequence
  source 1..20: mol_type = other RNA
tcctcagttgccttcttctc

SEQ ID NO: 454 - RNA, 20 nt, synthetic construct
  misc_feature 1..20: note = Guide sequence
  source 1..20: mol_type = other RNA
ttaccgctccccggagaaga

SEQ ID NO: 455 - RNA, 20 nt, synthetic construct
  misc_feature 1..20: note = Guide sequence
  source 1..20: mol_type = other RNA
cccggagaagaaggcaactg

SEQ ID NO: 456 - RNA, 20 nt, synthetic construct
  misc_feature 1..20: note = Guide sequence
  source 1..20: mol_type = other RNA
gaagaaggcaactgaggatg

SEQ ID NO: 457 - RNA, 20 nt, synthetic construct
  misc_feature 1..20: note = Guide sequence
  source 1..20: mol_type = other RNA
aagaaggcaactgaggatga

SEQ ID NO: 458 - RNA, 20 nt, synthetic construct
  misc_feature 1..20: note = Guide sequence
  source 1..20: mol_type = other RNA
gggctcagaacagaagatcc

SEQ ID NO: 459 - RNA, 20 nt, synthetic construct
  misc_feature 1..20: note = Guide sequence
  source 1..20: mol_type = other RNA
ctcagaacagaagatcccgg

SEQ ID NO: 460 - RNA, 20 nt, synthetic construct
  misc_feature 1..20: note = Guide sequence
  source 1..20: mol_type = other RNA
cacgccggttggtggcctcc

SEQ ID NO: 461 - RNA, 20 nt, synthetic construct
  misc_feature 1..20: note = Guide sequence
  source 1..20: mol_type = other RNA
acacgccggttggtggcctc

SEQ ID NO: 462 - RNA, 20 nt, synthetic construct
  misc_feature 1..20: note = Guide sequence
  source 1..20: mol_type = other RNA
agatcccggaggccaccaac

SEQ ID NO: 463 - RNA, 20 nt, synthetic construct
  misc_feature 1..20: note = Guide sequence
  source 1..20: mol_type = other RNA
ttcccagacacgccggttgg

SEQ ID NO: 464 - RNA, 20 nt, synthetic construct
  misc_feature 1..20: note = Guide sequence
  source 1..20: mol_type = other RNA
cagttcccagacacgccggt

SEQ ID NO: 465 - RNA, 20 nt, synthetic construct
  misc_feature 1..20: note = Guide sequence
  source 1..20: mol_type = other RNA
tggacagttcccagacacgc

SEQ ID NO: 466 - RNA, 20 nt, synthetic construct
  misc_feature 1..20: note = Guide sequence
  source 1..20: mol_type = other RNA
aggccaccaaccggcgtgtc

SEQ ID NO: 467 - RNA, 20 nt, synthetic construct
  misc_feature 1..20: note = Guide sequence
  source 1..20: mol_type = other RNA
ggccaccaaccggcgtgtct

SEQ ID NO: 468 - RNA, 20 nt, synthetic construct
  misc_feature 1..20: note = Guide sequence
  source 1..20: mol_type = other RNA
gcgtgtctgggaactgtcca

SEQ ID NO: 469 - RNA, 20 nt, synthetic construct
  misc_feature 1..20: note = Guide sequence
  source 1..20: mol_type = other RNA
agcaaagcgggaattggcct

SEQ ID NO: 470 - RNA, 20 nt, synthetic construct
  misc_feature 1..20: note = Guide sequence
  source 1..20: mol_type = other RNA
agtggtagcaaagcgggaat

SEQ ID NO: 471 - RNA, 20 nt, synthetic construct
  misc_feature 1..20: note = Guide sequence
  source 1..20: mol_type = other RNA
atagaaagtggtagcaaagc

SEQ ID NO: 472 - RNA, 20 nt, synthetic construct
  misc_feature 1..20: note = Guide sequence
  source 1..20: mol_type = other RNA
gatagaaagtggtagcaaag

SEQ ID NO: 473 - RNA, 20 nt, synthetic construct
  misc_feature 1..20: note = Guide sequence
  source 1..20: mol_type = other RNA
tgccaggtgctgatagaaag

SEQ ID NO: 474 - RNA, 20 nt, synthetic construct
  misc_feature 1..20: note = Guide sequence
  source 1..20: mol_type = other RNA
taccactttctatcagcacc

SEQ ID NO: 475 - RNA, 20 nt, synthetic construct
  misc_feature 1..20: note = Guide sequence
  source 1..20: mol_type = other RNA
tgtcattcttggaatctgcc

SEQ ID NO: 476 - RNA, 20 nt, synthetic construct
  misc_feature 1..20: note = Guide sequence
  source 1..20: mol_type = other RNA
aatgttatcattgtcattct

SEQ ID NO: 477 - RNA, 20 nt, synthetic construct
  misc_feature 1..20: note = Guide sequence
  source 1..20: mol_type = other RNA
tggagatactcaggggtgac

SEQ ID NO: 478 - RNA, 20 nt, synthetic construct
  misc_feature 1..20: note = Guide sequence
  source 1..20: mol_type = other RNA
aaagccgtggagatactcag

SEQ ID NO: 479 - RNA, 20 nt, synthetic construct
  misc_feature 1..20: note = Guide sequence
  source 1..20: mol_type = other RNA
aaaagccgtggagatactca

SEQ ID NO: 480 - RNA, 20 nt, synthetic construct
  misc_feature 1..20: note = Guide sequence
  source 1..20: mol_type = other RNA
caaaagccgtggagatactc

SEQ ID NO: 481 - RNA, 20 nt, synthetic construct
  misc_feature 1..20: note = Guide sequence
  source 1..20: mol_type = other RNA
gtcacccctgagtatctcca

SEQ ID NO: 482 - RNA, 20 nt, synthetic construct
  misc_feature 1..20: note = Guide sequence
  source 1..20: mol_type = other RNA
cttggtcatagcaaaagccg

SEQ ID NO: 483 - RNA, 20 nt, synthetic construct
  misc_feature 1..20: note = Guide sequence
  source 1..20: mol_type = other RNA
ggcttttgctatgaccaagc

SEQ ID NO: 484 - RNA, 20 nt, synthetic construct
  misc_feature 1..20: note = Guide sequence
  source 1..20: mol_type = other RNA
gcttttgctatgaccaagct

SEQ ID NO: 485 - RNA, 20 nt, synthetic construct
  misc_feature 1..20: note = Guide sequence
  source 1..20: mol_type = other RNA
gtcattacaggcacccagct

SEQ ID NO: 486 - RNA, 20 nt, synthetic construct
  misc_feature 1..20: note = Guide sequence
  source 1..20: mol_type = other RNA
ttgctggagggtgtcattac

SEQ ID NO: 487 - RNA, 20 nt, synthetic construct
  misc_feature 1..20: note = Guide sequence
  source 1..20: mol_type = other RNA
tacctccatcagttgctgga

SEQ ID NO: 488 - RNA, 20 nt, synthetic construct
  misc_feature 1..20: note = Guide sequence
  source 1..20: mol_type = other RNA
gtacctccatcagttgctgg

SEQ ID NO: 489 - RNA, 20 nt, synthetic construct
  misc_feature 1..20: note = Guide sequence
  source 1..20: mol_type = other RNA
gtcgtacctccatcagttgc

SEQ ID NO: 490 - RNA, 20 nt, synthetic construct
  misc_feature 1..20: note = Guide sequence
  source 1..20: mol_type = other RNA
tgacaccctccagcaactga

SEQ ID NO: 491 - RNA, 20 nt, synthetic construct
  misc_feature 1..20: note = Guide sequence
  source 1..20: mol_type = other RNA
caccctccagcaactgatgg

SEQ ID NO: 492 - RNA, 20 nt, synthetic construct
  misc_feature 1..20: note = Guide sequence
  source 1..20: mol_type = other RNA
atcagatgttttctcagata

SEQ ID NO: 493 - RNA, 20 nt, synthetic construct
  misc_feature 1..20: note = Guide sequence
  source 1..20: mol_type = other RNA
tcagtttggcaaagaagaag

SEQ ID NO: 494 - RNA, 20 nt, synthetic construct
  misc_feature 1..20: note = Guide sequence
  source 1..20: mol_type = other RNA
atagagtcggcagttcagtt

SEQ ID NO: 495 - RNA, 20 nt, synthetic construct
  misc_feature 1..20: note = Guide sequence
  source 1..20: mol_type = other RNA
tgttggcttttcgatagagt

SEQ ID NO: 496 - RNA, 20 nt, synthetic construct
  misc_feature 1..20: note = Guide sequence
  source 1..20: mol_type = other RNA
tactaacttggaggatttgt

SEQ ID NO: 497 - RNA, 20 nt, synthetic construct
  misc_feature 1..20: note = Guide sequence
  source 1..20: mol_type = other RNA
attggctgatactaacttgg

SEQ ID NO: 498 - RNA, 20 nt, synthetic construct
  misc_feature 1..20: note = Guide sequence
  source 1..20: mol_type = other RNA
gcgattggctgatactaact

SEQ ID NO: 499 - RNA, 20 nt, synthetic construct
  misc_feature 1..20: note = Guide sequence
  source 1..20: mol_type = other RNA
tttgtctccaaaaaggcgat

SEQ ID NO: 500 - RNA, 20 nt, synthetic construct
  misc_feature 1..20: note = Guide sequence
  source 1..20: mol_type = other RNA
gtatcagccaatcgcctttt

SEQ ID NO: 501 - RNA, 20 nt, synthetic construct
  misc_feature 1..20: note = Guide sequence
  source 1..20: mol_type = other RNA
taagggatttgtctccaaaa

SEQ ID NO: 502 - RNA, 20 nt, synthetic construct
  misc_feature 1..20: note = Guide sequence
  source 1..20: mol_type = other RNA
gtaggtctcattgaaggtaa

SEQ ID NO: 503 - RNA, 20 nt, synthetic construct
  misc_feature 1..20: note = Guide sequence
  source 1..20: mol_type = other RNA
ggtaggtctcattgaaggta

SEQ ID NO: 504 - RNA, 20 nt, synthetic construct
  misc_feature 1..20: note = Guide sequence
  source 1..20: mol_type = other RNA
gtcctggtaggtctcattga

SEQ ID NO: 505 - RNA, 20 nt, synthetic construct
  misc_feature 1..20: note = Guide sequence
  source 1..20: mol_type = other RNA
taccttcaatgagacctacc

SEQ ID NO: 506 - RNA, 20 nt, synthetic construct
  misc_feature 1..20: note = Guide sequence
  source 1..20: mol_type = other RNA
caactcactgatgtcctggt

SEQ ID NO: 507 - RNA, 20 nt, synthetic construct
  misc_feature 1..20: note = Guide sequence
  source 1..20: mol_type = other RNA
ataccaactcactgatgtcc

SEQ ID NO: 508 - RNA, 20 nt, synthetic construct
  misc_feature 1..20: note = Guide sequence
  source 1..20: mol_type = other RNA
ctaccaggacatcagtgagt

SEQ ID NO: 509 - RNA, 20 nt, synthetic construct
  misc_feature 1..20: note = Guide sequence
  source 1..20: mol_type = other RNA
gacatcagtgagttggtata

SEQ ID NO: 510 - RNA, 20 nt, synthetic construct
  misc_feature 1..20: note = Guide sequence
  source 1..20: mol_type = other RNA
gaagtccaggggctggagct

SEQ ID NO: 511 - RNA, 20 nt, synthetic construct
  misc_feature 1..20: note = Guide sequence
  source 1..20: mol_type = other RNA
tggagccaagctccagcccc

SEQ ID NO: 512 - RNA, 20 nt, synthetic construct
  misc_feature 1..20: note = Guide sequence
  source 1..20: mol_type = other RNA
tcaccttgaagtccaggggc

SEQ ID NO: 513 - RNA, 20 nt, synthetic construct
  misc_feature 1..20: note = Guide sequence
  source 1..20: mol_type = other RNA
caactcaccttgaagtccag

SEQ ID NO: 514 - RNA, 20 nt, synthetic construct
  misc_feature 1..20: note = Guide sequence
  source 1..20: mol_type = other RNA
gcaactcaccttgaagtcca

SEQ ID NO: 515 - RNA, 20 nt, synthetic construct
  misc_feature 1..20: note = Guide sequence
  source 1..20: mol_type = other RNA
tgcaactcaccttgaagtcc

SEQ ID NO: 516 - RNA, 20 nt, synthetic construct
  misc_feature 1..20: note = Guide sequence
  source 1..20: mol_type = other RNA
gctccagcccctggacttca

SEQ ID NO: 517 - RNA, 20 nt, synthetic construct
  misc_feature 1..20: note = Guide sequence
  source 1..20: mol_type = other RNA
gattgctctgcattttcctg

SEQ ID NO: 518 - RNA, 20 nt, synthetic construct
  misc_feature 1..20: note = Guide sequence
  source 1..20: mol_type = other RNA
aaatgcagagcaatccagag

SEQ ID NO: 519 - RNA, 20 nt, synthetic construct
  misc_feature 1..20: note = Guide sequence
  source 1..20: mol_type = other RNA
ccatttgttgatggccgctc

SEQ ID NO: 520 - RNA, 20 nt, synthetic construct
  misc_feature 1..20: note = Guide sequence
  source 1..20: mol_type = other RNA
attggacacccatttgttga

SEQ ID NO: 521 - RNA, 20 nt, synthetic construct
  misc_feature 1..20: note = Guide sequence
  source 1..20: mol_type = other RNA
ccagagcggccatcaacaaa

SEQ ID NO: 522 - RNA, 20 nt, synthetic construct
  misc_feature 1..20: note = Guide sequence
  source 1..20: mol_type = other RNA
cagagcggccatcaacaaat

SEQ ID NO: 523 - RNA, 20 nt, synthetic construct
  misc_feature 1..20: note = Guide sequence
  source 1..20: mol_type = other RNA
gattcggccttcggtcttat

SEQ ID NO: 524 - RNA, 20 nt, synthetic construct
  misc_feature 1..20: note = Guide sequence
  source 1..20: mol_type = other RNA
tgggtgtccaataagaccga

SEQ ID NO: 525 - RNA, 20 nt, synthetic construct
  misc_feature 1..20: note = Guide sequence
  source 1..20: mol_type = other RNA
gacatcggtgattcggcctt

SEQ ID NO: 526 - RNA, 20 nt, synthetic construct
  misc_feature 1..20: note = Guide sequence
  source 1..20: mol_type = other RNA
agggaatgacatcggtgatt

SEQ ID NO: 527 - RNA, 20 nt, synthetic construct
  misc_feature 1..20: note = Guide sequence
  source 1..20: mol_type = other RNA
ggcttccgagggaatgacat

SEQ ID NO: 528 - RNA, 20 nt, synthetic construct
  misc_feature 1..20: note = Guide sequence
  source 1..20: mol_type = other RNA
aatcaccgatgtcattccct

SEQ ID NO: 529 - RNA, 20 nt, synthetic construct
  misc_feature 1..20: note = Guide sequence
  source 1..20: mol_type = other RNA
agctcattgatggcttccga

SEQ ID NO: 530 - RNA, 20 nt, synthetic construct
  misc_feature 1..20: note = Guide sequence
  source 1..20: mol_type = other RNA
gagctcattgatggcttccg

SEQ ID NO: 531 - RNA, 20 nt, synthetic construct
  misc_feature 1..20: note = Guide sequence
  source 1..20: mol_type = other RNA
cagaacagtgagctcattga

SEQ ID NO: 532 - RNA, 20 nt, synthetic construct
  misc_feature 1..20: note = Guide sequence
  source 1..20: mol_type = other RNA
catcaatgagctcactgttc

SEQ ID NO: 533 - RNA, 20 nt, synthetic construct
  misc_feature 1..20: note = Guide sequence
  source 1..20: mol_type = other RNA
tgagctcactgttctggtgc

SEQ ID NO: 534 - RNA, 20 nt, synthetic construct
  misc_feature 1..20: note = Guide sequence
  source 1..20: mol_type = other RNA
tctgagtaccttgaagtaaa

SEQ ID NO: 535 - RNA, 20 nt, synthetic construct
  misc_feature 1..20: note = Guide sequence
  source 1..20: mol_type = other RNA
ggttaacaccatttacttca

SEQ ID NO: 536 - RNA, 20 nt, synthetic construct
  misc_feature 1..20: note = Guide sequence
  source 1..20: mol_type = other RNA
actttgacttccacaggccc

SEQ ID NO: 537 - RNA, 20 nt, synthetic construct
  misc_feature 1..20: note = Guide sequence
  source 1..20: mol_type = other RNA
tgattctcttccagggcctg

SEQ ID NO: 538 - RNA, 20 nt, synthetic construct
  misc_feature 1..20: note = Guide sequence
  source 1..20: mol_type = other RNA
ggctgaactttgacttccac

SEQ ID NO: 539 - RNA, 20 nt, synthetic construct
  misc_feature 1..20: note = Guide sequence
  source 1..20: mol_type = other RNA
gttccttccttgtgttctca

SEQ ID NO: 540 - RNA, 20 nt, synthetic construct
  misc_feature 1..20: note = Guide sequence
  source 1..20: mol_type = other RNA
agttccttccttgtgttctc

SEQ ID NO: 541 - RNA, 20 nt, synthetic construct
  misc_feature 1..20: note = Guide sequence
  source 1..20: mol_type = other RNA
agttcagccctgagaacaca

SEQ ID NO: 542 - RNA, 20 nt, synthetic construct
  misc_feature 1..20: note = Guide sequence
  source 1..20: mol_type = other RNA
cagccctgagaacacaagga

SEQ ID NO: 543 - RNA, 20 nt, synthetic construct
  misc_feature 1..20: note = Guide sequence
  source 1..20: mol_type = other RNA
aaggaaggaactgttctaca

SEQ ID NO: 544 - RNA, 20 nt, synthetic construct
  misc_feature 1..20: note = Guide sequence
  source 1..20: mol_type = other RNA
gaactgttctacaaggctga

SEQ ID NO: 545 - RNA, 20 nt, synthetic construct
  misc_feature 1..20: note = Guide sequence
  source 1..20: mol_type = other RNA
ttcagcatctatgatgtacc

SEQ ID NO: 546 - RNA, 20 nt, synthetic construct
  misc_feature 1..20: note = Guide sequence
  source 1..20: mol_type = other RNA
gcatctatgatgtaccagga

SEQ ID NO: 547 - RNA, 20 nt, synthetic construct
  misc_feature 1..20: note = Guide sequence
  source 1..20: mol_type = other RNA
gataacggaacttgccttcc

SEQ ID NO: 548 - RNA, 20 nt, synthetic construct
  misc_feature 1..20: note = Guide sequence
  source 1..20: mol_type = other RNA
aggaaggcaagttccgttat

SEQ ID NO: 549 - RNA, 20 nt, synthetic construct
  misc_feature 1..20: note = Guide sequence
  source 1..20: mol_type = other RNA
cttcagccacgcgccgataa

SEQ ID NO: 550 - RNA, 20 nt, synthetic construct
  misc_feature 1..20: note = Guide sequence
  source 1..20: mol_type = other RNA
caagttccgttatcggcgcg

SEQ ID NO: 551 - RNA, 20 nt, synthetic construct
  misc_feature 1..20: note = Guide sequence
  source 1..20: mol_type = other RNA
cgttatcggcgcgtggctga

SEQ ID NO: 552 - RNA, 20 nt, synthetic construct
  misc_feature 1..20: note = Guide sequence
  source 1..20: mol_type = other RNA
gcgcgtggctgaaggcaccc

SEQ ID NO: 553 - RNA, 20 nt, synthetic construct
  misc_feature 1..20: note = Guide sequence
  source 1..20: mol_type = other RNA
gaagggcaactcaagcacct

SEQ ID NO: 554 - RNA, 20 nt, synthetic construct
  misc_feature 1..20: note = Guide sequence
  source 1..20: mol_type = other RNA
tgaagggcaactcaagcacc

SEQ ID NO: 555 - RNA, 20 nt, synthetic construct
  misc_feature 1..20: note = Guide sequence
  source 1..20: mol_type = other RNA
gtgcttgagttgcccttcaa

SEQ ID NO: 556 - RNA, 20 nt, synthetic construct
  misc_feature 1..20: note = Guide sequence
  source 1..20: mol_type = other RNA
gtgatgtcatcacctttgaa

SEQ ID NO: 557 - RNA, 20 nt, synthetic construct
  misc_feature 1..20: note = Guide sequence
  source 1..20: mol_type = other RNA
ggtgatgtcatcacctttga

SEQ ID NO: 558 - RNA, 20 nt, synthetic construct
  misc_feature 1..20: note = Guide sequence
  source 1..20: mol_type = other RNA
caaaggtgatgacatcacca

SEQ ID NO: 559 - RNA, 20 nt, synthetic construct
  misc_feature 1..20: note = Guide sequence
  source 1..20: mol_type = other RNA
cttgggcaagatgaggacca

SEQ ID NO: 560 - RNA, 20 nt, synthetic construct
  misc_feature 1..20: note = Guide sequence
  source 1..20: mol_type = other RNA
tctcaggcttgggcaagatg

SEQ ID NO: 561 - RNA, 20 nt, synthetic construct
  misc_feature 1..20: note = Guide sequence
  source 1..20: mol_type = other RNA
gccaggctcttctcaggctt

SEQ ID NO: 562 - RNA, 20 nt, synthetic construct
  misc_feature 1..20: note = Guide sequence
  source 1..20: mol_type = other RNA
ggccaggctcttctcaggct

SEQ ID NO: 563 - RNA, 20 nt, synthetic construct
  misc_feature 1..20: note = Guide sequence
  source 1..20: mol_type = other RNA
accttggccaggctcttctc

SEQ ID NO: 564 - RNA, 20 nt, synthetic construct
  misc_feature 1..20: note = Guide sequence
  source 1..20: mol_type = other RNA
gcccaagcctgagaagagcc

SEQ ID NO: 565 - RNA, 20 nt, synthetic construct
  misc_feature 1..20: note = Guide sequence
  source 1..20: mol_type = other RNA
gcctgagaagagcctggcca

SEQ ID NO: 566 - RNA, 20 nt, synthetic construct
  misc_feature 1..20: note = Guide sequence
  source 1..20: mol_type = other RNA
gttccttctctaccttggcc

SEQ ID NO: 567 - RNA, 20 nt, synthetic construct
  misc_feature 1..20: note = Guide sequence
  source 1..20: mol_type = other RNA
ggtgagttccttctctacct

SEQ ID NO: 568 - RNA, 20 nt, synthetic construct
  misc_feature 1..20: note = Guide sequence
  source 1..20: mol_type = other RNA
gagcctggccaaggtagaga

SEQ ID NO: 569 - RNA, 20 nt, synthetic construct
  misc_feature 1..20: note = Guide sequence
  source 1..20: mol_type = other RNA
agagaaggaactcaccccag

SEQ ID NO: 570 - RNA, 20 nt, synthetic construct
  misc_feature 1..20: note = Guide sequence
  source 1..20: mol_type = other RNA
ccactcttgcagcacctctg

SEQ ID NO: 571 - RNA, 20 nt, synthetic construct
  misc_feature 1..20: note = Guide sequence
  source 1..20: mol_type = other RNA
gccactcttgcagcacctct

SEQ ID NO: 572 - RNA, 20 nt, synthetic construct
  misc_feature 1..20: note = Guide sequence
  source 1..20: mol_type = other RNA
agccactcttgcagcacctc

SEQ ID NO: 573 - RNA, 20 nt, synthetic construct
  misc_feature 1..20: note = Guide sequence
  source 1..20: mol_type = other RNA
ccccagaggtgctgcaagag

SEQ ID NO: 574 - RNA, 20 nt, synthetic construct
  misc_feature 1..20: note = Guide sequence
  source 1..20: mol_type = other RNA
agaggtgctgcaagagtggc

SEQ ID NO: 575 - RNA, 20 nt, synthetic construct
  misc_feature 1..20: note = Guide sequence
  source 1..20: mol_type = other RNA
gcaagagtggctggatgaat

SEQ ID NO: 576 - RNA, 20 nt, synthetic construct
  misc_feature 1..20: note = Guide sequence
  source 1..20: mol_type = other RNA
agagtggctggatgaattgg

SEQ ID NO: 577 - RNA, 20 nt, synthetic construct
  misc_feature 1..20: note = Guide sequence
  source 1..20: mol_type = other RNA
tgaattggaggagatgatgc

SEQ ID NO: 578 - RNA, 20 nt, synthetic construct
  misc_feature 1..20: note = Guide sequence
  source 1..20: mol_type = other RNA
attggaggagatgatgctgg

SEQ ID NO: 579 - RNA, 20 nt, synthetic construct
  misc_feature 1..20: note = Guide sequence
  source 1..20: mol_type = other RNA
caatgcggaagcggggcatg

SEQ ID NO: 580 - RNA, 20 nt, synthetic construct
  misc_feature 1..20: note = Guide sequence
  source 1..20: mol_type = other RNA
ccgtcctcaatgcggaagcg

SEQ ID NO: 581 - RNA, 20 nt, synthetic construct
  misc_feature 1..20: note = Guide sequence
  source 1..20: mol_type = other RNA
gccgtcctcaatgcggaagc

SEQ ID NO: 582 - RNA, 20 nt, synthetic construct
  misc_feature 1..20: note = Guide sequence
  source 1..20: mol_type = other RNA
agccgtcctcaatgcggaag

SEQ ID NO: 583 - RNA, 20 nt, synthetic construct
  misc_feature 1..20: note = Guide sequence
  source 1..20: mol_type = other RNA
catgccccgcttccgcattg

SEQ ID NO: 584 - RNA, 20 nt, synthetic construct
  misc_feature 1..20: note = Guide sequence
  source 1..20: mol_type = other RNA
aactgaagccgtcctcaatg

SEQ ID NO: 585 - RNA, 20 nt, synthetic construct
  misc_feature 1..20: note = Guide sequence
  source 1..20: mol_type = other RNA
ccccgcttccgcattgagga

SEQ ID NO: 586 - RNA, 20 nt, synthetic construct
  misc_feature 1..20: note = Guide sequence
  source 1..20: mol_type = other RNA
tgaggacggcttcagtttga

SEQ ID NO: 587 - RNA, 20 nt, synthetic construct
  misc_feature 1..20: note = Guide sequence
  source 1..20: mol_type = other RNA
gaaggagcagctgcaagaca

SEQ ID NO: 588 - RNA, 20 nt, synthetic construct
  misc_feature 1..20: note = Guide sequence
  source 1..20: mol_type = other RNA
aaggagcagctgcaagacat

SEQ ID NO: 589 - RNA, 20 nt, synthetic construct
  misc_feature 1..20: note = Guide sequence
  source 1..20: mol_type = other RNA
cagggctgaacagatcgaca

SEQ ID NO: 590 - RNA, 20 nt, synthetic construct
  misc_feature 1..20: note = Guide sequence
  source 1..20: mol_type = other RNA
ctgggagtttggacttttca

SEQ ID NO: 591 - RNA, 20 nt, synthetic construct
  misc_feature 1..20: note = Guide sequence
  source 1..20: mol_type = other RNA
cctgggagtttggacttttc

SEQ ID NO: 592 - RNA, 20 nt, synthetic construct
  misc_feature 1..20: note = Guide sequence
  source 1..20: mol_type = other RNA
cctagacaaacctgggagtt

SEQ ID NO: 593 - RNA, 20 nt, synthetic construct
  misc_feature 1..20: note = Guide sequence
  source 1..20: mol_type = other RNA
cctgaaaagtccaaactccc

SEQ ID NO: 594 - RNA, 20 nt, synthetic construct
  misc_feature 1..20: note = Guide sequence
  source 1..20: mol_type = other RNA
cggccttctgcaacaatacc

SEQ ID NO: 595 - RNA, 20 nt, synthetic construct
  misc_feature 1..20: note = Guide sequence
  source 1..20: mol_type = other RNA
cttccaggtattgttgcaga

SEQ ID NO: 596 - RNA, 20 nt, synthetic construct
  misc_feature 1..20: note = Guide sequence
  source 1..20: mol_type = other RNA
ctgagacatagaggtcatct

SEQ ID NO: 597 - RNA, 20 nt, synthetic construct
  misc_feature 1..20: note = Guide sequence
  source 1..20: mol_type = other RNA
ggaatgcatctgagacatag

SEQ ID NO: 598 - RNA, 20 nt, synthetic construct
  misc_feature 1..20: note = Guide sequence
  source 1..20: mol_type = other RNA
tgtctcagatgcattccata

SEQ ID NO: 599 - RNA, 20 nt, synthetic construct
  misc_feature 1..20: note = Guide sequence
  source 1..20: mol_type = other RNA
tcacctcaagaaatgcctta

SEQ ID NO: 600 - RNA, 20 nt, synthetic construct
  misc_feature 1..20: note = Guide sequence
  source 1..20: mol_type = other RNA
attccataaggcatttcttg

SEQ ID NO: 601 - RNA, 20 nt, synthetic construct
  misc_feature 1..20: note = Guide sequence
  source 1..20: mol_type = other RNA
gacctgcaggtaaatgaaga

SEQ ID NO: 602 - RNA, 20 nt, synthetic construct
  misc_feature 1..20: note = Guide sequence
  source 1..20: mol_type = other RNA
acggccagcaatcacaacag

SEQ ID NO: 603 - RNA, 20 nt, synthetic construct
  misc_feature 1..20: note = Guide sequence
  source 1..20: mol_type = other RNA
agtaccgctgttgtgattgc

SEQ ID NO: 604 - RNA, 20 nt, synthetic construct
  misc_feature 1..20: note = Guide sequence
  source 1..20: mol_type = other RNA
ccctgttggggtttagcgaa

SEQ ID NO: 605 - RNA, 20 nt, synthetic construct
  misc_feature 1..20: note = Guide sequence
  source 1..20: mol_type = other RNA
gccgttcgctaaaccccaac

SEQ ID NO: 606 - RNA, 20 nt, synthetic construct
  misc_feature 1..20: note = Guide sequence
  source 1..20: mol_type = other RNA
ccgttcgctaaaccccaaca

SEQ ID NO: 607 - RNA, 20 nt, synthetic construct
  misc_feature 1..20: note = Guide sequence
  source 1..20: mol_type = other RNA
ccttgaaagtcaccctgttg

SEQ ID NO: 608 - RNA, 20 nt, synthetic construct
  misc_feature 1..20: note = Guide sequence
  source 1..20: mol_type = other RNA
gccttgaaagtcaccctgtt

SEQ ID NO: 609 - RNA, 20 nt, synthetic construct
  misc_feature 1..20: note = Guide sequence
  source 1..20: mol_type = other RNA
ggccttgaaagtcaccctgt

SEQ ID NO: 610 - RNA, 20 nt, synthetic construct
  misc_feature 1..20: note = Guide sequence
  source 1..20: mol_type = other RNA
ccccaacagggtgactttca

SEQ ID NO: 611 - RNA, 20 nt, synthetic construct
  misc_feature 1..20: note = Guide sequence
  source 1..20: mol_type = other RNA
gggtgactttcaaggccaac

SEQ ID NO: 612 - RNA, 20 nt, synthetic construct
  misc_feature 1..20: note = Guide sequence
  source 1..20: mol_type = other RNA
aaaaaccaggaaaggcctgt

SEQ ID NO: 613 - RNA, 20 nt, synthetic construct
  misc_feature 1..20: note = Guide sequence
  source 1..20: mol_type = other RNA
caaggccaacaggcctttcc

SEQ ID NO: 614 - RNA, 20 nt, synthetic construct
  misc_feature 1..20: note = Guide sequence
  source 1..20: mol_type = other RNA
tctcttataaaaaccaggaa

SEQ ID NO: 615 - RNA, 20 nt, synthetic construct
  misc_feature 1..20: note = Guide sequence
  source 1..20: mol_type = other RNA
gaacttctcttataaaaacc

SEQ ID NO: 616 - RNA, 20 nt, synthetic construct
  misc_feature 1..20: note = Guide sequence
  source 1..20: mol_type = other RNA
atgaagataatagtgttcag

SEQ ID NO: 617 - RNA, 20 nt, synthetic construct
  misc_feature 1..20: note = Guide sequence
  source 1..20: mol_type = other RNA
tctgaacactattatcttca

SEQ ID NO: 618 - RNA, 20 nt, synthetic construct
  misc_feature 1..20: note = Guide sequence
  source 1..20: mol_type = other RNA
ctgaacactattatcttcat

SEQ ID NO: 619 - RNA, 20 nt, synthetic construct
  misc_feature 1..20: note = Guide sequence
  source 1..20: mol_type = other RNA
attttacttaacacaagggt

SEQ ID NO: 620 - RNA, 20 nt, synthetic construct
  misc_feature 1..20: note = Guide sequence
  source 1..20: mol_type = other RNA
gaacattttacttaacacaa

SEQ ID NO: 621 - RNA, 20 nt, synthetic construct
  misc_feature 1..20: note = Guide sequence
  source 1..20: mol_type = other RNA
agaacattttacttaacaca

SEQ ID NO: 622 - RNA, 20 nt, synthetic construct
  misc_feature 1..20: note = Guide sequence
  source 1..20: mol_type = other RNA
ggttacagttcctatcacat

SEQ ID NO: 623 - RNA, 20 nt, synthetic construct
  misc_feature 1..20: note = Guide sequence
  source 1..20: mol_type = other RNA
ccaagccgcgggacattccc

SEQ ID NO: 624 - RNA, 20 nt, synthetic construct
  misc_feature 1..20: note = Guide sequence
  source 1..20: mol_type = other RNA
taaatgcacatgggattcat

SEQ ID NO: 625 - RNA, 20 nt, synthetic construct
  misc_feature 1..20: note = Guide sequence
  source 1..20: mol_type = other RNA
cggggagcggtaaatgcaca

SEQ ID NO: 626 - RNA, 20 nt, synthetic construct
  misc_feature 1..20: note = Guide sequence
  source 1..20: mol_type = other RNA
ccccggagaagaaggcaact

SEQ ID NO: 627 - RNA, 20 nt, synthetic construct
  misc_feature 1..20: note = Guide sequence
  source 1..20: mol_type = other RNA
acacgccggttggtggcctc

SEQ ID NO: 628 - RNA, 20 nt, synthetic construct
  misc_feature 1..20: note = Guide sequence
  source 1..20: mol_type = other RNA
atagaaagtggtagcaaagc

SEQ ID NO: 629 - RNA, 20 nt, synthetic construct
  misc_feature 1..20: note = Guide sequence
  source 1..20: mol_type = other RNA
atcagcacctggcagattcc

SEQ ID NO: 630 - RNA, 20 nt, synthetic construct
  misc_feature 1..20: note = Guide sequence
  source 1..20: mol_type = other RNA
aatgttatcattgtcattct

SEQ ID NO: 631 - RNA, 20 nt, synthetic construct
  misc_feature 1..20: note = Guide sequence
  source 1..20: mol_type = other RNA
ataacattttcctgtcaccc

SEQ ID NO: 632 - RNA, 20 nt, synthetic construct
  misc_feature 1..20: note = Guide sequence
  source 1..20: mol_type = other RNA
caaaagccgtggagatactc

SEQ ID NO: 633 - RNA, 20 nt, synthetic construct
  misc_feature 1..20: note = Guide sequence
  source 1..20: mol_type = other RNA
cggcttttgctatgaccaag

SEQ ID NO: 634 - RNA, 20 nt, synthetic construct
  misc_feature 1..20: note = Guide sequence
  source 1..20: mol_type = other RNA
cgtacctccatcagttgctg

SEQ ID NO: 635 - RNA, 20 nt, synthetic construct
  misc_feature 1..20: note = Guide sequence
  source 1..20: mol_type = other RNA
ttcagtttggcaaagaagaa

SEQ ID NO: 636 - RNA, 20 nt, synthetic construct
  misc_feature 1..20: note = Guide sequence
  source 1..20: mol_type = other RNA
aggatttgttggcttttcga

SEQ ID NO: 637 - RNA, 20 nt, synthetic construct
  misc_feature 1..20: note = Guide sequence
  source 1..20: mol_type = other RNA
gattggctgatactaacttg

SEQ ID NO: 638 - RNA, 20 nt, synthetic construct
  misc_feature 1..20: note = Guide sequence
  source 1..20: mol_type = other RNA
ggtaggtctcattgaaggta

SEQ ID NO: 639 - RNA, 20 nt, synthetic construct
  misc_feature 1..20: note = Guide sequence
  source 1..20: mol_type = other RNA
tgagacctaccaggacatca

SEQ ID NO: 640 - RNA, 20 nt, synthetic construct
  misc_feature 1..20: note = Guide sequence
  source 1..20: mol_type = other RNA
tccagcccctggacttcaag

SEQ ID NO: 641 - RNA, 20 nt, synthetic construct
  misc_feature 1..20: note = Guide sequence
  source 1..20: mol_type = other RNA
cccatttgttgatggccgct

SEQ ID NO: 642 - RNA, 20 nt, synthetic construct
  misc_feature 1..20: note = Guide sequence
  source 1..20: mol_type = other RNA
tccagagcggccatcaacaa

SEQ ID NO: 643 - RNA, 20 nt, synthetic construct
  misc_feature 1..20: note = Guide sequence
  source 1..20: mol_type = other RNA
gtgtccaataagaccgaagg

SEQ ID NO: 644 - RNA, 20 nt, synthetic construct
  misc_feature 1..20: note = Guide sequence
  source 1..20: mol_type = other RNA
agctcattgatggcttccga

SEQ ID NO: 645 - RNA, 20 nt, synthetic construct
  misc_feature 1..20: note = Guide sequence
  source 1..20: mol_type = other RNA
actgttctacaaggctgatg

SEQ ID NO: 646 - RNA, 20 nt, synthetic construct
  misc_feature 1..20: note = Guide sequence
  source 1..20: mol_type = other RNA
ggctgaaggcacccaggtgc

SEQ ID NO: 647 - RNA, 20 nt, synthetic construct
  misc_feature 1..20: note = Guide sequence
  source 1..20: mol_type = other RNA
ttgaagggcaactcaagcac

SEQ ID NO: 648 - RNA, 20 nt, synthetic construct
  misc_feature 1..20: note = Guide sequence
  source 1..20: mol_type = other RNA
actcttgcagcacctctggg

SEQ ID NO: 649 - RNA, 20 nt, synthetic construct
  misc_feature 1..20: note = Guide sequence
  source 1..20: mol_type = other RNA
agccactcttgcagcacctc

SEQ ID NO: 650 - RNA, 20 nt, synthetic construct
  misc_feature 1..20: note = Guide sequence
  source 1..20: mol_type = other RNA
actcaccccagaggtgctgc

SEQ ID NO: 651 - RNA, 20 nt, synthetic construct
  misc_feature 1..20: note = Guide sequence
  source 1..20: mol_type = other RNA
cagaggtgctgcaagagtgg

SEQ ID NO: 652 - RNA, 20 nt, synthetic construct
  misc_feature 1..20: note = Guide sequence
  source 1..20: mol_type = other RNA
ggtgctgcaagagtggctgg

SEQ ID NO: 653 - RNA, 20 nt, synthetic construct
  misc_feature 1..20: note = Guide sequence
  source 1..20: mol_type = other RNA
tcacctcaagaaatgcctta

SEQ ID NO: 654 - RNA, 20 nt, synthetic construct
  misc_feature 1..20: note = Guide sequence
  source 1..20: mol_type = other RNA
tccataaggcatttcttgag

SEQ ID NO: 655 - RNA, 20 nt, synthetic construct
  misc_feature 1..20: note = Guide sequence
  source 1..20: mol_type = other RNA
ggccgttcgctaaaccccaa

SEQ ID NO: 656 - RNA, 20 nt, synthetic construct
  misc_feature 1..20: note = Guide sequence
  source 1..20: mol_type = other RNA
ggccttgaaagtcaccctgt

SEQ ID NO: 657 - RNA, 20 nt, synthetic construct
  misc_feature 1..20: note = Guide sequence
  source 1..20: mol_type = other RNA
aacactattatcttcatggg

SEQ ID NO: 658 - RNA, 20 nt, synthetic construct
  misc_feature 1..20: note = Guide sequence
  source 1..20: mol_type = other RNA
aagaacattttacttaacac

SEQ ID NO: 659 - RNA, 22 nt, synthetic construct
  misc_feature 1..22: note = Guide sequence
  source 1..22: mol_type = other RNA
gaggttacagttcctatcacat

SEQ ID NO: 660 - RNA, 22 nt, synthetic construct
  misc_feature 1..22: note = Guide sequence
  source 1..22: mol_type = other RNA
ctgtcacgggagccctgtggac

SEQ ID NO: 661 - RNA, 22 nt, synthetic construct
  misc_feature 1..22: note = Guide sequence
  source 1..22: mol_type = other RNA
gccttcttctccggggagcggt

SEQ ID NO: 662 - RNA, 22 nt, synthetic construct
  misc_feature 1..22: note = Guide sequence
  source 1..22: mol_type = other RNA
gggaattggccttggacagttc

SEQ ID NO: 663 - RNA, 22 nt, synthetic construct
  misc_feature 1..22: note = Guide sequence
  source 1..22: mol_type = other RNA
tcccgctttgctaccactttct

SEQ ID NO: 664 - RNA, 22 nt, synthetic construct
  misc_feature 1..22: note = Guide sequence
  source 1..22: mol_type = other RNA
gcttggtcatagcaaaagccgt

SEQ ID NO: 665 - RNA, 22 nt, synthetic construct
  misc_feature 1..22: note = Guide sequence
  source 1..22: mol_type = other RNA
tatgaccaagctgggtgcctgt

SEQ ID NO: 666 - RNA, 22 nt, synthetic construct
  misc_feature 1..22: note = Guide sequence
  source 1..22: mol_type = other RNA
tcagttgctggagggtgtcatt

SEQ ID NO: 667 - RNA, 22 nt, synthetic construct
  misc_feature 1..22: note = Guide sequence
  source 1..22: mol_type = other RNA
atgacaccctccagcaactgat

SEQ ID NO: 668 - RNA, 22 nt, synthetic construct
  misc_feature 1..22: note = Guide sequence
  source 1..22: mol_type = other RNA
ttgacttctataggtatttaag

SEQ ID NO: 669 - RNA, 22 nt, synthetic construct
  misc_feature 1..22: note = Guide sequence
  source 1..22: mol_type = other RNA
tttctcagatatggtgtcaaac

SEQ ID NO: 670 - RNA, 22 nt, synthetic construct
  misc_feature 1..22: note = Guide sequence
  source 1..22: mol_type = other RNA
tttgtctccaaaaaggcgattg

SEQ ID NO: 671 - RNA, 22 nt, synthetic construct
  misc_feature 1..22: note = Guide sequence
  source 1..22: mol_type = other RNA
gtccaggggctggagcttggct

SEQ ID NO: 672 - RNA, 22 nt, synthetic construct
  misc_feature 1..22: note = Guide sequence
  source 1..22: mol_type = other RNA
ggtgattcggccttcggtctta

SEQ ID NO: 673 - RNA, 22 nt, synthetic construct
  misc_feature 1..22: note = Guide sequence
  source 1..22: mol_type = other RNA
tgagctcactgttctggtgctg

SEQ ID NO: 674 - RNA, 22 nt, synthetic construct
  misc_feature 1..22: note = Guide sequence
  source 1..22: mol_type = other RNA
taccttgaagtaaatggtgtta

SEQ ID NO: 675 - RNA, 22 nt, synthetic construct
  misc_feature 1..22: note = Guide sequence
  source 1..22: mol_type = other RNA
tgctggttaacaccatttactt

SEQ ID NO: 676 - RNA, 22 nt, synthetic construct
  misc_feature 1..22: note = Guide sequence
  source 1..22: mol_type = other RNA
gtggaagtcaaagttcagccct

SEQ ID NO: 677 - RNA, 22 nt, synthetic construct
  misc_feature 1..22: note = Guide sequence
  source 1..22: mol_type = other RNA
ggagagtcgtgttcagcatcta

SEQ ID NO: 678 - RNA, 22 nt, synthetic construct
  misc_feature 1..22: note = Guide sequence
  source 1..22: mol_type = other RNA
ccttcctggtacatcatagatg

SEQ ID NO: 679 - RNA, 22 nt, synthetic construct
  misc_feature 1..22: note = Guide sequence
  source 1..22: mol_type = other RNA
cgccgataacggaacttgcctt

SEQ ID NO: 680 - RNA, 22 nt, synthetic construct
  misc_feature 1..22: note = Guide sequence
  source 1..22: mol_type = other RNA
gttccgttatcggcgcgtggct

SEQ ID NO: 681 - RNA, 22 nt, synthetic construct
  misc_feature 1..22: note = Guide sequence
  source 1..22: mol_type = other RNA
gtcatcacctttgaagggcaac

SEQ ID NO: 682 - RNA, 22 nt, synthetic construct
  misc_feature 1..22: note = Guide sequence
  source 1..22: mol_type = other RNA
ctccaattcatccagccactct

SEQ ID NO: 683 - RNA, 22 nt, synthetic construct
  misc_feature 1..22: note = Guide sequence
  source 1..22: mol_type = other RNA
agaggtcatctcggccttctgc

SEQ ID NO: 684 - RNA, 22 nt, synthetic construct
  misc_feature 1..22: note = Guide sequence
  source 1..22: mol_type = other RNA
attccataaggcatttcttgag

SEQ ID NO: 685 - RNA, 22 nt, synthetic construct
  misc_feature 1..22: note = Guide sequence
  source 1..22: mol_type = other RNA
tgaagaaggcagtgaagcagct

SEQ ID NO: 686 - RNA, 22 nt, synthetic construct
  misc_feature 1..22: note = Guide sequence
  source 1..22: mol_type = other RNA
gcgaacggccagcaatcacaac

SEQ ID NO: 687 - RNA, 22 nt, synthetic construct
  misc_feature 1..22: note = Guide sequence
  source 1..22: mol_type = other RNA
ggtttttataagagaagttcct

SEQ ID NO: 688 - RNA, 22 nt, synthetic construct
  misc_feature 1..22: note = Guide sequence
  source 1..22: mol_type = other RNA
tgcaaagaataagaacatttta

SEQ ID NO: 689 - RNA, 20 nt, synthetic construct
  misc_feature 1..20: note = Guide sequence
  source 1..20: mol_type = other RNA
tgaagaaagtacatgcactt

SEQ ID NO: 690 - RNA, 20 nt, synthetic construct
  misc_feature 1..20: note = Guide sequence
  source 1..20: mol_type = other RNA
gaagaaagtacatgcacttt

SEQ ID NO: 691 - RNA, 20 nt, synthetic construct
  misc_feature 1..20: note = Guide sequence
  source 1..20: mol_type = other RNA
caggggcaagattaagcagc

SEQ ID NO: 692 - RNA, 20 nt, synthetic construct
  misc_feature 1..20: note = Guide sequence
  source 1..20: mol_type = other RNA
atcagcattaagaggggcag

SEQ ID NO: 693 - RNA, 20 nt, synthetic construct
  misc_feature 1..20: note = Guide sequence
  source 1..20: mol_type = other RNA
aatcagcattaagaggggca

SEQ ID NO: 694 - RNA, 20 nt, synthetic construct
  misc_feature 1..20: note = Guide sequence
  source 1..20: mol_type = other RNA
gaatcagcattaagaggggc

SEQ ID NO: 695 - RNA, 20 nt, synthetic construct
  misc_feature 1..20: note = Guide sequence
  source 1..20: mol_type = other RNA
ctcagaatcagcattaagag

SEQ ID NO: 696 - RNA, 20 nt, synthetic construct
  misc_feature 1..20: note = Guide sequence
  source 1..20: mol_type = other RNA
cctcagaatcagcattaaga

SEQ ID NO: 697 - RNA, 20 nt, synthetic construct
  misc_feature 1..20: note = Guide sequence
  source 1..20: mol_type = other RNA
tcctcagaatcagcattaag

SEQ ID NO: 698 - RNA, 20 nt, synthetic construct
  misc_feature 1..20: note = Guide sequence
  source 1..20: mol_type = other RNA
ccctcttaatgctgattctg

SEQ ID NO: 699 - RNA, 20 nt, synthetic construct
  misc_feature 1..20: note = Guide sequence
  source 1..20: mol_type = other RNA
gaagaacacacaattatcac

SEQ ID NO: 700 - RNA, 20 nt, synthetic construct
  misc_feature 1..20: note = Guide sequence
  source 1..20: mol_type = other RNA
ttatttttactttatagata

SEQ ID NO: 701 - RNA, 20 nt, synthetic construct
  misc_feature 1..20: note = Guide sequence
  source 1..20: mol_type = other RNA
gaatgcataagtttcagtgg

SEQ ID NO: 702 - RNA, 20 nt, synthetic construct
  misc_feature 1..20: note = Guide sequence
  source 1..20: mol_type = other RNA
aatgaatgcataagtttcag

SEQ ID NO: 703 - RNA, 20 nt, synthetic construct
  misc_feature 1..20: note = Guide sequence
  source 1..20: mol_type = other RNA
gcattcattttgtgcattca

SEQ ID NO: 704 - RNA, 20 nt, synthetic construct
  misc_feature 1..20: note = Guide sequence
  source 1..20: mol_type = other RNA
ttcattttgtgcattcaagg

SEQ ID NO: 705 - RNA, 20 nt, synthetic construct
  misc_feature 1..20: note = Guide sequence
  source 1..20: mol_type = other RNA
tgtgcattcaaggcggatga

SEQ ID NO: 706 - RNA, 20 nt, synthetic construct
  misc_feature 1..20: note = Guide sequence
  source 1..20: mol_type = other RNA
ttttcatgattgctttacat

SEQ ID NO: 707 - RNA, 20 nt, synthetic construct
  misc_feature 1..20: note = Guide sequence
  source 1..20: mol_type = other RNA
cttttcatgattgctttaca

SEQ ID NO: 708 - RNA, 20 nt, synthetic construct
  misc_feature 1..20: note = Guide sequence
  source 1..20: mol_type = other RNA
cagtgcgaagaatttatata

SEQ ID NO: 709 - RNA, 20 nt, synthetic construct
  misc_feature 1..20: note = Guide sequence
  source 1..20: mol_type = other RNA
agtgcgaagaatttatatat

SEQ ID NO: 710 - RNA, 20 nt, synthetic construct
  misc_feature 1..20: note = Guide sequence
  source 1..20: mol_type = other RNA
gtgcgaagaatttatatatg

SEQ ID NO: 711 - RNA, 20 nt, synthetic construct
  misc_feature 1..20: note = Guide sequence
  source 1..20: mol_type = other RNA
tgcgaagaatttatatatgg

SEQ ID NO: 712 - RNA, 20 nt, synthetic construct
  misc_feature 1..20: note = Guide sequence
  source 1..20: mol_type = other RNA
tttatatatgggggatgtga

SEQ ID NO: 713 - RNA, 20 nt, synthetic construct
  misc_feature 1..20: note = Guide sequence
  source 1..20: mol_type = other RNA
tcagaatcgatttgaaagtc

SEQ ID NO: 714 - RNA, 20 nt, synthetic construct
  misc_feature 1..20: note = Guide sequence
  source 1..20: mol_type = other RNA
tgcaaaaaaatgtgtacaag

SEQ ID NO: 715 - RNA, 20 nt, synthetic construct
  misc_feature 1..20: note = Guide sequence
  source 1..20: mol_type = other RNA
aaaaaatgtgtacaagaggt

SEQ ID NO: 716 - RNA, 20 nt, synthetic construct
  misc_feature 1..20: note = Guide sequence
  source 1..20: mol_type = other RNA
tgattacagataatgcaaac

SEQ ID NO: 717 - RNA, 20 nt, synthetic construct
  misc_feature 1..20: note = Guide sequence
  source 1..20: mol_type = other RNA
ataaagacaacattgcaaca

SEQ ID NO: 718 - RNA, 20 nt, synthetic construct
  misc_feature 1..20: note = Guide sequence
  source 1..20: mol_type = other RNA
tcttccaaaaagcagaaatc

SEQ ID NO: 719 - RNA, 20 nt, synthetic construct
  misc_feature 1..20: note = Guide sequence
  source 1..20: mol_type = other RNA
aaagccagatttctgctttt

SEQ ID NO: 720 - RNA, 20 nt, synthetic construct
  misc_feature 1..20: note = Guide sequence
  source 1..20: mol_type = other RNA
tgctttttggaagaagatcc

SEQ ID NO: 721 - RNA, 20 nt, synthetic construct
  misc_feature 1..20: note = Guide sequence
  source 1..20: mol_type = other RNA
atataacctcgacatattcc

SEQ ID NO: 722 - RNA, 20 nt, synthetic construct
  misc_feature 1..20: note = Guide sequence
  source 1..20: mol_type = other RNA
gaagatcctggaatatgtcg

SEQ ID NO: 723 - RNA, 20 nt, synthetic construct
  misc_feature 1..20: note = Guide sequence
  source 1..20: mol_type = other RNA
tatgtcgaggttatattacc

SEQ ID NO: 724 - RNA, 20 nt, synthetic construct
  misc_feature 1..20: note = Guide sequence
  source 1..20: mol_type = other RNA
ctgattgttataaaaatacc

SEQ ID NO: 725 - RNA, 20 nt, synthetic construct
  misc_feature 1..20: note = Guide sequence
  source 1..20: mol_type = other RNA
cagtgtgaacgtttcaagta

SEQ ID NO: 726 - RNA, 20 nt, synthetic construct
  misc_feature 1..20: note = Guide sequence
  source 1..20: mol_type = other RNA
tgtgaacgtttcaagtatgg

SEQ ID NO: 727 - RNA, 20 nt, synthetic construct
  misc_feature 1..20: note = Guide sequence
  source 1..20: mol_type = other RNA
tttcaagtatggtggatgcc

SEQ ID NO: 728 - RNA, 20 nt, synthetic construct
  misc_feature 1..20: note = Guide sequence
  source 1..20: mol_type = other RNA
ttcaagtatggtggatgcct

SEQ ID NO: 729 - RNA, 20 nt, synthetic construct
  misc_feature 1..20: note = Guide sequence
  source 1..20: mol_type = other RNA
caaaattgttcatattgccc

SEQ ID NO: 730 - RNA, 20 nt, synthetic construct
  misc_feature 1..20: note = Guide sequence
  source 1..20: mol_type = other RNA
tatgaacaattttgagacac

SEQ ID NO: 731 - RNA, 20 nt, synthetic construct
  misc_feature 1..20: note = Guide sequence
  source 1..20: mol_type = other RNA
tgcaagaacatttgtgaaga

SEQ ID NO: 732 - RNA, 20 nt, synthetic construct
  misc_feature 1..20: note = Guide sequence
  source 1..20: mol_type = other RNA
ctgtatttttttccagcgaa

SEQ ID NO: 733 - RNA, 20 nt, synthetic construct
  misc_feature 1..20: note = Guide sequence
  source 1..20: mol_type = other RNA
tccacctggaaaccattcgc

SEQ ID NO: 734 - RNA, 20 nt, synthetic construct
  misc_feature 1..20: note = Guide sequence
  source 1..20: mol_type = other RNA
ttttccagcgaatggtttcc

SEQ ID NO: 735 - RNA, 20 nt, synthetic construct
  misc_feature 1..20: note = Guide sequence
  source 1..20: mol_type = other RNA
tccagcgaatggtttccagg

SEQ ID NO: 736 - RNA, 20 nt, synthetic construct
  misc_feature 1..20: note = Guide sequence
  source 1..20: mol_type = other RNA
gggttccataattatccacc

SEQ ID NO: 737 - RNA, 20 nt, synthetic construct
  misc_feature 1..20: note = Guide sequence
  source 1..20: mol_type = other RNA
ggtttccaggtggataatta

SEQ ID NO: 738 - RNA, 20 nt, synthetic construct
  misc_feature 1..20: note = Guide sequence
  source 1..20: mol_type = other RNA
gttattcacagcattgagct

SEQ ID NO: 739 - RNA, 20 nt, synthetic construct
  misc_feature 1..20: note = Guide sequence
  source 1..20: mol_type = other RNA
agttattcacagcattgagc

SEQ ID NO: 740 - RNA, 20 nt, synthetic construct
  misc_feature 1..20: note = Guide sequence
  source 1..20: mol_type = other RNA
cttggttgattgcggagtca

SEQ ID NO: 741 - RNA, 20 nt, synthetic construct
  misc_feature 1..20: note = Guide sequence
  source 1..20: mol_type = other RNA
ccttggttgattgcggagtc

SEQ ID NO: 742 - RNA, 20 nt, synthetic construct
  misc_feature 1..20: note = Guide sequence
  source 1..20: mol_type = other RNA
ctgggaaccttggttgattg

SEQ ID NO: 743 - RNA, 20 nt, synthetic construct
  misc_feature 1..20: note = Guide sequence
  source 1..20: mol_type = other RNA
cctgactccgcaatcaacca

SEQ ID NO: 744 - RNA, 20 nt, synthetic construct
  misc_feature 1..20: note = Guide sequence
  source 1..20: mol_type = other RNA
accaaaaaggctgggaacct

SEQ ID NO: 745 - RNA, 20 nt, synthetic construct
  misc_feature 1..20: note = Guide sequence
  source 1..20: mol_type = other RNA
agattcttaccaaaaaggct

SEQ ID NO: 746 - RNA, 20 nt, synthetic construct
  misc_feature 1..20: note = Guide sequence
  source 1..20: mol_type = other RNA
aagattcttaccaaaaaggc

SEQ ID NO: 747 - RNA, 20 nt, synthetic construct
  misc_feature 1..20: note = Guide sequence
  source 1..20: mol_type = other RNA
accaaggttcccagcctttt

SEQ ID NO: 748 - RNA, 20 nt, synthetic construct
  misc_feature 1..20: note = Guide sequence
  source 1..20: mol_type = other RNA
ccacaagattcttaccaaaa

SEQ ID NO: 749 - RNA, 20 nt, synthetic construct
  misc_feature 1..20: note = Guide sequence
  source 1..20: mol_type = other RNA
cctttttggtaagaatcttg

SEQ ID NO: 750 - RNA, 20 nt, synthetic construct
  misc_feature 1..20: note = Guide sequence
  source 1..20: mol_type = other RNA
gtgaaattctaaaaacaatc

SEQ ID NO: 751 - RNA, 20 nt, synthetic construct
  misc_feature 1..20: note = Guide sequence
  source 1..20: mol_type = other RNA
tgattgtttttagaatttca

SEQ ID NO: 752 - RNA, 20 nt, synthetic construct
  misc_feature 1..20: note = Guide sequence
  source 1..20: mol_type = other RNA
tagaatttcacggtccctca

SEQ ID NO: 753 - RNA, 20 nt, synthetic construct
  misc_feature 1..20: note = Guide sequence
  source 1..20: mol_type = other RNA
gctggagtgagacaccatga

SEQ ID NO: 754 - RNA, 20 nt, synthetic construct
  misc_feature 1..20: note = Guide sequence
  source 1..20: mol_type = other RNA
tgctggagtgagacaccatg

SEQ ID NO: 755 - RNA, 20 nt, synthetic construct
  misc_feature 1..20: note = Guide sequence
  source 1..20: mol_type = other RNA
cgacacaatcctctgtctgc

SEQ ID NO: 756 - RNA, 20 nt, synthetic construct
  misc_feature 1..20: note = Guide sequence
  source 1..20: mol_type = other RNA
tgtctcactccagcagacag

SEQ ID NO: 757 - RNA, 20 nt, synthetic construct
  misc_feature 1..20: note = Guide sequence
  source 1..20: mol_type = other RNA
gtagtagaatctgttctcat

SEQ ID NO: 758 - RNA, 20 nt, synthetic construct
  misc_feature 1..20: note = Guide sequence
  source 1..20: mol_type = other RNA
ttctactacaattcagtcat

SEQ ID NO: 759 - RNA, 20 nt, synthetic construct
  misc_feature 1..20: note = Guide sequence
  source 1..20: mol_type = other RNA
tctactacaattcagtcatt

SEQ ID NO: 760 - RNA, 20 nt, synthetic construct
  misc_feature 1..20: note = Guide sequence
  source 1..20: mol_type = other RNA
atccactgtacttaaatggg

SEQ ID NO: 761 - RNA, 20 nt, synthetic construct
  misc_feature 1..20: note = Guide sequence
  source 1..20: mol_type = other RNA
cacatccactgtacttaaat

SEQ ID NO: 762 - RNA, 20 nt, synthetic construct
  misc_feature 1..20: note = Guide sequence
  source 1..20: mol_type = other RNA
ccacatccactgtacttaaa

SEQ ID NO: 763 - RNA, 20 nt, synthetic construct
  misc_feature 1..20: note = Guide sequence
  source 1..20: mol_type = other RNA
tgccgcccatttaagtacag

SEQ ID NO: 764 - RNA, 20 nt, synthetic construct
  misc_feature 1..20: note = Guide sequence
  source 1..20: mol_type = other RNA
ccatttaagtacagtggatg

SEQ ID NO: 765 - RNA, 20 nt, synthetic construct
  misc_feature 1..20: note = Guide sequence
  source 1..20: mol_type = other RNA
catttaagtacagtggatgt

SEQ ID NO: 766 - RNA, 20 nt, synthetic construct
  misc_feature 1..20: note = Guide sequence
  source 1..20: mol_type = other RNA
atttaagtacagtggatgtg

SEQ ID NO: 767 - RNA, 20 nt, synthetic construct
  misc_feature 1..20: note = Guide sequence
  source 1..20: mol_type = other RNA
tttaagtacagtggatgtgg

SEQ ID NO: 768 - RNA, 20 nt, synthetic construct
  misc_feature 1..20: note = Guide sequence
  source 1..20: mol_type = other RNA
tgccctcagacattcttgtt

SEQ ID NO: 769 - RNA, 20 nt, synthetic construct
  misc_feature 1..20: note = Guide sequence
  source 1..20: mol_type = other RNA
cttccaaacaagaatgtctg

SEQ ID NO: 770 - RNA, 20 nt, synthetic construct
  misc_feature 1..20: note = Guide sequence
  source 1..20: mol_type = other RNA
ttccaaacaagaatgtctga

SEQ ID NO: 771 - RNA, 20 nt, synthetic construct
  misc_feature 1..20: note = Guide sequence
  source 1..20: mol_type = other RNA
tgtctgagggcatgtaaaaa

SEQ ID NO: 772 - RNA, 20 nt, synthetic construct
  misc_feature 1..20: note = Guide sequence
  source 1..20: mol_type = other RNA
atgaaacctataagaggaag

SEQ ID NO: 773 - RNA, 20 nt, synthetic construct
  misc_feature 1..20: note = Guide sequence
  source 1..20: mol_type = other RNA
ctttggatgaaacctataag

SEQ ID NO: 774 - RNA, 20 nt, synthetic construct
  misc_feature 1..20: note = Guide sequence
  source 1..20: mol_type = other RNA
ggcctccttttgatattctt

SEQ ID NO: 775 - RNA, 20 nt, synthetic construct
  misc_feature 1..20: note = Guide sequence
  source 1..20: mol_type = other RNA
ttcatccaaagaatatcaaa

SEQ ID NO: 776 - RNA, 20 nt, synthetic construct
  misc_feature 1..20: note = Guide sequence
  source 1..20: mol_type = other RNA
atccaaagaatatcaaaagg

SEQ ID NO: 777 - RNA, 20 nt, synthetic construct
  misc_feature 1..20: note = Guide sequence
  source 1..20: mol_type = other RNA
tttttcttttggttttaatt

SEQ ID NO: 778 - RNA, 20 nt, synthetic construct
  misc_feature 1..20: note = Guide sequence
  source 1..20: mol_type = other RNA
ctgcttctttctttttcttt

SEQ ID NO: 779 - RNA, 20 nt, synthetic construct
  misc_feature 1..20: note = Guide sequence
  source 1..20: mol_type = other RNA
tgtgtgttcttcatcttcct

SEQ ID NO: 780 - RNA, 20 nt, synthetic construct
  misc_feature 1..20: note = Guide sequence
  source 1..20: mol_type = other RNA
ttatttttactttatagata

SEQ ID NO: 781 - RNA, 20 nt, synthetic construct
  misc_feature 1..20: note = Guide sequence
  source 1..20: mol_type = other RNA
atccgccttgaatgcacaaa

SEQ ID NO: 782 - RNA, 20 nt, synthetic construct
  misc_feature 1..20: note = Guide sequence
  source 1..20: mol_type = other RNA
attcattttgtgcattcaag

SEQ ID NO: 783 - RNA, 20 nt, synthetic construct
  misc_feature 1..20: note = Guide sequence
  source 1..20: mol_type = other RNA
tacatgggccatcatccgcc

SEQ ID NO: 784 - RNA, 20 nt, synthetic construct
  misc_feature 1..20: note = Guide sequence
  source 1..20: mol_type = other RNA
tatataaattcttcgcactg

SEQ ID NO: 785 - RNA, 20 nt, synthetic construct
  misc_feature 1..20: note = Guide sequence
  source 1..20: mol_type = other RNA
tattttcactcgacagtgcg

SEQ ID NO: 786 - RNA, 20 nt, synthetic construct
  misc_feature 1..20: note = Guide sequence
  source 1..20: mol_type = other RNA
gtgcgaagaatttatatatg

SEQ ID NO: 787 - RNA, 20 nt, synthetic construct
  misc_feature 1..20: note = Guide sequence
  source 1..20: mol_type = other RNA
atgggggatgtgaaggaaat

SEQ ID NO: 788 - RNA, 20 nt, synthetic construct
  misc_feature 1..20: note = Guide sequence
  source 1..20: mol_type = other RNA
gaatcgatttgaaagtctgg

SEQ ID NO: 789 - RNA, 20 nt, synthetic construct
  misc_feature 1..20: note = Guide sequence
  source 1..20: mol_type = other RNA
ttgattacagataatgcaaa

SEQ ID NO: 790 - RNA, 20 nt, synthetic construct
  misc_feature 1..20: note = Guide sequence
  source 1..20: mol_type = other RNA
tgctttttggaagaagatcc

SEQ ID NO: 791 - RNA, 20 nt, synthetic construct
  misc_feature 1..20: note = Guide sequence
  source 1..20: mol_type = other RNA
aatataacctcgacatattc

SEQ ID NO: 792 - RNA, 20 nt, synthetic construct
  misc_feature 1..20: note = Guide sequence
  source 1..20: mol_type = other RNA
gtgtgaacgtttcaagtatg

SEQ ID NO: 793 - RNA, 20 nt, synthetic construct
  misc_feature 1..20: note = Guide sequence
  source 1..20: mol_type = other RNA
gaacaattttgagacactgg

SEQ ID NO: 794 - RNA, 20 nt, synthetic construct
  misc_feature 1..20: note = Guide sequence
  source 1..20: mol_type = other RNA
ttccagcgaatggtttccag

SEQ ID NO: 795 - RNA, 20 nt, synthetic construct
  misc_feature 1..20: note = Guide sequence
  source 1..20: mol_type = other RNA
gagttattcacagcattgag

SEQ ID NO: 796 - RNA, 20 nt, synthetic construct
  misc_feature 1..20: note = Guide sequence
  source 1..20: mol_type = other RNA
atggaacccagctcaatgct

SEQ ID NO: 797 - RNA, 20 nt, synthetic construct
  misc_feature 1..20: note = Guide sequence
  source 1..20: mol_type = other RNA
cttggttgattgcggagtca

SEQ ID NO: 798 - RNA, 20 nt, synthetic construct
  misc_feature 1..20: note = Guide sequence
  source 1..20: mol_type = other RNA
ctgggaaccttggttgattg

SEQ ID NO: 799 - RNA, 20 nt, synthetic construct
  misc_feature 1..20: note = Guide sequence
  source 1..20: mol_type = other RNA
aggttcccagcctttttggt

SEQ ID NO: 800 - RNA, 20 nt, synthetic construct
  misc_feature 1..20: note = Guide sequence
  source 1..20: mol_type = other RNA
cgacacaatcctctgtctgc

SEQ ID NO: 801 - RNA, 20 nt, synthetic construct
  misc_feature 1..20: note = Guide sequence
  source 1..20: mol_type = other RNA
gtgtctcactccagcagaca

SEQ ID NO: 802 - RNA, 20 nt, synthetic construct
  misc_feature 1..20: note = Guide sequence
  source 1..20: mol_type = other RNA
tcccaatgactgaattgtag

SEQ ID NO: 803 - RNA, 20 nt, synthetic construct
  misc_feature 1..20: note = Guide sequence
  source 1..20: mol_type = other RNA
tgggcggcatttcccaatga

SEQ ID NO: 804 - RNA, 20 nt, synthetic construct
  misc_feature 1..20: note = Guide sequence
  source 1..20: mol_type = other RNA
atgccgcccatttaagtaca

SEQ ID NO: 805 - RNA, 20 nt, synthetic construct
  misc_feature 1..20: note = Guide sequence
  source 1..20: mol_type = other RNA
aaacaattttacttccaaac

SEQ ID NO: 806 - RNA, 20 nt, synthetic construct
  misc_feature 1..20: note = Guide sequence
  source 1..20: mol_type = other RNA
cctcttataggtttcatcca

SEQ ID NO: 807 - RNA, 20 nt, synthetic construct
  misc_feature 1..20: note = Guide sequence
  source 1..20: mol_type = other RNA
aggcctccttttgatattct

SEQ ID NO: 808 - RNA, 20 nt, synthetic construct
  misc_feature 1..20: note = Guide sequence
  source 1..20: mol_type = other RNA
gaaatttttgttaaaaatat

SEQ ID NO: 809 - RNA, 22 nt, synthetic construct
  misc_feature 1..22: note = Guide sequence
  source 1..22: mol_type = other RNA
tgggttctgtatttcagagatg

SEQ ID NO: 810 - RNA, 22 nt, synthetic construct
  misc_feature 1..22: note = Guide sequence
  source 1..22: mol_type = other RNA
tcttcattgtgtaaatcatctc

SEQ ID NO: 811 - RNA, 22 nt, synthetic construct
  misc_feature 1..22: note = Guide sequence
  source 1..22: mol_type = other RNA
cagagatgatttacacaatgaa

SEQ ID NO: 812 - RNA, 22 nt, synthetic construct
  misc_feature 1..22: note = Guide sequence
  source 1..22: mol_type = other RNA
tgatttacacaatgaagaaagt

SEQ ID NO: 813 - RNA, 22 nt, synthetic construct
  misc_feature 1..22: note = Guide sequence
  source 1..22: mol_type = other RNA
caggcatacagaagcccaaagt

SEQ ID NO: 814 - RNA, 22 nt, synthetic construct
  misc_feature 1..22: note = Guide sequence
  source 1..22: mol_type = other RNA
ggcaggggcaagattaagcagc

SEQ ID NO: 815 - RNA, 22 nt, synthetic construct
  misc_feature 1..22: note = Guide sequence
  source 1..22: mol_type = other RNA
aatgctgattctgaggaagatg

SEQ ID NO: 816 - RNA, 22 nt, synthetic construct
  misc_feature 1..22: note = Guide sequence
  source 1..22: mol_type = other RNA
tgctgattctgaggaagatgaa

SEQ ID NO: 817 - RNA, 22 nt, synthetic construct
  misc_feature 1..22: note = Guide sequence
  source 1..22: mol_type = other RNA
tacatgggccatcatccgcctt

SEQ ID NO: 818 - RNA, 22 nt, synthetic construct
  misc_feature 1..22: note = Guide sequence
  source 1..22: mol_type = other RNA
tcacatcccccatatataaatt

SEQ ID NO: 819 - RNA, 22 nt, synthetic construct
  misc_feature 1..22: note = Guide sequence
  source 1..22: mol_type = other RNA
aagtctggaagagtgcaaaaaa

SEQ ID NO: 820 - RNA, 22 nt, synthetic construct
  misc_feature 1..22: note = Guide sequence
  source 1..22: mol_type = other RNA
aacctacctcttgtacacattt

SEQ ID NO: 821 - RNA, 22 nt, synthetic construct
  misc_feature 1..22: note = Guide sequence
  source 1..22: mol_type = other RNA
agggttcccagaaacctacctc

SEQ ID NO: 822 - RNA, 22 nt, synthetic construct
  misc_feature 1..22: note = Guide sequence
  source 1..22: mol_type = other RNA
cactgttttgtctgattgttat

SEQ ID NO: 823 - RNA, 22 nt, synthetic construct
  misc_feature 1..22: note = Guide sequence
  source 1..22: mol_type = other RNA
aggcatccaccatacttgaaac

SEQ ID NO: 824 - RNA, 22 nt, synthetic construct
  misc_feature 1..22: note = Guide sequence
  source 1..22: mol_type = other RNA
ttgttcatattgcccaggcatc

SEQ ID NO: 825 - RNA, 22 nt, synthetic construct
  misc_feature 1..22: note = Guide sequence
  source 1..22: mol_type = other RNA
gcctgggcaatatgaacaattt

SEQ ID NO: 826 - RNA, 22 nt, synthetic construct
  misc_feature 1..22: note = Guide sequence
  source 1..22: mol_type = other RNA
cacaatcctctgtctgctggag

SEQ ID NO: 827 - RNA, 22 nt, synthetic construct
  misc_feature 1..22: note = Guide sequence
  source 1..22: mol_type = other RNA
tagtagaatctgttctcattgg

SEQ ID NO: 828 - RNA, 22 nt, synthetic construct
  misc_feature 1..22: note = Guide sequence
  source 1..22: mol_type = other RNA
aattgtagtagaatctgttctc

SEQ ID NO: 829 - RNA, 22 nt, synthetic construct
  misc_feature 1..22: note = Guide sequence
  source 1..22: mol_type = other RNA
gtcattgggaaatgccgcccat

SEQ ID NO: 830 - RNA, 22 nt, synthetic construct
  misc_feature 1..22: note = Guide sequence
  source 1..22: mol_type = other RNA
ttgttttcatttcccccacatc

SEQ ID NO: 831 - protein, 7 aa, synthetic construct
  REGION 1..7: note = Nuclear localization sequence
PKKKRKV

SEQ ID NO: 832 - protein, 16 aa, synthetic construct
  REGION 1..16: note = Nuclear localization sequence
KRPAATKKAGQAKKKK

SEQ ID NO: 833 - protein, 9 aa, synthetic construct
  REGION 1..9: note = Nuclear localization sequence
PAAKRVKLD

SEQ ID NO: 834 - protein, 11 aa, synthetic construct
  REGION 1..11: note = Nuclear localization sequence
RQRRNELKRSP

SEQ ID NO: 835 - protein, 38 aa, synthetic construct
  REGION 1..38: note = Nuclear localization sequence
NQSSNFGPMKGGNFGGRSSGPYGGGGQYFAKPRNQGGY

SEQ ID NO: 836 - protein, 42 aa, synthetic construct
  REGION 1..42: note = Nuclear localization sequence
RMRIZFKNKGKDTAELRRRRVEVSVELRKAKKDEQILKRRNV

SEQ ID NO: 837 - protein, 8 aa, synthetic construct
  REGION 1..8: note = Nuclear localization sequence
VSRKRPRP

SEQ ID NO: 838 - protein, 8 aa, synthetic construct
  REGION 1..8: note = Nuclear localization sequence
PPKKARED

SEQ ID NO: 839 - protein, 8 aa, synthetic construct
  REGION 1..8: note = Nuclear localization sequence
PQPKKKPL

SEQ ID NO: 840 - protein, 12 aa, synthetic construct
  REGION 1..12: note = Nuclear localization sequence
SALIKKKKKMAP

SEQ ID NO: 841 - protein, 5 aa, synthetic construct
  REGION 1..5: note = Nuclear localization sequence
DRLRR

SEQ ID NO: 842 - protein, 7 aa, synthetic construct
  REGION 1..7: note = Nuclear localization sequence
PKQKKRK

SEQ ID NO: 843 - protein, 10 aa, synthetic construct
  REGION 1..10: note = Nuclear localization sequence
RKLKKKIKKL

SEQ ID NO: 844 - protein, 10 aa, synthetic construct
  REGION 1..10: note = Nuclear localization sequence
REKKKFLKRR

SEQ ID NO: 845 - protein, 20 aa, synthetic construct
  REGION 1..20: note = Nuclear localization sequence
KRKGDEVDGVDEVAKKKSKK

SEQ ID NO: 846 - protein, 17 aa, synthetic construct
  REGION 1..17: note = Nuclear localization sequence
RKCLQAGMNLEARKTKK

SEQ ID NO: 847 - DNA, 20 bp, Homo sapiens
  source 1..20: mol_type = genomic DNA
gtccatctcctccgccagcg

SEQ ID NO: 848 - DNA, 22 bp, Homo sapiens
  source 1..22: mol_type = genomic DNA
gagagggccagaaatcacccaa

(19) United States
(12) Patent Application Publication
(10) Pub. No.: US 2023/0190889 A1
(43) Pub. Date: Jun. 22, 2023

(54) GENE EDITING OF ANTICOAGULANT FACTORS

(71) Applicant: TOOLGEN INCORPORATED, SEOUL (KR)

(72) Inventors: DONG WOO SONG, SEOUL (KR); BEOMSEOK CHOI, DONGJAK-GU (KR); HYEKYUNG OH, GANGDONG-GU (KR); NANYEONG GO, GYEONGGI-DO (KR); HYERIM LEE, SEOUL (KR); KYU JUN LEE, SEOUL (KR); UN GI KIM, SEOUL (KR); JAEYOUNG LEE, SEOUL (KR); JUNG MIN LEE, GYEONGSANGBUK-DO (KR)

(21) Appl. No.: 18/157,329

(22) Filed: Jan. 20, 2023

Related U.S. Application Data
(63) Continuation-in-part of application No. 17/262,342, filed on Jan. 22, 2021.
(60) Provisional application No. 62/703,578, filed on Jul. 26, 2018.

(30) Foreign Application Priority Data
Jan. 20, 2022 (KR) 10-2022-0008819

(51) Int. Cl. A61K 38/46; C12N 9/22; C12N 15/11; C12N 15/86; A61K 31/7105; A61P 7/04

(52) U.S. Cl. CPC A61K 38/465; A61K 31/7105; A61P 7/04; C12N 9/22; C12N 15/11; C12N 15/86; C12N 2310/20; C12N 2750/14143; C12N 2750/14171; C12N 2800/80

(57) ABSTRACT

The present disclosure relates to a composition for gene manipulation for artificially manipulating a blood coagulation inhibitory gene present in the genome of a cell to regulate a blood coagulation system. More particularly, the present invention relates to a composition for gene manipulation, which includes a guide nucleic acid capable of targeting a blood coagulation inhibitory gene, and an editor protein. Also, the present invention relates to a method of treating or improving coagulopathy using the composition for gene manipulation for artificially manipulating a blood coagulation inhibitory gene.

### CROSS-REFERENCE TO RELATED APPLICATIONS

This application is a continuation-in-part of U.S. Pat. Application No. 17/262,342, filed Jan. 22, 2021, which claims the benefit of and priority to PCT Application No. PCT/KR2019/009238, filed Jul. 25, 2019, which claims the benefit of and priority to U.S. Provisional Application No. 62/703578, filed Jul. 26, 2018 and also claims priority to Korean Patent Application No. 10-2022-0008819, filed Jan. 20, 2022. The entire disclosures of the above applications are incorporated herein by references.

### FIELD

The present disclosure relates to artificial manipulation or modification of a blood coagulation inhibitory gene for a normal blood coagulation system. More particularly, the present invention relates to a system capable of artificially regulating blood coagulation, which includes a composition capable of artificially manipulating a blood coagulation inhibitory gene to activate an abnormal blood coagulation system, that is, an inactivated blood coagulation system.

### BACKGROUND

Hemophilia is a hemorrhagic disease caused by the deficiency of blood coagulation factors. Coagulation factors in blood consists of factors I to XIII, and hemophilia is caused by the genetic defects of the corresponding factors. Hemophilia A is caused by the deficiency of factor VIII and is known to affect about one in every 5000 male babies. Hemophilia B is caused by the deficiency of factor IX and is known to affect about one in every 20,000 male babies. Hemophilia patients are estimated to exceed approximately 700,000 all over the world.

Therapies known so far are mainly to continuously administer the deficient factors, and there are no basic therapeutic methods.

Therefore, there is a need for a therapeutic agent capable of achieving a long-term effect in a single therapy, which can essentially knock out the expression of proteins that inactivate a blood coagulation system using the gene editing.

### SUMMARY

### Technical Problem

One embodiment of the present invention is to provide a method of treating coagulopathy.

Another embodiment of the present invention is to provide a composition for gene manipulation.

Still another embodiment of the present invention is to provide a guide nucleic acid capable of targeting a blood coagulation inhibitory gene.

### Technical Solution

To solve the above problems, the present invention provides a composition for gene manipulation for artificially manipulating a blood coagulation inhibitory gene present in the genome of a cell to regulate a blood coagulation system. More particularly, the present invention provides a composition for gene manipulation, which includes a guide nucleic acid capable of targeting a blood coagulation inhibitory gene, and an editor protein. Also, the present invention provides a method of treating or improving coagulopathy using the composition for gene manipulation for artificially manipulating a blood coagulation inhibitory gene.

To treat hemophilia, the present invention provides a method of treating a hemophilia including administration(introduction) of a composition for gene manipulation into a subject to be treated.

In one embodiment, the method of treating a hemophilia may include an introduction (administration) of a composition for gene manipulation into a subject to be treated.

The composition for gene manipulation may include the following:
a guide nucleic acid including a guide sequence forming a complementary bond with a target sequence located in a blood coagulation inhibitory gene, or a nucleic acid sequence encoding the same; and
an editor protein, or a nucleic acid sequence encoding the same,

The blood coagulation inhibitory gene may be an AT(antithrombin) gene or TFPI(tissue factor pathway inhibitor) gene.

The guide sequence may be one or more guide sequences selected from a SEC ID NO:425 to 830.

The complementary bond may include mismatching bonds of 0 to 5.

In one embodiment, the guide sequence may be one or more sequences selected from a SEQ ID NO : 427, 428, 436, 437, 443, 444, 447, 448, 449, 454, 458, 460, 461, 463, 464, 466, 467, 469, 473, 474, 622, 623, 624, 625, 626, 627, 628, 630, 632, 634, 635, 638, 639, 641, 642, 659, 660, 661, 662, 663, 664, 665, 666, 667, 668, 669, 670, 671, 672, 673, 674, 675, 676, 677, 678, 679, 680, 681, 682, 683, 684, 686, 687 and 688.

In one embodiment, the guide sequence may be one or more sequences selected from a SEQ ID NO : 692, 694, 705, 709, 710, 721, 726, 731, 733, 735, 740, 741, 742, 748, 781, 783, 786, 788, 791, 792, 794, 795, 796, 797, 798, 800, 802, 803, 804, 809, 810, 811, 813, 814, 815, 816, 817, 818, 819, 820, 821, 822, 823, 824, 825, 826, 827, 828, 829 and 830.

The guide sequence may form a complementary bond with a target sequence located in exon 1, exon 2, exon 3, exon 4, exon 5, exon 6 or exon 7 of AT gene.

The guide sequence may form a complementary bond with a target sequence located in exon 2, exon 3, exon 5, exon 6 or exon 7 of TFPI gene.

The editor protein may be a Streptococcus pyogenes-derived Cas9 protein, a Staphylococcus aureus-derived Cas9 protein, or a Campylobacter jejuni-derived Cas9 protein.

The composition for gene manipulation may be a form of guide nucleic acid-editor protein complex combined guide nucleic acid and editor protein.

Here, the guide nucleic acid may be a guide RNA(gRNA).

The guide nucleic acid and the editor protein may be present in one or more vectors in a form of a nucleic acid sequence, respectively.

Here, the vector may be a plasmid or viral vector.

Here, the viral vector may be one or more viral vectors selected from the group consisting of a retrovirus, a lentivirus, an adenovirus, an adeno-associated virus (AAV), a vaccinia virus, a poxvirus and a herpes simplex virus.

The composition for gene manipulation may optionally further include a donor including a nucleic acid sequence to be inserted, or a nucleic acid sequence encoding the same.

Here, the nucleic acid sequence to be inserted may be a partial nucleic acid sequence of blood coagulation inhibitory gene.

Here, the nucleic acid sequence to be inserted may be a complete or partial sequence of a gene encoding a blood coagulation associated protein.

For example, the blood coagulation associated protein may be one or more proteins selected from the group consisting of a factor XII, factor XIIa, factor XI, factor XIa, factor IX, factor IXa, factor X, factor Xa, factor VIII, factor VIIIa, factor VII, factor VIIa, factor V, factor Va, prothrombin, thrombin, factor XIII, factor XIIIa, fibrinogen, fibrin and tissue factor.

The guide nucleic acid, the editor protein and donor may be present in one or more vectors in a form of a nucleic acid sequence, respectively.

Here, the vector may be a plasmid or viral vector.

Here, the viral vector may be one or more viral vectors selected from the group consisting of a retrovirus, a lentivirus, an adenovirus, an adeno-associated virus (AAV), a vaccinia virus, a poxvirus and a herpes simplex virus.

The administration(introduction) may be performed by injection, transfusion, implantation or transplantation.

The administration(introduction) may be performed via an administration route selected from intrahepatic, subcutaneous, intradermal, intraocular, intravitreal, intratumoral, intranodal, intramedullary, intramuscular, intravenous, intralymphatic and intraperitoneal route.

The hemophilia may be a hemophilia A, hemophilia B or hemophilia C.

The subject to be treated is a mammal including a human, a monkey, a mouse and a rat.

Alternatively, the present invention provides a composition for gene manipulation for a specific purpose.

In one embodiment, the composition for gene manipulation may include the following:
a guide nucleic acid including a guide sequence forming a complementary bond with a target sequence located in blood coagulation inhibitory gene, or a nucleic acid sequence encoding the same; and
an editor protein, or a nucleic acid sequence encoding the same,

The blood coagulation inhibitory gene may be an AT(antithrombin) gene or TFPI(tissue factor pathway inhibition) gene.

The guide sequence may be one or more guide sequences selected from a SEQ ID NO:425 to 830.

The complementary bond may include mismatching bonds of 0 to 5.

The target sequence may be one or more sequences selected from a SEQ ID NO:19 to 424.

In one embodiment, the target sequence may be one or more sequences selected from a SEQ ID NO: 21, 22, 30, 31, 37, 38, 41, 42, 43, 48, 52, 54, 55, 57, 58, 60, 61, 63, 67, 68, 216, 217, 218, 219, 220, 221, 222, 224, 226, 228, 229, 232, 233, 235, 236, 253, 254, 255, 256, 257, 258, 259, 260, 261, 262, 263, 264, 265, 266, 267, 268, 269, 270, 271, 272, 273, 274, 275, 276, 277, 278, 280, 281 and 282.

In one embodiment, the target sequence may be one or more sequences selected from a SEQ ID NO: 286, 288, 299, 303, 304, 315, 320, 325, 327, 329, 334, 335, 336, 342, 375, 377, 380, 382, 385, 386, 388, 389, 390, 391, 392, 394, 396, 397, 398, 403, 404, 405, 407, 408, 409, 410, 411, 412, 413, 414, 415, 416, 417, 418, 419, 420, 421, 422, 423 and 424.

The guide sequence may be one or more sequences selected from a SEQ ID NO: 427, 428, 436, 437, 443, 444, 447, 448, 449, 454, 458, 460, 461, 463, 464, 466, 467, 469, 473, 474, 622, 623, 624, 625, 626, 627, 628, 630, 632, 634, 635, 638, 639, 641, 642, 659, 660, 661, 662, 663, 664, 665, 666, 667, 668, 669, 670, 671, 672, 673, 674, 675, 676, 677, 678, 679, 680, 681, 682, 683, 684, 686, 687 and 688.

The guide sequence may be one or more sequences selected from a SEQ ID NO: 692, 694, 705, 709, 710, 721, 726, 731, 733, 735, 740, 741, 742, 748, 781, 783, 786, 788, 791, 792, 794, 795, 796, 797, 798, 800, 802, 803, 804, 809, 810, 811, 813, 814, 815, 816, 817, 818, 819, 820, 821, 822, 823, 824, 825, 826, 827, 828, 829 and 830.

The guide sequence may form a complementary bond with a target sequence located in exon 1, exon 2, exon 3, exon 4, exon 5, exon 6 or exon 7 of AT gene.

The guide sequence may form a complementary bond with a target sequence located in exon 2, exon 3, exon 5, exon 6 or exon 7 of TFPI gene.

The guide nucleic acid may include a guide domain.

Here, the guide sequence may be included in the guide domain.

The guide nucleic acid may include one or more domains selected from a first complementary domain, a second complementary domain, a linker domain, a proximal domain and a tail domain.

The editor protein may be a Streptococcus pyogenes-derived Cas9 protein, a Staphylococcus aureus-derived Cas9 protein, or a Campylobacter jejuni-derived Cas9 protein.

The composition for gene manipulation may be a form of guide nucleic acid-editor protein complex combined guide nucleic acid and editor protein.

Here, the guide nucleic acid may be a guide RNA(gRNA).

The guide nucleic acid and the editor protein may be present in one or more vectors in a form of a nucleic acid sequence, respectively.

Here, the vector may be a plasmid or viral vector.

Here, the viral vector may be one or more viral vectors selected from the group consisting of a retrovirus, a lentivirus, an adenovirus, an adeno-associated virus (AAV), a vaccinia virus, a poxvirus and a herpes simplex virus.

The composition for gene manipulation may optionally further include a donor including a nucleic acid sequence to be inserted, or a nucleic acid sequence encoding the same.

Here, the nucleic acid sequence to be inserted may be a partial nucleic acid sequence of blood coagulation inhibitory gene.

Here, the nucleic acid sequence to be inserted may be a complete or partial sequence of a gene encoding a blood coagulation associated protein.

For example, the blood coagulation associated protein may be one or more proteins selected from the group consisting of a factor XII, factor XIIa, factor XI, factor XIa, factor IX, factor IXa, factor X, factor Xa, factor VIII, factor VIIIa, factor VII, factor VIIa, factor V, factor Va, prothrombin, thrombin, factor XIII, factor XIIIa, fibrinogen, fibrin and tissue factor.

The guide nucleic acid, the editor protein and donor may be present in one or more vectors in a form of a nucleic acid sequence, respectively.

Here, the vector may be a plasmid or viral vector.

Here, the viral vector may be one or more viral vectors selected from the group consisting of a retrovirus, a lentivirus, an adenovirus, an adeno-associated virus (AAV), a vaccinia virus, a poxvirus and a herpes simplex virus.

The present invention provides a guide nucleic acid capable of targeting a blood coagulation inhibitory gene for a specific purpose.

In one embodiment, the guide nucleic acid may include a guide sequence forming a complementary bond with a target sequence located in blood coagulation inhibitory gene.

The blood coagulation inhibitory gene may be an AT(antithrombin) gene or TFPI(tissue factor pathway inhibition) gene.

The guide sequence may be one or more guide sequences selected from a SEQ ID NO:425 to 830

The complementary bond may include mismatching bonds of 0 to 5.

The guide nucleic acid may form a complex with an editor protein.

In one embodiment, the guide sequence may be one or more sequences selected from a SEQ ID NO: 427, 428, 436, 437, 443, 444, 447, 448, 449, 454, 458, 460, 461, 463, 464, 466, 467, 469, 473, 474, 622, 623, 624, 625, 626, 627, 628, 630, 632, 634, 635, 638, 639, 641, 642, 659, 660, 661, 662, 663, 664, 665, 666, 667, 668, 669, 670, 671, 672, 673, 674, 675, 676, 677, 678, 679, 680, 681, 682, 683, 684, 686, 687 and 688.

In one embodiment, the guide sequence may be one or more sequences selected from a SEQ ID NO: 692, 694, 705, 709, 710, 721, 726, 731, 733, 735, 740, 741, 742, 748, 781, 783, 786, 788, 791, 792, 794, 795, 796, 797, 798, 800, 802, 803, 804, 809, 810, 811, 813, 814, 815, 816, 817, 818, 819, 820, 821, 822, 823, 824, 825, 826, 827, 828, 829 and 830.

The guide sequence may form a complementary bond with a target sequence located in exon 1, exon 2, exon 3, exon 4, exon 5, exon 6 or exon 7 of AT gene

The guide sequence may form a complementary bond with a target sequence located in exon 2, exon 3, exon 5, exon 6 or exon 7 of TFPI gene.

The guide nucleic acid may include a guide domain

Here, the guide sequence may be included in the guide domain.

The guide nucleic acid may include one or more domains selected from a first complementary domain, a second complementary domain, a linker domain, a proximal domain and a tail domain.

### Advantageous Effects

According to the present invention, a blood coagulation system can be regulated using a composition for gene manipulation. More particularly, the blood coagulation system can be regulated using the composition for gene manipulation, which includes a guide nucleic acid targeting a blood coagulation inhibitory gene, and an editor protein, by artificially manipulating and/or modifying the blood coagulation inhibitory gene to regulate the function and/or expression of the blood coagulation inhibitory gene. Also, coagulopathy can be treated or improved using the composition for gene manipulation for artificially manipulating a blood coagulation inhibitory gene.

### DETAILED DESCRIPTION

Unless defined otherwise, all technical and scientific terms used herein have the same meaning as commonly understood by those of ordinary skill in the art to which the present invention belongs. Methods and materials similar or identical to those described herein can be used in practice or testing of the present invention. All publications, patent applications, patents and other references mentioned herein are incorporated by reference in their entirety. In addition, materials, methods and examples are merely illustrative, and not intended to be limitive.

One aspect of the disclosure of the present specification relates to a guide nucleic acid.

The “guide nucleic acid” refers to a nucleotide sequence that recognizes a target nucleic acid, gene or chromosome, and interacts with an editor protein. Here, the guide nucleic acid may complementarily bind to a partial nucleotide sequence in the target nucleic acid, gene or chromosome. In addition, a partial nucleotide sequence of the guide nucleic acid may interact with some amino acids of the editor protein, thereby forming a guide nucleic acid-editor protein complex.

The guide nucleic acid may perform a function to induce a guide nucleic acid-editor protein complex to be located in a target region of a target nucleic acid, gene or chromosome.

The guide nucleic acid may be present in the form of DNA, RNA or a DNA/RNA hybrid, and may have a nucleic acid sequence of 5 to 150 nt.

The guide nucleic acid may have one continuous nucleic acid sequence.

For example, the one continuous nucleic acid sequence may be (N)m, where N represents A, T, C or G, or A, U, C or G, and m is an integer of 1 to 150.

The guide nucleic acid may have two or more continuous nucleic acid sequences.

For example, the two or more continuous nucleic acid sequences may be (N)m and (N)o, where N represents A, T, C or G, or A, U, C or G, m and o are an integer of 1 to 150, and m and o may be the same as or different from each other.

The guide nucleic acid may include one or more domains.

The domains may be a functional domain which is a guide domain, a first complementary domain, a linker domain, a second complementary domain, a proximal domain, or a tail domain, but are not limited to.

Here, one guide nucleic acid may have two or more functional domains. Here, the two or more functional domains may be different from each other. Alternatively, the two or more functional domains included in one guide nucleic acid may be the same as each other. For one example, one guide nucleic acid may have two or more proximal domains. For another example, one guide nucleic acid may have two or more tail domains. However, the description that the functional domains included in one guide nucleic acid are the same domains does not mean that the sequences of the two functional domains are the same. Even if the sequences are different, the two functional domain can be the same domain when perform functionally the same function.

The functional domain will be described in detail below.

### I) Guide Domain

The term “guide domain” is a domain capable of complementary binding with partial sequence of either strand of a double strand of a target gene or a nucleic acid, and acts for specific interaction with a target gene or a nucleic acid. For example, the guide domain may perform a function to induce a guide nucleic acid-editor protein complex to be located to a specific nucleotide sequence of a target gene or a nucleic acid.

The guide domain may be a sequence of 10 to 35 nucleotides.

In an example, the guide domain may be a sequence of 10 to 35, 15 to 35, 20 to 35, 25 to 35 or 30 to 35 nucleotides.

In another example, the guide domain may be a sequence of 10 to 15, 15 to 20, 20 to 25, 25 to 30 or 30 to 35 nucleotides.

The guide domain may include a guide sequence.

The “guide sequence” is a nucleotide sequence complementary to partial sequence of either strand of a double strand of a target gene or a nucleic acid. Here, the guide sequence may be a nucleotide sequence having at least 50%, 55%, 60%, 65%, 70%, 75%, 80%, 85%, 90%, 95% or more complementarity or complete complementarity.

The guide sequence may be a sequence of 10 to 25 nucleotides.

In an example, the guide sequence may be a sequence of 10 to 25, 15 to 25 or 20 to 25 nucleotides.

In another example, the guide sequence may be a sequence of 10 to 15, 15 to 20 or 20 to 25 nucleotides.

In addition, the guide domain may further include an additional nucleotide sequence.

The additional nucleotide sequence may be utilized to improve or degrade the function of the guide domain.

The additional nucleotide sequence may be utilized to improve or degrade the function of the guide sequence.

The additional nucleotide sequence may be a sequence of 1 to 10 nucleotides.

In one example, the additional nucleotide sequence may be a sequence of 2 to 10, 4 to 10, 6 to 10 or 8 to 10 nucleotides.

In another example, the additional nucleotide sequence may be a sequence of 1 to 3, 3 to 6 or 7 to 10 nucleotides.

In one embodiment, the additional nucleotide sequence may be a sequence of 1, 2, 3, 4, 5, 6, 7, 8, 9 or 10 nucleotides.

For example, the additional nucleotide sequence may be one nucleotide sequence G (guanine), or two nucleotide sequence GG.

The additional nucleotide sequence may be located at the 5′ end of the guide sequence.

The additional nucleotide sequence may be located at the 3′ end of the guide sequence.

### II) First Complementary Domain

The term “first complementary domain” is a domain including a nucleotide sequence complementary to a second complementary domain to be described in below, and has enough complementarity so as to form a double strand with the second complementary domain. For example, the first complementary domain may be a nucleotide sequence having at least 50%, 55%, 60%, 65%, 70%, 75%, 80%, 85%, 90%, 95% or more complementarity or complete complementarity to a second complementary domain.

The first complementary domain may form a double strand with a second complementary domain by a complementary binding. Here, the formed double strand may act to form a guide nucleic acid-editor protein complex by interacting with some amino acids of the editor protein.

The first complementary domain may be a sequence of 5 to 35 nucleotides.

In an example, the first complementary domain may be a sequence of 5 to 35, 10 to 35, 15 to 35, 20 to 35, 25 to 35, or 30 to 35 nucleotides.

In another example, the first complementary domain may be a sequence of 1 to 5, 5 to 10, 10 to 15, 15 to 20, 20 to 25, 25 to 30 or 30 to 35 nucleotides.

### III) Linker Domain

The term “linker domain” is a nucleotide sequence connecting two or more domains, which are two or more identical or different domains. The linker domain may be connected with two or more domains by covalent bonding or non-covalent bonding, or may connect two or more domains covalently or non-covalently.

The linker domain may be a sequence of 1 to 30 nucleotides.

In one example, the linker domain may be a sequence of 1 to 5, 5 to 10, 10 to 15, 15 to 20, 20 to 25, or 25 to 30 nucleotides.

In another example, the linker domain may be a sequence of 1 to 30, 5 to 30, 10 to 30, 15 to 30, 20 to 30, or 25 to 30 nucleotides.

### IV) Second Complementary Domain

The term “second complementary domain” is a domain including a nucleotide sequence complementary to the first complementary domain described above, and has enough complementarity so as to form a double strand with the first complementary domain. For example, the second complementary domain may be a nucleotide sequence having at least 50%, 55%, 60%, 65%, 70%, 75%, 80%, 85%, 90%, 95% or more complementarity or complete complementarity to a first complementary domain.

The second complementary domain may form a double strand with a first complementary domain by a complementary binding. Here, the formed double strand may act to form a guide nucleic acid-editor protein complex by interacting with some amino acids of the editor protein. The second complementary domain may have a nucleotide sequence complementary to a first complementary domain, and a nucleotide sequence having no complementarity to the first complementary domain, for example, a nucleotide sequence not forming a double strand with the first complementary domain, and may have a longer sequence than the first complementary domain.

The second complementary domain may be a sequence of 5 to 35 nucleotides.

In an example, the second complementary domain may be a sequence of 1 to 35, 5 to 35, 10 to 35, 15 to 35, 20 to 35, 25 to 35, or 30 to 35 nucleotides.

In another example, the second complementary domain may be a sequence of 1 to 5, 5 to 10, 10 to 15, 15 to 20, 20 to 25, 25 to 30 or 30 to 35 nucleotides.

### V) Proximal Domain

The term “proximal domain” is a nucleotide sequence located adjacent to a second complementary domain.

The proximal domain may include a complementary nucleotide sequence therein, and may form a double strand due to a complementary nucleotide sequence.

The proximal domain may be a sequence of 1 to 20 nucleotides.

In one example, the proximal domain may be a sequence of 1 to 20, 5 to 20, 10 to 20 or 15 to 20 nucleotide.

In another example, the proximal domain may be a sequence of 1 to 5, 5 to 10, 10 to 15 or 15 to 20 nucleotides.

### VI) Tail Domain

The term “tail domain” is a nucleotide sequence located at one or more ends of the both ends of the guide nucleic acid.

The tail domain may include a complementary nucleotide sequence therein, and may form a double strand due to a complementary nucleotide sequence.

The tail domain may be a sequence of 1 to 50 nucleotides.

In an example, the tail domain may be a sequence of 5 to 50, 10 to 50, 15 to 50, 20 to 50, 25 to 50, 30 to 50, 35 to 50, 40 to 50, or 45 to 50 nucleotides.

In another example, the tail domain may be a sequence of 1 to 5, 5 to 10, 10 to 15, 15 to 20, 20 to 25, 25 to 30, 30 to 35, 35 to 40, 40 to 45, or 45 to 50 nucleotides.

Meanwhile, a part or all of the nucleic acid sequences included in the domains, that is, the guide domain, the first complementary domain, the linker domain, the second complementary domain, the proximal domain and the tail domain may selectively or additionally include a chemical modification.

The chemical modification may be, but is not limited to, methylation, acetylation, phosphorylation, phosphorothioate linkage, a locked nucleic acid (LNA), 2′-O-methyl 3′phosphorothioate (MS) or 2′-O-methyl 3′thioPACE (MSP).

The guide nucleic acid includes one or more domains.

The guide nucleic acid may include a guide domain.

The guide nucleic acid may include a first complementary domain.

The guide nucleic acid may include a linker domain.

The guide nucleic acid may include a second complementary domain.

The guide nucleic acid may include a proximal domain.

The guide nucleic acid may include a tail domain.

Here, the guide nucleic acid may include 1, 2, 3, 4, 5, 6 or more domains.

The guide nucleic acid may include 1, 2, 3, 4, 5, 6 or more guide domains.

The guide nucleic acid may include 1, 2, 3, 4, 5, 6 or more first complementary domains.

The guide nucleic acid may include 1, 2, 3, 4, 5, 6 or more linker domains.

The guide nucleic acid may include 1, 2, 3, 4, 5, 6 or more second complementary domains.

The guide nucleic acid may include 1, 2, 3, 4, 5, 6 or more proximal domains.

The guide nucleic acid may include 1, 2, 3, 4, 5, 6 or more tail domains.

Here, the guide nucleic acid may include one type of domain doubly.

The guide nucleic acid may include several domains singly or doubly.

The guide nucleic acid may include the same type of domain. Here, the same type of domain may have the same nucleic acid sequence or different nucleic acid sequences.

The guide nucleic acid may include two types of domains. Here, the two different types of domains may have different nucleic acid sequences or the same nucleic acid sequence.

The guide nucleic acid may include three types of domains. Here, the three different types of domains may have different nucleic acid sequences or the same nucleic acid sequence.

The guide nucleic acid may include four types of domains. Here, the four different types of domains may have different nucleic acid sequences, or the same nucleic acid sequence.

The guide nucleic acid may include five types of domains. Here, the five different types of domains may have different nucleic acid sequences, or the same nucleic acid sequence.

The guide nucleic acid may include six types of domains. Here, the six different types of domains may have different nucleic acid sequences, or the same nucleic acid sequence.

For example, the guide nucleic acid may consist of [guide domain]-[first complementary domain]-[linker domain]-[second complementary domain]-[linker domain]-[guide domain]-[first complementary domain]-[linker domain]-[second complementary domain]. Here, the two guide domains may include guide sequences for different or the same targets, the two first complementary domains and the two second complementary domains may have the same or different nucleic acid sequences. When the guide domains include guide sequences for different targets, the guide nucleic acids may specifically bind to two different targets, and here, the specific bindings may be performed simultaneously or sequentially. In addition, the linker domains may be cleaved by specific enzymes, and the guide nucleic acids may be divided into two or three parts in the presence of specific enzymes.

In one embodiment of the disclosure of the present specification, the guide nucleic acid may be a gRNA.

### gRNA

The “gRNA” refers to an RNA capable of specifically targeting a gRNA-CRISPR enzyme complex, that is, a CRISPR complex, with respect to a target gene or a nucleic acid. In addition, the gRNA refers to an RNA specific to a target gene or a nucleic acid, which may bind to a CRISPR enzyme and guide the CRISPR enzyme to the target gene or the nucleic acid.

The gRNA may include multiple domains. Due to each domain, interactions may occur in a three-dimensional structure or active form of a gRNA strand, or between these strands.

The gRNA may be called single-stranded gRNA (single RNA molecule, single gRNA or sgRNA); or double-stranded gRNA (including more than one, generally, two discrete RNA molecules).

In one exemplary embodiment, the single-stranded gRNA may include a guide domain, that is, a domain including a guide sequence capable of forming a complementary bond with a target gene or a nucleic acid; a first complementary domain; a linker domain; a second complementary domain, which is a domain having a sequence complementary to the first complementary domain sequence, thereby forming a double-stranded nucleic acid with the first complementary domain; a proximal domain; and optionally a tail domain in the 5′ to 3′ direction.

In another embodiment, the double-stranded gRNA may include a first strand which includes a guide domain, that is, a domain including a guide sequence capable of forming a complementary bond with a target gene or a nucleic acid and a first complementary domain; and a second strand which includes a second complementary domain, which is a domain having a sequence complementary to the first complementary domain sequence, thereby forming a double-stranded nucleic acid with the first complementary domain, a proximal domain; and optionally a tail domain in the 5′ to 3′ direction.

Here, the first strand may be referred to as crRNA, and the second strand may be referred to as tracrRNA. The crRNA may include a guide domain and a first complementary domain, and the tracrRNA may include a second complementary domain, a proximal domain and optionally a tail domain.

In still another embodiment, the single-stranded gRNA may include a guide domain, that is, a domain including a guide sequence capable of forming a complementary bond with a target gene or a nucleic acid; a first complementary domain; a second complementary domain, and a domain having a sequence complementary to the first complementary domain sequence, thereby forming a double-stranded nucleic acid with the first complementary domain in the 3′ to 5′ direction.

Here, the first complementary domain may have homology with a natural first complementary domain or may be derived from a natural first complementary domain. In addition, the first complementary domain may have a difference in the nucleotide sequence of a first complementary domain depending on the species existing in nature, may be derived from a first complementary domain contained in the species existing in nature, or may have partial or complete homology with the first complementary domain contained in the species existing in nature.

In one exemplary embodiment, the first complementary domain may have partial, that is, at least 50% or more, or complete homology with a first complementary domain of Streptococcus pyogenes,Campylobacter jejuni, Streptococcus thermophilus, Staphylococcus aureus or Neisseria meningitides, or a first complementary domain derived therefrom.

For example, when the first complementary domain is the first complementary domain of Streptococcus pyogenes or a first complementary domain derived therefrom, the first complementary domain may be 5′-GUUUUAGAGCUA-3′ (SEQ ID NO: 1) or a nucleotide sequence having partial, that is, at least 50% or more, or complete homology with 5′-GUUUUAGAGCUA-3′ (SEQ ID NO: 1). Here, the first complementary domain may further include (X)ₙ, resulting in 5′-GUUUUAGAGCUA(X)ₙ-3′ (SEQ ID NO: 1). The X may be selected from the group consisting of nucleotides A, T, U and G, and the n may represent the number of nucleotide, which is an integer of 5 to 15. Here, the (X)ₙ may be an integer n repeats of the same nucleotide, or an integer n number of nucleotide sequences in which nucleotides of A, T, U and G are mixed.

In another example, when the first complementary domain is the first complementary domain of Campylobacter jejuni or a first complementary domain derived therefrom, the first complementary domain may be 5′-GUUUUAGUCCCUUUUUAAAUUUCUU-3′ (SEQ ID NO: 2) or 5′-GUUUUAGUCCCUU- 3′(SEQ ID NO: 3), or a nucleotide sequence having partial, that is, at least 50% or more, or complete homology with 5′-GUUUUAGUCCCUUUUUAAAUUUCUU-3′ (SEQ ID NO: 2) or 5′-GUUUUAGUCCCUU-3′(SEQ ID NO: 3). Here, the first complementary domain may further include (X)ₙ, resulting in 5′-GUUUUAGUCCCUUUUUAAAUUUCUU(X)ₙ- 3′(SEQ ID NO: 2) or 5′-GUUUUAGUCCCUU(X)n-3′ (SEQ ID NO: 3). The X may be selected from the group consisting of nucleotides A, T, U and G, and the n may represent the number of nucleotide, which is an integer of 5 to 15. Here, the (X)ₙ may be an integer n repeats of the same nucleotide, or an integer n number of nucleotide sequences in which nucleotides of A, T, U and G are mixed.

In another embodiment, the first complementary domain may have partial, that is, at least 50% or more, or complete homology with a first complementary domain of Parcubacteria bacterium (GWC201 1_GWC2_44_17), Lachnospiraceae bacterium (MC2017), Butyrivibrio proteoclasiicus, Peregrinibacteria bacterium (GW2011_GWA_33_10), Acidaminococcus sp. (BV3L6), Porphyromonas macacae, Lachnospiraceae bacterium (ND2006), Porphyromonas crevioricanis, Prevotella disiens, Moraxella bovoculi (237), Smiihella sp. (SC_KO8D17), Leptospira inadai, Lachnospiraceae bacterium (MA2020), Francisella novicida (U112), Candidatus Methanoplasma termitum or Eubacterium eligens, or a first complementary domain derived therefrom.

For example, when the first complementary domain is the first complementary domain of Parcubacteria bacterium or a first complementary domain derived therefrom, the first complementary domain may be 5′-UUUGUAGAU-3′ (SEQ ID NO: 4), or a nucleotide sequence having partial, that is, at least 50% or more homology with 5′- UUUGUAGAU-3′ (SEQ ID NO: 4). Here, the first complementary domain may further include (X)ₙ, resulting in 5′-(X)ₙUUUGUAGAU-3′ (SEQ ID NO: 4). The X may be selected from the group consisting of nucleotides A, T, U and G, and the n may represent the number of nucleotide, which is an integer of 1 to 5. Here, the (X)ₙ may be an integer n repeats of the same nucleotide, or an integer n number of nucleotide sequences in which nucleotides of A, T, U and G are mixed.

Here, the linker domain may be a nucleotide sequence connecting a first complementary domain with a second complementary domain.

The linker domain may form a covalent or non-covalent bonding with a first complementary domain and a second complementary domain, respectively.

The linker domain may connect the first complementary domain with the second complementary domain covalently or non-covalently.

The linker domain is suitable to be used in a single-stranded gRNA molecule, and may be used to produce single-stranded gRNA by being connected with a first strand and a second strand of double-stranded gRNA or connecting the first strand with the second strand by covalent or non-covalent bonding.

The linker domain may be used to produce single-stranded gRNA by being connected with crRNA and tracrRNA of double-stranded gRNA or connecting the crRNA with the tracrRNA by covalent or non-covalent bonding.

Here, the second complementary domain may have homology with a natural second complementary domain, or may be derived from the natural second complementary domain. In addition, the second complementary domain may have a difference in nucleotide sequence of a second complementary domain according to a species existing in nature, and may be derived from a second complementary domain contained in the species existing in nature, or may have partial or complete homology with the second complementary domain contained in the species existing in nature.

In an exemplary embodiment, the second complementary domain may have partial, that is, at least 50% or more, or complete homology with a second complementary domain of Streptococcus pyogenes, Campylobacter jejuni, Streptococcus thermophilus, Staphylococcus aureus or Neisseria meningitides, or a second complementary domain derived therefrom.

For example, when the second complementary domain is a second complementary domain of Streptococcus pyogenes or a second complementary domain derived therefrom, the second complementary domain may be 5′-UAGCAAGUUAAAAU- 3′ (SEQ ID NO: 5), or a nucleotide sequence having partial, that is, at least 50% or more homology with 5′-UAGCAAGUUAAAAU-3′ (SEQ ID NO: 5) (a nucleotide sequence forming a double strand with the first complementary domain is underlined). Here, the second complementary domain may further include (X)ₙ and/or (X)ₘ, resulting in 5′- (X)ₙUAGCAAGUUAAAAU(X)ₘ-3′ (SEQ ID NO: 5). The X may be selected from the group consisting of nucleotides A, T, U and G, and each of the n and m may represent the number of nucleotide, in which the n may be an integer of 1 to 15, and the m may be an integer of 1 to 6. Here, the (X)ₙ may be an integer n repeats of the same nucleotide, or an integer n number of nucleotide sequences in which nucleotides of A, T, U and G are mixed. In addition, the (X)ₘ may be an integer m repeats of the same nucleotide, or an integer m number of nucleotide sequences in which nucleotides of A, T, U and G are mixed.

In another example, when the second complementary domain is the second complementary domain of Campylobacter jejuni or a second complementary domain derived therefrom, the second complementary domain may be 5′-AAGAAAUUUAAAAAGGGACUAAAAU-3′ (SEQ ID NO: 6) or 5′-AAGGGACUAAAAU- 3′(SEQ ID NO: 7), or a nucleotide sequence having partial, that is, at least 50% or more homology with 5′-AAGAAAUUUAAAAAGGGACUAAAAU -3′(SEQ ID NO: 6) or 5′- AAGGGACUAAAAU-3′(SEQ ID NO: 7) (a nucleotide sequence forming a double strand with the first complementary domain is underlined). Here, the second complementary domain may further include (X)ₙ and/or (X)ₘ, resulting in 5′-(X)ₙAAGAAAUUUAAAAAGGGACUAAAAU(X)ₘ-3′(SEQ ID NO: 6) or 5′- (X)ₙAAGGGACUAAAAU(X)ₘ-3′(SEQ ID NO: 7). The X may be selected from the group consisting of nucleotides A, T, U and G, in which the n may be an integer of 1 to 15, and the m may be an integer of 1 to 6. Here, the (X)ₙ may be an integer n repeats of the same nucleotide, or an integer n number of nucleotide sequences in which nucleotides of A, T, U and G are mixed. In addition, the (X)ₘ may be an integer m repeats of the same nucleotide, or an integer m number of nucleotide sequences in which nucleotides of A, T, U and G are mixed.

In another embodiment, the second complementary domain may have partial, that is, at least 50% or more, or complete homology with a second complementary domain of Parcubacteria bacterium (GWC2011_GWC2_44_17), Lachnospiraceae bacterium (MC2017), Butyrivibrio proteoclasiicus, Peregrinibacteria bacterium (GW2011_GWA_33_10), Acidaminococcus sp. (BV3L6), Porphyromonas macacae, Lachnospiraceae bacterium (ND2006), Porphyromonas crevioricanis, Prevotella disiens, Moraxella bovoculi (237), Smiihella sp. (SC_KO8D17), Leptospira inadai, Lachnospiraceae bacterium (MA2020), Francisella novicida (U112), Candidatus Methanoplasma termitum or Eubacterium eligens, or a second complementary domain derived therefrom.

For example, when the second complementary domain is a second complementary domain of Parcubacteria bacterium or a second complementary domain derived therefrom, the second complementary domain may be 5′-AAAUUUCUACU-3′ (SEQ ID NO: 8), or a nucleotide sequence having partial, that is, at least 50% or more homology with 5′-AAAUUUCUACU-3′ (SEQ ID NO: 8) (a nucleotide sequence forming a double strand with the first complementary domain is underlined). Here, the second complementary domain may further include (X)ₙ and/or (X)ₘ, resulting in 5′- (X)ₙAAAUUUCUACU(X)ₘ-3′ (SEQ ID NO: 8). The X may be selected from the group consisting of nucleotides A, T, U and G, and each of the n and m may represent the number of nucleotides sequences, in which the n may be an integer of 1 to 10, and the m may be an integer of 1 to 6. Here, the (X)ₙ may be an integer n repeats of the same nucleotide, or an integer n number of nucleotide sequences in which nucleotides of A, T, U and G are mixed. In addition, the (X)ₘ may be an integer m repeats of the same nucleotide, or an integer m number of nucleotide sequences in which nucleotides of A, T, U and G are mixed.

Here, the first complementary domain and the second complementary domain may complementarily bind to each other.

The first complementary domain and the second complementary domain may form a double strand by the complementary binding.

The formed double strand may interact with a CRISPR enzyme.

Optionally, the first complementary domain may include an additional nucleotide sequence that does not complementarily bind to a second complementary domain of a second strand.

Here, the additional nucleotide sequence may be a sequence of 1 to 15 nucleotides. For example, the additional nucleotide sequence may be a sequence of 1 to 5, 5 to 10 or 10 to 15 nucleotides.

Here, the proximal domain may be a domain located at the 3′end direction of the second complementary domain.

The proximal domain may have homology with a natural proximal domain, or may be derived from the natural proximal domain. In addition, the proximal domain may have a difference in nucleotide sequence according to a species existing in nature, may be derived from a proximal domain contained in the species existing in nature, or may have partial or complete homology with the proximal domain contained in the species existing in nature.

In an exemplary embodiment, the proximal domain may have partial, that is, at least 50% or more, or complete homology with a proximal domain of Streptococcus pyogenes, Campylobacter jejuni, Streptococcus thermophilus, Staphylococcus aureus or Neisseria meningitides, or a proximal domain derived therefrom.

For example, when the proximal domain is a proximal domain of Streptococcus pyogenes or a proximal domain derived therefrom, the proximal domain may be 5′-AAGGCUAGUCCG-3′ (SEQ ID NO: 9), or a nucleotide sequence having partial, that is, at least 50% or more homology with 5′-AAGGCUAGUCCG-3′ (SEQ ID NO: 9). Here, the proximal domain may further include (X)ₙ, resulting in 5′- AAGGCUAGUCCG(X)ₙ-3′ (SEQ ID NO: 9). The X may be selected from the group consisting of nucleotides A, T, U and G, and the n may represent the number of nucleotide, which is an integer of 1 to 15. Here, the (X)ₙ may be an integer n repeats of the same nucleotide, or an integer n number of nucleotide sequences in which nucleotides of A, T, U and G are mixed.

In yet another example, when the proximal domain is a proximal domain of Campylobacter jejuni or a proximal domain derived therefrom, the proximal domain may be 5′-AAAGAGUUUGC-3′ (SEQ ID NO: 10), or a nucleotide sequence having at least 50% or more homology with 5′-AAAGAGUUUGC-3′ (SEQ ID NO: 10). Here, the proximal domain may further include (X)ₙ, resulting in 5′-AAAGAGUUUGC(X)ₙ-3′ (SEQ ID NO: 10). The X may be selected from the group consisting of nucleotides A, T, U and G, and the n may represent the number of nucleotide, which is an integer of 1 to 40. Here, the (X)ₙ may be an integer n repeats of the same nucleotide, or an integer n number of nucleotide sequences in which nucleotides of A, T, U and G are mixed.

Here, the tail domain is a domain which is able to be selectively added to the 3′ end of single-stranded gRNA or the first or second strand of double-stranded gRNA.

The tail domain may have homology with a natural tail domain, or may be derived from the natural tail domain. In addition, the tail domain may have a difference in nucleotide sequence according to a species existing in nature, may be derived from a tail domain contained in a species existing in nature, or may have partial or complete homology with a tail domain contained in a species existing in nature.

In one exemplary embodiment, the tail domain may have partial, that is, at least 50% or more, or complete homology with a tail domain of Streptococcus pyogenes, Campylobacter jejuni, Streptococcus thermophilus, Staphylococcus aureus or Neisseria meningitides or a tail domain derived therefrom.

For example, when the tail domain is a tail domain of Streptococcus pyogenes or a tail domain derived therefrom, the tail domain may be 5′- UUAUCAACUUGAAAAAGUGGCACCGAGUCGGUGC-3′ (SEQ ID NO: 11), or a nucleotide sequence having partial, that is, at least 50% or more homology with 5′- UUAUCAACUUGAAAAAGUGGCACCGAGUCGGUGC-3′ (SEQ ID NO: 11). Here, the tail domain may further include (X)ₙ, resulting in 5′- UUAUCAACUUGAAAAAGUGGCACCGAGUCGGUGC(X)n-3′ (SEQ ID NO: 11). The X may be selected from the group consisting of nucleotides A, T, U and G, and the n may represent the number of nucleotide, which is an integer of 1 to 15. Here, the (X)ₙ may be an integer n repeats of the same nucleotide, or an integer n number of nucleotide sequences in which nucleotides of A, T, U and G are mixed.

In another example, when the tail domain is a tail domain of Campylobacter jejuni or a tail domain derived therefrom, the tail domain may be 5′- GGGACUCUGCGGGGUUACAAUCCCCUAAAACCGCUUUU-3′ (SEQ ID NO: 12), or a nucleotide sequence having partial, that is, at least 50% or more homology with 5′- GGGACUCUGCGGGGUUACAAUCCCCUAAAACCGCUUUU-3′ (SEQ ID NO: 12). Here, the tail domain may further include (X)ₙ, resulting in 5′- GGGACUCUGCGGGGUUACAAUCCCCUAAAACCGCUUUU(X)n-3′ (SEQ ID NO: 12). The X may be selected from the group consisting of nucleotides A, T, U and G, and the n may represent the number of nucleotide, which is an integer of 1 to 15. Here, the (X)ₙ may be an integer n repeats of the same nucleotide, or an integer n number of nucleotide sequences in which nucleotides of A, T, U and G are mixed.

In another embodiment, the tail domain may include a 1 to 10-nt sequence at the 3′ end involved in an in vitro or in vivo transcription method.

For example, when a T7 promoter is used in in vitro transcription of gRNA, the tail domain may be an arbitrary nucleotide sequence present at the 3′ end of a DNA template. In addition, when a U6 promoter is used in in vivo transcription, the tail domain may be UUUUUU, when an H1 promoter is used in transcription, the tail domain may be UUUU, and when a pol-III promoter is used, the tail domain may be several uracil nucleotides or may include alternative nucleotides.

The gRNA may include a plurality of domains as described above, and therefore, the length of the nucleic acid sequence may be regulated according to a domain contained in the gRNA, and interactions may occur in strands in a three-dimensional structure or active form of gRNA or between theses strands due to each domain.

The gRNA may be referred to as single-stranded gRNA (single RNA molecule); or double-stranded gRNA (including more than one, generally two discrete RNA molecules).

### Double-Stranded gRNA

The double-stranded gRNA consists of a first strand and a second strand.

Here, the first strand may consist of
5′-[guide domain]-[first complementary domain]-3′, and
the second strand may consist of
5′-[second complementary domain]-[proximal domain]-3′ or
5′-[second complementary domain]-[proximal domain]-[tail domain]-3′.

Here, the first strand may be referred to as crRNA, and the second strand may be referred to as tracrRNA.

Here, the first strand and the second strand may optionally include an additional nucleotide sequence.

In one embodiment, the first strand may be

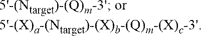

Here, the N_{target} is a nucleotide sequence complementary to partial sequence of either strand of a double strand of a target gene or a nucleic acid, and a nucleotide sequence region which may be changed according to a target sequence on a target gene or a nucleic acid.

Here, the (Q)ₘ is a nucleotide sequence including a first complementary domain. It includes a nucleotide sequence which is able to form a complementary bond with the second complementary domain of the second strand. The (Q)ₘ may be a sequence having partial or complete homology with the first complementary domain of a species existing in nature, and the nucleotide sequence of the first complementary domain may be changed according to the species of origin. The Q may be each independently selected from the group consisting of A, U, C and G, and the m may be the number of nucleotide sequences, which is an integer of 5 to 35.

For example, when the first complementary domain has partial or complete homology with a first complementary domain of Streptococcus pyogenes or a Streptococcus pyogenes-derived first complementary domain, the (Q)ₘ may be 5′- GUUUUAGAGCUA-3′ (SEQ ID NO: 1), or a nucleotide sequence having at least 50% or more homology with 5′-GUUUUAGAGCUA-3′ (SEQ ID NO: 1).

In another example, when the first complementary domain has partial or complete homology with a first complementary domain of Campylobacter jejuni or a Campylobacter jejuni-derived first complementary domain, the (Q)ₘ may be 5′- GUUUUAGUCCCUUUUUAAAUUUCUU-3′ (SEQ ID NO: 2) or 5′-GUUUUAGUCCCUU- 3′(SEQ ID NO: 3), or a nucleotide sequence having at least 50% or more homology with 5′-GUUUUAGUCCCUUUUUAAAUUUCUU-3′ (SEQ ID NO: 2) or 5′- GUUUUAGUCCCUU-3′(SEQ ID NO: 3).

In still another example, when the first complementary domain has partial or complete homology with a first complementary domain of Streptococcus thermophilus or a Streptococcus thermophilus-derived first complementary domain, the (Q)ₘ may be 5′- GUUUUAGAGCUGUGUUGUUUCG-3′ (SEQ ID NO: 13), or a nucleotide sequence having at least 50% or more homology with 5′-GUUUUAGAGCUGUGUUGUUUCG-3′ (SEQ ID NO: 13).

In addition, each of the (X)ₐ, (X)_{b} and (X)_{c} is selectively an additional nucleotide sequence, where the X may be each independently selected from the group consisting of A, U, C and G, and each of the a, b and c may be the number of nucleotide sequences, which is 0 or an integer of 1 to 20.

In one exemplary embodiment, the second strand may be 5′-(Z)ₕ-(P)ₖ-3′; or 5′-(X)_{d}-(Z)ₕ-(X)ₑ(P)ₖ-(X)_{f}-3′.

In another embodiment, the second strand may be 5′-(Z)ₕ-(P)ₖ-(F)ᵣ3′; or 5′- (X)_{d}-(Z)ₕ-(X)ₑ-(P)ₖ-(X)_{f}-(F)ᵢ-3′.

Here, the (Z)ₕ is a nucleotide sequence including a second complementary domain. It includes a nucleotide sequence which is able to form a complementary bond with the first complementary domain of the first strand. The (Z)ₕ may be a sequence having partial or complete homology with the second complementary domain of a species existing in nature, and the nucleotide sequence of the second complementary domain may be modified according to the species of origin. The Z may be each independently selected from the group consisting of A, U, C and G, and the h may be the number of nucleotide sequences, which is an integer of 5 to 50.

For example, when the second complementary domain has partial or complete homology with a second complementary domain of Streptococcus pyogenes or a second complementary domain derived therefrom, the (Z)ₕ may be 5′-UAGCAAGUUAAAAU-3′ (SEQ ID NO: 5), or a nucleotide sequence having at least 50% or more homology with 5′- UAGCAAGUUAAAAU-3′ (SEQ ID NO: 5).

In another example, when the second complementary domain has partial or complete homology with a second complementary domain of Campylobacter jejuni or a second complementary domain derived therefrom, the (Z)ₕ may be 5′- AAGAAAUUUAAAAAGGGACUAAAAU-3′ (SEQ ID NO: 6) or 5′-AAGGGACUAAAAU- 3′(SEQ ID NO: 7), or a nucleotide sequence having at least 50% or more homology with 5′-AAGAAAUUUAAAAAGGGACUAAAAU-3′ or 5′-AAGGGACUAAAAU-3′(SEQ ID NO: 7).

In still another example, when the second complementary domain has partial or complete homology with a second complementary domain of Streptococcus thermophilus or a second complementary domain derived therefrom, the (Z)ₕ may be 5′- CGAAACAACACAGCGAGUUAAAAU-3′ (SEQ ID NO: 14), or a nucleotide sequence having at least 50% or more homology with 5′-CGAAACAACACAGCGAGUUAAAAU-3′ (SEQ ID NO: 14).

The (P)ₖ is a nucleotide sequence including a proximal domain. It may be a sequence having partial or complete homology with a proximal domain of a species existing in nature, and the nucleotide sequence of the proximal domain may be modified according to the species of origin. The P may be each independently selected from the group consisting of A, U, C and G, and the k may be the number of nucleotide sequences, which is an integer of 1 to 20.

For example, when the proximal domain has partial or complete homology with a proximal domain of Streptococcus pyogenes or a proximal domain derived therefrom, the (P)ₖ may be 5′-AAGGCUAGUCCG-3′ (SEQ ID NO: 9), or a nucleotide sequence having at least 50% or more homology with 5′-AAGGCUAGUCCG-3′ (SEQ ID NO: 9).

In another example, when the proximal domain has partial or complete homology with a proximal domain of Campylobacter jejuni or a proximal domain derived therefrom, the (P)ₖ may be 5′-AAAGAGUUUGC-3′ (SEQ ID NO: 10), or a nucleotide sequence having at least 50% or more homology with 5′-AAAGAGUUUGC-3′ (SEQ ID NO: 10).

In still another example, when the proximal domain has partial or complete homology with a proximal domain of Streptococcus thermophilus or a proximal domain derived therefrom, the (P)ₖ may be 5′-AAGGCUUAGUCCG-3′ (SEQ ID NO: 15), or a nucleotide sequence having at least 50% or more homology with 5′- AAGGCUUAGUCCG-3′ (SEQ ID NO: 15).

The (F)ᵢ may be a nucleotide sequence including a tail domain. It may be a sequence having partial or complete homology with a tail domain of a species existing in nature, and the nucleotide sequence of the tail domain may be modified according to the species of origin. The F may be each independently selected from the group consisting of A, U, C and G, and the i may be the number of nucleotide sequences, which is an integer of 1 to 50.

For example, when the tail domain has partial or complete homology with a tail domain of Streptococcus pyogenes or a tail domain derived therefrom, the (F)ᵢ may be 5′-UUAUCAACUUGAAAAAGUGGCACCGAGUCGGUGC-3′ (SEQ ID NO: 11), or a nucleotide sequence having at least 50% or more homology with 5′- UUAUCAACUUGAAAAAGUGGCACCGAGUCGGUGC-3′ (SEQ ID NO: 11).

In another example, when the tail domain has partial or complete homology with a tail domain of Campylobacter jejuni or a tail domain derived therefrom, the (F)ᵢ may be 5′-GGGACUCUGCGGGGUUACAAUCCCCUAAAACCGCUUUU-3′ (SEQ ID NO: 12), or a nucleotide sequence having at least 50% or more homology with 5′- GGGACUCUGCGGGGUUACAAUCCCCUAAAACCGCUUUU-3′ (SEQ ID NO: 12).

In still another example, when the tail domain has partial or complete homology with a tail domain of Streptococcus thermophilus or a tail domain derived therefrom, the (F)ᵢ may be 5′-UACUCAACUUGAAAAGGUGGCACCGAUUCGGUGUUUUU-3′ (SEQ ID NO: 16), or a nucleotide sequence having at least 50% or more homology with 5′- UACUCAACUUGAAAAGGUGGCACCGAUUCGGUGUUUUU-3′ (SEQ ID NO: 16).

In addition, the (F)ᵢ may include a sequence of 1 to 10 nucleotides at the 3′ end involved in an in vitro or in vivo transcription method.

For example, when a T7 promoter is used in in vitro transcription of gRNA, the tail domain may be an arbitrary nucleotide sequence present at the 3′ end of a DNA template. In addition, when a U6 promoter is used in in vivo transcription, the tail domain may be UUUUUU, when an H1 promoter is used in transcription, the tail domain may be UUUU, and when a pol-III promoter is used, the tail domain may be several uracil nucleotides or may include alternative nucleotides.

In addition, the (X)_{d}, (X)ₑ and (X)_{f} may be nucleotide sequences selectively added, where the X may be each independently selected from the group consisting of A, U, C and G, and each of the d, e and f may be the number of nucleotides, which is 0 or an integer of 1 to 20.

### Single-Stranded gRNA

Single-stranded gRNA may be classified into a first single-stranded gRNA and a second single-stranded gRNA.

### First Single-Stranded gRNA

First single-stranded gRNA is single-stranded gRNA in which a first strand and a second strand of the double-stranded gRNA is linked by a linker domain.

Specifically, the single-stranded gRNA may consist of
5′-[guide domain]-[first complementary domain]-[linker domain]-[second complementary domain]-3′,
5′-[guide domain]-[first complementary domain]-[linker domain]-[second complementary domain]-[proximal domain]-3′ or
5′-[guide domain]-[first complementary domain]-[linker domain]-[second complementary domain]-[proximal domain]-[tail domain]-3′.

The first single-stranded gRNA may selectively include an additional nucleotide sequence.

In one exemplary embodiment, the first single-stranded gRNA may be

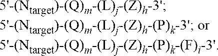

In another embodiment, the single-stranded gRNA may be

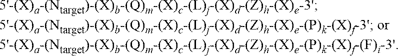

Here, the N_{target} is a nucleotide sequence complementary to partial sequence of either strand of a double strand of a target gene or a nucleic acid, and a nucleotide sequence region capable of being changed according to a target sequence on a target gene or a nucleic acid.

The (Q)ₘ includes a nucleotide sequence including the first complementary domain. It includes a nucleotide sequence which is able to form a complementary bond with a second complementary domain. The (Q)ₘ may be a sequence having partial or complete homology with a first complementary domain of a species existing in nature, and the nucleotide sequence of the first complementary domain may be changed according to the species of origin. The Q may be each independently selected from the group consisting of A, U, C and G, and the m may be the number of nucleotide sequences, which is an integer of 5 to 35.

For example, when the first complementary domain has partial or complete homology with a first complementary domain of Streptococcus pyogenes or a first complementary domain derived therefrom, the (Q)ₘ may be 5′-GUUUUAGAGCUA-3′ (SEQ ID NO: 1), or a nucleotide sequence having at least 50% or more homology with 5′- GUUUUAGAGCUA-3′ (SEQ ID NO: 1).

In another example, when the first complementary domain has partial or complete homology with a first complementary domain of Campylobacter jejuni or a first complementary domain derived therefrom, the (Q)ₘ may be 5′- GUUUUAGUCCCUUUUUAAAUUUCUU-3′ (SEQ ID NO: 2) or 5′-GUUUUAGUCCCUU- 3′(SEQ ID NO: 3), or a nucleotide sequence having at least 50% or more homology with 5′-GUUUUAGUCCCUUUUUAAAUUUCUU-3′ (SEQ ID NO: 2) or 5′- GUUUUAGUCCCUU-3′(SEQ ID NO: 3).

In still another example, when the first complementary domain has partial or complete homology with a first complementary domain of Streptococcus thermophilus or a first complementary domain derived therefrom, the (Q)ₘ may be 5′- GUUUUAGAGCUGUGUUGUUUCG-3′ (SEQ ID NO: 13), or a nucleotide sequence having at least 50% or more homology with 5′-GUUUUAGAGCUGUGUUGUUUCG-3′ (SEQ ID NO: 13).

In addition, the (L)ⱼ is a nucleotide sequence including the linker domain, and connecting the first complementary domain with the second complementary domain, thereby producing single-stranded gRNA. Here, the L may be each independently selected from the group consisting of A, U, C and G, and the j may be the number of nucleotide sequences, which is an integer of 1 to 30.

The (Z)ₕ is a nucleotide sequence including the second complementary domain, and includes a nucleotide sequence capable of complementary binding with the first complementary domain. The (Z)ₕ may be a sequence having partial or complete homology with the second complementary domain of a species existing in nature, and the nucleotide sequence of the second complementary domain may be changed according to the species of origin. The Z may be each independently selected from the group consisting of A, U, C and G, and the h is the number of nucleotide sequences, which may be an integer of 5 to 50.

For example, when the second complementary domain has partial or complete homology with a second complementary domain of Streptococcus pyogenes or a second complementary domain derived therefrom, the (Z)ₕ may be 5′-UAGCAAGUUAAAAU-3′ (SEQ ID NO: 5), or a nucleotide sequence having at least 50% or more homology with 5′- UAGCAAGUUAAAAU-3′ (SEQ ID NO: 5).

In another example, when the second complementary domain has partial or complete homology with a second complementary domain of Campylobacter jejuni or a second complementary domain derived therefrom, the (Z)h may be 5′- AAGAAAUUUAAAAAGGGACUAAAAU-3′ (SEQ ID NO: 6) or 5′-AAGGGACUAAAAU 3′(SEQ ID NO: 7), or a nucleotide sequence having at least 50% or more homology with 5′-AAGAAAUUUAAAAAGGGACUAAAAU-3′ (SEQ ID NO: 6) or 5′-AAGGGACUAAAAU- 3′(SEQ ID NO: 7).

In still another example, when the second complementary domain has partial or complete homology with a second complementary domain of Streptococcus thermophilus or a second complementary domain derived therefrom, the (Z)h may be 5′- CGAAACAACACAGCGAGUUAAAAU-3′ (SEQ ID NO: 14), or a nucleotide sequence having at least 50% or more homology with 5′-CGAAACAACACAGCGAGUUAAAAU-3′ (SEQ ID NO: 14).

The (P)k is a nucleotide sequence including a proximal domain. It may be a sequence having partial or complete homology with a proximal domain of a species existing in nature, and the nucleotide sequence of the proximal domain may be modified according to the species of origin. The P may be each independently selected from the group consisting of A, U, C and G, and the k may be the number of nucleotide sequences, which is an integer of 1 to 20.

For example, when the proximal domain has partial or complete homology with a proximal domain of Streptococcus pyogenes or a proximal domain derived therefrom, the (P)ₖ may be 5′-AAGGCUAGUCCG-3′ (SEQ ID NO: 9), or a nucleotide sequence having at least 50% or more homology with 5′-AAGGCUAGUCCG-3′ (SEQ ID NO: 9).

In another example, when the proximal domain has partial or complete homology with a proximal domain of Campylobacter jejuni or a proximal domain derived therefrom, the (P)ₖ may be 5′-AAAGAGUUUGC-3′ (SEQ ID NO: 10), or a nucleotide sequence having at least 50% or more homology with 5′-AAAGAGUUUGC-3′ (SEQ ID NO: 10).

In still another example, when the proximal domain has partial or complete homology with a proximal domain of Streptococcus thermophilus or a proximal domain derived therefrom, the (P)ₖ may be 5′-AAGGCUUAGUCCG-3′ (SEQ ID NO: 15), or a nucleotide sequence having at least 50% or more homology with 5′- AAGGCUUAGUCCG-3′ (SEQ ID NO: 15).

The (F)ᵢ may be a nucleotide sequence including a tail domain, and having partial or complete homology with a tail domain of a species existing in nature, and the nucleotide sequence of the tail domain may be modified according to the species of origin. The F may be each independently selected from the group consisting of A, U, C and G, and the i may be the number of nucleotide sequences, which is an integer of 1 to 50.

For example, when the tail domain has partial or complete homology with a tail domain of Streptococcus pyogenes or a tail domain derived therefrom, the (F)ᵢ may be 5′-UUAUCAACUUGAAAAAGUGGCACCGAGUCGGUGC-3′ (SEQ ID NO: 11), or a nucleotide sequence having at least 50% or more homology with 5′- UUAUCAACUUGAAAAAGUGGCACCGAGUCGGUGC-3′ (SEQ ID NO: 11).

In another example, when the tail domain has partial or complete homology with a tail domain of Campylobacter jejuni or a tail domain derived therefrom, the (F)ᵢ may be 5′-GGGACUCUGCGGGGUUACAAUCCCCUAAAACCGCUUUU-3′ (SEQ ID NO: 12), or a nucleotide sequence having at least 50% or more homology with 5′- GGGACUCUGCGGGGUUACAAUCCCCUAAAACCGCUUUU-3′ (SEQ ID NO: 12).

In still another example, when the tail domain has partial or complete homology with a tail domain of Streptococcus thermophilus or a tail domain derived therefrom, the (F)ᵢ may be 5′-UACUCAACUUGAAAAGGUGGCACCGAUUCGGUGUUUUU-3′ (SEQ ID NO: 16), or a nucleotide sequence having at least 50% or more homology with 5′- UACUCAACUUGAAAAGGUGGCACCGAUUCGGUGUUUUU-3′ (SEQ ID NO: 16).

In addition, the (F)ᵢ may include a sequence of 1 to 10 nucleotides at the 3′ end involved in an in vitro or in vivo transcription method.

For example, when a T7 promoter is used in in vitro transcription of gRNA, the tail domain may be an arbitrary nucleotide sequence present at the 3′ end of a DNA template. In addition, when a U6 promoter is used in in vivo transcription, the tail domain may be UUUUUU, when an H1 promoter is used in transcription, the tail domain may be UUUU, and when a pol-III promoter is used, the tail domain may be several uracil nucleotides or may include alternative nucleotides.

In addition, the (X)ₐ, (X)_{b}, (X)_{c}, (X)_{d}, (X)ₑ and (X)_{f} may be nucleotide sequences selectively added, where the X may be each independently selected from the group consisting of A, U, C and G, and each of the a, b, c, d, e and f may be the number of nucleotide sequences, which is 0 or an integer of 1 to 20.

### Second Single-Stranded gRNA

Second single-stranded gRNA may be single-stranded gRNA consisting of a guide domain, a first complementary domain and a second complementary domain.

Here, the second single-stranded gRNA may consist of:
5′-[second complementary domain]-[first complementary domain]-[guide domain]-3′; or
5′-[second complementary domain]-[linker domain]-[first complementary domain]-[guide domain]-3′.

The second single-stranded gRNA may selectively include an additional nucleotide sequence.

In one exemplary embodiment, the second single-stranded gRNA may be

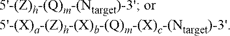

In another embodiment, the single-stranded gRNA may be

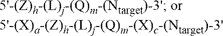

Here, the N_{target} is a nucleotide sequence complementary to partial sequence of either strand of a double strand of a target gene or a nucleic acid, and a nucleotide sequence region capable of being changed according to a target sequence on a target gene or a nucleic acid.

The (Q)ₘ is a nucleotide sequence including the first complementary domain, and includes a nucleotide sequence capable of complementary binding with a second complementary domain. The (Q)ₘ may be a sequence having partial or complete homology with the first complementary domain of a species existing in nature, and the nucleotide sequence of the first complementary domain may be changed according to the species of origin. The Q may be each independently selected from the group consisting of A, U, C and G, and the m may be the number of nucleotide sequences, which is an integer of 5 to 35.

For example, when the first complementary domain has partial or complete homology with a first complementary domain of Parcubacteria bacterium or a first complementary domain derived therefrom, the (Q)ₘ may be 5′-UUUGUAGAU-3′ (SEQ ID NO: 4), or a nucleotide sequence having at least 50% or more homology with 5′- UUUGUAGAU-3′ (SEQ ID NO: 4).

The (Z)ₕ is a nucleotide sequence including a second complementary domain, and includes a nucleotide sequence capable of complementary binding with a second complementary domain. The (Z)ₕ may be a sequence having partial or complete homology with the second complementary domain of a species existing in nature, and the nucleotide sequence of the second complementary domain may be modified according to the species of origin. The Z may be each independently selected from the group consisting of A, U, C and G, and the h may be the number of nucleotide sequences, which is an integer of 5 to 50.

For example, when the second complementary domain has partial or complete homology with a second complementary domain of Parcubacteria bacterium or a Parcubacteria bacterium-derived second complementary domain, the (Z)ₕ may be 5′- AAAUUUCUACU-3′ (SEQ ID NO: 8), or a nucleotide sequence having at least 50% or more homology with 5′-AAAUUUCUACU-3′ (SEQ ID NO: 8).

In addition, the (L)ⱼ is a nucleotide sequence including the linker domain. It is a nucleotide sequence connecting the first complementary domain with the second complementary domain. Here, the L may be each independently selected from the group consisting of A, U, C and G, and the j may be the number of nucleotide sequences, which is an integer of 1 to 30.

In addition, each of the (X)ₐ, (X)_{b} and (X)_{c} may be nucleotide selectively added, where the X may be each independently selected from the group consisting of A, U, C and G, and the a, b and c may be the number of nucleotide, which is 0 or an integer of 1 to 20.

In one aspect of the disclosure in the present specification, the guide nucleic acid may be gRNA capable of complementarily binding to a target sequence of a blood coagulation inhibitory gene.

The term “blood coagulation inhibitory gene” refers to all types of genes that directly participate in or have an indirect effect of inhibiting blood coagulation or the formation of blood clots or inactivating a blood coagulation system. In this case, the blood coagulation inhibitory gene may function to inhibit or suppress the activities of various genes or various proteins which are involved in the blood coagulation system due to the blood coagulation inhibitory gene itself or a protein expressed by the blood coagulation inhibitory gene and function to directly inhibit blood coagulation. The term “blood coagulation system” refers to the overall process of coagulating blood, which occurs in vivo. In this case, the blood coagulation system includes all types of normal blood coagulation systems and abnormal blood coagulation systems in which blood coagulation is delayed. A protein expressed by the blood coagulation inhibitory gene may be used interchangeably with the term “blood coagulation inhibitory factor” or “anticoagulant factor.”

The blood coagulation inhibitory gene may inhibit or suppress the blood coagulation.

The blood coagulation inhibitory gene may inhibit or suppress the expression of blood coagulation-associated proteins.

The blood coagulation inhibitory gene may inhibit or suppress the activities of the blood coagulation-associated proteins.

In this case, the blood coagulation-associated proteins may include factor XII, factor XIIa, factor XI, factor XIa,factor IX, factor IXa, factor X, factor Xa, factor VIII, factor VIIIa, factor VII, factor VIIa, factor V, factor Va, prothrombin, thrombin, factor XIII, factor XIIIa, fibrinogen, fibrin, or a tissue factor.

The blood coagulation inhibitory gene may be an antithrombin (AT) gene.

The AT gene refers to a gene that encodes a protein that consisting of 432 amino acids, which can inactivate various enzymes in the blood coagulation system, and is also referred to as a SERPINC1 gene. As one example, the AT gene may include one or more selected from the group consisting of the following, but the present invention is not limited thereto: genes encoding human ATs (e.g., NCBI Accession No. NP_000479, and the like), for example, AT genes represented by NCBI Accession No. NM_000488, and the like.

The blood coagulation inhibitory gene may be a tissue factor pathway inhibitor (TFPI).

The TFPI gene refers to a gene (full-length DNA, cDNA, or mRNA) that encodes a protein capable of reversibly suppressing factor Xa. In the case of a human being, the TFPI gene is located on chromosomes 2q31-q32.1, and has 9 exons. As one example, the TFPI gene may include one or more selected from the group consisting of the following, but the present invention is not limited thereto: genes encoding human TFPIs (e.g., NCBI Accession Nos. NP_001027452, NP_001305870, NP_001316168, NP_001316169, NP_001316170, and the like), for example TFPI genes represented by NCBI Accession Nos. NM_001032281, NM_006287, NM_001318941, NM_001329239, NM_001329240, and the like.

The blood coagulation inhibitory gene may be derived from mammals, which include primates such as a human, a monkey, and the like, rodents such as a rat, a mouse, and the like.

The genetic information may be obtained from the known databases such as GenBank of NCBI (National Center for Biotechnology Information).

In one embodimentof the disclosure in the present specification, the guide nucleic acid may be gRNA targeting a target sequence of a AT gene and/or a TFP1 gene.

The “target sequence” is a nucleotide sequence present in a target gene or nucleic acid, and specifically, a partial nucleotide sequence of a target region in a target gene or a nucleic acid, and here, the “target region” is a region that can be modified by a guide nucleic acid-editor protein in a target gene or a nucleic acid.

Hereinafter, the target sequence may be used to refer to both of two types of nucleotide sequence information. For example, in the case of a target gene, the target sequence may refer to the nucleotide sequence information of a transcribed strand of target gene DNA, or the nucleotide sequence information of a non-transcribed strand.

For example, the target sequence may refer to a partial nucleotide sequence (transcribed strand), that is, 5′-ATCATTGGCAGACTAGTTCG-3′ (SEQ ID NO: 17), in the target region of target gene A, or a nucleotide sequence complementary thereto (non-transcribed strand), that is, 5′-CGAACTAGTCTGCCAATGAT-3′ (SEQ ID NO: 18).

The target sequence may be a 5 to 50-nt sequence.

In one exemplary embodiment, the target sequence may be a 16-nt sequence, a 17-nt sequence, a 18-nt sequence, a 19-nt sequence, a 20-nt sequence, a 21-nt sequence, a 22-nt sequence, a 23-nt sequence, a 24-nt sequence or a 25-nt sequence.

The target sequence includes a guide nucleic acid-binding sequence or a guide nucleic acid-non binding sequence.

The “guide nucleic acid-binding sequence” is a nucleotide sequence having partial or complete complementarity with a guide sequence included in the guide domain of the guide nucleic acid, and may be complementarily bonded with the guide sequence included in the guide domain of the guide nucleic acid. The target sequence and guide nucleic acid-binding sequence are nucleotide sequences that may vary according to a target gene or a nucleic acid, that is, the subject to be genetically manipulated or edited, and may be designed variously according to a target gene or a nucleic acid.

The “guide nucleic acid-non binding sequence” is a nucleotide sequence having partial or complete homology with a guide sequence included in the guide domain of the guide nucleic acid, and may not be complementarily bonded with the guide sequence included in the guide domain of the guide nucleic acid. In addition, the guide nucleic acid-non binding sequence may be a nucleotide sequence having complementarity with the guide nucleic acid-binding sequence, and may be complementarily bonded with the guide nucleic acid-binding sequence.

The guide nucleic acid-binding sequence may be a partial nucleotide sequence of a target sequence, and one nucleotide sequence of two nucleotide sequences having different sequence order to each other the target sequence, that is, one of the two nucleotide sequences capable of complementary binding to each other. Here, the guide nucleic acid-non binding sequence may be a nucleotide sequence other than the guide nucleic acid-binding sequence of the target sequence.

For example, in a target region of the target gene A, when target sequences are designed as a partial nucleotide sequence, that is, 5′-ATCATTGGCAGACTAGTTCG- 3′ (SEQ ID NO: 17), and a complementary nucleotide sequence thereof, that is, 5′- CGAACTAGTCTGCCAATGAT-3′ (SEQ ID NO: 18), the guide nucleic acid-binding sequence may be one of the two target sequences, that is, 5′- ATCATTGGCAGACTAGTTCG-3′(SEQ ID NO: 17) or 5′-CGAACTAGTCTGCCAATGAT- 3′(SEQ ID NO: 18). Here, when the guide nucleic acid-binding sequence is 5′- ATCATTGGCAGACTAGTTCG-3′(SEQ ID NO: 17), the guide nucleic acid-non binding sequence may be 5′-CGAACTAGTCTGCCAATGAT-3′ (SEQ ID NO: 18), or when the guide nucleic acid-binding sequence is 5′-CGAACTAGTCTGCCAATGAT-3′ (SEQ ID NO: 18), the guide nucleic acid-non binding sequence may be 5′- ATCATTGGCAGACTAGTTCG-3′ (SEQ ID NO: 17).

The guide nucleic acid-binding sequence may be one of the target sequences, that is, a nucleotide sequence which is the same as a transcribed strand and a nucleotide sequence which is the same as a non-transcribed strand. Here, the guide nucleic acid-non binding sequence may be a nucleotide sequence other than the guide nucleic acid-binding sequence of the target sequences. It may be the nucleotide sequence other than a nucleotide sequence which is the same as a transcribed strand or a non-transcribed strand.

The guide nucleic acid-binding sequence may have the same length as the target sequence.

The guide nucleic acid-non binding sequence may have the same length as the target sequence or the guide nucleic acid-binding sequence.

The guide nucleic acid-binding sequence may be a 5 to 50-nt sequence.

In one exemplary embodiment, the guide nucleic acid-binding sequence may be a 16-nt sequence, a 17-nt sequence, a 18-nt sequence, a 19-nt sequence, a 20-nt sequence, a 21-nt sequence, a 22-nt sequence, a 23-nt sequence, a 24-nt sequence or a 25-nt sequence.

The guide nucleic acid-non binding sequence may be a 5 to 50-nt sequence.

In one exemplary embodiment, the guide nucleic acid-non binding sequence may be a 16-nt sequence, a 17-nt sequence, a 18-nt sequence, a 19-nt sequence, a 20-nt sequence, a 21-nt sequence, a 22-nt sequence, a 23-nt sequence, a 24-nt sequence or a 25-nt sequence.

The guide nucleic acid-binding sequence may partially or completely complementarily bind to the guide sequence included in the guide domain of the guide nucleic acid, and the length of the guide nucleic acid-binding sequence may be the same as that of the guide sequence.

The guide nucleic acid-binding sequence may be a nucleotide sequence complementary to the guide sequence included in the guide domain of the guide nucleic acid, and for example, a nucleotide sequence which has at least 70%, 75%, 80%, 85%, 90%, 95% or more complementarity or complete complementarity.

As an example, the guide nucleic acid-binding sequence may have or include a 1 to 8-nt sequence which is not complementary to the guide sequence included in the guide domain of the guide nucleic acid.

The guide nucleic acid-non binding sequence may have partial or complete homology with the guide sequence included in the guide domain of the guide nucleic acid, and the length of the guide nucleic acid-non binding sequence may be the same as that of the guide sequence.

The guide nucleic acid-non binding sequence may be a nucleotide sequence having homology with the guide sequence included in the guide domain of the guide nucleic acid, and for example, a nucleotide sequence which has at least 70%, 75%, 80%, 85%, 90%, 95% or more homology or complete homology.

In one example, the guide nucleic acid-non binding sequence may have or include a 1 to 8-nt sequence which is not homologous to the guide sequence included in the guide domain of the guide nucleic acid.

The guide nucleic acid-non binding sequence may complementarily bind with the guide nucleic acid-binding sequence, and the guide nucleic acid-non binding sequence may have the same length as the guide nucleic acid-binding sequence.

The guide nucleic acid-non binding sequence may be a nucleotide sequence complementary to the guide nucleic acid-binding sequence, and for example, a nucleotide sequence having at least 90%, 95% or more complementarity or complete complementarity.

In one example, the guide nucleic acid-non binding sequence may have or include a 1 to 2-nt sequence which is not complementary to the guide nucleic acid-binding sequence.

In addition, the guide nucleic acid-binding sequence may be a nucleotide sequence located near a nucleotide sequence recognized by an editor protein.

In one example, the guide nucleic acid-binding sequence may be a consecutive 5 to 50-nt sequence located adjacent to the 5′ end and/or 3′ end of a nucleotide sequence recognized by an editor protein.

In addition, the guide nucleic acid-non binding sequence may be a nucleotide sequence located near a nucleotide sequence recognized by an editor protein.

In one example, the guide nucleic acid-non binding sequence may be a 5 to 50-nt contiguous sequence located adjacent to the 5′ end and/or 3′ end of a nucleotide sequence recognized by an editor protein.

The “targeting” refers to complementary binding with the guide nucleic acid-binding sequence of the target sequence present in a target gene or a nucleic acid. Here, the complementary binding may be 100% completely complementary binding, or 70% or more and less than 100%, incomplete complementary binding. Therefore, the “targeting gRNA” refers to gRNA complementarily binding to the guide nucleic acid-binding sequence of the target sequence present in a target gene or a nucleic acid.

The target gene disclosed in the specification may be a blood coagulation inhibitory gene.

The target gene disclosed in the specification may be a AT gene and/or TFPI gene.

In an embodiment, the target sequence disclosed in the present specification may be a 10 to 35-nt contiguous sequence located in the promoter region of the blood coagulation inhibitory gene.

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence located in the promoter region of the AT gene.

In another example, the target sequence may be a 10 to 25 contiguous nucleotide sequence located in the promoter region of the TFPI gene.

The target sequence disclosed in the present specification may be a 10 to 35- nt contiguous sequence located in an intron region of the blood coagulation inhibitory gene.

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence located in an intron region of the AT gene.

In another example, the target sequence may be a 10 to 25-nt contiguous sequence located in an intron region of the TFPI gene.

The target sequence disclosed in the present specification may be a 10 to 35- nt contiguous sequence located in an exon region of the blood coagulation inhibitory gene.

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence located in an exon region of the AT gene.

In another example, the target sequence may be a 10 to 25-nt contiguous sequence located in an exon region of the TFPI gene.

The target sequence disclosed in the present specification may be a 10 to 35- nt contiguous sequence located in an enhancer region of the blood coagulation inhibitory gene.

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence located in an enhancer region of the AT gene.

In another example, the target sequence may be a 10 to 25-nt contiguous sequence located in an enhancer region of the TFPI gene.

The target sequence disclosed in the present specification may be a 10 to 35- nt contiguous sequence located in a coding region, a non-coding region or a mixed region of the blood coagulation inhibitory gene.

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence located in a coding region, a non-coding region or a mixed region of the AT gene.

In another example, the target sequence may be a 10 to 25-nt contiguous sequence located in a coding region, a non-coding region or a mixed region of the TFPI gene.

The target sequence disclosed in the present specification may be a 10 to 35- nt contiguous sequence located in a promoter, an enhancer, a 3′ UTR, a polyA region or a mixed region of the blood coagulation inhibitory gene.

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence located in a promoter, an enhancer, a 3′ UTR, a polyA region or a mixed region of the AT gene.

In another example the target sequence may be a 10 to 25-nt contiguous sequence located in a promoter, an enhancer, a 3′ UTR, a polyA region or a mixed region of the TFPI gene.

The target sequence disclosed in the present specification may be a 10 to 35- nt contiguous sequence located in an exon, an intron or a mixed region of the blood coagulation inhibitory gene.

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence located in an exon, an intron or a mixed region of the AT gene.

In another example, the target sequence may be a 10 to 25-nt contiguous sequence located in an exon, an intron or a mixed region of the TFPI gene.

The target sequence disclosed in the present specification may be a 10 to 35- nt contiguous sequence which includes or is adjacent to a mutant part (e.g., a part different from a wild-type gene) of the blood coagulation inhibitory gene.

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence which includes or is adjacent to a mutant part (e. g, a part different from a wild- type gene) of the AT gene.

In another example, the target sequence may be a 10 to 25-nt contiguous sequence which includes or is adjacent to a mutant part (e. g, a part different from a wild- type gene) of the TFPI gene.

The target sequence disclosed in the present specification may be a 10 to 35- nt contiguous sequence which is adjacent to the 5′ end and/or 3′ end of a proto-spacer- adjacent motif (PAM) sequence in the nucleic acid sequence of the blood coagulation inhibitory gene.

The “proto-spacer-adjacent motif (PAM) sequence” is a nucleotide sequence that can be recognized by an editor protein. Here, the PAM sequence may have different nucleotide sequences according to the type of the editor protein and an editor protein-derived species.

Here, the PAM sequence may be, for example, one or more sequences of the following sequences (described in a 5′ to 3′ direction).
NGG (N is A, T, C or G);
NNNNRYAC (N is each independently A, T, C or G, R is A or G, and Y is C or T);
NNAGAAW (N is each independently A, T, C or G, and W is A or T);
NNNNGATT (N is each independently A, T, C or G);
NNGRR(T) (N is each independently A, T, C or G, and R is A or G); and
TTN (N is A, T, C or G).

Here, the target sequence may be a 10 to 35-nt sequence, a 15 to 35-nt sequence, a 20 to 35-nt sequence, a 25 to 35-nt sequence or a 30 to 35-nt sequence.

Alternatively, the target sequence may be a 10 to 15-nt sequence, a 15 to 20- nt sequence, a 20 to 25-nt sequence, a 25 to 30-nt sequence or a 30 to 35-nt sequence.

In one example, the target sequence may be a 10 to 25-nt contiguous sequence adjacent to the 5′ end and/or 3′ end of a PAM sequence in the nucleic acid sequence of the AT gene.

In one embodiment, when the PAM sequence recognized by an editor protein is 5′-NGG-3′, 5′-NAG-3′ and/or 5′-NGA-3′ (N= A, T, G or C; or A, U, G or C), the target sequence may be a 10 to 25-nt contiguous sequence adjacent to the 5′ end and/or 3′ end of the 5′-NGG-3′, 5′-NAG-3′ and/or 5′-NGA-3′ (N= A, T, G or C; or A, U, G or C) sequence in the nucleic acid sequence of the AT gene.

In another embodiment, when the PAM sequence recognized by an editor protein is 5′-NGGNG-3′ and/or 5′-NNAGAAW-3′ (W = A or T, N= A, T, G or C; or A, U, G or C), the target sequence may be a 10 to 25-nt contiguous sequence adjacent to the 5′ end and/or 3′ end of the 5′-NGGNG-3′ and/or 5′-NNAGAAW-3′ (W = A or T, N= A, T, G or C; or A, U, G or C) sequence in the nucleic acid sequence of the AT gene.

In still another embodiment, when the PAM sequence recognized by an editor protein is 5′-NNNNGATT-3′ and/or 5′-NNNGCTT-3′ (N= A, T, G or C; or A, U, G or C), the target sequence may be a 10 to 25-nt contiguous sequence adjacent to the 5′ end and/or 3′ end of the 5′-NNNNGATT-3′ and/or 5′-NNNGCTT-3′ (N= A, T, G or C; or A, U, G or C) sequence in the nucleic acid sequence of the AT gene.

In one embodiment, when the PAM sequence recognized by an editor protein is 5′-NNNVRYAC-3′ (V = G, C or A; R = A or G, Y = C or T, N= A, T, G or C; or A, U, G or C), the target sequence may be a 10 to 25-nt contiguous sequence adjacent to the 5′ end and/or 3′ end of the 5′-NNNVRYAC-3′ (V = G, C or A; R = A or G, Y = C or T, N= A, T, G or C; or A, U, G or C) sequence in the nucleic acid sequence of the AT gene.

In another embodiment, when the PAM sequence recognized by an editor protein is 5′-NAAR-3′(R = A or G, N= A, T, G or C; or A, U, G or C), the target sequence may be a 10 to 25-nt contiguous sequence adjacent to the 5′ end and/or 3′ end of the 5′- NAAR-3′(R = A or G, N= A, T, G or C; or A, U, G or C) sequence in the nucleic acid sequence of the AT gene.

In still another embodiment, when the PAM sequence recognized by an editor protein is 5′-NNGRR-3′, 5′-NNGRRT-3′ and/or 5′-NNGRRV-3′ (R = A or G, V = G, C or A, N= A, T, G or C; or A, U, G or C), the target sequence may be a 10 to 25-nt contiguous sequence adjacent to the 5′ end and/or 3′ end of the 5′-NNGRR-3′, 5′-NNGRRT-3′ and/or 5′-NNGRRV-3′ (R = A or G, V = G, C or A, N= A, T, G or C; or A, U, G or C) sequence in the nucleic acid sequence of the AT gene.

In one embodiment, when the PAM sequence recognized by an editor protein is 5′-TTN-3′ (N= A, T, G or C; or A, U, G or C), the target sequence may be a 10 to 25-nt contiguous sequence adjacent to the 5′ end and/or 3′ end of the 5′-TTN-3′ (N= A, T, G or C; or A, U, G or C) sequence in the nucleic acid sequence of the AT gene.

In another example, the target sequence may be a 10 to 25-nt contiguous sequence adjacent to the 5′ end and/or 3′ end of a PAM sequence in the nucleic acid sequence of the TFPI gene.

In one embodiment, when the PAM sequence recognized by an editor protein is 5′-NGG-3′, 5′-NAG-3′ and/or 5′-NGA-3′ (N= A, T, G or C; or A, U, G or C), the target sequence may be a 10 to 25-nt contiguous sequence adjacent to the 5′ end and/or 3′ end of the 5′-NGG-3′, 5′-NAG-3′ and/or 5′-NGA-3′ (N= A, T, G or C; or A, U, G or C) sequence in the nucleic acid sequence of the TFPI gene.

In another embodiment, when the PAM sequence recognized by an editor protein is 5′-NGGNG-3′ and/or 5′-NNAGAAW-3′ (W = A or T, N= A, T, G or C; or A, U, G or C), the target sequence may be a 10 to 25-nt contiguous sequence adjacent to the 5′ end and/or 3′ end of the 5′-NGGNG-3′ and/or 5′-NNAGAAW-3′ (W = A or T, N= A, T, G or C; or A, U, G or C) sequence in the nucleic acid sequence of the TFPI gene.

In still another embodiment, when the PAM sequence recognized by an editor protein is 5′-NNNNGATT-3′ and/or 5′-NNNGCTT-3′ (N= A, T, G or C; or A, U, G or C), the target sequence may be a 10 to 25-nt contiguous sequence adjacent to the 5′ end and/or 3′ end of the 5′-NNNNGATT-3′ and/or 5′-NNNGCTT-3′ (N= A, T, G or C; or A, U, G or C) sequence in the nucleic acid sequence of the TFPI gene.

In one embodiment, when the PAM sequence recognized by an editor protein is 5′-NNNVRYAC-3′ (V = G, C or A; R = A or G, Y = C or T, N= A, T, G or C; or A, U, G or C), the target sequence may be a 10 to 25-nt contiguous sequence adjacent to the 5′ end and/or 3′ end of the 5′-NNNVRYAC-3′ (V = G, C or A; R = A or G, Y = C or T, N= A, T, G or C; or A, U, G or C) sequence in the nucleic acid sequence of the TFPI gene.

In another embodiment, when the PAM sequence recognized by an editor protein is 5′-NAAR-3′(R = A or G, N= A, T, G or C; or A, U, G or C), the target sequence may be a 10 to 25-nt contiguous sequence adjacent to the 5′ end and/or 3′ end of the 5′- NAAR-3′(R = A or G, N= A, T, G or C; or A, U, G or C) sequence in the nucleic acid sequence of the TFPI gene.

In still another embodiment, when the PAM sequence recognized by an editor protein is 5′-NNGRR-3′, 5′-NNGRRT-3′ and/or 5′-NNGRRV-3′ (R = A or G, V = G, C or A, N= A, T, G or C; or A, U, G or C), the target sequence may be a 10 to 25-nt contiguous sequence adjacent to the 5′ end and/or 3′ end of the 5′-NNGRR-3′, 5′-NNGRRT-3′ and/or 5′-NNGRRV-3′ (R = A or G, V = G, C or A, N= A, T, G or C; or A, U, G or C) sequence in the nucleic acid sequence of the TFPI gene.

In one embodiment, when the PAM sequence recognized by an editor protein is 5′-TTN-3′ (N= A, T, G or C; or A, U, G or C), the target sequence may be a 10 to 25-nt contiguous sequence adjacent to the 5′ end and/or 3′ end of the 5′-TTN-3′ (N= A, T, G or C; or A, U, G or C) sequence in the nucleic acid sequence of the TFPI gene.

Hereinafter, examples of target sequences that can be used in an exemplary embodiment disclosed in the specification are listed in Tables 1 and 2. The target sequences disclosed in Tables 1 and 2 are guide nucleic acid-non binding sequences, and complementary sequences thereof, that is, guide nucleic acid-binding sequences may be predicted from the sequences listed in the tables. In addition, target names shown in Tables 1 and 2 were named Sp for SpCas9, Sa for SaCas9 and Cj for CjCas9 according to an editor protein.

TABLE 1

Target sequences of SERPINC1 gene(AT gene)

| Target name | Loci. | Target(w/o PAM) | GC Contents (%) | SEQ ID NO |
|---|---|---|---|---|
| hSerpinc1-Sp-1 | Exon01 (E01) | TCCTATCACATTGGAATACA | 35 | SEQ ID NO: 19 |
| hSerpinc1-Sp-2 | E01 | GGTTACAGTTCCTATCACAT | 40 | SEQ ID NO: 20 |
| hSerpinc1-Sp-3 | E01 | GCCATGTATTCCAATGTGAT | 40 | SEQ ID NO: 21 |
| hSerpinc1-Sp-4 | E01 | GTGATAGGAACTGTAACCTC | 45 | SEQ ID NO: 22 |
| hSerpinc1-Sp-5 | E01 | CCCCTCTTACCTTTTTCCAG | 50 | SEQ ID NO: 23 |
| hSerpinc1-Sp-6 | E01 | GAACTGTAACCTCTGGAAAA | 40 | SEQ ID NO: 24 |
| hSerpinc1-Sp-7 | E02 | CCAGAAGCCAATGAGCAGCA | 55 | SEQ ID NO: 25 |
| hSerpinc1-Sp-8 | E02 | CTTTTGTCCTTGCTGCTCAT | 45 | SEQ ID NO: 26 |
| hSerpinc1-Sp-9 | E02 | CCTTGCTGCTCATTGGCTTC | 55 | SEQ ID NO: 27 |
| hSerpinc1-Sp-10 | E02 | CTTGCTGCTCATTGGCTTCT | 50 | SEQ ID NO: 28 |
| hSerpinc1-Sp-11 | E02 | TGGGACTGCGTGACCTGTCA | 60 | SEQ ID NO: 29 |
| hSerpinc1-Sp-12 | E02 | GGGACTGCGTGACCTGTCAC | 65 | SEQ ID NO: 30 |
| hSerpinc1-Sp-13 | E02 | GTCCACAGGGCTCCCGTGAC | 70 | SEQ ID NO: 31 |
| hSerpinc1-Sp-14 | E02 | GACCTGTCACGGGAGCCCTG | 70 | SEQ ID NO: 32 |
| hSerpinc1-Sp-15 | E02 | TGGCTGTGCAGATGTCCACA | 55 | SEQ ID NO: 33 |
| hSerpinc1-Sp-16 | E02 | TTGGCTGTGCAGATGTCCAC | 55 | SEQ ID NO: 34 |
| hSerpinc1-Sp-17 | E02 | ACATCTGCACAGCCAAGCCG | 60 | SEQ ID NO: 35 |
| hSerpinc1-Sp-18 | E02 | CATCTGCACAGCCAAGCCGC | 65 | SEQ ID NO: 36 |
| hSerpinc1-Sp-19 | E02 | CATGGGAATGTCCCGCGGCT | 65 | SEQ ID NO: 37 |
| hSerpinc1-Sp-20 | E02 | GGATTCATGGGAATGTCCCG | 55 | SEQ ID NO: 38 |
| hSerpinc1-Sp-21 | E02 | TAAATGCACATGGGATTCAT | 35 | SEQ ID NO: 39 |
| hSerpinc1-Sp-22 | E02 | GTAAATGCACATGGGATTCA | 40 | SEQ ID NO: 40 |
| hSerpinc1-Sp-23 | E02 | GGGGAGCGGTAAATGCACAT | 55 | SEQ ID NO: 41 |
| hSerpinc1-Sp-24 | E02 | CGGGGAGCGGTAAATGCACA | 60 | SEQ ID NO: 42 |
| hSerpinc1-Sp-25 | E02 | CATGTGCATTTACCGCTCCC | 55 | SEQ ID NO: 43 |
| hSerpinc1-Sp-26 | E02 | TTGCCTTCTTCTCCGGGGAG | 60 | SEQ ID NO: 44 |
| hSerpinc1-Sp-27 | E02 | CTCAGTTGCCTTCTTCTCCG | 55 | SEQ ID NO: 45 |
| hSerpinc1-Sp-28 | E02 | CCTCAGTTGCCTTCTTCTCC | 55 | SEQ ID NO: 46 |
| hSerpinc1-Sp-29 | E02 | TCCTCAGTTGCCTTCTTCTC | 50 | SEQ ID NO: 47 |
| hSerpinc1-Sp-30 | E02 | TTACCGCTCCCCGGAGAAGA | 60 | SEQ ID NO: 48 |
| hSerpinc1-Sp-31 | E02 | CCCGGAGAAGAAGGCAACTG | 60 | SEQ ID NO: 49 |
| hSerpinc1-Sp-32 | E02 | GAAGAAGGCAACTGAGGATG | 50 | SEQ ID NO: 50 |
| hSerpinc1-Sp-33 | E02 | AAGAAGGCAACTGAGGATGA | 45 | SEQ ID NO: 51 |
| hSerpinc1-Sp-34 | E02 | GGGCTCAGAACAGAAGATCC | 55 | SEQ ID NO: 52 |
| hSerpinc1-Sp-35 | E02 | CTCAGAACAGAAGATCCCGG | 55 | SEQ ID NO: 53 |
| hSerpinc1-Sp-36 | E02 | CACGCCGGTTGGTGGCCTCC | 75 | SEQ ID NO: 54 |
| hSerpinc1-Sp-37 | E02 | ACACGCCGGTTGGTGGCCTC | 70 | SEQ ID NO: 55 |
| hSerpinc1-Sp-38 | E02 | AGATCCCGGAGGCCACCAAC | 65 | SEQ ID NO: 56 |
| hSerpinc1-Sp-39 | E02 | TTCCCAGACACGCCGGTTGG | 65 | SEQ ID NO: 57 |
| hSerpinc1-Sp-40 | E02 | CAGTTCCCAGACACGCCGGT | 65 | SEQ ID NO: 58 |
| hSerpinc1-Sp-41 | E02 | TGGACAGTTCCCAGACACGC | 60 | SEQ ID NO: 59 |
| hSerpinc1-Sp-42 | E02 | AGGCCACCAACCGGCGTGTC | 70 | SEQ ID NO: 60 |
| hSerpinc1-Sp-43 | E02 | GGCCACCAACCGGCGTGTCT | 70 | SEQ ID NO: 61 |
| hSerpinc1-Sp-44 | E02 | GCGTGTCTGGGAACTGTCCA | 60 | SEQ ID NO: 62 |
| hSerpinc1-Sp-45 | E02 | AGCAAAGCGGGAATTGGCCT | 55 | SEQ ID NO: 63 |
| hSerpinc1-Sp-46 | E02 | AGTGGTAGCAAAGCGGGAAT | 50 | SEQ ID NO: 64 |
| hSerpinc1-Sp-47 | E02 | ATAGAAAGTGGTAGCAAAGC | 40 | SEQ ID NO: 65 |
| hSerpinc1-Sp-48 | E02 | GATAGAAAGTGGTAGCAAAG | 40 | SEQ ID NO: 66 |
| hSerpinc1-Sp-49 | E02 | TGCCAGGTGCTGATAGAAAG | 50 | SEQ ID NO: 67 |
| hSerpinc1-Sp-50 | E02 | TACCACTTTCTATCAGCACC | 45 | SEQ ID NO: 68 |
| hSerpinc1-Sp-51 | E02 | TGTCATTCTTGGAATCTGCC | 45 | SEQ ID NO: 69 |
| hSerpinc1-Sp-52 | E02 | AATGTTATCATTGTCATTCT | 25 | SEQ ID NO: 70 |
| hSerpinc1-Sp-53 | E02 | TGGAGATACTCAGGGGTGAC | 55 | SEQ ID NO: 71 |
| hSerpinc1-Sp-54 | E02 | AAAGCCGTGGAGATACTCAG | 50 | SEQ ID NO: 72 |
| hSerpinc1-Sp-55 | E02 | AAAAGCCGTGGAGATACTCA | 45 | SEQ ID NO: 73 |
| hSerpinc1-Sp-56 | E02 | CAAAAGCCGTGGAGATACTC | 50 | SEQ ID NO: 74 |
| hSerpinc1-Sp-57 | E02 | GTCACCCCTGAGTATCTCCA | 55 | SEQ ID NO: 75 |
| hSerpinc1-Sp-58 | E02 | CTTGGTCATAGCAAAAGCCG | 50 | SEQ ID NO: 76 |
| hSerpinc1-Sp-59 | E02 | GGCTTTTGCTATGACCAAGC | 50 | SEQ ID NO: 77 |
| hSerpinc1-Sp-60 | E02 | GCTTTTGCTATGACCAAGCT | 45 | SEQ ID NO: 78 |
| hSerpinc1-Sp-61 | E02 | GTCATTACAGGCACCCAGCT | 55 | SEQ ID NO: 79 |
| hSerpinc1-Sp-62 | E02 | TTGCTGGAGGGTGTCATTAC | 50 | SEQ ID NO: 80 |
| hSerpinc1-Sp-63 | E02 | TACCTCCATCAGTTGCTGGA | 50 | SEQ ID NO: 81 |
| hSerpinc1-Sp-64 | E02 | GTACCTCCATCAGTTGCTGG | 55 | SEQ ID NO: 82 |
| hSerpinc1-Sp-65 | E02 | GTCGTACCTCCATCAGTTGC | 55 | SEQ ID NO: 83 |
| hSerpinc1-Sp-66 | E02 | TGACACCCTCCAGCAACTGA | 55 | SEQ ID NO: 84 |
| hSerpinc1-Sp-67 | E02 | CACCCTCCAGCAACTGATGG | 60 | SEQ ID NO: 85 |
| hSerpinc1-Sp-68 | E03 | ATCAGATGTTTTCTCAGATA | 30 | SEQ ID NO: 86 |
| hSerpinc1-Sp-69 | E03 | TCAGTTTGGCAAAGAAGAAG | 40 | SEQ ID NO: 87 |
| hSerpinc1-Sp-70 | E03 | ATAGAGTCGGCAGTTCAGTT | 45 | SEQ ID NO: 88 |
| hSerpinc1-Sp-71 | E03 | TGTTGGCTTTTCGATAGAGT | 40 | SEQ ID NO: 89 |
| hSerpinc1-Sp-72 | E03 | TACTAACTTGGAGGATTTGT | 35 | SEQ ID NO: 90 |
| hSerpinc1-Sp-73 | E03 | ATTGGCTGATACTAACTTGG | 40 | SEQ ID NO: 91 |
| hSerpinc1-Sp-74 | E03 | GCGATTGGCTGATACTAACT | 45 | SEQ ID NO: 92 |
| hSerpinc1-Sp-75 | E03 | TTTGTCTCCAAAAAGGCGAT | 40 | SEQ ID NO: 93 |
| hSerpinc1-Sp-76 | E03 | GTATCAGCCAATCGCCTTTT | 45 | SEQ ID NO: 94 |
| hSerpinc1-Sp-77 | E03 | TAAGGGATTTGTCTCCAAAA | 35 | SEQ ID NO: 95 |
| hSerpinc1-Sp-78 | E03 | GTAGGTCTCATTGAAGGTAA | 40 | SEQ ID NO: 96 |
| hSerpinc1-Sp-79 | E03 | GGTAGGTCTCATTGAAGGTA | 45 | SEQ ID NO: 97 |
| hSerpinc1-Sp-80 | E03 | GTCCTGGTAGGTCTCATTGA | 50 | SEQ ID NO: 98 |
| hSerpinc1-Sp-81 | E03 | TACCTTCAATGAGACCTACC | 45 | SEQ ID NO: 99 |
| hSerpinc1-Sp-82 | E03 | CAACTCACTGATGTCCTGGT | 50 | SEQ ID NO: 100 |
| hSerpinc1-Sp-83 | E03 | ATACCAACTCACTGATGTCC | 45 | SEQ ID NO: 101 |
| hSerpinc1-Sp-84 | E03 | CTACCAGGACATCAGTGAGT | 50 | SEQ ID NO: 102 |
| hSerpinc1-Sp-85 | E03 | GACATCAGTGAGTTGGTATA | 40 | SEQ ID NO: 103 |
| hSerpinc1-Sp-86 | E03 | GAAGTCCAGGGGCTGGAGCT | 65 | SEQ ID NO: 104 |
| hSerpinc1-Sp-87 | E03 | TGGAGCCAAGCTCCAGCCCC | 70 | SEQ ID NO: 105 |
| hSerpinc1-Sp-88 | E03 | TCACCTTGAAGTCCAGGGGC | 60 | SEQ ID NO: 106 |
| hSerpinc1-Sp-89 | E03 | CAACTCACCTTGAAGTCCAG | 50 | SEQ ID NO: 107 |
| hSerpinc1-Sp-90 | E03 | GCAACTCACCTTGAAGTCCA | 50 | SEQ ID NO: 108 |
| hSerpinc1-Sp-91 | E03 | TGCAACTCACCTTGAAGTCC | 50 | SEQ ID NO: 109 |
| hSerpinc1-Sp-92 | E03 | GCTCCAGCCCCTGGACTTCA | 65 | SEQ ID NO: 110 |
| hSerpinc1-Sp-93 | E04 | GATTGCTCTGCATTTTCCTG | 45 | SEQ ID NO: 111 |
| hSerpinc1-Sp-94 | E04 | AAATGCAGAGCAATCCAGAG | 45 | SEQ ID NO: 112 |
| hSerpinc1-Sp-95 | E04 | CCATTTGTTGATGGCCGCTC | 55 | SEQ ID NO: 113 |
| hSerpinc1-Sp-96 | E04 | ATTGGACACCCATTTGTTGA | 40 | SEQ ID NO: 114 |
| hSerpinc1-Sp-97 | E04 | CCAGAGCGGCCATCAACAAA | 55 | SEQ ID NO: 115 |
| hSerpinc1-Sp-98 | E04 | CAGAGCGGCCATCAACAAAT | 50 | SEQ ID NO: 116 |
| hSerpinc1-Sp-99 | E04 | GATTCGGCCTTCGGTCTTAT | 50 | SEQ ID NO: 117 |
| hSerpinc1-Sp-100 | E04 | TGGGTGTCCAATAAGACCGA | 50 | SEQ ID NO: 118 |
| hSerpinc1-Sp-101 | E04 | GACATCGGTGATTCGGCCTT | 55 | SEQ ID NO: 119 |
| hSerpinc1-Sp-102 | E04 | AGGGAATGACATCGGTGATT | 45 | SEQ ID NO: 120 |
| hSerpinc1-Sp-103 | E04 | GGCTTCCGAGGGAATGACAT | 55 | SEQ ID NO: 121 |
| hSerpinc1-Sp-104 | E04 | AATCACCGATGTCATTCCCT | 45 | SEQ ID NO: 122 |
| hSerpinc1-Sp-105 | E04 | AGCTCATTGATGGCTTCCGA | 50 | SEQ ID NO: 123 |
| hSerpinc1-Sp-106 | E04 | GAGCTCATTGATGGCTTCCG | 55 | SEQ ID NO: 124 |
| hSerpinc1-Sp-107 | E04 | CAGAACAGTGAGCTCATTGA | 45 | SEQ ID NO: 125 |
| hSerpinc1-Sp-108 | E04 | CATCAATGAGCTCACTGTTC | 45 | SEQ ID NO: 126 |
| hSerpinc1-Sp-109 | E04 | TGAGCTCACTGTTCTGGTGC | 55 | SEQ ID NO: 127 |
| hSerpinc1-Sp-110 | E04 | TCTGAGTACCTTGAAGTAAA | 35 | SEQ ID NO: 128 |
| hSerpinc1-Sp-111 | E04 | GGTTAACACCATTTACTTCA | 35 | SEQ ID NO: 129 |
| hSerpinc1-Sp-112 | E05 | ACTTTGACTTCCACAGGCCC | 55 | SEQ ID NO: 130 |
| hSerpinc1-Sp-113 | E05 | TGATTCTCTTCCAGGGCCTG | 55 | SEQ ID NO: 131 |
| hSerpinc1-Sp-114 | E05 | GGCTGAACTTTGACTTCCAC | 50 | SEQ ID NO: 132 |
| hSerpinc1-Sp-115 | E05 | GTTCCTTCCTTGTGTTCTCA | 45 | SEQ ID NO: 133 |
| hSerpinc1-Sp-116 | E05 | AGTTCCTTCCTTGTGTTCTC | 45 | SEQ ID NO: 134 |
| hSerpinc1-Sp-117 | E05 | AGTTCAGCCCTGAGAACACA | 50 | SEQ ID NO: 135 |
| hSerpinc1-Sp-118 | E05 | CAGCCCTGAGAACACAAGGA | 55 | SEQ ID NO: 136 |
| hSerpinc1-Sp-119 | E05 | AAGGAAGGAACTGTTCTACA | 40 | SEQ ID NO: 137 |
| hSerpinc1-Sp-120 | E05 | GAACTGTTCTACAAGGCTGA | 45 | SEQ ID NO: 138 |
| hSerpinc1-Sp-121 | E05 | TTCAGCATCTATGATGTACC | 40 | SEQ ID NO: 139 |
| hSerpinc1-Sp-122 | E05 | GCATCTATGATGTACCAGGA | 45 | SEQ ID NO: 140 |
| hSerpinc1-Sp-123 | E05 | GATAACGGAACTTGCCTTCC | 50 | SEQ ID NO: 141 |
| hSerpinc1-Sp-124 | E05 | AGGAAGGCAAGTTCCGTTAT | 45 | SEQ ID NO: 142 |
| hSerpinc1-Sp-125 | E05 | CTTCAGCCACGCGCCGATAA | 60 | SEQ ID NO: 143 |
| hSerpinc1-Sp-126 | E05 | CAAGTTCCGTTATCGGCGCG | 60 | SEQ ID NO: 144 |
| hSerpinc1-Sp-127 | E05 | CGTTATCGGCGCGTGGCTGA | 65 | SEQ ID NO: 145 |
| hSerpinc1-Sp-128 | E05 | GCGCGTGGCTGAAGGCACCC | 75 | SEQ ID NO: 146 |
| hSerpinc1-Sp-129 | E05 | GAAGGGCAACTCAAGCACCT | 55 | SEQ ID NO: 147 |
| hSerpinc1-Sp-130 | E05 | TGAAGGGCAACTCAAGCACC | 55 | SEQ ID NO: 148 |
| hSerpinc1-Sp-131 | E05 | GTGCTTGAGTTGCCCTTCAA | 50 | SEQ ID NO: 149 |
| hSerpinc1-Sp-132 | E05 | GTGATGTCATCACCTTTGAA | 40 | SEQ ID NO: 150 |
| hSerpinc1-Sp-133 | E05 | GGTGATGTCATCACCTTTGA | 45 | SEQ ID NO: 151 |
| hSerpinc1-Sp-134 | E05 | CAAAGGTGATGACATCACCA | 45 | SEQ ID NO: 152 |
| hSerpinc1-Sp-135 | E05 | CTTGGGCAAGATGAGGACCA | 55 | SEQ ID NO: 153 |
| hSerpinc1-Sp-136 | E05 | TCTCAGGCTTGGGCAAGATG | 55 | SEQ ID NO: 154 |
| hSerpinc1-Sp-137 | E05 | GCCAGGCTCTTCTCAGGCTT | 60 | SEQ ID NO: 155 |
| hSerpinc1-Sp-138 | E05 | GGCCAGGCTCTTCTCAGGCT | 65 | SEQ ID NO: 156 |
| hSerpinc1-Sp-139 | E05 | ACCTTGGCCAGGCTCTTCTC | 60 | SEQ ID NO: 157 |
| hSerpinc1-Sp-140 | E05 | GCCCAAGCCTGAGAAGAGCC | 65 | SEQ ID NO: 158 |
| hSerpinc1-Sp-141 | E05 | GCCTGAGAAGAGCCTGGCCA | 65 | SEQ ID NO: 159 |
| hSerpinc1-Sp-142 | E05 | GTTCCTTCTCTACCTTGGCC | 55 | SEQ ID NO: 160 |
| hSerpinc1-Sp-143 | E05 | GGTGAGTTCCTTCTCTACCT | 50 | SEQ ID NO: 161 |
| hSerpinc1-Sp-144 | E05 | GAGCCTGGCCAAGGTAGAGA | 60 | SEQ ID NO: 162 |
| hSerpinc1-Sp-145 | E05 | AGAGAAGGAACTCACCCCAG | 55 | SEQ ID NO: 163 |
| hSerpinc1-Sp-146 | E05 | CCACTCTTGCAGCACCTCTG | 60 | SEQ ID NO: 164 |
| hSerpinc1-Sp-147 | E05 | GCCACTCTTGCAGCACCTCT | 60 | SEQ ID NO: 165 |
| hSerpinc1-Sp-148 | E05 | AGCCACTCTTGCAGCACCTC | 60 | SEQ ID NO: 166 |
| hSerpinc1-Sp-149 | E05 | CCCCAGAGGTGCTGCAAGAG | 65 | SEQ ID NO: 167 |
| hSerpinc1-Sp-150 | E05 | AGAGGTGCTGCAAGAGTGGC | 60 | SEQ ID NO: 168 |
| hSerpinc1-Sp-151 | E05 | GCAAGAGTGGCTGGATGAAT | 50 | SEQ ID NO: 169 |
| hSerpinc1-Sp-152 | E05 | AGAGTGGCTGGATGAATTGG | 50 | SEQ ID NO: 170 |
| hSerpinc1-Sp-153 | E05 | TGAATTGGAGGAGATGATGC | 45 | SEQ ID NO: 171 |
| hSerpinc1-Sp-154 | E05 | ATTGGAGGAGATGATGCTGG | 50 | SEQ ID NO: 172 |
| hSerpinc1-Sp-155 | E05 | CAATGCGGAAGCGGGGCATG | 65 | SEQ ID NO: 173 |
| hSerpinc1-Sp-156 | E05 | CCGTCCTCAATGCGGAAGCG | 65 | SEQ ID NO: 174 |
| hSerpinc1-Sp-157 | E05 | GCCGTCCTCAATGCGGAAGC | 65 | SEQ ID NO: 175 |
| hSerpinc1-Sp-158 | E05 | AGCCGTCCTCAATGCGGAAG | 60 | SEQ ID NO: 176 |
| hSerpinc1-Sp-159 | E05 | CATGCCCCGCTTCCGCATTG | 65 | SEQ ID NO: 177 |
| hSerpinc1-Sp-160 | E05 | AACTGAAGCCGTCCTCAATG | 50 | SEQ ID NO: 178 |
| hSerpinc1-Sp-161 | E05 | CCCCGCTTCCGCATTGAGGA | 65 | SEQ ID NO: 179 |
| hSerpinc1-Sp-162 | E05 | TGAGGACGGCTTCAGTTTGA | 50 | SEQ ID NO: 180 |
| hSerpinc1-Sp-163 | E05 | GAAGGAGCAGCTGCAAGACA | 55 | SEQ ID NO: 181 |
| hSerpinc1-Sp-164 | E05 | AAGGAGCAGCTGCAAGACAT | 50 | SEQ ID NO: 182 |
| hSerpinc1-Sp-165 | E05 | CAGGGCTGAACAGATCGACA | 55 | SEQ ID NO: 183 |
| hSerpinc1-Sp-166 | E05 | CTGGGAGTTTGGACTTTTCA | 45 | SEQ ID NO: 184 |
| hSerpinc1-Sp-167 | E05 | CCTGGGAGTTTGGACTTTTC | 50 | SEQ ID NO: 185 |
| hSerpinc1-Sp-168 | E05 | CCTAGACAAACCTGGGAGTT | 50 | SEQ ID NO: 186 |
| hSerpinc1-Sp-169 | E05 | CCTGAAAAGTCCAAACTCCC | 50 | SEQ ID NO: 187 |
| hSerpinc1-Sp-170 | E06 | CGGCCTTCTGCAACAATACC | 55 | SEQ ID NO: 188 |
| hSerpinc1-Sp-171 | E06 | CTTCCAGGTATTGTTGCAGA | 45 | SEQ ID NO: 189 |
| hSerpinc1-Sp-172 | E06 | CTGAGACATAGAGGTCATCT | 45 | SEQ ID NO: 190 |
| hSerpinc1-Sp-173 | E06 | GGAATGCATCTGAGACATAG | 45 | SEQ ID NO: 191 |
| hSerpinc1-Sp-174 | E06 | TGTCTCAGATGCATTCCATA | 40 | SEQ ID NO: 192 |
| hSerpinc1-Sp-175 | E06 | TCACCTCAAGAAATGCCTTA | 40 | SEQ ID NO: 193 |
| hSerpinc1-Sp-176 | E06 | ATTCCATAAGGCATTTCTTG | 35 | SEQ ID NO: 194 |
| hSerpinc1-Sp-177 | E07 | GACCTGCAGGTAAATGAAGA | 45 | SEQ ID NO: 195 |
| hSerpinc1-Sp-178 | E07 | ACGGCCAGCAATCACAACAG | 55 | SEQ ID NO: 196 |
| hSerpinc1-Sp-179 | E07 | AGTACCGCTGTTGTGATTGC | 50 | SEQ ID NO: 197 |
| hSerpinc1-Sp-180 | E07 | CCCTGTTGGGGTTTAGCGAA | 55 | SEQ ID NO: 198 |
| hSerpinc1-Sp-181 | E07 | GCCGTTCGCTAAACCCCAAC | 60 | SEQ ID NO: 199 |
| hSerpinc1-Sp-182 | E07 | CCGTTCGCTAAACCCCAACA | 55 | SEQ ID NO: 200 |
| hSerpinc1-Sp-183 | E07 | CCTTGAAAGTCACCCTGTTG | 50 | SEQ ID NO: 201 |
| hSerpinc1-Sp-184 | E07 | GCCTTGAAAGTCACCCTGTT | 50 | SEQ ID NO: 202 |
| hSerpinc1-Sp-185 | E07 | GGCCTTGAAAGTCACCCTGT | 55 | SEQ ID NO: 203 |
| hSerpinc1-Sp-186 | E07 | CCCCAACAGGGTGACTTTCA | 55 | SEQ ID NO: 204 |
| hSerpinc1-Sp-187 | E07 | GGGTGACTTTCAAGGCCAAC | 55 | SEQ ID NO: 205 |
| hSerpinc1-Sp-188 | E07 | AAAAACCAGGAAAGGCCTGT | 45 | SEQ ID NO: 206 |
| hSerpinc1-Sp-189 | E07 | CAAGGCCAACAGGCCTTTCC | 60 | SEQ ID NO: 207 |
| hSerpinc1-Sp-190 | E07 | TCTCTTATAAAAACCAGGAA | 30 | SEQ ID NO: 208 |
| hSerpinc1-Sp-191 | E07 | GAACTTCTCTTATAAAAACC | 30 | SEQ ID NO: 209 |
| hSerpinc1-Sp-192 | E07 | ATGAAGATAATAGTGTTCAG | 30 | SEQ ID NO: 210 |
| hSerpinc1-Sp-193 | E07 | TCTGAACACTATTATCTTCA | 30 | SEQ ID NO: 211 |
| hSerpinc1-Sp-194 | E07 | CTGAACACTATTATCTTCAT | 30 | SEQ ID NO: 212 |
| hSerpinc1-Sp-195 | E07 | ATTTTACTTAACACAAGGGT | 30 | SEQ ID NO: 213 |
| hSerpinc1-Sp-196 | E07 | GAACATTTTACTTAACACAA | 25 | SEQ ID NO: 214 |
| hSerpinc1-Sp-197 | E07 | AGAACATTTTACTTAACACA | 25 | SEQ ID NO: 215 |
| hSerpinc1-Sa-1 | E01 | GGTTACAGTTCCTATCACAT | 40 | SEQ ID NO: 216 |
| hSerpinc1-Sa-2 | E02 | CCAAGCCGCGGGACATTCCC | 70 | SEQ ID NO: 217 |
| hSerpinc1-Sa-3 | E02 | TAAATGCACATGGGATTCAT | 35 | SEQ ID NO: 218 |
| hSerpinc1-Sa- | E02 | CGGGGAGCGGTAAATGCACA | 60 | SEQ ID NO: 219 |
| Serpinc1-Sa-5 | E02 | CCCCGGAGAAGAAGGCAACT | 60 | SEQ ID NO: 220 |
| hSerpinc1-Sa-6 | E02 | ACACGCCGGTTGGTGGCCTC | 70 | SEQ ID NO: 221 |
| hSerpinc1-Sa-7 | E02 | ATAGAAAGTGGTAGCAAAGC | 40 | SEQ ID NO: 222 |
| hSerpinc1-Sa-8 | E02 | ATCAGCACCTGGCAGATTCC | 55 | SEQ ID NO: 223 |
| hSerpinc1-Sa-9 | E02 | AATGTTATCATTGTCATTCT | 25 | SEQ ID NO: 224 |
| hSerpinc1-Sa-10 | E02 | ATAACATTTTCCTGTCACCC | 40 | SEQ ID NO: 225 |
| hSerpinc1-Sa-11 | E02 | CAAAAGCCGTGGAGATACTC | 50 | SEQ ID NO: 226 |
| hSerpinc1-Sa-12 | E02 | CGGCTTTTGCTATGACCAAG | 50 | SEQ ID NO: 227 |
| hSerpinc1-Sa-13 | E02 | CGTACCTCCATCAGTTGCTG | 55 | SEQ ID NO: 228 |
| hSerpinc1-Sa-14 | E03 | TTCAGTTTGGCAAAGAAGAA | 35 | SEQ ID NO: 229 |
| hSerpinc1-Sa-15 | E03 | AGGATTTGTTGGCTTTTCGA | 40 | SEQ ID NO: 230 |
| hSerpinc1-Sa-16 | E03 | GATTGGCTGATACTAACTTG | 40 | SEQ ID NO: 231 |
| hSerpinc1-Sa-17 | E03 | GGTAGGTCTCATTGAAGGTA | 45 | SEQ ID NO: 232 |
| hSerpinc1-Sa-18 | E03 | TGAGACCTACCAGGACATCA | 50 | SEQ ID NO: 233 |
| hSerpinc1-Sa-19 | E03 | TCCAGCCCCTGGACTTCAAG | 60 | SEQ ID NO: 234 |
| hSerpinc1-Sa-20 | E04 | CCCATTTGTTGATGGCCGCT | 55 | SEQ ID NO: 235 |
| hSerpinc1-Sa-21 | E04 | TCCAGAGCGGCCATCAACAA | 55 | SEQ ID NO: 236 |
| hSerpinc1-Sa-22 | E04 | GTGTCCAATAAGACCGAAGG | 50 | SEQ ID NO: 237 |
| hSerpinc1-Sa-23 | E04 | AGCTCATTGATGGCTTCCGA | 50 | SEQ ID NO: 238 |
| hSerpinc1-Sa-24 | E05 | ACTGTTCTACAAGGCTGATG | 45 | SEQ ID NO: 239 |
| hSerpinc1-Sa-25 | E05 | GGCTGAAGGCACCCAGGTGC | 70 | SEQ ID NO: 240 |
| hSerpinc1-Sa-26 | E05 | TTGAAGGGCAACTCAAGCAC | 50 | SEQ ID NO: 241 |
| hSerpinc1-Sa-27 | E05 | ACTCTTGCAGCACCTCTGGG | 60 | SEQ ID NO: 242 |
| hSerpinc1-Sa-28 | E05 | AGCCACTCTTGCAGCACCTC | 60 | SEQ ID NO: 243 |
| hSerpinc1-Sa-29 | E05 | ACTCACCCCAGAGGTGCTGC | 65 | SEQ ID NO: 244 |
| hSerpinc1-Sa-30 | E05 | CAGAGGTGCTGCAAGAGTGG | 60 | SEQ ID NO: 245 |
| hSerpinc1-Sa-31 | E05 | GGTGCTGCAAGAGTGGCTGG | 65 | SEQ ID NO: 246 |
| hSerpinc1-Sa-32 | E06 | TCACCTCAAGAAATGCCTTA | 40 | SEQ ID NO: 247 |
| hSerpinc1-Sa-33 | E06 | TCCATAAGGCATTTCTTGAG | 40 | SEQ ID NO: 248 |
| hSerpinc1-Sa-34 | E07 | GGCCGTTCGCTAAACCCCAA | 60 | SEQ ID NO: 249 |
| hSerpinc1-Sa-35 | E07 | GGCCTTGAAAGTCACCCTGT | 55 | SEQ ID NO: 250 |
| hSerpinc1-Sa-36 | E07 | AACACTATTATCTTCATGGG | 35 | SEQ ID NO: 251 |
| hSerpinc1-Sa-37 | E07 | AAGAACATTTTACTTAACAC | 25 | SEQ ID NO: 252 |
| hSerpinc1-Cj-1 | E01 | GAGGTTACAGTTCCTATCACAT | 41 | SEQ ID NO: 253 |
| hSerpinc1-Cj-2 | E02 | CTGTCACGGGAGCCCTGTGGAC | 68 | SEQ ID NO: 254 |
| hSerpinc1-Cj-3 | E02 | GCCTTCTTCTCCGGGGAGCGGT | 68 | SEQ ID NO: 255 |
| hSerpinc1-Cj-4 | E02 | GGGAATTGGCCTTGGACAGTTC | 55 | SEQ ID NO: 256 |
| Serpinc1-Cj-5 | E02 | TCCCGCTTTGCTACCACTTTCT | 50 | SEQ ID NO: 257 |
| hSerpinc1-Cj-6 | E02 | GCTTGGTCATAGCAAAAGCCGT | 50 | SEQ ID NO: 258 |
| hSerpinc1-Cj-7 | E02 | TATGACCAAGCTGGGTGCCTGT | 55 | SEQ ID NO: 259 |
| hSerpinc1-Cj-8 | E02 | TCAGTTGCTGGAGGGTGTCATT | 50 | SEQ ID NO: 260 |
| hSerpinc1-Cj-9 | E02 | ATGACACCCTCCAGCAACTGAT | 50 | SEQ ID NO: 261 |
| hSerpinc1-Cj-10 | E03 | TTGACTTCTATAGGTATTTAAG | 27 | SEQ ID NO: 262 |
| hSerpinc1-Cj-11 | E03 | TTTCTCAGATATGGTGTCAAAC | 36 | SEQ ID NO: 263 |
| hSerpinc1-Cj-12 | E03 | TTTGTCTCCAAAAAGGCGATTG | 41 | SEQ ID NO: 264 |
| hSerpinc1-Cj-13 | E03 | GTCCAGGGGCTGGAGCTTGGCT | 68 | SEQ ID NO: 265 |
| hSerpinc1-Cj-14 | E04 | GGTGATTCGGCCTTCGGTCTTA | 55 | SEQ ID NO: 266 |
| hSerpinc1-Cj-15 | E04 | TGAGCTCACTGTTCTGGTGCTG | 55 | SEQ ID NO: 267 |
| hSerpinc1-Cj-16 | E04 | TACCTTGAAGTAAATGGTGTTA | 32 | SEQ ID NO: 268 |
| hSerpinc1-Cj-17 | E04 | TGCTGGTTAACACCATTTACTT | 36 | SEQ ID NO: 269 |
| hSerpinc1-Cj-18 | E05 | GTGGAAGTCAAAGTTCAGCCCT | 50 | SEQ ID NO: 270 |
| hSerpinc1-Cj-19 | E05 | GGAGAGTCGTGTTCAGCATCTA | 50 | SEQ ID NO: 271 |
| hSerpinc1-Cj-20 | E05 | CCTTCCTGGTACATCATAGATG | 45 | SEQ ID NO: 272 |
| hSerpinc1-Cj-21 | E05 | CGCCGATAACGGAACTTGCCTT | 55 | SEQ ID NO: 273 |
| hSerpinc1-Cj-22 | E05 | GTTCCGTTATCGGCGCGTGGCT | 64 | SEQ ID NO: 274 |
| hSerpinc1-Cj-23 | E05 | GTCATCACCTTTGAAGGGCAAC | 50 | SEQ ID NO: 275 |
| hSerpinc1-Cj-24 | E05 | CTCCAATTCATCCAGCCACTCT | 50 | SEQ ID NO: 276 |
| hSerpinc1-Cj-25 | E06 | AGAGGTCATCTCGGCCTTCTGC | 59 | SEQ ID NO: 277 |
| hSerpinc1-Cj-26 | E06 | ATTCCATAAGGCATTTCTTGAG | 36 | SEQ ID NO: 278 |
| hSerpinc1-Cj-27 | E07 | TGAAGAAGGCAGTGAAGCAGCT | 50 | SEQ ID NO: 279 |
| hSerpinc1-Cj-28 | E07 | GCGAACGGCCAGCAATCACAAC | 59 | SEQ ID NO: 280 |
| hSerpinc1-Cj-29 | E07 | GGTTTTTATAAGAGAAGTTCCT | 32 | SEQ ID NO: 281 |
| hSerpinc1-Cj-30 | E07 | TGCAAAGAATAAGAACATTTTA | 23 | SEQ ID NO: 282 |

TABLE 2

Target sequences of TFPI gene

| Target name | Loci. | Target(w/o PAM) | GC Contents (%) | SEQ ID NO |
|---|---|---|---|---|
| hTfpi-Sp-1 | E02 | TGAAGAAAGTACATGCACTT | 35 | SEQ ID NO: 283 |
| hTfpi-Sp-2 | E02 | GAAGAAAGTACATGCACTTT | 35 | SEQ ID NO: 284 |
| hTfpi-Sp-3 | E02 | CAGGGGCAAGATTAAGCAGC | 55 | SEQ ID NO: 285 |
| hTfpi-Sp-4 | E02 | ATCAGCATTAAGAGGGGCAG | 50 | SEQ ID NO: 286 |
| hTfpi-Sp-5 | E02 | AATCAGCATTAAGAGGGGCA | 45 | SEQ ID NO: 287 |
| hTfpi-Sp-6 | E02 | GAATCAGCATTAAGAGGGGC | 50 | SEQ ID NO: 288 |
| hTfpi-Sp-7 | E02 | CTCAGAATCAGCATTAAGAG | 40 | SEQ ID NO: 289 |
| hTfpi-Sp-8 | E02 | CCTCAGAATCAGCATTAAGA | 40 | SEQ ID NO: 290 |
| hTfpi-Sp-9 | E02 | TCCTCAGAATCAGCATTAAG | 40 | SEQ ID NO: 291 |
| hTfpi-Sp-10 | E02 | CCCTCTTAATGCTGATTCTG | 45 | SEQ ID NO: 292 |
| hTfpi-Sp-11 | E02 | GAAGAACACACAATTATCAC | 35 | SEQ ID NO: 293 |
| hTfpi-Sp-12 | E03 | TTATTTTTACTTTATAGATA | 10 | SEQ ID NO: 294 |
| hTfpi-Sp-13 | E03 | GAATGCATAAGTTTCAGTGG | 40 | SEQ ID NO: 295 |
| hTfpi-Sp-14 | E03 | AATGAATGCATAAGTTTCAG | 30 | SEQ ID NO: 296 |
| hTfpi-Sp-15 | E03 | GCATTCATTTTGTGCATTCA | 35 | SEQ ID NO: 297 |
| hTfpi-Sp-16 | E03 | TTCATTTTGTGCATTCAAGG | 35 | SEQ ID NO: 298 |
| hTfpi-Sp-17 | E03 | TGTGCATTCAAGGCGGATGA | 50 | SEQ ID NO: 299 |
| hTfpi-Sp-18 | E03 | TTTTCATGATTGCTTTACAT | 25 | SEQ ID NO: 300 |
| hTfpi-Sp-19 | E03 | CTTTTCATGATTGCTTTACA | 30 | SEQ ID NO: 301 |
| hTfpi-Sp-20 | E03 | CAGTGCGAAGAATTTATATA | 30 | SEQ ID NO: 302 |
| hTfpi-Sp-21 | E03 | AGTGCGAAGAATTTATATAT | 25 | SEQ ID NO: 303 |
| hTfpi-Sp-22 | E03 | GTGCGAAGAATTTATATATG | 30 | SEQ ID NO: 304 |
| hTfpi-Sp-23 | E03 | TGCGAAGAATTTATATATGG | 30 | SEQ ID NO: 305 |
| hTfpi-Sp-24 | E03 | TTTATATATGGGGGATGTGA | 35 | SEQ ID NO: 306 |
| hTfpi-Sp-25 | E03 | TCAGAATCGATTTGAAAGTC | 35 | SEQ ID NO: 307 |
| hTfpi-Sp-26 | E03 | TGCAAAAAAATGTGTACAAG | 30 | SEQ ID NO: 308 |
| hTfpi-Sp-27 | E03 | AAAAAATGTGTACAAGAGGT | 30 | SEQ ID NO: 309 |
| hTfpi-Sp-28 | E04 | TGATTACAGATAATGCAAAC | 30 | SEQ ID NO: 310 |
| hTfpi-Sp-29 | E04 | ATAAAGACAACATTGCAACA | 30 | SEQ ID NO: 311 |
| hTfpi-Sp-30 | E05 | TCTTCCAAAAAGCAGAAATC | 35 | SEQ ID NO: 312 |
| hTfpi-Sp-31 | E05 | AAAGCCAGATTTCTGCTTTT | 35 | SEQ ID NO: 313 |
| hTfpi-Sp-32 | E05 | TGCTTTTTGGAAGAAGATCC | 40 | SEQ ID NO: 314 |
| hTfpi-Sp-33 | E05 | ATATAACCTCGACATATTCC | 35 | SEQ ID NO: 315 |
| hTfpi-Sp-34 | E05 | GAAGATCCTGGAATATGTCG | 45 | SEQ ID NO: 316 |
| hTfpi-Sp-35 | E05 | TATGTCGAGGTTATATTACC | 35 | SEQ ID NO: 317 |
| hTfpi-Sp-36 | E05 | CTGATTGTTATAAAAATACC | 25 | SEQ ID NO: 318 |
| hTfpi-Sp-37 | E05 | CAGTGTGAACGTTTCAAGTA | 40 | SEQ ID NO: 319 |
| hTfpi-Sp-38 | E05 | TGTGAACGTTTCAAGTATGG | 40 | SEQ ID NO: 320 |
| hTfpi-Sp-39 | E05 | TTTCAAGTATGGTGGATGCC | 45 | SEQ ID NO: 321 |
| hTfpi-Sp-40 | E05 | TTCAAGTATGGTGGATGCCT | 45 | SEQ ID NO: 322 |
| hTfpi-Sp-41 | E05 | CAAAATTGTTCATATTGCCC | 35 | SEQ ID NO: 323 |
| hTfpi-Sp-42 | E05 | TATGAACAATTTTGAGACAC | 30 | SEQ ID NO: 324 |
| hTfpi-Sp-43 | E05 | TGCAAGAACATTTGTGAAGA | 35 | SEQ ID NO: 325 |
| hTfpi-Sp-44 | E06 | CTGTATTTTTTTCCAGCGAA | 35 | SEQ ID NO: 326 |
| hTfpi-Sp-45 | E06 | TCCACCTGGAAACCATTCGC | 55 | SEQ ID NO: 327 |
| hTfpi-Sp-46 | E06 | TTTTCCAGCGAATGGTTTCC | 45 | SEQ ID NO: 328 |
| hTfpi-Sp-47 | E06 | TCCAGCGAATGGTTTCCAGG | 55 | SEQ ID NO: 329 |
| hTfpi-Sp-48 | E06 | GGGTTCCATAATTATCCACC | 45 | SEQ ID NO: 330 |
| hTfpi-Sp-49 | E06 | GGTTTCCAGGTGGATAATTA | 40 | SEQ ID NO: 331 |
| hTfpi-Sp-50 | E06 | GTTATTCACAGCATTGAGCT | 40 | SEQ ID NO: 332 |
| hTfpi-Sp-51 | E06 | AGTTATTCACAGCATTGAGC | 40 | SEQ ID NO: 333 |
| hTfpi-Sp-52 | E06 | CTTGGTTGATTGCGGAGTCA | 50 | SEQ ID NO: 334 |
| hTfpi-Sp-53 | E06 | CCTTGGTTGATTGCGGAGTC | 55 | SEQ ID NO: 335 |
| hTfpi-Sp-54 | E06 | CTGGGAACCTTGGTTGATTG | 50 | SEQ ID NO: 336 |
| hTfpi-Sp-55 | E06 | CCTGACTCCGCAATCAACCA | 55 | SEQ ID NO: 337 |
| hTfpi-Sp-56 | E06 | ACCAAAAAGGCTGGGAACCT | 50 | SEQ ID NO: 338 |
| hTfpi-Sp-57 | E06 | AGATTCTTACCAAAAAGGCT | 35 | SEQ ID NO: 339 |
| hTfpi-Sp-58 | E06 | AAGATTCTTACCAAAAAGGC | 35 | SEQ ID NO: 340 |
| hTfpi-Sp-59 | E06 | ACCAAGGTTCCCAGCCTTTT | 50 | SEQ ID NO: 341 |
| hTfpi-Sp-60 | E06 | CCACAAGATTCTTACCAAAA | 35 | SEQ ID NO: 342 |
| hTfpi-Sp-61 | E06 | CCTTTTTGGTAAGAATCTTG | 35 | SEQ ID NO: 343 |
| hTfpi-Sp-62 | E07 | GTGAAATTCTAAAAACAATC | 25 | SEQ ID NO: 344 |
| hTfpi-Sp-63 | E07 | TGATTGTTTTTAGAATTTCA | 20 | SEQ ID NO: 345 |
| hTfpi-Sp-64 | E07 | TAGAATTTCACGGTCCCTCA | 45 | SEQ ID NO: 346 |
| hTfpi-Sp-65 | E07 | GCTGGAGTGAGACACCATGA | 55 | SEQ ID NO: 347 |
| hTfpi-Sp-66 | E07 | TGCTGGAGTGAGACACCATG | 55 | SEQ ID NO: 348 |
| hTfpi-Sp-67 | E07 | CGACACAATCCTCTGTCTGC | 55 | SEQ ID NO: 349 |
| hTfpi-Sp-68 | E07 | TGTCTCACTCCAGCAGACAG | 55 | SEQ ID NO: 350 |
| hTfpi-Sp-69 | E07 | GTAGTAGAATCTGTTCTCAT | 35 | SEQ ID NO: 351 |
| hTfpi-Sp-70 | E07 | TTCTACTACAATTCAGTCAT | 30 | SEQ ID NO: 352 |
| hTfpi-Sp-71 | E07 | TCTACTACAATTCAGTCATT | 30 | SEQ ID NO: 353 |
| hTfpi-Sp-72 | E07 | ATCCACTGTACTTAAATGGG | 40 | SEQ ID NO: 354 |
| hTfpi-Sp-73 | E07 | CACATCCACTGTACTTAAAT | 35 | SEQ ID NO: 355 |
| hTfpi-Sp-74 | E07 | CCACATCCACTGTACTTAAA | 40 | SEQ ID NO: 356 |
| hTfpi-Sp-75 | E07 | TGCCGCCCATTTAAGTACAG | 50 | SEQ ID NO: 357 |
| hTfpi-Sp-76 | E07 | CCATTTAAGTACAGTGGATG | 40 | SEQ ID NO: 358 |
| hTfpi-Sp-77 | E07 | CATTTAAGTACAGTGGATGT | 35 | SEQ ID NO: 359 |
| hTfpi-Sp-78 | E07 | ATTTAAGTACAGTGGATGTG | 35 | SEQ ID NO: 360 |
| hTfpi-Sp-79 | E07 | TTTAAGTACAGTGGATGTGG | 40 | SEQ ID NO: 361 |
| hTfpi-Sp-80 | E07 | TGCCCTCAGACATTCTTGTT | 45 | SEQ ID NO: 362 |
| hTfpi-Sp-81 | E07 | CTTCCAAACAAGAATGTCTG | 40 | SEQ ID NO: 363 |
| hTfpi-Sp-82 | E07 | TTCCAAACAAGAATGTCTGA | 35 | SEQ ID NO: 364 |
| hTfpi-Sp-83 | E07 | TGTCTGAGGGCATGTAAAAA | 40 | SEQ ID NO: 365 |
| hTfpi-Sp-84 | E08 | ATGAAACCTATAAGAGGAAG | 35 | SEQ ID NO: 366 |
| hTfpi-Sp-85 | E08 | CTTTGGATGAAACCTATAAG | 35 | SEQ ID NO: 367 |
| hTfpi-Sp-86 | E08 | GGCCTCCTTTTGATATTCTT | 40 | SEQ ID NO: 368 |
| hTfpi-Sp-87 | E08 | TTCATCCAAAGAATATCAAA | 25 | SEQ ID NO: 369 |
| hTfpi-Sp-88 | E08 | ATCCAAAGAATATCAAAAGG | 30 | SEQ ID NO: 370 |
| hTfpi-Sp-89 | E08 | TTTTTCTTTTGGTTTTAATT | 15 | SEQ ID NO: 371 |
| hTfpi-Sp-90 | E08 | CTGCTTCTTTCTTTTTCTTT | 30 | SEQ ID NO: 372 |
| hTfpi-Sa-1 | E02 | TGTGTGTTCTTCATCTTCCT | 40 | SEQ ID NO: 373 |
| hTfpi-Sa-2 | E03 | TTATTTTTACTTTATAGATA | 10 | SEQ ID NO: 374 |
| hTfpi-Sa-3 | E03 | ATCCGCCTTGAATGCACAAA | 45 | SEQ ID NO: 375 |
| hTfpi-Sa-4 | E03 | ATTCATTTTGTGCATTCAAG | 30 | SEQ ID NO: 376 |
| hTfpi-Sa-5 | E03 | TACATGGGCCATCATCCGCC | 60 | SEQ ID NO: 377 |
| hTfpi-Sa-6 | E03 | TATATAAATTCTTCGCACTG | 30 | SEQ ID NO: 378 |
| hTfpi-Sa-7 | E03 | TATTTTCACTCGACAGTGCG | 45 | SEQ ID NO: 379 |
| hTfpi-Sa-8 | E03 | GTGCGAAGAATTTATATATG | 30 | SEQ ID NO: 380 |
| hTfpi-Sa-9 | E03 | ATGGGGGATGTGAAGGAAAT | 45 | SEQ ID NO: 381 |
| hTfpi-Sa-10 | E03 | GAATCGATTTGAAAGTCTGG | 40 | SEQ ID NO: 382 |
| hTfpi-Sa-11 | E04 | TTGATTACAGATAATGCAAA | 25 | SEQ ID NO: 383 |
| hTfpi-Sa-12 | E05 | TGCTTTTTGGAAGAAGATCC | 40 | SEQ ID NO: 384 |
| hTfpi-Sa-13 | E05 | AATATAACCTCGACATATTC | 30 | SEQ ID NO: 385 |
| hTfpi-Sa-14 | E05 | GTGTGAACGTTTCAAGTATG | 40 | SEQ ID NO: 386 |
| hTfpi-Sa-15 | E05 | GAACAATTTTGAGACACTGG | 40 | SEQ ID NO: 387 |
| hTfpi-Sa-16 | E06 | TTCCAGCGAATGGTTTCCAG | 50 | SEQ ID NO: 388 |
| hTfpi-Sa-17 | E06 | GAGTTATTCACAGCATTGAG | 40 | SEQ ID NO: 389 |
| hTfpi-Sa-18 | E06 | ATGGAACCCAGCTCAATGCT | 50 | SEQ ID NO: 390 |
| hTfpi-Sa-19 | E06 | CTTGGTTGATTGCGGAGTCA | 50 | SEQ ID NO: 391 |
| hTfpi-Sa-20 | E06 | CTGGGAACCTTGGTTGATTG | 50 | SEQ ID NO: 392 |
| hTfpi-Sa-21 | E06 | AGGTTCCCAGCCTTTTTGGT | 50 | SEQ ID NO: 393 |
| hTfpi-Sa-22 | E07 | CGACACAATCCTCTGTCTGC | 55 | SEQ ID NO: 394 |
| hTfpi-Sa-23 | E07 | GTGTCTCACTCCAGCAGACA | 55 | SEQ ID NO: 395 |
| hTfpi-Sa-24 | E07 | TCCCAATGACTGAATTGTAG | 40 | SEQ ID NO: 396 |
| hTfpi-Sa-25 | E07 | TGGGCGGCATTTCCCAATGA | 55 | SEQ ID NO: 397 |
| hTfpi-Sa-26 | E07 | ATGCCGCCCATTTAAGTACA | 45 | SEQ ID NO: 398 |
| hTfpi-Sa-27 | E07 | AAACAATTTTACTTCCAAAC | 25 | SEQ ID NO: 399 |
| hTfpi-Sa-28 | E08 | CCTCTTATAGGTTTCATCCA | 40 | SEQ ID NO: 400 |
| hTfpi-Sa-29 | E08 | AGGCCTCCTTTTGATATTCT | 40 | SEQ ID NO: 401 |
| hTfpi-Sa-30 | E08 | GAAATTTTTGTTAAAAATAT | 10 | SEQ ID NO: 402 |
| hTfpi-Cj-1 | E02 | TGGGTTCTGTATTTCAGAGATG | 41 | SEQ ID NO: 403 |
| hTfpi-Cj-2 | E02 | TCTTCATTGTGTAAATCATCTC | 32 | SEQ ID NO: 404 |
| hTfpi-Cj-3 | E02 | CAGAGATGATTTACACAATGAA | 32 | SEQ ID NO: 405 |
| hTfpi-Cj-4 | E02 | TGATTTACACAATGAAGAAAGT | 27 | SEQ ID NO: 406 |
| hTfpi-Cj-5 | E02 | CAGGCATACAGAAGCCCAAAGT | 50 | SEQ ID NO: 407 |
| hTfpi-Cj-6 | E02 | GGCAGGGGCAAGATTAAGCAGC | 59 | SEQ ID NO: 408 |
| hTfpi-Cj-7 | E02 | AATGCTGATTCTGAGGAAGATG | 41 | SEQ ID NO: 409 |
| hTfpi-Cj-8 | E02 | TGCTGATTCTGAGGAAGATGAA | 41 | SEQ ID NO: 410 |
| hTfpi-Cj-9 | E03 | TACATGGGCCATCATCCGCCTT | 55 | SEQ ID NO: 411 |
| hTfpi-Cj-10 | E03 | TCACATCCCCCATATATAAATT | 32 | SEQ ID NO: 412 |
| hTfpi-Cj-11 | E03 | AAGTCTGGAAGAGTGCAAAAAA | 36 | SEQ ID NO: 413 |
| hTfpi-Cj-12 | E03 | AACCTACCTCTTGTACACATTT | 36 | SEQ ID NO: 414 |
| hTfpi-Cj-13 | E03 | AGGGTTCCCAGAAACCTACCTC | 55 | SEQ ID NO: 415 |
| hTfpi-Cj-14 | E05 | CACTGTTTTGTCTGATTGTTAT | 32 | SEQ ID NO: 416 |
| hTfpi-Cj-15 | E05 | AGGCATCCACCATACTTGAAAC | 45 | SEQ ID NO: 417 |
| hTfpi-Cj-16 | E05 | TTGTTCATATTGCCCAGGCATC | 45 | SEQ ID NO: 418 |
| hTfpi-Cj-17 | E05 | GCCTGGGCAATATGAACAATTT | 41 | SEQ ID NO: 419 |
| hTfpi-Cj-18 | E07 | CACAATCCTCTGTCTGCTGGAG | 55 | SEQ ID NO: 420 |
| hTfpi-Cj-19 | E07 | TAGTAGAATCTGTTCTCATTGG | 36 | SEQ ID NO: 421 |
| hTfpi-Cj-20 | E07 | AATTGTAGTAGAATCTGTTCTC | 32 | SEQ ID NO: 422 |
| hTfpi-Cj-21 | E07 | GTCATTGGGAAATGCCGCCCAT | 55 | SEQ ID NO: 423 |
| hTfpi-Cj-22 | E07 | TTGTTTTCATTTCCCCCACATC | 41 | SEQ ID NO: 424 |

As one embodiment of the disclosure in the present specification, a guide nucleic acid may be gRNA including a guide sequence complementarily binding to a target sequence of a AT gene and/or TFPI gene.

The “guide sequence” is a nucleotide sequence complementary to partial sequence of either strand of a double strand of a target gene or a nucleic acid. Here, the guide sequence may be a nucleotide sequence having at least 50%, 55%, 60%, 65%, 70%, 75%, 80%, 85%, 90%, 95% or more complementarity or complete complementarity.

The guide sequence may be a sequence of 10 to 25 nucleotides.

In an example, the guide sequence may be a sequence of 10 to 25, 15 to 25 or 20 to 25 nucleotides.

In another example, the guide sequence may be a sequence of 10 to 15, 15 to 20 or 20 to 25 nucleotides.

The target gene disclosed in the specification may be a blood coagulation inhibitory gene.

The target gene disclosed in the specification may be a AT gene (SERPINC1 gene) and/or TFPI gene.

The guide sequence is capable of forming a complementary bond with a target sequence.

The target sequence may be a guide nucleic acid-binding sequence.

The “guide nucleic acid-binding sequence” is a nucleotide sequence having complementarity with a guide sequence included in the guide domain of the guide nucleic acid, and may be complementarily bonded with the guide sequence included in the guide domain of the guide nucleic acid. The target sequence and guide nucleic acid-binding sequence are nucleotide sequences that may vary according to a target gene or a nucleic acid, that is, the subject to be genetically manipulated or edited, and may be designed in various ways according to a target gene or a nucleic acid.

The description related to the target sequence and guide nucleic acid-binding sequence is the same as described above.

The guide nucleic acid-binding sequence may have the same length as a guide sequence.

The guide nucleic acid-binding sequence may have shorter length than a guide sequence.

The guide nucleic acid-binding sequence may have longer length than a guide sequence.

The guide sequence may be a nucleotide sequence having at least 50%, 55%, 60%, 65%, 70%, 75%, 80%, 85%, 90% , 95% or more complementarity or complete complementarity to the guide nucleic acid-binding sequence.

In one example, the guide sequence may have or include a 1 to 8-nucleotide sequence which is not complementary to guide nucleic acid-binding sequence.

In one embodiment, the guide sequence disclosed in the present specification may form a complementary bond with a 10 to 35-nt contiguous sequence located in a promoter region of a blood coagulation inhibitory gene.

In one example, the guide sequence may form a complementary bond with a 10 to 25-nt contiguous sequence located in a promoter region of an AT gene.

In another example, the guide sequence may form a complementary bond with a 10 to 25-nt contiguous sequence located in a promoter region of an TFPI gene.

The guide sequence disclosed in the present specification may form a complementary bond with a 10 to 35-nt contiguous sequence located in an intron region of the blood coagulation inhibitory gene.

In one example, the guide sequence may form a complementary bond with a 10 to 25-nt contiguous sequence located in an intron region of an AT gene.

In another example, the guide sequence may form a complementary bond with a 10 to 25-nt contiguous sequence located in an intron region of an TFPI gene.

The guide sequence disclosed in the present specification may form a complementary bond with a 10 to 35-nt contiguous sequence located in an exon region of the blood coagulation inhibitory gene.

In one example, the guide sequence may form a complementary bond with a 10 to 25-nt contiguous sequence located in an exon region of an AT gene.

In another example, the guide sequence may form a complementary bond with a 10 to 25-nt contiguous sequence located in an exon region of an TFPI gene.

The guide sequence disclosed in the present specification may form a complementary bond with a 10 to 35-nt contiguous sequence located in an enhancer region of the blood coagulation inhibitory gene.

In one example, the guide sequence may form a complementary bond with a 10 to 25-nt contiguous sequence located in an enhancer region of an AT gene.

In another example, the guide sequence may form a complementary bond with a 10 to 25-nt contiguous sequence located in an enhancer region of an TFPI gene.

The guide sequence disclosed in the present specification may form a complementary bond with a 10 to 35-nt contiguous sequence located in a coding region, a non-coding region or a mixed region of the blood coagulation inhibitory gene.

In one example, the guide sequence may form a complementary bond with a 10 to 25-nt contiguous sequence located in a coding region, a non-coding region or a mixed region of an AT gene.

In another example, the guide sequence may form a complementary bond with a 10 to 25-nt contiguous sequence located in a coding region, a non-coding region or a mixed region of an TFPI gene.

The guide sequence disclosed in the present specification may form a complementary bond with a 10 to 35-nt contiguous sequence located in a promoter, an enhancer, a 3′ UTR, a polyA region or a mixed region of the blood coagulation inhibitory gene.

In one example, the guide sequence may form a complementary bond with a 10 to 25-nt contiguous sequence located in a promoter, an enhancer, a 3′ UTR, a polyA region or a mixed region of an AT gene.

In another example, the guide sequence may form a complementary bond with a 10 to 25-nt contiguous sequence located in a promoter, an enhancer, a 3′ UTR, a polyA region or a mixed region of an TFPI gene.

The guide sequence disclosed in the present specification may form a complementary bond with a 10 to 35-nt contiguous sequence located in an exon, an intron or a mixed region of the blood coagulation inhibitory gene.

In one example, the guide sequence may form a complementary bond with a 10 to 25-nt contiguous sequence located in an exon, an intron or a mixed region of an AT gene.

In another example, the guide sequence may form a complementary bond with a 10 to 25-nt contiguous sequence located in an exon, an intron or a mixed region of an TFPI gene.

The guide sequence disclosed in the present specification may form a complementary bond with a 10 to 35-nt contiguous sequence which includes or is adjacent to a mutant part (e. g, a part different from a wild-type gene) of the blood coagulation inhibitory gene.

In one example, the guide sequence may form a complementary bond with a 10 to 25-nt contiguous sequence which includes or is adjacent to a mutant part (e. g, a part different from a wild-type gene) of an AT gene.

In another example, the guide sequence may form a complementary bond with a 10 to 25-nt contiguous sequence which includes or is adjacent to a mutant part (e. g, a part different from a wild-type gene) of an TFPI gene.

The guide sequence disclosed in the present specification may form a complementary bond with a 10 to 35-nt contiguous sequence which is adjacent to the 5′ end and/or 3′ end of a proto-spacer-adjacent motif (PAM) sequence in the nucleic acid sequence of the blood coagulation inhibitory gene.

The “proto-spacer-adjacent motif (PAM) sequence” is a nucleotide sequence that can be recognized by an editor protein. Here, the PAM sequence may have different nucleotide sequences according to the type of the editor protein and an editor protein-derived species.

Here, the PAM sequence may be, for example, one or more sequences of the following sequences (described in a 5′ to 3′ direction).
NGG (N is A, T, C or G);
NNNNRYAC (N is each independently A, T, C or G, R is A or G, and Y is C or T);
NNAGAAW (N is each independently A, T, C or G, and W is A or T);
NNNNGATT (N is each independently A, T, C or G);
NNGRR(T) (N is each independently A, T, C or G, and R is A or G); and
TTN (N is A, T, C or G).

In one example, the guide sequence may form a complementary bond with a 10 to 25-nt contiguous sequence which is adjacent to the 5′ end and/or 3′ end of a proto-spacer-adjacent motif (PAM) sequence in the nucleic acid sequence of an AT gene.

In one exemplary embodiment, when the PAM sequence recognized by an editor protein is 5′-NGG-3′, 5′-NAG-3′ and/or 5′-NGA-3′ (N= A, T, G or C; or A, U, G or C), the guide sequence may form a complementary bond with a 10 to 25-nt contiguous sequence which is adjacent to the 5′ end and/or 3′ end of 5′-NGG-3′, 5′-NAG-3′ and/or 5′-NGA-3′ (N= A, T, G or C; or A, U, G or C) sequence in the nucleic acid sequence of an AT gene.

In another exemplary embodiment, when the PAM sequence recognized by an editor protein is 5′-NGGNG-3′ and/or 5′-NNAGAAW-3′ (W = A or T, N= A, T, G or C; or A, U, G or C), the guide sequence may form a complementary bond with a 10 to 25-nt contiguous sequence which is adjacent to the 5′ end and/or 3′ end of 5′-NGGNG-3′ and/or 5′-NNAGAAW-3′ (W = A or T, N= A, T, G or C; or A, U, G or C) sequence in the nucleic acid sequence of an AT gene. In another exemplary embodiment, when the PAM sequence recognized by an editor protein is 5′-NNNNGATT-3′ and/or 5′-NNNGCTT-3′ (N= A, T, G or C; or A, U, G or C), the guide sequence may form a complementary bond with a 10 to 25-nt contiguous sequence which is adjacent to the 5′ end and/or 3′ end of 5′-NNNNGATT-3′ and/or 5′-NNNGCTT-3′ (N= A, T, G or C; or A, U, G or C) sequence in the nucleic acid sequence of an AT gene.

In one exemplary embodiment, when the PAM sequence recognized by an editor protein is 5′-NNNVRYAC-3′ (V = G, C or A; R = A or G, Y = C or T, N= A, T, G or C; or A, U, G or C), the guide sequence may form a complementary bond with a 10 to 25-nt contiguous sequence which is adjacent to the 5′ end and/or 3′ end of 5′-NNNVRYAC-3′ (V = G, C or A; R = A or G, Y = C or T, N= A, T, G or C; or A, U, G or C) sequence in the nucleic acid sequence of an AT gene.

In another exemplary embodiment, when the PAM sequence recognized by an editor protein is 5′-NAAR-3′(R = A or G, N= A, T, G or C; or A, U, G or C), the guide sequence may form a complementary bond with a 10 to 25-nt contiguous sequence which is adjacent to the 5′ end and/or 3′ end of 5′-NAAR-3′(R = A or G, N= A, T, G or C; or A, U, G or C) sequence in the nucleic acid sequence of an AT gene.

In another exemplary embodiment, when the PAM sequence recognized by an editor protein is 5′-NNGRR-3′, 5′-NNGRRT-3′ and/or 5′-NNGRRV-3′ (R = A or G, V = G, C or A, N= A, T, G or C; or A, U, G or C), the guide sequence may form a complementary bond with a 10 to 25-nt contiguous sequence which is adjacent to the 5′ end and/or 3′ end of 5′-NNGRR-3′, 5′-NNGRRT-3′ and/or 5′-NNGRRV-3′ (R = A or G, V = G, C or A, N= A, T, G or C; or A, U, G or C) sequence in the nucleic acid sequence of an AT gene.

In one exemplary embodiment, when the PAM sequence recognized by an editor protein is 5′-TTN-3′ (N= A, T, G or C; or A, U, G or C), the guide sequence may form a complementary bond with a 10 to 25-nt contiguous sequence which is adjacent to the 5′ end and/or 3′ end of 5′-TTN-3′ (N= A, T, G or C; or A, U, G or C) sequence in the nucleic acid sequence of an AT gene.

In another example, the guide sequence may form a complementary bond with a 10 to 25-nt contiguous sequence which is adjacent to the 5′ end and/or 3′ end of a proto- spacer-adjacent motif (PAM) sequence in the nucleic acid sequence of an TFPI gene.

In one exemplary embodiment, when the PAM sequence recognized by an editor protein is 5′-NGG-3′, 5′-NAG-3′ and/or 5′-NGA-3′ (N= A, T, G or C; or A, U, G or C), the guide sequence may form a complementary bond with a 10 to 25-nt contiguous sequence which is adjacent to the 5′ end and/or 3′ end of 5′-NGG-3′, 5′-NAG-3′ and/or 5′- NGA-3′ (N= A, T, G or C; or A, U, G or C) sequence in the nucleic acid sequence of an TFPI gene.

In another exemplary embodiment, when the PAM sequence recognized by an editor protein is 5′-NGGNG-3′ and/or 5′-NNAGAAW-3′ (W = A or T, N= A, T, G or C; or A, U, G or C), the guide sequence may form a complementary bond with a 10 to 25-nt contiguous sequence which is adjacent to the 5′ end and/or 3′ end of 5′-NGGNG-3′ and/or 5′-NNAGAAW-3′ (W = A or T, N= A, T, G or C; or A, U, G or C) sequence in the nucleic acid sequence of an TFPI gene.

In another exemplary embodiment, when the PAM sequence recognized by an editor protein is 5′-NNNNGATT-3′ and/or 5′-NNNGCTT-3′ (N= A, T, G or C; or A, U, G or C), the guide sequence may form a complementary bond with a 10 to 25-nt contiguous sequence which is adjacent to the 5′ end and/or 3′ end of 5′-NNNNGATT-3′ and/or 5′- NNNGCTT-3′ (N= A, T, G or C; or A, U, G or C) sequence in the nucleic acid sequence of an TFPI gene.

In one exemplary embodiment, when the PAM sequence recognized by an editor protein is 5′-NNNVRYAC-3′ (V = G, C or A; R = A or G, Y = C or T, N= A, T, G or C; or A, U, G or C), the guide sequence may form a complementary bond with a 10 to 25- nt contiguous sequence which is adjacent to the 5′ end and/or 3′ end of 5′-NNNVRYAC- 3′ (V = G, C or A; R = A or G, Y = C or T, N= A, T, G or C; or A, U, G or C) sequence in the nucleic acid sequence of an TFPI gene.

In another exemplary embodiment, when the PAM sequence recognized by an editor protein is 5′-NAAR-3′(R = A or G, N= A, T, G or C; or A, U, G or C), the guide sequence may form a complementary bond with a 10 to 25-nt contiguous sequence which is adjacent to the 5′ end and/or 3′ end of 5′-NAAR-3′(R = A or G, N= A, T, G or C; or A, U, G or C) sequence in the nucleic acid sequence of an TFPI gene.

In another exemplary embodiment, when the PAM sequence recognized by an editor protein is 5′-NNGRR-3′, 5′-NNGRRT-3′ and/or 5′-NNGRRV-3′ (R = A or G, V = G, C or A, N= A, T, G or C; or A, U, G or C), the guide sequence may form a complementary bond with a 10 to 25-nt contiguous sequence which is adjacent to the 5′ end and/or 3′ end of 5′-NNGRR-3′, 5′-NNGRRT-3′ and/or 5′-NNGRRV-3′ (R = A or G, V = G, C or A, N= A, T, G or C; or A, U, G or C) sequence in the nucleic acid sequence of an TFPI gene.

In one exemplary embodiment, when the PAM sequence recognized by an editor protein is 5′-TTN-3′ (N= A, T, G or C; or A, U, G or C), the guide sequence may form a complementary bond with a 10 to 25-nt contiguous sequence which is adjacent to the 5′ end and/or 3′ end of 5′-TTN-3′ (N= A, T, G or C; or A, U, G or C) sequence in the nucleic acid sequence of an TFPI gene.

Hereinafter, examples of guide sequences that can be used in an exemplary embodiment disclosed in the specification are listed in Tables 3 and 4. The guide sequences disclosed in Tables 3 and 4 are guide sequences capable of targeting an AT(SERPINC1 gene) or TFPI gene, and may form a complementary bond with a target sequence located in an AT(SERPINC1 gene) or TFPI gene. The guide sequences disclosed in table 3 and 4 are guide sequences capable of targeting the target sequence of table 1 and 2, respectively. In addition, target names shown in Tables 3 and 4 were named Sp for SpCas9, Sa for SaCas9 and Cj for CjCas9 according to an editor protein.

TABLE 3

Guide sequences capable of targeting SERPINC1 gene(AT gene)

| | | | |
|---|---|---|---|
| Target name | Loci. | Guide sequence | SEQ ID NO |
| hSerpinc1-Sp-1 | Exon01 (E01) | UCCUAUCACAUUGGAAUACA | SEQ ID NO: 425 |
| hSerpinc1-Sp-2 | E01 | GGUUACAGUUCCUAUCACAU | SEQ ID NO: 426 |
| hSerpinc1-Sp-3 | E01 | GCCAUGUAUUCCAAUGUGAU | SEQ ID NO: 427 |
| hSerpinc1-Sp-4 | E01 | GUGAUAGGAACUGUAACCUC | SEQ ID NO: 428 |
| hSerpinc1-Sp-5 | E01 | CCCCUCUUACCUUUUUCCAG | SEQ ID NO: 429 |
| .hSerpinc1-Sp-6 | E01 | GAACUGUAACCUCUGGAAAA | SEQ ID NO: 430 |
| hSerpinc1-Sp-7 | E02 | CCAGAAGCCAAUGAGCAGCA | SEQ ID NO: 431 |
| hSerpinc1-Sp-8 | E02 | CUUUUGUCCUUGCUGCUCAU | SEQ ID NO: 432 |
| hSerpinc1-Sp-9 | E02 | CCUUGCUGCUCAUUGGCUUC | SEQ ID NO: 433 |
| hSerpinc1-Sp-10 | E02 | CUUGCUGCUCAUUGGCUUCU | SEQ ID NO: 434 |
| hSerpinc1-Sp-11 | E02 | UGGGACUGCGUGACCUGUCA | SEQ ID NO: 435 |
| hSerpinc1-Sp-12 | E02 | GGGACUGCGUGACCUGUCAC | SEQ ID NO: 436 |
| hSerpinc1-Sp-13 | E02 | GUCCACAGGGCUCCCGUGAC | SEQ ID NO: 437 |
| hSerpinc1-Sp-14 | E02 | GACCUGUCACGGGAGCCCUG | SEQ ID NO: 438 |
| hSerpinc1-Sp-15 | E02 | UGGCUGUGCAGAUGUCCACA | SEQ ID NO: 439 |
| hSerpinc1-Sp-16 | E02 | UUGGCUGUGCAGAUGUCCAC | SEQ ID NO: 440 |
| hSerpinc1-Sp-17 | E02 | ACAUCUGCACAGCCAAGCCG | SEQ ID NO: 441 |
| hSerpinc1-Sp-18 | E02 | CAUCUGCACAGCCAAGCCGC | SEQ ID NO: 442 |
| hSerpinc1-Sp-19 | E02 | CAUGGGAAUGUCCCGCGGCU | SEQ ID NO: 443 |
| hSerpinc1-Sp-20 | E02 | GGAUUCAUGGGAAUGUCCCG | SEQ ID NO: 444 |
| hSerpinc1-Sp-21 | E02 | UAAAUGCACAUGGGAUUCAU | SEQ ID NO: 445 |
| hSerpinc1-Sp-22 | E02 | GUAAAUGCACAUGGGAUUCA | SEQ ID NO: 446 |
| hSerpinc1-Sp-23 | E02 | GGGGAGCGGUAAAUGCACAU | SEQ ID NO: 447 |
| hSerpinc1-Sp-24 | E02 | CGGGGAGCGGUAAAUGCACA | SEQ ID NO: 448 |
| hSerpinc1-Sp-25 | E02 | CAUGUGCAUUUACCGCUCCC | SEQ ID NO: 449 |
| hSerpinc1-Sp-26 | E02 | UUGCCUUCUUCUCCGGGGAG | SEQ ID NO: 450 |
| hSerpinc1-Sp-27 | E02 | CUCAGUUGCCUUCUUCUCCG | SEQ ID NO: 451 |
| hSerpinc1-Sp-28 | E02 | CCUCAGUUGCCUUCUUCUCC | SEQ ID NO: 452 |
| hSerpinc1-Sp-29 | E02 | UCCUCAGUUGCCUUCUUCUC | SEQ ID NO: 453 |
| hSerpinc1-Sp-30 | E02 | UUACCGCUCCCCGGAGAAGA | SEQ ID NO: 454 |
| hSerpinc1-Sp-31 | E02 | CCCGGAGAAGAAGGCAACUG | SEQ ID NO: 455 |
| hSerpinc1-Sp-32 | E02 | GAAGAAGGCAACUGAGGAUG | SEQ ID NO: 456 |
| hSerpinc1-Sp-33 | E02 | AAGAAGGCAACUGAGGAUGA | SEQ ID NO: 457 |
| hSerpinc1-Sp-34 | E02 | GGGCUCAGAACAGAAGAUCC | SEQ ID NO: 458 |
| hSerpinc1-Sp-35 | E02 | CUCAGAACAGAAGAUCCCGG | SEQ ID NO: 459 |
| hSerpinc1-Sp-36 | E02 | CACGCCGGUUGGUGGCCUCC | SEQ ID NO: 460 |
| hSerpinc1-Sp-37 | E02 | ACACGCCGGUUGGUGGCCUC | SEQ ID NO: 461 |
| hSerpinc1-Sp-38 | E02 | AGAUCCCGGAGGCCACCAAC | SEQ ID NO: 462 |
| hSerpinc1-Sp-39 | E02 | UUCCCAGACACGCCGGUUGG | SEQ ID NO: 463 |
| hSerpinc1-Sp-40 | E02 | CAGUUCCCAGACACGCCGGU | SEQ ID NO: 464 |
| hSerpinc1-Sp-41 | E02 | UGGACAGUUCCCAGACACGC | SEQ ID NO: 465 |
| hSerpinc1-Sp-42 | E02 | AGGCCACCAACCGGCGUGUC | SEQ ID NO: 466 |
| hSerpinc1-Sp-43 | E02 | GGCCACCAACCGGCGUGUCU | SEQ ID NO: 467 |
| hSerpinc1-Sp-44 | E02 | GCGUGUCUGGGAACUGUCCA | SEQ ID NO: 468 |
| hSerpinc1-Sp-45 | E02 | AGCAAAGCGGGAAUUGGCCU | SEQ ID NO: 469 |
| hSerpinc1-Sp-46 | E02 | AGUGGUAGCAAAGCGGGAAU | SEQ ID NO: 470 |
| hSerpinc1-Sp-47 | E02 | AUAGAAAGUGGUAGCAAAGC | SEQ ID NO: 471 |
| hSerpinc1-Sp-48 | E02 | GAUAGAAAGUGGUAGCAAAG | SEQ ID NO: 472 |
| hSerpinc1-Sp-49 | E02 | UGCCAGGUGCUGAUAGAAAG | SEQ ID NO: 473 |
| hSerpinc1-Sp-50 | E02 | UACCACUUUCUAUCAGCACC | SEQ ID NO: 474 |
| hSerpinc1-Sp-51 | E02 | UGUCAUUCUUGGAAUCUGCC | SEQ ID NO: 475 |
| hSerpinc1-Sp-52 | E02 | AAUGUUAUCAUUGUCAUUCU | SEQ ID NO: 476 |
| hSerpinc1-Sp-53 | E02 | UGGAGAUACUCAGGGGUGAC | SEQ ID NO: 477 |
| hSerpinc1-Sp-54 | E02 | AAAGCCGUGGAGAUACUCAG | SEQ ID NO: 478 |
| hSerpinc1-Sp-55 | E02 | AAAAGCCGUGGAGAUACUCA | SEQ ID NO: 479 |
| hSerpinc1-Sp-56 | E02 | CAAAAGCCGUGGAGAUACUC | SEQ ID NO: 480 |
| hSerpinc1-Sp-57 | E02 | GUCACCCCUGAGUAUCUCCA | SEQ ID NO: 481 |
| hSerpinc1-Sp-58 | E02 | CUUGGUCAUAGCAAAAGCCG | SEQ ID NO: 482 |
| hSerpinc1-Sp-59 | E02 | GGCUUUUGCUAUGACCAAGC | SEQ ID NO: 483 |
| hSerpinc1-Sp-60 | E02 | GCUUUUGCUAUGACCAAGCU | SEQ ID NO: 484 |
| hSerpinc1-Sp-61 | E02 | GUCAUUACAGGCACCCAGCU | SEQ ID NO: 485 |
| hSerpinc1-Sp-62 | E02 | UUGCUGGAGGGUGUCAUUAC | SEQ ID NO: 486 |
| hSerpinc1-Sp-63 | E02 | UACCUCCAUCAGUUGCUGGA | SEQ ID NO: 487 |
| hSerpinc1-Sp-64 | E02 | GUACCUCCAUCAGUUGCUGG | SEQ ID NO: 488 |
| hSerpinc1-Sp-65 | E02 | GUCGUACCUCCAUCAGUUGC | SEQ ID NO: 489 |
| hSerpinc1-Sp-66 | E02 | UGACACCCUCCAGCAACUGA | SEQ ID NO: 490 |
| hSerpinc1-Sp-67 | E02 | CACCCUCCAGCAACUGAUGG | SEQ ID NO: 491 |
| hSerpinc1-Sp-68 | E03 | AUCAGAUGUUUUCUCAGAUA | SEQ ID NO: 492 |
| hSerpinc1-Sp-69 | E03 | UCAGUUUGGCAAAGAAGAAG | SEQ ID NO: 493 |
| hSerpinc1-Sp-70 | E03 | AUAGAGUCGGCAGUUCAGUU | SEQ ID NO: 494 |
| hSerpinc1-Sp-71 | E03 | UGUUGGCUUUUCGAUAGAGU | SEQ ID NO: 495 |
| hSerpinc1-Sp-72 | E03 | UACUAACUUGGAGGAUUUGU | SEQ ID NO: 496 |
| hSerpinc1-Sp-73 | E03 | AUUGGCUGAUACUAACUUGG | SEQ ID NO: 497 |
| hSerpinc1-Sp-74 | E03 | GCGAUUGGCUGAUACUAACU | SEQ ID NO: 498 |
| hSerpinc1-Sp-75 | E03 | UUUGUCUCCAAAAAGGCGAU | SEQ ID NO: 499 |
| hSerpinc1-Sp-76 | E03 | GUAUCAGCCAAUCGCCUUUU | SEQ ID NO: 500 |
| hSerpinc1-Sp-77 | E03 | UAAGGGAUUUGUCUCCAAAA | SEQ ID NO: 501 |
| hSerpinc1-Sp-78 | E03 | GUAGGUCUCAUUGAAGGUAA | SEQ ID NO: 502 |
| hSerpinc1-Sp-79 | E03 | GGUAGGUCUCAUUGAAGGUA | SEQ ID NO: 503 |
| hSerpinc1-Sp-80 | E03 | GUCCUGGUAGGUCUCAUUGA | SEQ ID NO: 504 |
| hSerpinc1-Sp-81 | E03 | UACCUUCAAUGAGACCUACC | SEQ ID NO: 505 |
| hSerpinc1-Sp-82 | E03 | CAACUCACUGAUGUCCUGGU | SEQ ID NO: 506 |
| hSerpinc1-Sp-83 | E03 | AUACCAACUCACUGAUGUCC | SEQ ID NO: 507 |
| hSerpinc1-Sp-84 | E03 | CUACCAGGACAUCAGUGAGU | SEQ ID NO: 508 |
| hSerpinc1-Sp-85 | E03 | GACAUCAGUGAGUUGGUAUA | SEQ ID NO: 509 |
| hSerpinc1-Sp-86 | E03 | GAAGUCCAGGGGCUGGAGCU | SEQ ID NO: 510 |
| hSerpinc1-Sp-87 | E03 | UGGAGCCAAGCUCCAGCCCC | SEQ ID NO: 511 |
| hSerpinc1-Sp-88 | E03 | UCACCUUGAAGUCCAGGGGC | SEQ ID NO: 512 |
| hSerpinc1-Sp-89 | E03 | CAACUCACCUUGAAGUCCAG | SEQ ID NO: 513 |
| hSerpinc1-Sp-90 | E03 | GCAACUCACCUUGAAGUCCA | SEQ ID NO: 514 |
| hSerpinc1-Sp-91 | E03 | UGCAACUCACCUUGAAGUCC | SEQ ID NO: 515 |
| hSerpinc1-Sp-92 | E03 | GCUCCAGCCCCUGGACUUCA | SEQ ID NO: 516 |
| hSerpinc1-Sp-93 | E04 | GAUUGCUCUGCAUUUUCCUG | SEQ ID NO: 517 |
| hSerpinc1-Sp-94 | E04 | AAAUGCAGAGCAAUCCAGAG | SEQ ID NO: 518 |
| hSerpinc1-Sp-95 | E04 | CCAUUUGUUGAUGGCCGCUC | SEQ ID NO: 519 |
| hSerpinc1-Sp-96 | E04 | AUUGGACACCCAUUUGUUGA | SEQ ID NO: 520 |
| hSerpinc1-Sp-97 | E04 | CCAGAGCGGCCAUCAACAAA | SEQ ID NO: 521 |
| hSerpinc1-Sp-98 | E04 | CAGAGCGGCCAUCAACAAAU | SEQ ID NO: 522 |
| hSerpinc1-Sp-99 | E04 | GAUUCGGCCUUCGGUCUUAU | SEQ ID NO: 523 |
| hSerpinc1-Sp-100 | E04 | UGGGUGUCCAAUAAGACCGA | SEQ ID NO: 524 |
| hSerpinc1-Sp-101 | E04 | GACAUCGGUGAUUCGGCCUU | SEQ ID NO: 525 |
| hSerpinc1-Sp-102 | E04 | AGGGAAUGACAUCGGUGAUU | SEQ ID NO: 526 |
| hSerpinc1-Sp-103 | E04 | GGCUUCCGAGGGAAUGACAU | SEQ ID NO: 527 |
| hSerpinc1-Sp-104 | E04 | AAUCACCGAUGUCAUUCCCU | SEQ ID NO: 528 |
| hSerpinc1-Sp-105 | E04 | AGCUCAUUGAUGGCUUCCGA | SEQ ID NO: 529 |
| hSerpinc1-Sp-106 | E04 | GAGCUCAUUGAUGGCUUCCG | SEQ ID NO: 530 |
| hSerpinc1-Sp-107 | E04 | CAGAACAGUGAGCUCAUUGA | SEQ ID NO: 531 |
| hSerpinc1-Sp-108 | E04 | CAUCAAUGAGCUCACUGUUC | SEQ ID NO: 532 |
| hSerpinc1-Sp-109 | E04 | UGAGCUCACUGUUCUGGUGC | SEQ ID NO: 533 |
| hSerpinc1-Sp-110 | E04 | UCUGAGUACCUUGAAGUAAA | SEQ ID NO: 534 |
| hSerpinc1-Sp-111 | E04 | GGUUAACACCAUUUACUUCA | SEQ ID NO: 535 |
| hSerpinc1-Sp-112 | E05 | ACUUUGACUUCCACAGGCCC | SEQ ID NO: 536 |
| hSerpinc1-Sp-113 | E05 | UGAUUCUCUUCCAGGGCCUG | SEQ ID NO: 537 |
| hSerpinc1-Sp-114 | E05 | GGCUGAACUUUGACUUCCAC | SEQ ID NO: 538 |
| hSerpinc1-Sp-115 | E05 | GUUCCUUCCUUGUGUUCUCA | SEQ ID NO: 539 |
| hSerpinc1-Sp-116 | E05 | AGUUCCUUCCUUGUGUUCUC | SEQ ID NO: 540 |
| hSerpinc1-Sp-117 | E05 | AGUUCAGCCCUGAGAACACA | SEQ ID NO: 541 |
| hSerpinc1-Sp-118 | E05 | CAGCCCUGAGAACACAAGGA | SEQ ID NO: 542 |
| hSerpinc1-Sp-119 | E05 | AAGGAAGGAACUGUUCUACA | SEQ ID NO: 543 |
| hSerpinc1-Sp-120 | E05 | GAACUGUUCUACAAGGCUGA | SEQ ID NO: 544 |
| hSerpinc1-Sp-121 | E05 | UUCAGCAUCUAUGAUGUACC | SEQ ID NO: 545 |
| hSerpinc1-Sp-122 | E05 | GCAUCUAUGAUGUACCAGGA | SEQ ID NO: 546 |
| hSerpinc1-Sp-123 | E05 | GAUAACGGAACUUGCCUUCC | SEQ ID NO: 547 |
| hSerpinc1-Sp-124 | E05 | AGGAAGGCAAGUUCCGUUAU | SEQ ID NO: 548 |
| hSerpinc1-Sp-125 | E05 | CUUCAGCCACGCGCCGAUAA | SEQ ID NO: 549 |
| hSerpinc1-Sp-126 | E05 | CAAGUUCCGUUAUCGGCGCG | SEQ ID NO: 550 |
| hSerpinc1-Sp-127 | E05 | CGUUAUCGGCGCGUGGCUGA | SEQ ID NO: 551 |
| hSerpinc1-Sp-128 | E05 | GCGCGUGGCUGAAGGCACCC | SEQ ID NO: 552 |
| hSerpinc1-Sp-129 | E05 | GAAGGGCAACUCAAGCACCU | SEQ ID NO: 553 |
| hSerpinc1-Sp-130 | E05 | UGAAGGGCAACUCAAGCACC | SEQ ID NO: 554 |
| hSerpinc1-Sp-131 | E05 | GUGCUUGAGUUGCCCUUCAA | SEQ ID NO: 555 |
| hSerpinc1-Sp-132 | E05 | GUGAUGUCAUCACCUUUGAA | SEQ ID NO: 556 |
| hSerpinc1-Sp-133 | E05 | GGUGAUGUCAUCACCUUUGA | SEQ ID NO: 557 |
| hSerpinc1-Sp-134 | E05 | CAAAGGUGAUGACAUCACCA | SEQ ID NO: 558 |
| hSerpinc1-Sp-135 | E05 | CUUGGGCAAGAUGAGGACCA | SEQ ID NO: 559 |
| hSerpinc1-Sp-136 | E05 | UCUCAGGCUUGGGCAAGAUG | SEQ ID NO: 560 |
| hSerpinc1-Sp-137 | E05 | GCCAGGCUCUUCUCAGGCUU | SEQ ID NO: 561 |
| hSerpinc1-Sp-138 | E05 | GGCCAGGCUCUUCUCAGGCU | SEQ ID NO: 562 |
| hSerpinc1-Sp-139 | E05 | ACCUUGGCCAGGCUCUUCUC | SEQ ID NO: 563 |
| hSerpinc1-Sp-140 | E05 | GCCCAAGCCUGAGAAGAGCC | SEQ ID NO: 564 |
| hSerpinc1-Sp-141 | E05 | GCCUGAGAAGAGCCUGGCCA | SEQ ID NO: 565 |
| hSerpinc1-Sp-142 | E05 | GUUCCUUCUCUACCUUGGCC | SEQ ID NO: 566 |
| hSerpinc1-Sp-143 | E05 | GGUGAGUUCCUUCUCUACCU | SEQ ID NO: 567 |
| hSerpinc1-Sp-144 | E05 | GAGCCUGGCCAAGGUAGAGA | SEQ ID NO: 568 |
| hSerpinc1-Sp-145 | E05 | AGAGAAGGAACUCACCCCAG | SEQ ID NO: 569 |
| hSerpinc1-Sp-146 | E05 | CCACUCUUGCAGCACCUCUG | SEQ ID NO: 570 |
| hSerpinc1-Sp-147 | E05 | GCCACUCUUGCAGCACCUCU | SEQ ID NO: 571 |
| hSerpinc1-Sp-148 | E05 | AGCCACUCUUGCAGCACCUC | SEQ ID NO: 572 |
| hSerpinc1-Sp-149 | E05 | CCCCAGAGGUGCUGCAAGAG | SEQ ID NO: 573 |
| hSerpinc1-Sp-150 | E05 | AGAGGUGCUGCAAGAGUGGC | SEQ ID NO: 574 |
| hSerpinc1-Sp-151 | E05 | GCAAGAGUGGCUGGAUGAAU | SEQ ID NO: 575 |
| hSerpinc1-Sp-152 | E05 | AGAGUGGCUGGAUGAAUUGG | SEQ ID NO: 576 |
| hSerpinc1-Sp-153 | E05 | UGAAUUGGAGGAGAUGAUGC | SEQ ID NO: 577 |
| hSerpinc1-Sp-154 | E05 | AUUGGAGGAGAUGAUGCUGG | SEQ ID NO: 578 |
| hSerpinc1-Sp-155 | E05 | CAAUGCGGAAGCGGGGCAUG | SEQ ID NO: 579 |
| hSerpinc1-Sp-156 | E05 | CCGUCCUCAAUGCGGAAGCG | SEQ ID NO: 580 |
| hSerpinc1-Sp-157 | E05 | GCCGUCCUCAAUGCGGAAGC | SEQ ID NO: 581 |
| hSerpinc1-Sp-158 | E05 | AGCCGUCCUCAAUGCGGAAG | SEQ ID NO: 582 |
| hSerpinc1-Sp-159 | E05 | CAUGCCCCGCUUCCGCAUUG | SEQ ID NO: 583 |
| hSerpinc1-Sp-160 | E05 | AACUGAAGCCGUCCUCAAUG | SEQ ID NO: 584 |
| hSerpinc1-Sp-161 | E05 | CCCCGCUUCCGCAUUGAGGA | SEQ ID NO: 585 |
| hSerpinc1-Sp-162 | E05 | UGAGGACGGCUUCAGUUUGA | SEQ ID NO: 586 |
| hSerpinc1-Sp-163 | E05 | GAAGGAGCAGCUGCAAGACA | SEQ ID NO: 587 |
| hSerpinc1-Sp-164 | E05 | AAGGAGCAGCUGCAAGACAU | SEQ ID NO: 588 |
| hSerpinc1-Sp-165 | E05 | CAGGGCUGAACAGAUCGACA | SEQ ID NO: 589 |
| hSerpinc1-Sp-166 | E05 | CUGGGAGUUUGGACUUUUCA | SEQ ID NO: 590 |
| hSerpinc1-Sp-167 | E05 | CCUGGGAGUUUGGACUUUUC | SEQ ID NO: 591 |
| hSerpinc1-Sp-168 | E05 | CCUAGACAAACCUGGGAGUU | SEQ ID NO: 592 |
| hSerpinc1-Sp-169 | E05 | CCUGAAAAGUCCAAACUCCC | SEQ ID NO: 593 |
| hSerpinc1-Sp-170 | E06 | CGGCCUUCUGCAACAAUACC | SEQ ID NO: 594 |
| hSerpinc1-Sp-171 | E06 | CUUCCAGGUAUUGUUGCAGA | SEQ ID NO: 595 |
| hSerpinc1-Sp-172 | E06 | CUGAGACAUAGAGGUCAUCU | SEQ ID NO: 596 |
| hSerpinc1-Sp-173 | E06 | GGAAUGCAUCUGAGACAUAG | SEQ ID NO: 597 |
| hSerpinc1-Sp-174 | E06 | UGUCUCAGAUGCAUUCCAUA | SEQ ID NO: 598 |
| hSerpinc1-Sp-175 | E06 | UCACCUCAAGAAAUGCCUUA | SEQ ID NO: 599 |
| hSerpinc1-Sp-176 | E06 | AUUCCAUAAGGCAUUUCUUG | SEQ ID NO: 600 |
| hSerpinc1-Sp-177 | E07 | GACCUGCAGGUAAAUGAAGA | SEQ ID NO: 601 |
| hSerpinc1-Sp-178 | E07 | ACGGCCAGCAAUCACAACAG | SEQ ID NO: 602 |
| hSerpinc1-Sp-179 | E07 | AGUACCGCUGUUGUGAUUGC | SEQ ID NO: 603 |
| hSerpinc1-Sp-180 | E07 | CCCUGUUGGGGUUUAGCGAA | SEQ ID NO: 604 |
| hSerpinc1-Sp-181 | E07 | GCCGUUCGCUAAACCCCAAC | SEQ ID NO: 605 |
| hSerpinc1-Sp-182 | E07 | CCGUUCGCUAAACCCCAACA | SEQ ID NO: 606 |
| hSerpinc1-Sp-183 | E07 | CCUUGAAAGUCACCCUGUUG | SEQ ID NO: 607 |
| hSerpinc1-Sp-184 | E07 | GCCUUGAAAGUCACCCUGUU | SEQ ID NO: 608 |
| hSerpinc1-Sp-185 | E07 | GGCCUUGAAAGUCACCCUGU | SEQ ID NO: 609 |
| hSerpinc1-Sp-186 | E07 | CCCCAACAGGGUGACUUUCA | SEQ ID NO: 610 |
| hSerpinc1-Sp-187 | E07 | GGGUGACUUUCAAGGCCAAC | SEQ ID NO: 611 |
| hSerpinc1-Sp-188 | E07 | AAAAACCAGGAAAGGCCUGU | SEQ ID NO: 612 |
| hSerpinc1-Sp-189 | E07 | CAAGGCCAACAGGCCUUUCC | SEQ ID NO: 613 |
| hSerpinc1-Sp-190 | E07 | UCUCUUAUAAAAACCAGGAA | SEQ ID NO: 614 |
| hSerpinc1-Sp-191 | E07 | GAACUUCUCUUAUAAAAACC | SEQ ID NO: 615 |
| hSerpinc1-Sp-192 | E07 | AUGAAGAUAAUAGUGUUCAG | SEQ ID NO: 616 |
| hSerpinc1-Sp-193 | E07 | UCUGAACACUAUUAUCUUCA | SEQ ID NO: 617 |
| hSerpinc1-Sp-194 | E07 | CUGAACACUAUUAUCUUCAU | SEQ ID NO: 618 |
| hSerpinc1-Sp-195 | E07 | AUUUUACUUAACACAAGGGU | SEQ ID NO: 619 |
| hSerpinc1-Sp-196 | E07 | GAACAUUUUACUUAACACAA | SEQ ID NO: 620 |
| hSerpinc1-Sp-197 | E07 | AGAACAUUUUACUUAACACA | SEQ ID NO: 621 |
| hSerpinc1-Sa-1 | E01 | GGUUACAGUUCCUAUCACAU | SEQ ID NO: 622 |
| hSerpinc1-Sa-2 | E02 | CCAAGCCGCGGGACAUUCCC | SEQ ID NO: 623 |
| hSerpinc1-Sa-3 | E02 | UAAAUGCACAUGGGAUUCAU | SEQ ID NO: 624 |
| hSerpinc1-Sa-4 | E02 | CGGGGAGCGGUAAAUGCACA | SEQ ID NO: 625 |
| hSerpinc1-Sa-5 | E02 | CCCCGGAGAAGAAGGCAACU | SEQ ID NO: 626 |
| hSerpinc1-Sa-6 | E02 | ACACGCCGGUUGGUGGCCUC | SEQ ID NO: 627 |
| hSerpinc1-Sa-7 | E02 | AUAGAAAGUGGUAGCAAAGC | SEQ ID NO: 628 |
| hSerpinc1-Sa-8 | E02 | AUCAGCACCUGGCAGAUUCC | SEQ ID NO: 629 |
| hSerpinc1-Sa-9 | E02 | AAUGUUAUCAUUGUCAUUCU | SEQ ID NO: 630 |
| hSerpinc1-Sa-10 | E02 | AUAACAUUUUCCUGUCACCC | SEQ ID NO: 631 |
| hSerpinc1-Sa-11 | E02 | CAAAAGCCGUGGAGAUACUC | SEQ ID NO: 632 |
| hSerpinc1-Sa-12 | E02 | CGGCUUUUGCUAUGACCAAG | SEQ ID NO: 633 |
| hSerpinc1-Sa-13 | E02 | CGUACCUCCAUCAGUUGCUG | SEQ ID NO: 634 |
| hSerpinc1-Sa-14 | E03 | UUCAGUUUGGCAAAGAAGAA | SEQ ID NO: 635 |
| hSerpinc1-Sa-15 | E03 | AGGAUUUGUUGGCUUUUCGA | SEQ ID NO: 636 |
| hSerpinc1-Sa-16 | E03 | GAUUGGCUGAUACUAACUUG | SEQ ID NO: 637 |
| hSerpinc1-Sa-17 | E03 | GGUAGGUCUCAUUGAAGGUA | SEQ ID NO: 638 |
| hSerpinc1-Sa-18 | E03 | UGAGACCUACCAGGACAUCA | SEQ ID NO: 639 |
| hSerpinc1-Sa-19 | E03 | UCCAGCCCCUGGACUUCAAG | SEQ ID NO: 640 |
| hSerpinc1-Sa-20 | E04 | CCCAUUUGUUGAUGGCCGCU | SEQ ID NO: 641 |
| hSerpinc1-Sa-21 | E04 | UCCAGAGCGGCCAUCAACAA | SEQ ID NO: 642 |
| hSerpinc1-Sa-22 | E04 | GUGUCCAAUAAGACCGAAGG | SEQ ID NO: 643 |
| hSerpinc1-Sa-23 | E04 | AGCUCAUUGAUGGCUUCCGA | SEQ ID NO: 644 |
| hSerpinc1-Sa-24 | E05 | ACUGUUCUACAAGGCUGAUG | SEQ ID NO: 645 |
| hSerpinc1-Sa-25 | E05 | GGCUGAAGGCACCCAGGUGC | SEQ ID NO: 646 |
| hSerpinc1-Sa-26 | E05 | UUGAAGGGCAACUCAAGCAC | SEQ ID NO: 647 |
| hSerpinc1-Sa-27 | E05 | ACUCUUGCAGCACCUCUGGG | SEQ ID NO: 648 |
| hSerpinc1-Sa-28 | E05 | AGCCACUCUUGCAGCACCUC | SEQ ID NO: 649 |
| hSerpinc1-Sa-29 | E05 | ACUCACCCCAGAGGUGCUGC | SEQ ID NO: 650 |
| hSerpinc1-Sa-30 | E05 | CAGAGGUGCUGCAAGAGUGG | SEQ ID NO: 651 |
| hSerpinc1-Sa-31 | E05 | GGUGCUGCAAGAGUGGCUGG | SEQ ID NO: 652 |
| hSerpinc1-Sa-32 | E06 | UCACCUCAAGAAAUGCCUUA | SEQ ID NO: 653 |
| hSerpinc1-Sa-33 | E06 | UCCAUAAGGCAUUUCUUGAG | SEQ ID NO: 654 |
| hSerpinc1-Sa-34 | E07 | GGCCGUUCGCUAAACCCCAA | SEQ ID NO: 655 |
| hSerpinc1-Sa-35 | E07 | GGCCUUGAAAGUCACCCUGU | SEQ ID NO: 656 |
| hSerpinc1-Sa-36 | E07 | AACACUAUUAUCUUCAUGGG | SEQ ID NO: 657 |
| hSerpinc1-Sa-37 | E07 | AAGAACAUUUUACUUAACAC | SEQ ID NO: 658 |
| hSerpinc1-Cj-1 | E01 | GAGGUUACAGUUCCUAUCACAU | SEQ ID NO: 659 |
| hSerpinc1-Cj-2 | E02 | CUGUCACGGGAGCCCUGUGGAC | SEQ ID NO: 660 |
| hSerpinc1-Cj-3 | E02 | GCCUUCUUCUCCGGGGAGCGGU | SEQ ID NO: 661 |
| hSerpinc1-Cj-4 | E02 | GGGAAUUGGCCUUGGACAGUUC | SEQ ID NO: 662 |
| hSerpinc1-Cj-5 | E02 | UCCCGCUUUGCUACCACUUUCU | SEQ ID NO: 663 |
| hSerpinc1-Cj- | E02 | GCUUGGUCAUAGCAAAAGCCGU | SEQ ID NO: 664 |
| hSerpinc1-Cj-7 | E02 | UAUGACCAAGCUGGGUGCCUGU | SEQ ID NO: 665 |
| hSerpinc1-Cj-8 | E02 | UCAGUUGCUGGAGGGUGUCAUU | SEQ ID NO: 666 |
| hSerpinc1-Cj-9 | E02 | AUGACACCCUCCAGCAACUGAU | SEQ ID NO: 667 |
| hSerpinc1-Cj-10 | E03 | UUGACUUCUAUAGGUAUUUAAG | SEQ ID NO: 668 |
| hSerpinc1-Cj-11 | E03 | UUUCUCAGAUAUGGUGUCAAAC | SEQ ID NO: 669 |
| hSerpinc1-Cj-12 | E03 | UUUGUCUCCAAAAAGGCGAUUG | SEQ ID NO: 670 |
| hSerpinc1-Cj-13 | E03 | GUCCAGGGGCUGGAGCUUGGCU | SEQ ID NO: 671 |
| hSerpinc1-Cj-14 | E04 | GGUGAUUCGGCCUUCGGUCUUA | SEQ ID NO: 672 |
| hSerpinc1-Cj-15 | E04 | UGAGCUCACUGUUCUGGUGCUG | SEQ ID NO: 673 |
| hSerpinc1-Cj-16 | E04 | UACCUUGAAGUAAAUGGUGUUA | SEQ ID NO: 674 |
| hSerpinc1-Cj-17 | E04 | UGCUGGUUAACACCAUUUACUU | SEQ ID NO: 675 |
| hSerpinc1-Cj-18 | E05 | GUGGAAGUCAAAGUUCAGCCCU | SEQ ID NO: 676 |
| hSerpinc1-Cj-19 | E05 | GGAGAGUCGUGUUCAGCAUCUA | SEQ ID NO: 677 |
| hSerpinc1-Cj-20 | E05 | CCUUCCUGGUACAUCAUAGAUG | SEQ ID NO: 678 |
| hSerpinc1-Cj-21 | E05 | CGCCGAUAACGGAACUUGCCUU | SEQ ID NO: 679 |
| hSerpinc1-Cj-22 | E05 | GUUCCGUUAUCGGCGCGUGGCU | SEQ ID NO: 680 |
| hSerpinc1-Cj-23 | E05 | GUCAUCACCUUUGAAGGGCAAC | SEQ ID NO: 681 |
| hSerpinc1-Cj-24 | E05 | CUCCAAUUCAUCCAGCCACUCU | SEQ ID NO: 682 |
| hSerpinc1-Cj-25 | E06 | AGAGGUCAUCUCGGCCUUCUGC | SEQ ID NO: 683 |
| hSerpinc1-Cj-26 | E06 | AUUCCAUAAGGCAUUUCUUGAG | SEQ ID NO: 684 |
| hSerpinc1-Cj-27 | E07 | UGAAGAAGGCAGUGAAGCAGCU | SEQ ID NO: 685 |
| hSerpinc1-Cj-28 | E07 | GCGAACGGCCAGCAAUCACAAC | SEQ ID NO: 686 |
| hSerpinc1-Cj-29 | E07 | GGUUUUUAUAAGAGAAGUUCCU | SEQ ID NO: 687 |
| hSerpinc1-Cj-30 | E07 | UGCAAAGAAUAAGAACAUUUUA | SEQ ID NO: 688 |

TABLE 4

Guide sequences capable of targeting TFPI gene

| | | | |
|---|---|---|---|
| Target name | Loci. | Guide sequence | SEQ ID NO |
| hTfpi-Sp-1 | E02 | UGAAGAAAGUACAUGCACUU | SEQ ID NO: 689 |
| hTfpi-Sp-2 | E02 | GAAGAAAGUACAUGCACUUU | SEQ ID NO: 690 |
| hTfpi-Sp-3 | E02 | CAGGGGCAAGAUUAAGCAGC | SEQ ID NO: 691 |
| hTfpi-Sp-4 | E02 | AUCAGCAUUAAGAGGGGCAG | SEQ ID NO: 692 |
| hTfpi-Sp-5 | E02 | AAUCAGCAUUAAGAGGGGCA | SEQ ID NO: 693 |
| hTfpi-Sp-6 | E02 | GAAUCAGCAUUAAGAGGGGC | SEQ ID NO: 694 |
| hTfpi-Sp-7 | E02 | CUCAGAAUCAGCAUUAAGAG | SEQ ID NO: 695 |
| hTfpi-Sp-8 | E02 | CCUCAGAAUCAGCAUUAAGA | SEQ ID NO: 696 |
| hTfpi-Sp-9 | E02 | UCCUCAGAAUCAGCAUUAAG | SEQ ID NO: 697 |
| hTfpi-Sp-10 | E02 | CCCUCUUAAUGCUGAUUCUG | SEQ ID NO: 698 |
| hTfpi-Sp-11 | E02 | GAAGAACACACAAUUAUCAC | SEQ ID NO: 699 |
| hTfpi-Sp-12 | E03 | UUAUUUUUACUUUAUAGAUA | SEQ ID NO: 700 |
| hTfpi-Sp-13 | E03 | GAAUGCAUAAGUUUCAGUGG | SEQ ID NO: 701 |
| hTfpi-Sp-14 | E03 | AAUGAAUGCAUAAGUUUCAG | SEQ ID NO: 702 |
| hTfpi-Sp-15 | E03 | GCAUUCAUUUUGUGCAUUCA | SEQ ID NO: 703 |
| hTfpi-Sp-16 | E03 | UUCAUUUUGUGCAUUCAAGG | SEQ ID NO: 704 |
| hTfpi-Sp-17 | E03 | UGUGCAUUCAAGGCGGAUGA | SEQ ID NO: 705 |
| hTfpi-Sp-18 | E03 | UUUUCAUGAUUGCUUUACAU | SEQ ID NO: 706 |
| hTfpi-Sp-19 | E03 | CUUUUCAUGAUUGCUUUACA | SEQ ID NO: 707 |
| hTfpi-Sp-20 | E03 | CAGUGCGAAGAAUUUAUAUA | SEQ ID NO: 708 |
| hTfpi-Sp-21 | E03 | AGUGCGAAGAAUUUAUAUAU | SEQ ID NO: 709 |
| hTfpi-Sp-22 | E03 | GUGCGAAGAAUUUAUAUAUG | SEQ ID NO: 710 |
| hTfpi-Sp-23 | E03 | UGCGAAGAAUUUAUAUAUGG | SEQ ID NO: 711 |
| hTfpi-Sp-24 | E03 | UUUAUAUAUGGGGGAUGUGA | SEQ ID NO: 712 |
| hTfpi-Sp-25 | E03 | UCAGAAUCGAUUUGAAAGUC | SEQ ID NO: 713 |
| hTfpi-Sp-26 | E03 | UGCAAAAAAAUGUGUACAAG | SEQ ID NO: 714 |
| hTfpi-Sp-27 | E03 | AAAAAAUGUGUACAAGAGGU | SEQ ID NO: 715 |
| hTfpi-Sp-28 | E04 | UGAUUACAGAUAAUGCAAAC | SEQ ID NO: 716 |
| hTfpi-Sp-29 | E04 | AUAAAGACAACAUUGCAACA | SEQ ID NO: 717 |
| hTfpi-Sp-30 | E05 | UCUUCCAAAAAGCAGAAAUC | SEQ ID NO: 718 |
| hTfpi-Sp-31 | E05 | AAAGCCAGAUUUCUGCUUUU | SEQ ID NO: 719 |
| hTfpi-Sp-32 | E05 | UGCUUUUUGGAAGAAGAUCC | SEQ ID NO: 720 |
| hTfpi-Sp-33 | E05 | AUAUAACCUCGACAUAUUCC | SEQ ID NO: 721 |
| hTfpi-Sp-34 | E05 | GAAGAUCCUGGAAUAUGUCG | SEQ ID NO: 722 |
| hTfpi-Sp-35 | E05 | UAUGUCGAGGUUAUAUUACC | SEQ ID NO: 723 |
| hTfpi-Sp-36 | E05 | CUGAUUGUUAUAAAAAUACC | SEQ ID NO: 724 |
| hTfpi-Sp-37 | E05 | CAGUGUGAACGUUUCAAGUA | SEQ ID NO: 725 |
| hTfpi-Sp-38 | E05 | UGUGAACGUUUCAAGUAUGG | SEQ ID NO: 726 |
| hTfpi-Sp-39 | E05 | UUUCAAGUAUGGUGGAUGCC | SEQ ID NO: 727 |
| hTfpi-Sp-40 | E05 | UUCAAGUAUGGUGGAUGCCU | SEQ ID NO: 728 |
| hTfpi-Sp-41 | E05 | CAAAAUUGUUCAUAUUGCCC | SEQ ID NO: 729 |
| hTfpi-Sp-42 | E05 | UAUGAACAAUUUUGAGACAC | SEQ ID NO: 730 |
| hTfpi-Sp-43 | E05 | UGCAAGAACAUUUGUGAAGA | SEQ ID NO: 731 |
| hTfpi-Sp-44 | E06 | CUGUAUUUUUUUCCAGCGAA | SEQ ID NO: 732 |
| hTfpi-Sp-45 | E06 | UCCACCUGGAAACCAUUCGC | SEQ ID NO: 733 |
| hTfpi-Sp-46 | E06 | UUUUCCAGCGAAUGGUUUCC | SEQ ID NO: 734 |
| hTfpi-Sp-47 | E06 | UCCAGCGAAUGGUUUCCAGG | SEQ ID NO: 735 |
| hTfpi-Sp-48 | E06 | GGGUUCCAUAAUUAUCCACC | SEQ ID NO: 736 |
| hTfpi-Sp-49 | E06 | GGUUUCCAGGUGGAUAAUUA | SEQ ID NO: 737 |
| hTfpi-Sp-50 | E06 | GUUAUUCACAGCAUUGAGCU | SEQ ID NO: 738 |
| hTfpi-Sp-51 | E06 | AGUUAUUCACAGCAUUGAGC | SEQ ID NO: 739 |
| hTfpi-Sp-52 | E06 | CUUGGUUGAUUGCGGAGUCA | SEQ ID NO: 740 |
| hTfpi-Sp-53 | E06 | CCUUGGUUGAUUGCGGAGUC | SEQ ID NO: 741 |
| hTfpi-Sp-54 | E06 | CUGGGAACCUUGGUUGAUUG | SEQ ID NO: 742 |
| hTfpi-Sp-55 | E06 | CCUGACUCCGCAAUCAACCA | SEQ ID NO: 743 |
| hTfpi-Sp-56 | E06 | ACCAAAAAGGCUGGGAACCU | SEQ ID NO: 744 |
| hTfpi-Sp-57 | E06 | AGAUUCUUACCAAAAAGGCU | SEQ ID NO: 745 |
| hTfpi-Sp-58 | E06 | AAGAUUCUUACCAAAAAGGC | SEQ ID NO: 746 |
| hTfpi-Sp-59 | E06 | ACCAAGGUUCCCAGCCUUUU | SEQ ID NO: 747 |
| hTfpi-Sp-60 | E06 | CCACAAGAUUCUUACCAAAA | SEQ ID NO: 748 |
| hTfpi-Sp-61 | E06 | CCUUUUUGGUAAGAAUCUUG | SEQ ID NO: 749 |
| hTfpi-Sp-62 | E07 | GUGAAAUUCUAAAAACAAUC | SEQ ID NO: 750 |
| hTfpi-Sp-63 | E07 | UGAUUGUUUUUAGAAUUUCA | SEQ ID NO: 751 |
| hTfpi-Sp-64 | E07 | UAGAAUUUCACGGUCCCUCA | SEQ ID NO: 752 |
| hTfpi-Sp-65 | E07 | GCUGGAGUGAGACACCAUGA | SEQ ID NO: 753 |
| hTfpi-Sp-66 | E07 | UGCUGGAGUGAGACACCAUG | SEQ ID NO: 754 |
| hTfpi-Sp-67 | E07 | CGACACAAUCCUCUGUCUGC | SEQ ID NO: 755 |
| hTfpi-Sp-68 | E07 | UGUCUCACUCCAGCAGACAG | SEQ ID NO: 756 |
| hTfpi-Sp-69 | E07 | GUAGUAGAAUCUGUUCUCAU | SEQ ID NO: 757 |
| hTfpi-Sp-70 | E07 | UUCUACUACAAUUCAGUCAU | SEQ ID NO: 758 |
| hTfpi-Sp-71 | E07 | UCUACUACAAUUCAGUCAUU | SEQ ID NO: 759 |
| hTfpi-Sp-72 | E07 | AUCCACUGUACUUAAAUGGG | SEQ ID NO: 760 |
| hTfpi-Sp-73 | E07 | CACAUCCACUGUACUUAAAU | SEQ ID NO: 761 |
| hTfpi-Sp-74 | E07 | CCACAUCCACUGUACUUAAA | SEQ ID NO: 762 |
| hTfpi-Sp-75 | E07 | UGCCGCCCAUUUAAGUACAG | SEQ ID NO: 763 |
| hTfpi-Sp-76 | E07 | CCAUUUAAGUACAGUGGAUG | SEQ ID NO: 764 |
| hTfpi-Sp-77 | E07 | CAUUUAAGUACAGUGGAUGU | SEQ ID NO: 765 |
| hTfpi-Sp-78 | E07 | AUUUAAGUACAGUGGAUGUG | SEQ ID NO: 766 |
| hTfpi-Sp-79 | E07 | UUUAAGUACAGUGGAUGUGG | SEQ ID NO: 767 |
| hTfpi-Sp-80 | E07 | UGCCCUCAGACAUUCUUGUU | SEQ ID NO: 768 |
| hTfpi-Sp-81 | E07 | CUUCCAAACAAGAAUGUCUG | SEQ ID NO: 769 |
| hTfpi-Sp-82 | E07 | UUCCAAACAAGAAUGUCUGA | SEQ ID NO: 770 |
| hTfpi-Sp-83 | E07 | UGUCUGAGGGCAUGUAAAAA | SEQ ID NO: 771 |
| hTfpi-Sp-84 | E08 | AUGAAACCUAUAAGAGGAAG | SEQ ID NO: 772 |
| hTfpi-Sp-85 | E08 | CUUUGGAUGAAACCUAUAAG | SEQ ID NO: 773 |
| hTfpi-Sp-86 | E08 | GGCCUCCUUUUGAUAUUCUU | SEQ ID NO: 774 |
| hTfpi-Sp-87 | E08 | UUCAUCCAAAGAAUAUCAAA | SEQ ID NO: 775 |
| hTfpi-Sp-88 | E08 | AUCCAAAGAAUAUCAAAAGG | SEQ ID NO: 776 |
| hTfpi-Sp-89 | E08 | UUUUUCUUUUGGUUUUAAUU | SEQ ID NO: 777 |
| hTfpi-Sp-90 | E08 | CUGCUUCUUUCUUUUUCUUU | SEQ ID NO: 778 |
| hTfpi-Sa-1 | E02 | UGUGUGUUCUUCAUCUUCCU | SEQ ID NO: 779 |
| hTfpi-Sa-2 | E03 | UUAUUUUUACUUUAUAGAUA | SEQ ID NO: 780 |
| hTfpi-Sa-3 | E03 | AUCCGCCUUGAAUGCACAAA | SEQ ID NO: 781 |
| hTfpi-Sa-4 | E03 | AUUCAUUUUGUGCAUUCAAG | SEQ ID NO: 782 |
| hTfpi-Sa-5 | E03 | UACAUGGGCCAUCAUCCGCC | SEQ ID NO: 783 |
| hTfpi-Sa-6 | E03 | UAUAUAAAUUCUUCGCACUG | SEQ ID NO: 784 |
| hTfpi-Sa-7 | E03 | UAUUUUCACUCGACAGUGCG | SEQ ID NO: 785 |
| hTfpi-Sa-8 | E03 | GUGCGAAGAAUUUAUAUAUG | SEQ ID NO: 786 |
| hTfpi-Sa-9 | E03 | AUGGGGGAUGUGAAGGAAAU | SEQ ID NO: 787 |
| hTfpi-Sa-10 | E03 | GAAUCGAUUUGAAAGUCUGG | SEQ ID NO: 788 |
| hTfpi-Sa-11 | E04 | UUGAUUACAGAUAAUGCAAA | SEQ ID NO: 789 |
| hTfpi-Sa-12 | E05 | UGCUUUUUGGAAGAAGAUCC | SEQ ID NO: 790 |
| hTfpi-Sa-13 | E05 | AAUAUAACCUCGACAUAUUC | SEQ ID NO: 791 |
| hTfpi-Sa-14 | E05 | GUGUGAACGUUUCAAGUAUG | SEQ ID NO: 792 |
| hTfpi-Sa-15 | E05 | GAACAAUUUUGAGACACUGG | SEQ ID NO: 793 |
| hTfpi-Sa-16 | E06 | UUCCAGCGAAUGGUUUCCAG | SEQ ID NO: 794 |
| hTfpi-Sa-17 | E06 | GAGUUAUUCACAGCAUUGAG | SEQ ID NO: 795 |
| hTfpi-Sa-18 | E06 | AUGGAACCCAGCUCAAUGCU | SEQ ID NO: 796 |
| hTfpi-Sa-19 | E06 | CUUGGUUGAUUGCGGAGUCA | SEQ ID NO: 797 |
| hTfpi-Sa-20 | E06 | CUGGGAACCUUGGUUGAUUG | SEQ ID NO: 798 |
| hTfpi-Sa-21 | E06 | AGGUUCCCAGCCUUUUUGGU | SEQ ID NO: 799 |
| hTfpi-Sa-22 | E07 | CGACACAAUCCUCUGUCUGC | SEQ ID NO: 800 |
| hTfpi-Sa-23 | E07 | GUGUCUCACUCCAGCAGACA | SEQ ID NO: 801 |
| hTfpi-Sa-24 | E07 | UCCCAAUGACUGAAUUGUAG | SEQ ID NO: 802 |
| hTfpi-Sa-25 | E07 | UGGGCGGCAUUUCCCAAUGA | SEQ ID NO: 803 |
| hTfpi-Sa-26 | E07 | AUGCCGCCCAUUUAAGUACA | SEQ ID NO: 804 |
| hTfpi-Sa-27 | E07 | AAACAAUUUUACUUCCAAAC | SEQ ID NO: 805 |
| hTfpi-Sa-28 | E08 | CCUCUUAUAGGUUUCAUCCA | SEQ ID NO: 806 |
| hTfpi-Sa-29 | E08 | AGGCCUCCUUUUGAUAUUCU | SEQ ID NO: 807 |
| hTfpi-Sa-30 | E08 | GAAAUUUUUGUUAAAAAUAU | SEQ ID NO: 808 |
| hTfpi-Cj-1 | E02 | UGGGUUCUGUAUUUCAGAGAUG | SEQ ID NO: 809 |
| hTfpi-Cj-2 | E02 | UCUUCAUUGUGUAAAUCAUCUC | SEQ ID NO: 810 |
| hTfpi-Cj-3 | E02 | CAGAGAUGAUUUACACAAUGAA | SEQ ID NO: 811 |
| hTfpi-Cj-4 | E02 | UGAUUUACACAAUGAAGAAAGU | SEQ ID NO: 812 |
| hTfpi-Cj-5 | E02 | CAGGCAUACAGAAGCCCAAAGU | SEQ ID NO: 813 |
| hTfpi-Cj-6 | E02 | GGCAGGGGCAAGAUUAAGCAGC | SEQ ID NO: 814 |
| hTfpi-Cj-7 | E02 | AAUGCUGAUUCUGAGGAAGAUG | SEQ ID NO: 815 |
| hTfpi-Cj-8 | E02 | UGCUGAUUCUGAGGAAGAUGAA | SEQ ID NO: 816 |
| hTfpi-Cj-9 | E03 | UACAUGGGCCAUCAUCCGCCUU | SEQ ID NO: 817 |
| hTfpi-Cj-10 | E03 | UCACAUCCCCCAUAUAUAAAUU | SEQ ID NO: 818 |
| hTfpi-Cj-11 | E03 | AAGUCUGGAAGAGUGCAAAAAA | SEQ ID NO: 819 |
| hTfpi-Cj-12 | E03 | AACCUACCUCUUGUACACAUUU | SEQ ID NO: 820 |
| hTfpi-Cj-13 | E03 | AGGGUUCCCAGAAACCUACCUC | SEQ ID NO: 821 |
| hTfpi-Cj-14 | E05 | CACUGUUUUGUCUGAUUGUUAU | SEQ ID NO: 822 |
| hTfpi-Cj-15 | E05 | AGGCAUCCACCAUACUUGAAAC | SEQ ID NO: 823 |
| hTfpi-Cj-16 | E05 | UUGUUCAUAUUGCCCAGGCAUC | SEQ ID NO: 824 |
| hTfpi-Cj-17 | E05 | GCCUGGGCAAUAUGAACAAUUU | SEQ ID NO: 825 |
| hTfpi-Cj-18 | E07 | CACAAUCCUCUGUCUGCUGGAG | SEQ ID NO: 826 |
| hTfpi-Cj-19 | E07 | UAGUAGAAUCUGUUCUCAUUGG | SEQ ID NO: 827 |
| hTfpi-Cj-20 | E07 | AAUUGUAGUAGAAUCUGUUCUC | SEQ ID NO: 828 |
| hTfpi-Cj-21 | E07 | GUCAUUGGGAAAUGCCGCCCAU | SEQ ID NO: 829 |
| hTfpi-Cj-22 | E07 | UUGUUUUCAUUUCCCCCACAUC | SEQ ID NO: 830 |

One aspect of the disclosure of the present specification relates to a composition for gene manipulation for artificially manipulating a blood coagulation inhibitory gene.

The composition for gene manipulation may be used in the generation of an artificially manipulated blood coagulation inhibitory gene. In addition, the blood coagulation inhibitory gene artificially manipulated by the composition for gene manipulation may regulate a blood coagulation system.

The “artificially manipulated (artificially modified or engineered or artificially engineered)” refers to an artificially modified state, rather than as it exists in a naturally-occurring state. Hereinafter, an unnatural artificially manipulated or modified blood coagulation inhibitory gene may be used interchangeably with an artificial blood coagulation inhibitory gene.

Gene manipulation may be done in consideration of a gene expression regulation process.

In an embodiment, the gene manipulation may be achieved by selecting a manipulation tool suitable for each of the steps of transcriptional regulation, RNA processing regulation, RNA transport regulation, RNA cleavage regulation, translational regulation, or protein modification regulation.

For example, the expression of genetic information may be controlled using RNAi (RNA interference or RNA silencing) to allow small RNA (sRNA) to interfere with mRNA or reduce the stability of mRNA, and optionally break down the mRNA, thereby making it prevent from informing on protein synthesis.

In an embodiment, gene manipulation may be performed using a wild-type or variant enzyme that can catalyze the hydrolysis (cleavage) of bonds between nucleotides in DNA or RNA molecules, preferably DNA molecules. A guide nucleic acid-editor protein complex may be used.

For example, the expression of genetic information may be controlled by manipulating a gene using one or more nucleases selected from the group consisting of meganucleases, Zinc finger nucleases, CRISPR/Cas9 proteins, CRISPR-Cpf1 proteins, and TALE-nucleases.

The composition for gene manipulation disclosed in the present specification may include a guide nucleic acid and an editor protein.

In one embodiment, the composition for gene manipulation may include

a guide nucleic acid capable of targeting a target sequence of a blood coagulation inhibitory gene or a nucleic acid sequence encoding the same; and

one or more editor proteins or a nucleic acid sequence encoding the same.

The description related to the blood coagulation inhibitory gene is the same as described above.

The description related to the target sequence is the same as described above.

In another embodiment, the composition for gene manipulation may include

a guide nucleic acid including a guide sequence capable of forming a complementary bond with a target sequence of a blood coagulation inhibitory gene or a nucleic acid sequence encoding the same; and

one or more editor proteins or a nucleic acid sequence encoding the same.

The description related to the blood coagulation inhibitory gene is the same as described above.

The description related to the target sequence is the same as described above.

The description related to the guide sequence is the same as described above.

The composition for gene manipulation may include a guide nucleic acid-editor protein complex.

The term “guide nucleic acid-editor protein complex” refers to a complex formed through the interaction between a guide nucleic acid and an editor protein.

A description related to the guide nucleic acid is as described above.

The term “editor protein” refers to a peptide, polypeptide or protein which is able to directly bind to or interact with, without direct binding to, a nucleic acid.

Here, the nucleic acid may be a nucleic acid included in a target nucleic acid, gene or chromosome.

Here, the nucleic acid may be a guide nucleic acid.

The editor protein may be an enzyme.

Here, the term “enzyme” refers to a polypeptide or protein that contains a domain capable of cleaving a nucleic acid, gene or chromosome.

The enzyme may be a nuclease or restriction enzyme.

The editor protein may include a complete active enzyme.

Here, the “complete active enzyme” refers to an enzyme having the same function as the nucleic acid, gene or chromosome cleavage function of a wild-type enzyme. For example, the wild-type enzyme that cleaves double-stranded DNA may be a complete active enzyme that entirely cleaves double-stranded DNA. As another example, when the wild-type enzyme cleaving double-stranded DNA undergoes a deletion or substitution of a partial sequence of an amino acids sequence due to artificial engineering, the artificially engineered enzyme variant cleaves double-stranded DNA like the wild-type enzyme, the artificially engineered enzyme variant may be a complete active enzyme.

In addition, the complete active enzyme may include an enzyme having an improved function, compared to the wild-type enzyme. For example, a specific modified or manipulated form of the wild-type enzyme cleaving double-stranded DNA may have a complete enzyme activity, which is greater than the wild-type enzyme, that is, an increased activity of cleaving double-stranded DNA.

The editor protein may include an incomplete or partially active enzyme.

Here, the “incomplete or partially active enzyme” refers to an enzyme having some of the nucleic acid, gene or chromosome cleavage function of the wild-type enzyme. For example, a specific modified or manipulated form of the wild-type enzyme that cleaves double-stranded DNA may be a form having a first function or a form having a second function. Here, the first function is a function of cleaving the first strand of double-stranded DNA, and the second function may be a function of cleaving the second strand of double-stranded DNA. Here, the enzyme with the first function or the enzyme with the second function may be an incomplete or partially active enzyme.

The editor protein may include an inactive enzyme.

Here, the “inactive enzyme” refers to an enzyme in which the nucleic acid, gene or chromosome cleavage function of the wild-type enzyme is entirely inactivated. For example, a specific modified or manipulated form of the wild-type enzyme may be a form in which both of the first and second functions are lost, that is, both of the first function of cleaving the first strand of double-stranded DNA and the second function of cleaving the second strand thereof are lost. Here, the enzyme in which all of the first and second functions are lost may be inactive enzyme.

The editor protein may be a fusion protein.

Here, the term “fusion protein” refers to a protein produced by fusing an enzyme with an additional domain, peptide, polypeptide or protein.

The additional domain, peptide, polypeptide or protein may have a same or different function as a functional domain, peptide, polypeptide, or protein included in the enzyme.

The fusion protein may be a form in which the functional domain, peptide, polypeptide or protein is added to one or more of the amino end of an enzyme or the proximity thereof; the carboxyl end of the enzyme or the proximity thereof; the middle part of the enzyme; or a combination thereof.

Here, the functional domain, peptide, polypeptide or protein may be a domain, peptide, polypeptide or protein having methylase activity, demethylase activity, transcription activation activity, transcription repression activity, transcription release factor activity, histone modification activity, RNA cleavage activity or nucleic acid binding activity, or a tag or reporter gene for isolation and purification of a protein (including a peptide), but the present invention is not limited thereto.

The functional domain, peptide, polypeptide or protein may be a deaminase.

The tag includes a histidine (His) tag, a V5 tag, a FLAG tag, an influenza hemagglutinin (HA) tag, a Myc tag, a VSV-G tag and a thioredoxin (Trx) tag, and the reporter gene includes glutathione-S-transferase (GST), horseradish peroxidase (HRP), chloramphenicol acetyltransferase (CAT) β-galactosidase, β-glucoronidase, luciferase, auto fluorescent proteins including the green fluorescent protein (GFP), HcRed, DsRed, cyan fluorescent protein (CFP), yellow fluorescent protein (YFP) and blue fluorescent protein (BFP), but the present invention is not limited thereto.

In addition, the functional domain, peptide, polypeptide or protein may be a nuclear localization sequence or signal (NLS) or a nuclear export sequence or signal (NES).

The NLS may be NLS of SV40 virus large T-antigen with an amino acid sequence PKKKRKV (SEQ ID NO: 831); NLS derived from nucleoplasmin (e.g., nucleoplasmin bipartite NLS with a sequence KRPAATKKAGQAKKKK(SEQ ID NO: 832)); c-myc NLS with an amino acid sequence PAAKRVKLD(SEQ ID NO: 833) or RQRRNELKRSP(SEQ ID NO: 834); hRNPA1 M9 NLS with a sequence NQSSNFGPMKGGNFGGRSSGPYGGGGQYFAKPRNQGGY(SEQ ID NO: 835); an importin-α-derived IBB domain sequence RMRIZFKNKGKDTAELRRRRVEVSVELRKAKKDEQILKRRNV(SEQ ID NO: 836); myoma T protein sequences VSRKRPRP(SEQ ID NO: 837) and PPKKARED(SEQ ID NO: 838); human p53 sequence PQPKKKPL(SEQ ID NO: 839); a mouse c-abl IV sequence SALIKKKKKMAP(SEQ ID NO: 840); influenza virus NS1 sequences DRLRR(SEQ ID NO: 841) and PKQKKRK(SEQ ID NO: 842); a hepatitis virus-δ antigen sequence RKLKKKIKKL(SEQ ID NO: 843); a mouse Mx1 protein sequence REKKKFLKRR(SEQ ID NO: 844); a human poly(ADP-ribose) polymerase sequence KRKGDEVDGVDEVAKKKSKK(SEQ ID NO: 845); or steroid hormone receptor (human) glucocorticoid sequence RKCLQAGMNLEARKTKK(SEQ ID NO: 846), but the present invention is not limited thereto.

The additional domain, peptide, polypeptide or protein may be a non-functional domain, peptide, polypeptide or protein that does not perform a specific function. Here, the non-functional domain, peptide, polypeptide or protein may be a domain, peptide, polypeptide or protein that does not affect the enzyme function.

The fusion protein may be a form in which the non-functional domain, peptide, polypeptide or protein is added to one or more of the amino end of an enzyme or the proximity thereof; the carboxyl end of the enzyme or the proximity thereof; the middle part of the enzyme; or a combination thereof.

The editor protein may be a wild-type of enzyme or fusion protein that exists in nature.

The editor protein may be a form of a partially modified enzyme or fusion protein.

The editor protein may be an artificially produced enzyme or fusion protein, which does not exist in nature.

The editor protein may be a form of a partially modified artificial enzyme or fusion protein, which does not exist in nature.

Here, the modification may be substitution, removal, addition of amino acids contained in the editor protein, or a combination thereof.

Alternatively, the modification may be substitution, removal, addition of some nucleotides in the nucleotide sequence encoding the editor protein, or a combination thereof.

In addition, optionally, the composition for gene manipulation may further include a donor having a desired specific nucleotide sequence, which is to be inserted, or a nucleic acid sequence encoding the same.

Here, the nucleic acid sequence to be inserted may be a partial nucleotide sequence of the blood coagulation inhibitory gene.

Here, the nucleic acid sequence to be inserted may be a nucleic acid sequence for being introduced into the blood coagulation inhibitory gene to be manipulated and used to correct a mutation of the blood coagulation inhibitory gene.

The term “donor” refers to a nucleotide sequence that helps homologous recombination (HR)-based repair of a damaged gene or nucleic acid.

The donor may be a double- or single-stranded nucleic acid.

The donor may be present in a linear or circular shape.

The donor may include a nucleotide sequence having homology with a target gene or a nucleic acid.

For example, the donor may include a nucleotide sequence having homology with each of nucleotide sequences of upstream (left) and downstream (right) of a location where a specific nucleotide sequence is inserted, in which a damaged nucleic acid exists. Here, the specific nucleotide sequence to be inserted may be located between a nucleotide sequence having homology with a left nucleotide sequence of the damaged nucleic acid and a nucleotide sequence having homology with a right nucleotide sequence of the damaged nucleic acid. Here, the nucleotide sequence having homology may have at least 50%, 55%, 60%, 65%, 70%, 75%, 80%, 85%, 90% or 95% or more homology or complete homology.

The donor may selectively include an additional nucleotide sequence. Here, the additional nucleic acid sequence may play a role in increasing stability, insertion efficiency into a target or homologous recombination efficiency of the donor.

For example, the additional nucleotide sequence may be a nucleic acid sequence rich in nucleotides A and T, that is, an A-T rich domain. For example, the additional nucleotide sequence may be a scaffold/matrix attachment region (SMAR).

The guide nucleic acid, editor protein or guide nucleic acid-editor protein complex disclosed in the specification may be delivered or introduced into a subject in various ways.

Here, the term “subject” refers to an organism into which a guide nucleic acid, editor protein or guide nucleic acid-editor protein complex is introduced, an organism in which a guide nucleic acid, editor protein or guide nucleic acid-editor protein complex operates, or a specimen or sample obtained from the organism.

The subject may be an organism including a target gene or chromosome of a guide nucleic acid-editor protein complex.

The organism may be an animal, animal tissue or an animal cell.

The organism may be a human, human tissue or a human cell.

The tissue may be eyeball, skin, liver, kidney, heart, lung, brain, muscle tissue, or blood.

The cells may be a liver cell, an immune cell, a blood cell, or a stem cell.

The specimen or sample may be acquired from an organism including a target gene or chromosome such as saliva, blood, liver tissue, brain tissue, a liver cell, a nerve cell, a phagocyte, a macrophage, a T cell, a B cell, an astrocyte, a cancer cell or a stem cell, etc.

Preferably, the subject may be an organism including a blood coagulation inhibitory gene.

The guide nucleic acid, editor protein or guide nucleic acid-editor protein complex may be delivered or introduced into a subject in the form of DNA, RNA or a mixed form.

Here, the form of DNA, RNA or a mixture thereof, which encodes the guide nucleic acid and/or editor protein may be delivered or introduced into a subject by a method known in the art.

Or, the form of DNA, RNA or a mixture thereof, which encodes the guide nucleic acid and/or editor protein may be delivered or introduced into a subject by a vector, a non-vector or a combination thereof.

The vector may be a viral or non-viral vector (e.g., a plasmid).

The non-vector may be naked DNA, a DNA complex or mRNA.

In one embodiment, the nucleic acid sequence encoding the guide nucleic acid and/or editor protein may be delivered or introduced into a subject by a vector.

The vector may include a nucleic acid sequence encoding a guide nucleic acid and/or editor protein.

In one example, the vector may simultaneously include nucleic acid sequences, which encode the guide nucleic acid and the editor protein.

In another example, the vector may include the nucleic acid sequence encoding the guide nucleic acid.

As an example, domains included in the guide nucleic acid may be contained all in one vector, or may be divided and then contained in different vectors.

In another example, the vector may include the nucleic acid sequence encoding the editor protein.

As an example, in the case of the editor protein, the nucleic acid sequence encoding the editor protein may be contained in one vector, or may be divided and then contained in several vectors.

The vector may include one or more regulatory/control components.

Here, the regulatory/control components may include a promoter, an enhancer, an intron, a polyadenylation signal, a Kozak consensus sequence, an internal ribosome entry site (IRES), a splice acceptor and/or a 2A sequence.

The promoter may be a promoter recognized by RNA polymerase II.

The promoter may be a promoter recognized by RNA polymerase III.

The promoter may be an inducible promoter.

The promoter may be a subject-specific promoter.

The promoter may be a viral or non-viral promoter.

The promoter may use a suitable promoter according to a control region (that is, a nucleic acid sequence encoding a guide nucleic acid or editor protein).

For example, a promoter useful for the guide nucleic acid may be a H1, EF- 1a, tRNA or U6 promoter. For example, a promoter useful for the editor protein may be a CMV, EF-1a, EFS, MSCV, PGK or CAG promoter.

The vector may be a viral vector or recombinant viral vector.

The virus may be a DNA virus or an RNA virus.

Here, the DNA virus may be a double-stranded DNA (dsDNA) virus or single- stranded DNA (ssDNA) virus.

Here, the RNA virus may be a single-stranded RNA (ssRNA) virus.

The virus may be a retrovirus, a lentivirus, an adenovirus, adeno-associated virus (AAV), vaccinia virus, a poxvirus or a herpes simplex virus, but the present invention is not limited thereto.

Generally, the virus may infect a host (e.g., cells), thereby introducing a nucleic acid encoding the genetic information of the virus into the host or inserting a nucleic acid encoding the genetic information into the host genome. The guide nucleic acid and/or editor protein may be introduced into a subject using a virus having such a characteristic. The guide nucleic acid and/or editor protein introduced using the virus may be temporarily expressed in the subject (e.g., cells). Alternatively, the guide nucleic acid and/or editor protein introduced using the virus may be continuously expressed in a subject (e.g., cells) for a long time (e.g., 1, 2, 3 weeks, 1, 2, 3, 6, 9 months, 1, 2 years, or permanently).

The packaging capability of the virus may vary from at least 2 kb to 50 kb according to the type of virus. Depending on such a packaging capability, a viral vector including a guide nucleic acid or an editor protein or a viral vector including both of a guide nucleic acid and an editor protein may be designed. Alternatively, a viral vector including a guide nucleic acid, an editor protein and additional components may be designed.

In one example, a nucleic acid sequence encoding a guide nucleic acid and/or editor protein may be delivered or introduced by a recombinant lentivirus.

In another example, a nucleic acid sequence encoding a guide nucleic acid and/or editor protein may be delivered or introduced by a recombinant adenovirus.

In still another example, a nucleic acid sequence encoding a guide nucleic acid and/or editor protein may be delivered or introduced by recombinant AAV.

In yet another example, a nucleic acid sequence encoding a guide nucleic acid and/or editor protein may be delivered or introduced by a hybrid virus, for example, one or more hybrids of the virus listed herein.

In another embodiment, the nucleic acid sequence encoding the guide nucleic acid and/or editor protein may be delivered or introduced into a subject by a non-vector.

The non-vector may include a nucleic acid sequence encoding a guide nucleic acid and/or editor protein.

The non-vector may be naked DNA, a DNA complex, mRNA, or a mixture thereof.

The non-vector may be delivered or introduced into a subject by electroporation, gene gun, sonoporation, magnetofection, transient cell compression or squeezing (e.g., described in the literature [Lee, et al, (2012) Nano Lett., 12, 6322-6327]), lipid-mediated transfection, a dendrimer, nanoparticles, calcium phosphate, silica, a silicate (Ormosil), or a combination thereof.

In one example, the delivery through electroporation may be performed by mixing cells and a nucleic acid sequence encoding a guide nucleic acid and/or editor protein in a cartridge, chamber or cuvette, and applying electrical stimuli with a predetermined duration and amplitude to the cells.

In another example, the non-vector may be delivered using nanoparticles. The nanoparticles may be inorganic nanoparticles (e.g., magnetic nanoparticles, silica, etc.) or organic nanoparticles (e.g., a polyethylene glycol (PEG)-coated lipid, etc.). The outer surface of the nanoparticles may be conjugated with a positively-charged polymer which is attachable (e.g., polyethyleneimine, polylysine, polyserine, etc.).

In a certain embodiment, the non-vector may be delivered using a lipid shell.

In a certain embodiment, the non-vector may be delivered using an exosome. The exosome is an endogenous nano-vesicle for transferring a protein and RNA, which can deliver RNA to the brain and another target organ.

In a certain embodiment, the non-vector may be delivered using a liposome. The liposome is a spherical vesicle structure which is composed of single or multiple lamellar lipid bilayers surrounding internal aqueous compartments and an external, lipophilic phospholipid bilayer which is relatively non-transparent. While the liposome may be made from several different types of lipids; phospholipids are most generally used to produce the liposome as a drug carrier.

In addition, the composition for delivery of the non-vector may include other additives.

The editor protein may be delivered or introduced into a subject in the form of a peptide, polypeptide or protein.

The editor protein may be delivered or introduced into a subject in the form of a peptide, polypeptide or protein by a method known in the art.

The peptide, polypeptide or protein form may be delivered or introduced into a subject by electroporation, microinjection, transient cell compression or squeezing (e.g., described in the literature [Lee, et al, (2012) Nano Lett., 12, 6322-6327]), lipid-mediated transfection, nanoparticles, a liposome, peptide-mediated delivery or a combination thereof.

The peptide, polypeptide or protein may be delivered with a nucleic acid sequence encoding a guide nucleic acid.

In one example, the transfer through electroporation may be performed by mixing cells into which the editor protein will be introduced with or without a guide nucleic acid in a cartridge, chamber or cuvette, and applying electrical stimuli with a predetermined duration and amplitude to the cells.

The guide nucleic acid and the editor protein may be delivered or introduced into a subject in the form of mixing a nucleic acid and a protein.

The guide nucleic acid and the editor protein may be delivered or introduced into a subject in the form of a guide nucleic acid-editor protein complex.

For example, the guide nucleic acid may be a DNA, RNA or a mixture thereof. The editor protein may be a peptide, polypeptide or protein.

In one example, the guide nucleic acid and the editor protein may be delivered or introduced into a subject in the form of a guide nucleic acid-editor protein complex containing an RNA-type guide nucleic acid and a protein-type editor protein, that is, a ribonucleoprotein (RNP).

The guide nucleic acid-editor protein complex disclosed in the specification may modify a target nucleic acid, gene or chromosome.

For example, the guide nucleic acid-editor protein complex induces a modification in the sequence of a target nucleic acid, gene or chromosome. As a result, a protein expressed by the target nucleic acid, gene or chromosome may be modified in structure and/or function, or the expression of the protein may be controlled or inhibited.

The guide nucleic acid-editor protein complex may act at the DNA, RNA, gene or chromosome level.

In one example, the guide nucleic acid-editor protein complex may manipulate or modify the target gene to control (e.g., suppress, inhibit, reduce, increase or promote) the expression of a protein encoded by a target gene, or express a protein whose activity is controlled (e.g., suppressed, inhibited, reduced, increased or promoted) or modified.

The guide nucleic acid-editor protein complex may act at the transcription and translation stage of a gene.

In one example, the guide nucleic acid-editor protein complex may promote or inhibit the transcription of a target gene, thereby controlling (e.g., suppressing, inhibiting, reducing, increasing or promoting) the expression of a protein encoded by the target gene.

In another example, the guide nucleic acid-editor protein complex may promote or inhibit the translation of a target gene, thereby controlling (e.g., suppressing, inhibiting, reducing, increasing or promoting) the expression of a protein encoded by the target gene.

In one embodiment of the disclosure of the present specification, the composition for gene manipulation may include gRNA and a CRISPR enzyme.

In one embodiment, the composition for gene manipulation may include
a gRNA capable of targeting a target sequence of a blood coagulation inhibitory gene or a nucleic acid sequence encoding the same; and
one or more CRISPR enzymes or a nucleic acid sequence encoding the same.

The description related to the blood coagulation inhibitory gene is the same as described above.

The description related to the target sequence is the same as described above.

In another embodiment, the composition for gene manipulation may include

a gRNA including a guide sequence capable of forming a complementary bond with a target sequence of a blood coagulation inhibitory gene or a nucleic acid sequence encoding the same; and

one or more CRISPR enzymes or a nucleic acid sequence encoding the same.

The description related to the blood coagulation inhibitory gene is the same as described above.

The description related to the target sequence is the same as described above.

The description related to the guide sequence is the same as described above.

The composition for gene manipulation may include a gRNA-CRISPR enzyme complex.

The term “gRNA-CRISPR enzyme complex” refers to a complex formed by an interaction between gRNA and a CRISPR enzyme.

A description related to the gRNA is as described above.

The term “CRISPR enzyme” is a main protein component of a CRISPR-Cas system, and forms a complex with gRNA, resulting in the CRISPR-Cas system.

The CRISPR enzyme may be a nucleic acid having a sequence encoding the CRISPR enzyme or a polypeptide (or a protein).

The CRISPR enzyme may be a Type II CRISPR enzyme.

The crystal structure of the type II CRISPR enzyme was determined according to studies on two or more types of natural microbial type II CRISPR enzyme molecules (Jinek et al., Science, 343(6176):1247997, 2014) and studies on Streptococcus pyogenes Cas9 (SpCas9) complexed with gRNA (Nishimasu et al., Cell, 156:935-949, 2014; and Anders et al., Nature, 2014, doi: 10.1038/nature13579).

The type II CRISPR enzyme includes two lobes, that is, recognition (REC) and nuclease (NUC) lobes, and each lobe includes several domains.

The REC lobe includes an arginine-rich bridge helix (BH) domain, an REC1 domain and an REC2 domain.

Here, the BH domain is a long α-helix and arginine-rich region, and the REC1 and REC2 domains play an important role in recognizing a double strand formed in gRNA, for example, single-stranded gRNA, double-stranded gRNA or tracrRNA.

The NUC lobe includes a RuvC domain, an HNH domain and a PAM- interaction (PI) domain. Here, the RuvC domain encompasses RuvC-like domains, and the HNH domain encompasses HNH-like domains.

Here, the RuvC domain shares structural similarity with members of the microorganism family existing in nature including the type II CRISPR enzyme, and cleaves a single strand, for example, a non-complementary strand of a target gene or a nucleic acid, that is, a strand not forming a complementary bond with gRNA. The RuvC domain is sometimes referred to as a RuvCI domain, RuvCll domain or RuvClll domain in the art, and generally called an RuvC I, RuvCll or RuvClll.

The HNH domain shares structural similarity with the HNH endonuclease, and cleaves a single strand, for example, a complementary strand of a target nucleic acid molecule, that is, a strand forming a complementary bond with gRNA. The HNH domain is located between RuvC II and III motifs.

The PI domain recognizes a specific nucleotide sequence in a target gene or a nucleic acid, that is, a protospacer adjacent motif (PAM), or interacts with PAM. Here, the PAM may vary according to the origin of a Type II CRISPR enzyme. For example, when the CRISPR enzyme is SpCas9, the PAM may be 5′-NGG-3′, and when the CRISPR enzyme is Streptococcus thermophilus Cas9 (StCas9), the PAM may be 5′- NNAGAAW-3′ (W = A or T), when the CRISPR enzyme is Neisseria meningiditis Cas9 (NmCas9), the PAM may be 5′-NNNNGATT-3′, and when the CRISPR enzyme is Campylobacter jejuni Cas9 (CjCas9), the PAM may be 5′-NNNVRYAC-3′ (V = G or C or A, R = A or G, Y = C or T), herein, N is A, T, G or C; or A, U, G or C). However, while it is generally understood that PAM is determined according to the origin of the above- described enzyme, as the study of a mutant of an enzyme derived from the corresponding origin progresses, the PAM may be changed.

The Type II CRISPR enzyme may be Cas9.

The Cas9 may be derived from various microorganisms such as Streptococcus pyogenes, Streptococcus thermophilus, Streptococcus sp., Staphylococcus aureus, Campylobacter jejuni, Nocardiopsis dassonvillei, Streptomyces pristinaespiralis, Streptomyces viridochromogenes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii,, Cyanothece sp., Microcystis aeruginosa, Synechococcus sp., Acetohalobium arabaticum, Ammonifex degensii, Caldicelulosiruptor bescii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus and Acaryochloris marina.

The Cas9 is an enzyme which binds to gRNA so as to cleave or modify a target sequence or position of a target gene or a nucleic acid, and may consist of an HNH domain capable of cleaving a nucleic acid strand forming a complementary bond with gRNA, an RuvC domain capable of cleaving a nucleic acid strand forming a non- complementary bond with gRNA, an REC domain interacting with the target and a PI domain recognizing a PAM. Hiroshi Nishimasu et al. (2014) Cell 156:935-949 may be referenced for specific structural characteristics of Cas9.

The Cas9 may be isolated from a microorganism existing in nature or non- naturally produced by a recombinant or synthetic method.

In addition, the CRISPR enzyme may be a Type V CRISPR enzyme.

The type V CRISPR enzyme includes similar RuvC domains corresponding to the RuvC domains of the type II CRISPR enzyme, and the HNH domain of the Type II CRISPR enzyme is deficient. But it instead includes an Nuc domain, and may consist of REC and WED domains interacting with a target, and a PI domain recognizing PAM. For specific structural characteristics of the type V CRISPR enzyme, Takashi Yamano et al. (2016) Cell 165:949-962 may be referenced.

The type V CRISPR enzyme may interact with gRNA, thereby forming a gRNA-CRISPR enzyme complex, that is, a CRISPR complex, and may allow a guide sequence to approach a target sequence including a PAM sequence in cooperation with gRNA. Here, the ability of the type V CRISPR enzyme for interaction with a target gene or a nucleic acid is dependent on the PAM sequence.

The PAM sequence may be a sequence present in a target gene or a nucleic acid, and recognized by the PI domain of a Type V CRISPR enzyme. The PAM sequence may have different sequences according to the origin of the Type V CRISPR enzyme. That is, each species has a specifically recognizable PAM sequence. For example, the PAM sequence recognized by Cpf1 may be 5′-TTN-3′ (N is A, T, C or G). While it has been generally understood that PAM is determined according to the origin of the above-described enzyme, as the study of mutants of the enzyme derived from the corresponding origin progresses, the PAM may be changed.

The Type V CRISPR enzyme may be Cpf1.

The Cpf1 may be derived from Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacillus, Methylobacterium or Acidaminococcus.

The Cpf1 includes a RuvC-like domain corresponding to the RuvC domains of Cas9, and the HNH domain of the Cas9 is deficient. But it instead includes an Nuc domain, and may consist of REC and WED domains interacting with a target, and a PI domain recognizing PAM. For specific structural characteristics of Cpf1, Takashi Yamano et al. (2016) Cell 165:949-962 may be referenced.

The Cpf1 may be isolated from a microorganism existing in nature or non- naturally produced by a recombinant or synthetic method.

The CRISPR enzyme may be a nuclease or restriction enzyme having a function of cleaving a double-stranded nucleic acid of a target gene or a nucleic acid.

The CRISPR enzyme may be a complete active CRISPR enzyme.

The term “complete active” refers to a state in which an enzyme has the same function as that of a wild-type CRISPR enzyme, and the CRISPR enzyme in such a state is named a complete active CRISPR enzyme. Here, the “function of the wild-type CRISPR enzyme” refers to a state in which an enzyme has functions of cleaving double- stranded DNA, that is, the first function of cleaving the first strand of double-stranded DNA and a second function of cleaving the second strand of double-stranded DNA.

The complete active CRISPR enzyme may be a wild-type CRISPR enzyme that cleaves double-stranded DNA.

The complete active CRISPR enzyme may be a CRISPR enzyme variant formed by modifying or manipulating the wild-type CRISPR enzyme that cleaves double- stranded DNA.

The CRISPR enzyme variant may be an enzyme in which one or more amino acids of the amino acid sequence of the wild-type CRISPR enzyme are substituted with other amino acids, or one or more amino acids are removed.

The CRISPR enzyme variant may be an enzyme in which one or more amino acids are added to the amino acid sequence of the wild-type CRISPR enzyme. Here, the location of the added amino acids may be the N-end, the C-end or in the amino acid sequence of the wild-type enzyme.

The CRISPR enzyme variant may be a complete active enzyme with an improved function compared to the wild-type CRISPR enzyme.

For example, a specifically modified or manipulated form of the wild-type CRISPR enzyme, that is, the CRISPR enzyme variant may cleave double-stranded DNA while not binding to the double-stranded DNA to be cleaved or maintaining a certain distance therefrom. In this case, the modified or manipulated form may be a complete active CRISPR enzyme with an improved functional activity, compared to the wild-type CRISPR enzyme.

The CRISPR enzyme variant may be a complete active CRISPR enzyme with a reduced function, compared to the wild-type CRISPR enzyme.

For example, the specific modified or manipulated form of the wild-type CRISPR enzyme, that is, the CRISPR enzyme variant may cleave double-stranded DNA while very close to the double-stranded DNA to be cleaved or forming a specific bond therewith. Here, the specific bond may be, for example, a bond between an amino acid at a specific region of the CRISPR enzyme and a DNA nucleotide sequence at the cleavage location. In this case, the modified or manipulated form may be a complete active CRISPR enzyme with a reduced functional activity, compared to the wild-type CRISPR enzyme.

The CRISPR enzyme may be an incomplete or partially active CRISPR enzyme.

The term “incomplete or partially active” refers to a state in which an enzyme has one selected from the functions of the wild-type CRISPR enzyme, that is, a first function of cleaving the first strand of double-stranded DNA and a second function of cleaving the second strand of double-stranded DNA. The CRISPR enzyme in this state is named an incomplete or partially active CRISPR enzyme. In addition, the incomplete or partially active CRISPR enzyme may be referred to as a nickase.

The term “nickase” refers to a CRISPR enzyme manipulated or modified to cleave only one strand of the double strand of a target gene or a nucleic acid, and the nickase has nuclease activity of cleaving a single strand, for example, a strand that is complementary or non-complementary to gRNA of a target gene or a nucleic acid. Therefore, to cleave the double strand, nuclease activity of the two nickases is needed.

The nickase may have nuclease activity by the RuvC domain of a CRISPR enzyme. That is, the nickase may not include nuclease activity of the HNH domain of a CRISPR enzyme, and to this end, the HNH domain may be manipulated or modified.

In one example, when the CRISPR enzyme is a Type II CRISPR enzyme, the nickase may be a Type II CRISPR enzyme including a modified HNH domain.

For example, provided that the Type II CRISPR enzyme is a wild-type SpCas9, the nickase may be a SpCas9 variant in which nuclease activity of the HNH domain is inactived by mutation that the 840th amino acid in the amino acid sequence of the wild- type SpCas9 is mutated from histidine to alanine. Since the nickase produced thereby, that is, the SpCas9 variant has nuclease activity of the RuvC domain, it is able to cleave a strand which is a non-complementary strand of a target gene or a nucleic acid, that is, a strand not forming a complementary bond with gRNA.

For another example, provided that the Type II CRISPR enzyme is a wild-type CjCas9, the nickase may be a CjCas9 variant in which nuclease activity of the HNH domain is inactived by mutation that the 559th amino acid in the amino acid sequence of the wild-type CjCas9 is mutated from histidine to alanine. Since the nickase produced thereby, that is, the CjCas9 variant has nuclease activity of the RuvC domain, it is able to cleave a strand which is a non-complementary strand of a taget gene or a nucleic acid, that is, a strand not forming a complementary bond with gRNA.

In addition, the nickase may have nuclease activity by the HNH domain of a CRISPR enzyme. That is, the nickase may not include the nuclease activity of the RuvC domain, and to this end, the RuvC domain may be manipulated or modified.

In one example, when the CRISPR enzyme is a Type II CRISPR enzyme, the nickase may be a Type II CRISPR enzyme including a modified RuvC domain.

For example, provided that the Type II CRISPR enzyme is a wild-type SpCas9, the nickase may be a SpCas9 variant in which nuclease activity of the RuvC domain is inactived by mutation that the 10^{th} amino acid in the amino acid sequence of the wild-type SpCas9 is mutated from aspartic acid to alanine. Since the nickase produced thereby, that is the SpCas9 variant has nuclease activity of the HNH domain, it is able to cleave a strand which is a complementary strand of a target gene or a nucleic acid, that is, a strand forming a complementary bond with gRNA.

For another example, provided that the Type II CRISPR enzyme is a wild-type CjCas9, the nickase may be a CjCas9 variant in which nuclease activity of the RuvC domain is inactived by mutation that the 8th amino acid in the amino acid sequence of the wild-type CjCas9 is mutated from aspartic acid to alanine. Since the nickase produced thereby, that is, the CjCas9 variant has nuclease activity of the HNH domain, it is able to cleave a strand which is a complementary strand of a target gene or a nucleic acid, that is, a strand forming a complementary bond with gRNA.

The CRISPR enzyme may be an inactive CRISPR enzyme.

The term “inactive” refers to a state in which both of the functions of the wild- type CRISPR enzyme, that is, the first function of cleaving the first strand of double- stranded DNA and the second function of cleaving the second strand of double-stranded DNA are lost. The CRISPR enzyme in such a state is named an inactive CRISPR enzyme.

The inactive CRISPR enzyme may have nuclease inactivity due to variations in the domain having nuclease activity of a wild-type CRISPR enzyme.

The inactive CRISPR enzyme may have nuclease inactivity due to variations in a RuvC domain and an HNH domain. That is, the inactive CRISPR enzyme may not have nuclease activity generated by the RuvC domain and HNH domain of the CRISPR enzyme, and to this end, the RuvC domain and the HNH domain may be manipulated or modified.

In one example, when the CRISPR enzyme is a Type II CRISPR enzyme, the inactive CRISPR enzyme may be a Type II CRISPR enzyme having a modified RuvC domain and HNH domain.

For example, when the Type II CRISPR enzyme is a wild-type SpCas9, the inactive CRISPR enzyme may be a SpCas9 variant in which the nuclease activities of the RuvC domain and the HNH domain are inactivated by mutations of both aspartic acid 10 and histidine 840 in the amino acid sequence of the wild-type SpCas9 to alanine. Here, since, in the produced inactive CRISPR enzyme, that is, the SpCas9 variant, the nuclease activities of the RuvC domain and the HNH domain are inactivated, double-strand of a target gene or a nucleic acid may not be entirely cleaved.

In another example, when the Type II CRISPR enzyme is a wild-type CjCas9, the inactive CRISPR enzyme may be a CjCas9 variant in which the nuclease activities of the RuvC domain and the HNH domain are inactivated by mutations of both aspartic acid 8 and histidine 559 in the amino acid sequence of the wild-type CjCas9 to alanine. Here, since, in the produced inactive CRISPR enzyme, that is, the CjCas9 variant, the nuclease activities of the RuvC domain and HNH domain are inactivated, double-strand of a target gene or a nucleic acid may not be entirely cleaved.

The CRISPR enzyme may have helicase activity, that is, an ability to anneal the helix structure of the double-stranded nucleic acid, in addition to the above-described nuclease activity.

In addition, the CRISPR enzyme may be modified to complete activate, incomplete or partially activate, or inactivate the helicase activity.

The CRISPR enzyme may be a CRISPR enzyme variant produced by artificially manipulating or modifying the wild-type CRISPR enzyme.

The CRISPR enzyme variant may be an artificially manipulated or modified CRISPR enzyme variant for modifying the functions of the wild-type CRISPR enzyme, that is, the first function of cleaving the first strand of double-stranded DNA and/or the second function of cleaving the second strand of double-stranded DNA.

For example, the CRISPR enzyme variant may be a form in which the first function of the functions of the wild-type CRISPR enzyme is lost.

Alternatively, the CRISPR enzyme variant may be a form in which the second function of the functions of the wild-type CRISPR enzyme is lost.

For example, the CRISPR enzyme variant may be a form in which both of the functions of the wild-type CRISPR enzyme, that is, the first function and the second function, are lost.

The CRISPR enzyme variant may form a gRNA-CRISPR enzyme complex by interactions with gRNA.

The CRISPR enzyme variant may be an artificially manipulated or modified CRISPR enzyme variant for modifying a function of interacting with gRNA of the wild-type CRISPR enzyme.

For example, the CRISPR enzyme variant may be a form having reduced interactions with gRNA, compared to the wild-type CRISPR enzyme.

Alternatively, the CRISPR enzyme variant may be a form having increased interactions with gRNA, compared to the wild-type CRISPR enzyme.

For example, the CRISPR enzyme variant may be a form having the first function of the wild-type CRISPR enzyme and reduced interactions with gRNA.

Alternatively, the CRISPR enzyme variant may be a form having the first function of the wild-type CRISPR enzyme and increased interactions with gRNA.

For example, the CRISPR enzyme variant may be a form having the second function of the wild-type CRISPR enzyme and reduced interactions with gRNA.

Alternatively, the CRISPR enzyme variant may be a form having the second function of the wild-type CRISPR enzyme and increased interactions with gRNA.

For example, the CRISPR enzyme variant may be a form not having the first and second functions of the wild-type CRISPR enzyme, and having reduced interactions with gRNA.

Alternatively, the CRISPR enzyme variant may be a form not having the first and second functions of the wild-type CRISPR enzyme and having increased interactions with gRNA.

Here, according to the interaction strength between gRNA and the CRISPR enzyme variant, various gRNA-CRISPR enzyme complexes may be formed, and according to the CRISPR enzyme variant, there may be a difference in function of approaching or cleaving the target sequence.

For example, the gRNA-CRISPR enzyme complex formed by a CRISPR enzyme variant having reduced interactions with gRNA may cleave a double or single strand of a target sequence only when very close to or localized to the target sequence completely complementarily bind to gRNA.

The CRISPR enzyme variant may be in a form in which at least one amino acid of the amino acid sequence of the wild-type CRISPR enzyme is modified.

As an example, the CRISPR enzyme variant may be in a form in which at least one amino acid of the amino acid sequence of the wild-type CRISPR enzyme is substituted.

As another example, the CRISPR enzyme variant may be in a form in which at least one amino acid of the amino acid sequence of the wild-type CRISPR enzyme is deleted.

As still another example, the CRISPR enzyme variant may be in a form in which at least one amino acid of the amino acid sequence of the wild-type CRISPR enzyme is added.

In one example, the CRISPR enzyme variant may be in a form in which at least one amino acid of the amino acid sequence of the wild-type CRISPR enzyme is substituted, deleted and/or added.

In addition, optionally, the CRISPR enzyme variant may further include a functional domain, in addition to the original functions of the wild-type CRISPR enzyme, that is, the first function of cleaving the first strand of double-stranded DNA and the second function of cleaving the second strand thereof. Here, the CRISPR enzyme variant may have an additional function, in addition to the original functions of the wild-type CRISPR enzyme.

The functional domain may be a domain having methylase activity, demethylase activity, transcription activation activity, transcription repression activity, transcription release factor activity, histone modification activity, RNA cleavage activity or nucleic acid binding activity, or a tag or reporter gene for isolating and purifying a protein (including a peptide), but the present invention is not limited thereto.

The tag includes a histidine (His) tag, a V5 tag, a FLAG tag, an influenza hemagglutinin (HA) tag, a Myc tag, a VSV-G tag and a thioredoxin (Trx) tag, and the reporter gene includes glutathione-S-transferase (GST), horseradish peroxidase (HRP), chloramphenicol acetyltransferase (CAT) [3-galactosidase, [3-glucoronidase, luciferase, autofluorescent proteins including the green fluorescent protein (GFP), HcRed, DsRed, cyan fluorescent protein (CFP), yellow fluorescent protein (YFP) and blue fluorescent protein (BFP), but the present invention is not limited thereto.

The functional domain may be a deaminase.

For example, cytidine deaminase may be further included as a functional domain to an incomplete or partially-active CRISPR enzyme. In one exemplary embodiment, a fusion protein may be produced by adding a cytidine deaminase, for example, apolipoprotein B editing complex 1 (APOBEC1) to a SpCas9 nickase. The [SpCas9 nickase]-[APOBEC1] formed as described above may be used in nucleotide editing of C to T or U, or nucleotide editing of G to A.

In another example, adenine deaminase may be further included as a functional domain to the incomplete or partially-active CRISPR enzyme. In one exemplary embodiment, a fusion protein may be produced by adding adenine deaminases, for example, TadA variants, ADAR2 variants or ADAT2 variants to a SpCas9 nickase. The [SpCas9 nickase]-[TadA variant], [SpCas9 nickase]-[ADAR2 variant] or [SpCas9 nickase]-[ADAT2 variant] formed as described above may be used in nucleotide editing of A to G, or nucleotide editing of T to C, because the fusion protein modifies nucleotide A to inosine, the modified inosine is recognized as nucleotide G by a polymerase, thereby substantially exhibiting nucleotide editing of A to G.

The functional domain may be a nuclear localization sequence or signal (NLS) or a nuclear export sequence or signal (NES).

In one example, the CRISPR enzyme may include one or more NLSs. Here, the NLS may be included at an N-terminus of a CRISPR enzyme or the proximity thereof; a C-terminus of the enzyme or the proximity thereof; or a combination thereof. The NLS may be an NLS sequence derived from the following NLSs, but the present invention is not limited thereto: NLS of a SV40 virus large T-antigen having the amino acid sequence PKKKRKV(SEQ ID NO: 831); NLS from nucleoplasmin (e.g., nucleoplasmin bipartite NLS having the sequence KRPAATKKAGQAKKKK(SEQ ID NO: 832)); c-myc NLS having the amino acid sequence PAAKRVKLD(SEQ ID NO: 833) or RQRRNELKRSP(SEQ ID NO: 834); hRNPA1 M9 NLS having the sequence NQSSNFGPMKGGNFGGRSSGPYGGGGQYFAKPRNQGGY(SEQ ID NO: 835); the sequence RMRIZFKNKGKDTAELRRRRVEVSVELRKAKKDEQILKRRNV(SEQ ID NO: 836) of the IBB domain from importin-a; the sequences VSRKRPRP(SEQ ID NO: 837) and PPKKARED(SEQ ID NO: 838) of a myoma T protein; the sequence PQPKKKPL(SEQ ID NO: 839) of human p53; the sequence SALIKKKKKMAP(SEQ ID NO: 840) of mouse c-abl IV; the sequences DRLRR(SEQ ID NO: 841) and PKQKKRK(SEQ ID NO: 842) of influenza virus NS1; the sequence RKLKKKIKKL(SEQ ID NO: 843) of a hepatitis delta virus antigen; the sequence REKKKFLKRR(SEQ ID NO: 844) of a mouse Mx1 protein; the sequence KRKGDEVDGVDEVAKKKSKK(SEQ ID NO: 845) of a human poly (ADP- ribose) polymerase; or the sequence RKCLQAGMNLEARKTKK(SEQ ID NO: 846), derived from a sequence of a steroid hormone receptor (human) glucocorticoid.

In addition, the CRISPR enzyme mutant may include a split-type CRISPR enzyme prepared by dividing the CRISPR enzyme into two or more parts. The term “split” refers to functional or structural division of a protein or random division of a protein into two or more parts.

The split-type CRISPR enzyme may be a complete, incomplete or partially active enzyme or inactive enzyme.

For example, when the CRISPR enzyme is a SpCas9, the SpCas9 may be divided into two parts between the residue 656, tyrosine, and the residue 657, threonine, thereby generating split SpCas9.

The split-type CRISPR enzyme may selectively include an additional domain, peptide, polypeptide or protein for reconstitution.

The additional domain, peptide, polypeptide or protein for reconstitution may be assembled for formation of the split-type CRISPR enzyme to be structurally the same or similar to the wild-type CRISPR enzyme.

The additional domain, peptide, polypeptide or protein for reconstitution may be FRB and FKBP dimerization domains; intein; ERT and VPR domains; or domains which form a heterodimer under specific conditions.

For example, when the CRISPR enzyme is a SpCas9, the SpCas9 may be divided into two parts between the residue 713, serine, and the residue 714, glycine, thereby generating split SpCas9. The FRB domain may be connected to one of the two parts, and the FKBP domain may be connected to the other one. In the split SpCas9 produced thereby, the FRB domain and the FKBP domain may be formed in a dimer in an environment in which rapamycine is present, thereby producing a reconstituted CRISPR enzyme.

The CRISPR enzyme or CRISPR enzyme variant disclosed in the specification may be a polypeptide, protein or nucleic acid having a sequence encoding the same, and may be codon-optimized for a subject to introduce the CRISPR enzyme or CRISPR enzyme variant.

The term “codon optimization” refers to a process of modifying a nucleic acid sequence by maintaining a native amino acid sequence while replacing at least one codon of the native sequence with a codon more frequently or the most frequently used in host cells so as to improve expression in the host cells. A variety of species have a specific bias to a specific codon of a specific amino acid, and the codon bias (the difference in codon usage between organisms) is frequently correlated with efficiency of the translation of mRNA, which is considered to be dependent on the characteristic of a translated codon and availability of a specific tRNA molecule. The dominance of tRNA selected in cells generally reflects codons most frequently used in peptide synthesis. Therefore, a gene may be customized for optimal gene expression in a given organism based on codon optimization.

The gRNA, CRISPR enzyme or gRNA-CRISPR enzyme complex disclosed in the specification may be delivered or introduced into a subject by various forms.

The subject related description is as described above.

In one embodiment, a nucleic acid sequence encoding the gRNA and/or CRISPR enzyme may be delivered or introduced into a subject by a vector.

The vector may include the nucleic acid sequence encoding the gRNA and/or CRISPR enzyme.

In one example, the vector may simultaneously include the nucleic acid sequences encoding the gRNA and the CRISPR enzyme.

In another example, the vector may include the nucleic acid sequence encoding the gRNA.

For example, domains contained in the gRNA may be contained in one vector, or may be divided and then contained in different vectors.

In another example, the vector may include the nucleic acid sequence encoding the CRISPR enzyme.

For example, in the case of the CRISPR enzyme, the nucleic acid sequence encoding the CRISPR enzyme may be contained in one vector, or may be divided and then contained in several vectors.

The vector may include one or more regulatory/control components.

Here, the regulatory/control components may include a promoter, an enhancer, an intron, a polyadenylation signal, a Kozak consensus sequence, an internal ribosome entry site (IRES), a splice acceptor and/or a 2A sequence.

The promoter may be a promoter recognized by RNA polymerase II.

The promoter may be a promoter recognized by RNA polymerase III.

The promoter may be an inducible promoter.

The promoter may be a subject-specific promoter.

The promoter may be a viral or non-viral promoter.

The promoter may use a suitable promoter according to a control region (that is, a nucleic acid sequence encoding the gRNA and/or CRISPR enzyme).

For example, a promoter useful for the gRNA may be a H1, EF-1a, tRNA or U6 promoter. For example, a promoter useful for the CRISPR enzyme may be a CMV, EF-1a, EFS, MSCV, PGK or CAG promoter.

The vector may be a viral vector or recombinant viral vector.

The virus may be a DNA virus or an RNA virus.

Here, the DNA virus may be a double-stranded DNA (dsDNA) virus or single- stranded DNA (ssDNA) virus.

Here, the RNA virus may be a single-stranded RNA (ssRNA) virus.

The virus may be a retrovirus, a lentivirus, an adenovirus, adeno-associated virus (AAV), vaccinia virus, a poxvirus or a herpes simplex virus, but the present invention is not limited thereto.

In one example, a nucleic acid sequence encoding gRNA and/or a CRISPR enzyme may be delivered or introduced by a recombinant lentivirus.

In another example, a nucleic acid sequence encoding gRNA and/or a CRISPR enzyme may be delivered or introduced by a recombinant adenovirus.

In still another example, a nucleic acid sequence encoding gRNA and/or a CRISPR enzyme may be delivered or introduced by recombinant AAV.

In yet another example, a nucleic acid sequence encoding gRNA and/or a CRISPR enzyme may be delivered or introduced by one or more hybrids of hybrid viruses, for example, the viruses described herein.

In one embodiment, the gRNA-CRISPR enzyme complex may be delivered or introduced into a subject.

For example, the gRNA may be present in the form of DNA, RNA or a mixture thereof. The CRISPR enzyme may be present in the form of a peptide, polypeptide or protein.

In one example, the gRNA and CRISPR enzyme may be delivered or introduced into a subject in the form of a gRNA-CRISPR enzyme complex including RNA- type gRNA and a protein-type CRISPR, that is, a ribonucleoprotein (RNP).

The gRNA-CRISPR enzyme complex may be delivered or introduced into a subject by electroporation, microinjection, transient cell compression or squeezing (e.g., described in the literature [Lee, et al, (2012) Nano Lett., 12, 6322-6327]), lipid-mediated transfection, nanoparticles, a liposome, peptide-mediated delivery or a combination thereof.

The gRNA-CRISPR enzyme complex disclosed in the present specification may be used to artificially manipulate or modify a target gene, that is, a blood coagulation inhibitory gene.

A target gene may be manipulated or modified using the above-described gRNA-CRISPR enzyme complex, that is, the CRISPR complex. Here, the manipulation or modification of a target gene may include both of i) cleaving or damaging of a target gene and ii) repairing of the damaged target gene.

The i) cleaving or damaging of the target gene may be cleavage or damage of the target gene using the CRISPR complex, and particularly, cleavage or damage of a target sequence of the target gene.

The target sequence may become a target of the gRNA-CRISPR enzyme complex, and the target sequence may or may not include a PAM sequence recognized by the CRISPR enzyme. Such a target sequence may provide a critical standard to one who is involved in the designing of gRNA.

The target sequence may be specifically recognized by gRNA of the gRNA- CRISPR enzyme complex, and therefore, the gRNA-CRISPR enzyme complex may be located near the recognized target sequence.

The “cleavage” at a target site refers to the breakage of a covalent backbone of a polynucleotide. The cleavage includes enzymatic or chemical hydrolysis of a phosphodiester bond, but the present invention is not limited thereto. Other than this, the cleavage may be performed by various methods. Both of single strand cleavage and double strand cleavage are possible, wherein the double strand cleavage may result from two distinct single strand cleavages. The double strand cleavage may produce a blunt end or a staggered end (or a sticky end).

In one example, the cleavage or damage of a target gene using the CRISPR complex may be the entire cleavage or damage of the double strand of a target sequence.

In one exemplary embodiment, when the CRISPR enzyme is a wild-type SpCas9, the double strand of a target sequence forming a complementary bond with gRNA may be completely cleaved by the CRISPR complex.

In another exemplary embodiment, when the CRISPR enzymes are SpCas9 nickase (D10A) and SpCas9 nickase (H840A), the two single strands of a target sequence forming a complementary bond with gRNA may be respectively cleaved by the each CRISPR complex. That is, a complementary single strand of a target sequence forming a complementary bond with gRNA may be cleaved by the SpCas9 nickase (D10A), and a non-complementary single strand of the target sequence forming a complementary bond with gRNA may be cleaved by the SpCas9 nickase (H840A), and the cleavages may take place sequentially or simultaneously.

In another example, the cleavage or damage of a target gene or a nucleic acid using the CRISPR complex may be the cleavage or damage of only a single strand of the double strand of a target sequence. Here, the single strand may be a guide nucleic acid- binding sequence of the target sequence complementarily binding to gRNA, that is, a complementary single strand, or a guide nucleic acid non-binding sequence not complementarily binding to gRNA, that is, a non-complementary single strand to gRNA.

In one exemplary embodiment, when the CRISPR enzyme is a SpCas9 nickase (D10A), the CRISPR complex may cleave the guide nucleic acid-binding sequence of a target sequence complementarily binding to gRNA, that is, a complementary single strand, by a SpCas9 nickase (D10A), and may not cleave a guide nucleic acid non-binding sequence not complementarily binding to gRNA, that is, a non- complementary single strand to gRNA.

In another exemplary embodiment, when the CRISPR enzyme is a SpCas9 nickase (H840A), the CRISPR complex may cleave the guide nucleic acid non-binding sequence of a target sequence not complementarily binding to gRNA, that is, a non- complementary single strand to gRNA by a SpCas9 nickase (H840A), and may not cleave the guide nucleic acid-binding sequence of a target sequence complementarily binding to gRNA, that is, a complementary single strand.

In still another example, the cleavage or damage of a target gene or a nucleic acid using the CRISPR complex may be a removal of partial fragment of nucleic acid sequences.

In one exemplary embodiment, when the CRISPR complexes consist of wild- type SpCas9 and two gRNAs complementary binding to different target sequences, a double strand of a target sequence forming a complementary bond with the first gRNA may be cleaved, and a double strand of a target sequence forming a complementary bond with the second gRNA may be cleaved, resulting in the removal of nucleic acid fragments by the first and second gRNAs and SpCas9.

The ii) repairing of the damaged target gene may be repairing or restoring performed through non-homologous end joining (NHEJ) and homology-directed repair (HDR).

The non-homologous end joining (NHEJ) is a method of restoration or repairing double strand breaks in DNA by joining both ends of a cleaved double or single strand together, and generally, when two compatible ends formed by breaking of the double strand (for example, cleavage) are frequently in contact with each other to completely join the two ends, the broken double strand is recovered. The NHEJ is a restoration method that is able to be used in the entire cell cycle, and usually occurs when there is no homologous genome to be used as a template in cells, like the G1 phase.

In the repair process of the damaged gene or nucleic acid using NHEJ, some insertions and/or deletions (indels) in the nucleic acid sequence occur in the NHEJ- repaired region, such insertions and/or deletions cause the leading frame to be shifted, resulting in frame-shifted transcriptome mRNA. As a result, innate functions are lost because of nonsense-mediated decay or the failure to synthesize normal proteins. In addition, while the leading frame is maintained, mutations in which insertion or deletion of a considerable amount of sequence may be caused to destroy the functionality of the proteins. The mutation is locus-dependent because mutation in a significant functional domain is probably less tolerated than mutations in a non-significant region of a protein.

While it is impossible to expect indel mutations produced by NHEJ in a natural state, a specific indel sequence is preferred in a given broken region, and can come from a small region of micro homology. Conventionally, the deletion length ranges from 1 bp to 50 bp, insertions tend to be shorter, and frequently include a short repeat sequence directly surrounding a broken region.

In addition, the NHEJ is a process causing a mutation, and when it is not necessary to produce a specific final sequence, may be used to delete a motif of the small sequence.

A specific knockout of a gene targeted by the CRISPR complex may be performed using such NHEJ. A double strand or two single strands of a target gene or a nucleic acid may be cleaved using the CRISPR enzyme such as Cas9 or Cpf1, and the broken double strand or two single strands may have indels through the NHEJ, thereby inducing specific knockout of the target gene or nucleic acid. Here, the site of a target gene or a nucleic acid cleaved by the CRISPR enzyme may be a non-coding or coding region, and in addition, the site of the target gene or nucleic acid restored by NHEJ may be a non-coding or coding region.

In one example, the double strand of a target gene may be cleaved using the CRISPR complex, and various indels (insertions and deletions) may be generated at a repaired region by repairing through NHEJ.

The term “indel” refers to a variation formed by inserting or deleting a partial nucleotide into/from the nucleotide sequence of DNA. Indels may be introduced into the target sequence during repair by HDR or NHEJ, when the gRNA-CRISPR enzyme complex, as described above, cleaves a nucleic acid (DNA, RNA) of a blood coagulation inhibitory gene.

The homology directed repairing (HDR) is a correction method without an error, which uses a homologous sequence as a template to repair or restore the damaged gene or nucleic acid, and generally, to repair or restoration broken DNA, that is, to restore innate information of cells, the broken DNA is repaired using information of a complementary nucleotide sequence which is not modified or information of a sister chromatid. The most common type of HDR is homologous recombination (HR). HDR is a repair or restore method usually occurring in the S or G2/M phase of actively dividing cells.

To repair or restore damaged DNA using HDR, rather than using a complementary nucleotide sequence or sister chromatin of the cells, a DNA template artificially synthesized using information of a complementary nucleotide sequence or homologous nucleotide sequence, that is, a nucleic acid template including a complementary nucleotide sequence or homologous nucleotide sequence may be provided to the cells, thereby repairing or restoring the broken DNA. Here, when a nucleic acid sequence or nucleic acid fragment is further added to the nucleic acid template to repair or restore the broken DNA, the nucleic acid sequence or nucleic acid fragment further added to the broken DNA may be subjected to knockin. The further added nucleic acid sequence or nucleic acid fragment may be a nucleic acid sequence or nucleic acid fragment for correcting the target gene or nucleic acid modified by a mutation to a normal gene, or a gene or nucleic acid to be expressed in cells, but the present invention is not limited thereto.

In one example, a double or single strand of a target gene or a nucleic acid may be cleaved using the CRISPR complex, a nucleic acid template including a nucleotide sequence complementary to a nucleotide sequence adjacent to the cleavage site may be provided to cells, and the cleaved nucleotide sequence of the target gene or nucleic acid may be repaired or restored through HDR.

Here, the nucleic acid template including the complementary nucleotide sequence may have a complementary nucleotide sequence of the broken DNA, that is, a cleaved double or single strand, and further include a nucleic acid sequence or nucleic acid fragment to be inserted into the broken DNA. An additional nucleic acid sequence or nucleic acid fragment may be inserted into the broken DNA, that is, a cleaved site of the target gene or nucleic acid using the nucleic acid template including the complementary nucleotide sequence and a nucleic acid sequence or nucleic acid fragment to be inserted. Here, the nucleic acid sequence or nucleic acid fragment to be inserted and the additional nucleic acid sequence or nucleic acid fragment may be a nucleic acid sequence or nucleic acid fragment for correcting a target gene or a nucleic acid modified by a mutation to a normal gene or nucleic acid, or a gene or nucleic acid to be expressed in cells. The complementary nucleotide sequence may be a nucleotide sequence complementary binding with broken DNA, that is, right and left nucleotide sequences of the cleaved double or single strand of the target gene or nucleic acid. Alternatively, the complementary nucleotide sequence may be a nucleotide sequence complementary binding with broken DNA, that is, 3′ and 5′ ends of the cleaved double or single strand of the target gene or nucleic acid. The complementary nucleotide sequence may be a 15 to 3000-nt sequence, a length or size of the complementary nucleotide sequence may be suitably designed according to a size of the nucleic acid template or the target gene or the nucleic acid. Here, the nucleic acid template may be a double- or single-stranded nucleic acid, and may be linear or circular, but the present invention is not limited thereto.

In another example, a double- or single-strand of a target gene or a nucleic acid is cleaved using the CRISPR complex, a nucleic acid template including a nucleotide sequence having homology with a nucleotide sequence adjacent to a cleavage site is provided to cells, and the cleaved nucleotide sequence of the target gene or nucleic acid may be repaired or restored by HDR.

Here, the nucleic acid template including the homologous nucleotide sequence may have a nucleotide sequence having homology with the broken DNA, that is, a cleaved double- or single-strand, and further include a nucleic acid sequence or nucleic acid fragment to be inserted into the broken DNA. An additional nucleic acid sequence or nucleic acid fragment may be inserted into broken DNA, that is, a cleaved site of a target gene or a nucleic acid using the nucleic acid template including a homologous nucleotide sequence and a nucleic acid sequence or nucleic acid fragment to be inserted. Here, the nucleic acid sequence or nucleic acid fragment to be inserted and the additional nucleic acid sequence or nucleic acid fragment may be a nucleic acid sequence or nucleic acid fragment for correcting the target gene or nucleic acid modified by a mutation to a normal gene or nucleic acid, or a gene or nucleic acid to be expressed in cells. The homologous nucleotide sequence may be a nucleotide sequence having homology with the broken DNA, that is, the right and left nucleotide sequence of the cleaved double-strand or single-strand of the target gene or nucleic acid. Alternatively, the complementary nucleotide sequence may be a nucleotide sequence having homology with broken DNA, that is, the 3′ and 5′ ends of a cleaved double or single strand of the target gene or nucleic acid. The homologous nucleotide sequence may be a 15 to 3000- nt sequence, and a length or size of the homologous nucleotide sequence may be suitably designed according to a size of the nucleic acid template or the target gene or the nucleic acid. Here, the nucleic acid template may be a double- or single-stranded nucleic acid, and may be linear or circular, but the present invention is not limited thereto.

Other than the NHEJ and HDR, there are various methods for repairing or restoring a damaged target gene. For example, the method of repairing or restoring a damaged target gene may be single-strand annealing, single-strand break repair, mismatch repair, nucleotide cleavage repair or a method using the nucleotide cleavage repair.

The single-strand annealing (SSA) is a method of repairing double strand breaks between two repeat sequences present in a target nucleic acid, and generally uses a repeat sequence of more than 30 nucleotides. The repeat sequence is cleaved (to have sticky ends) to have a single strand with respect to a double strand of the target nucleic acid at each of the broken ends, and after the cleavage, a single-strand overhang containing the repeat sequence is coated with an RPA protein such that it is prevented from inappropriately annealing the repeat sequences to each other. RAD52 binds to each repeat sequence on the overhang, and a sequence capable of annealing a complementary repeat sequence is arranged. After annealing, a single-stranded flap of the overhang is cleaved, and synthesis of new DNA fills a certain gap to restore a DNA double strand. As a result of this repair or restore, a DNA sequence between two repeats is deleted, and a deletion length may be dependent on various factors including the locations of the two repeats used herein, and a path or degree of the progress of cleavage.

The SSA, similar to HDR, utilizes a complementary sequence, that is, a complementary repeat sequence, and in contrast, does not requires a nucleic acid template for modifying or correcting a target nucleic acid sequence.

Single strand breaks in a genome are repaired or restored through a separate mechanism, single-strand break repair (SSBR), from the above-described repair mechanisms. In the case of single-strand DNA breaks, PARP1 and/or PARP2 recognizes the breaks and recruits a repair mechanism. PARP1 binding and activity with respect to the DNA breaks are temporary, and SSBR is promoted by promoting the stability of an SSBR protein complex in the damaged regions. The most important protein in the SSBR complex is XRCC1, which interacts with a protein promoting 3′ and 5′ end processing of DNA to stabilize the DNA. End processing is generally involved in repairing the damaged 3′ end to a hydroxylated state, and/or the damaged 5′ end to a phosphatic moiety, and after the ends are processed, DNA gap filling takes place. There are two methods for the DNA gap filling, that is, short patch repair and long patch repair, and the short patch repair involves insertion of a single nucleotide. After DNA gap filling, a DNA ligase promotes end joining.

The mismatch repair (MMR) works on mismatched DNA nucleotides. Each of an MSH2/6 or MSH2/3 complex has ATPase activity and thus plays an important role in recognizing a mismatch and initiating a repair, and the MSH2/6 primarily recognizes nucleotide-nucleotide mismatches and identifies one or two nucleotide mismatches, but the MSH2/3 primarily recognizes a larger mismatch.

The base excision repair (BER) is a repair method which is active throughout the entire cell cycle, and used to remove a small non-helix-distorting base damaged region from the genome. In the damaged DNA, damaged nulceotides are removed by cleaving an N-glycoside bond joining a nucleotide to the phosphate-deoxyribose backbone, and then the phosphodiester backbone is cleaved, thereby generating breaks in single-strand DNA. The broken single strand ends formed thereby were removed, a gap generated due to the removed single strand is filled with a new complementary nucleotide, and then an end of the newly-filled complementary nucleotide is ligated with the backbone by a DNA ligase, resulting in repair or restore of the damaged DNA.

The nucleotide excision repair (NER) is an excision mechanism important for removing large helix-distorting damage from DNA, and when the damage is recognized, a short single-strand DNA segment containing the damaged region is removed, resulting in a single strand gap of 22 to 30 nucleotides. The generated gap is filled with a new complementary nucleotide, and an end of the newly filled complementary nucleotide is ligated with the backbone by a DNA ligase, resulting in the repair or restore of the damaged DNA.

Effects of artificially manipulating a target gene, that is, a blood coagulation inhibitory gene, by the gRNA-CRISPR enzyme complex may be largely knockout (knock- out), knockdown, knockin (knock-in).

The “knockout” refers to inactivation of a target gene or nucleic acid, and the “inactivation of a target gene or nucleic acid” refers to a state in which transcription and/or translation of a target gene or nucleic acid does not occur. Transcription and translation of a gene causing a disease or a gene having an abnormal function may be inhibited through knockout, resulting in the prevention of protein expression.

For example, when a target gene or a chromosome is edited using the gRNA- CRISPR enzyme complex, that is, the CRISPR complex, the target gene or the chromosome may be cleaved using the CRISPR complex. The target gene or the chromosome damaged using the CRISPR complex may be repaired by NHEJ. In the damaged target gene or chromosome, an indel is generated by NHEJ and thereby inducing target gene or chromosome-specific knockout.

In another example, when a target gene or a chromosome is edited using the gRNA-CRISPR enzyme complex, that is, the CRISPR complex, and a donor, the target gene or nucleic acid may be cleaved using the CRISPR complex. The target gene or nucleic acid damaged using the CRISPR complex may be repaired by HDR using a donor. Here, the donor includes a homologous nucleotide sequence and a nucleotide sequence desired to be inserted. Here, the number of nucleotide sequences to be inserted may vary according to an insertion location or purpose. When the damaged target gene or chromosome is repaired using a donor, a nucleotide sequence to be inserted is inserted into the damaged nucleotide sequence region, and thereby inducing target gene or chromosome-specific knockout.

The “knockdown” refers to a decrease in transcription and/or translation of a target gene or nucleic acid or the expression of a target protein. The onset of a disease may be prevented or a disease may be treated by regulating the overexpression of a gene or protein through the knockdown.

For example, when a target gene or a chromosome is edited using a gRNA- CRISPR inactive enzyme-transcription inhibitory activity domain complex, that is, a CRISPR-inactive complex including a transcription inhibitory activity domain, the CRISPR-inactive complex may specifically bind to the target gene or chromosome, and the transcription of the target gene or chromosome is inhibited by the transcription inhibitory activity domain included in the CRISPR-inactive complex, thereby inducing knockdown in which the expression of a target gene or chromosome is inhibited.

In another example, when a target gene or a chromosome is edited using the gRNA-CRISPR enzyme complex, that is, the CRISPR complex, the promoter and/or enhancer region(s) of the target gene or chromosome may be cleaved using the CRISPR complex. Here, the gRNA may recognize a partial nucleotide sequence of the promoter and/or enhancer region(s) of the target gene or chromosome as a target sequence. The target gene or chromosome damaged using the CRISPR complex may be repaired by NHEJ. In the damaged target gene or chromosome, an indel is generated by NHEJ, thereby inducing target gene or chromosome-specific knockdown. Alternatively, when a donor is optionally used, the target gene or chromosome damaged using the CRISPR complex may be repaired by HDR. When the damaged target gene or chromosome is repaired using a donor, a nucleotide sequence to be inserted is inserted into the damaged nucleotide sequence region, thereby inducing target gene or chromosome-specific knockdown.

The “knockin” refers to insertion of a specific nucleic acid or gene into a target gene or nucleic acid, and here, the “specific nucleic acid or gene” refers to a gene or nucleic acid of interest to be inserted or expressed. A mutant gene triggering a disease may be utilized in disease treatment by correction to normal or insertion of a normal gene to induce expression of the normal gene through the knockin.

In addition, the knockin may further need a donor.

For example, when a target gene or nucleic acid is edited using the gRNA- CRISPR enzyme complex, that is, the CRISPR complex, and a donor, the target gene or nucleic acid may be cleaved using the CRISPR complex. The target gene or nucleic acid damaged using the CRISPR complex may be repaired by HDR using a donor. Here, the donor may include a specific nucleic acid or gene, and may be used to insert a specific nucleic acid or gene into the damaged gene or chromosome. Here, the inserted specific nucleic acid or gene may induce the expression of a protein.

In one embodiment of the disclosure of the present specification, the gRNA-CRISPR enzyme complex may impart an artificial manipulation or modification to a AT gene and/or a TFPI gene.

The gRNA-CRISPR enzyme complex may specifically recognize a target sequence of the AT gene and/or the TFPI gene.

The target sequence may be specifically recognized by gRNA of the gRNA- CRISPR enzyme complex, and therefore, the gRNA-CRISPR enzyme complex may be located near the recognized target sequence.

The target sequence may be a region or area in which an artificial modification occurs in the AT gene and/or the TFPI gene.

The target sequence may be a contiguous nucleotide sequence of 10 to 25 bp, located in a promoter region of the AT gene and/or the TFPI gene.

The target sequence may be a contiguous nucleotide sequence of 10 to 25 bp, located in an intron region of the AT gene and/or the TFPI gene.

The target sequence may be a contiguous nucleotide sequence of 10 to 25 bp, located in an exon region of the AT gene and/or the TFPI gene.

The target sequence may be a contiguous nucleotide sequence of 10 to 25 bp, located in an enhancer region of the AT gene and/or the TFPI gene.

The target sequence may be a contiguous nucleotide sequence of 10 to 25 bp, located near the 5′ end and/or 3′ end of a PAM sequence in the nucleic acid sequence of the AT gene and/or the TFPI gene.

Here, the PAM sequence may be one or more of the following sequences (described in a 5′ to 3′ direction):
NGG (N is A, T, C or G);
NNNNRYAC (N is each independently A, T, C or G, R is A or G, and Y is C or T);
NNAGAAW (N is each independently A, T, C or G, and W is A or T);
NNNNGATT (N is each independently A, T, C or G);
NNGRR(T) (N is each independently A, T, C or G, and R is A or G); and
TTN (N is A, T, C or G).

In one embodiment, the target sequence may be one or more nucleotide sequences selected from the nucleotide sequences described in Table 1.

The gRNA-CRISPR enzyme complex may consist of a gRNA and a CRISPR enzyme.

The gRNA may include a guide domain capable of partial or complete complementary binding with a guide nucleic acid-binding sequence of a target sequence of the AT gene and/or the TFPI gene.

The guide domain may have at least 70%, 75%, 80%, 85%, 90% or 95% complementarity or complete complementarity to the guide nucleic acid-binding sequence.

The guide domain may include a nucleotide sequence complementary to the guide nucleic acid-binding sequence of the target sequence of the AT gene. Here, the complementary nucleotide sequence may include 0 to 5, 0 to 4, 0 to 3, or 0 to 2 mismatches.

The guide domain may include a nucleotide sequence complementary to the guide nucleic acid-binding sequence of the target sequence of the TFPI gene. Here, the complementary nucleotide sequence may include 0 to 5, 0 to 4, 0 to 3, or 0 to 2 mismatches.

The gRNA may include one or more domains selected from the group consisting of a first complementary domain, a linker domain, a second complementary domain, a proximal domain and a tail domain.

The CRISPR enzyme may be one or more proteins selected from the group consisting of a Streptococcus pyogenes-derived Cas9 protein, a Campylobacter jejuni- derived Cas9 protein, a Streptococcus thermophilus-derived Cas9 protein, a Staphylococcus aureus-derived Cas9 protein, a Neisseria meningitidis-derived Cas9 protein and a Cpf1 protein. In one example, the editor protein may be a Campylobacter jejuni-derived Cas9 protein or a Staphylococcus aureus-derived Cas9 protein.

The gRNA-CRISPR enzyme complex may impart various artificial manipulations or modifications to the AT gene and/or the TFPI gene.

In the artificially manipulated or modified AT gene and/or the TFPI gene, one or more modifications may occur in a contiguous nucleotide sequence region of 1 to 50 bp, located in a target sequence or near the 5′ end and/or 3′ end of a target sequence. The modifications are as follows:
i) deletion of one or more nucleotides,
ii) substitution of one or more nucleotides with nucleotide(s) different from a wild-type gene,
iii) insertion of one or more nucleotides, or
iv) a combination of two or more selected from i) to iii).

For example, in the artificially manipulated or modified AT gene and/or the TFPI gene, one or more nucleotides may be deleted in a contiguous nucleotide sequence region of 1 to 50 bp, located in a target sequence or near the 5′ end and/or 3′ end of a target sequence. In one example, the deleted nucleotides may be a 1, 2, 3, 4 or 5 bp sequential or non-sequential nucleotides. In another example, the deleted nucleotides may be a nucleotide fragment consisting of sequential nucleotides of 2 bp or more. Here, the nucleotide fragment may be 2 to 5 bp, 6 to 10 bp, 11 to 15 bp, 16 to 20 bp, 21 to 25 bp, 26 to 30 bp, 31 to 35 bp, 36 to 40 bp, 41 to 45 bp or 46 to 50 bp in size. In still another example, the deleted nucleotides may be two or more nucleotide fragments. Here, two or more nucleotide fragments may be independent nucleotide fragments, which are not contiguous, that is, have one or more nucleotide sequence gaps, and two or more deletion sites may be generated due to the two or more deleted nucleotide fragments.

Alternatively, for example, in the artificially manipulated or modified AT gene and/or the TFPI gene, the insertion of one or more nucleotides may occur in a contiguous nucleotide sequence region of 1 to 50 bp, located in a target sequence or near the 5′ end and/or 3′ end of a target sequence. In one example, the inserted nucleotides may be a 1, 2, 3, 4 or 5 bp sequential nucleotides. In another example, the inserted nucleotides may be a nucleotide fragment consisting of 5 bp or more sequential nucleotides. Here, the nucleotide fragment may be 5 to 10 bp, 11 to 50 bp, 50 to 100 bp, 100 to 200 bp, 200 to 300 bp, 300 to 400 bp, 400 to 500 bp, 500 to 750 bp or 750 to 1000 bp in size. In still another example, the inserted nucleotides may be a partial or complete nucleotide sequence of a specific gene. Here, the specific gene may be a gene introduced from the outside, which is not included in an object including a AT gene and/or the TFPI gene, for example, a human cell. Alternatively, the specific gene may be a gene included in an object including a AT gene and/or the TFPI gene, for example, a human cell. For example, the specific gene may be a gene present in the genome of a human cell.

Alternatively, for example, in the artificially manipulated or modified AT gene and/or the TFPI gene, one or more nucleotides may be deleted and inserted in a contiguous nucleotide sequence region of 1 to 50 bp, located in a target sequence or near the 5′ end and/or 3′ end of a target sequence. In one example, the deleted nucleotides may be a 1, 2, 3, 4 or 5 bp sequential or non-sequential nucleotides. Here, the inserted nucleotides may be a 1, 2, 3, 4 or 5 bp nucleotides; a nucleotide fragment; or a partial or complete nucleotide sequence of a specific gene, and the deletion and insertion may sequentially or simultaneously occur. Here, the inserted nucleotide fragment may be 5 to 10 bp, 11 to 50 bp, 50 to 100 bp, 100 to 200 bp, 200 to 300 bp, 300 to 400 bp, 400 to 500 bp, 500 to 750 bp or 750 to 1000 bp in size. Here, the specific gene may be a gene introduced from the outside, which is not included in an object including a AT gene and/or the TFPI gene, for example, a human cell. Alternatively, the specific gene may be a gene included in an object including a AT gene and/or the TFPI gene, for example, a human cell. For example, the specific gene may be a gene present in the genome of a human cell. In another example, the deleted nucleotides may be a nucleotide fragment consisting of 2 bp or more nucleotides. Here, the deleted nucleotide fragment may be 2 to 5 bp, 6 to 10 bp, 11 to 15 bp, 16 to 20 bp, 21 to 25 bp, 26 to 30 bp, 31 to 35 bp, 36 to 40 bp, 41 to 45 bp or 46 to 50 bp in size. Here, the inserted nucleotides may be 1, 2, 3, 4 or 5 bp nucleotides; a nucleotide fragment; or a partial or complete nucleotide sequence of a specific gene, and the deletion and insertion may sequentially or simultaneously occur. In still another example, the deleted nucleotides may be two or more nucleotide fragments. Here, the inserted nucleotides may be 1, 2, 3, 4 or 5 bp nucleotides; a nucleotide fragment; or a partial or complete nucleotide sequence of a specific gene, and the deletion and insertion may sequentially or simultaneously occur. In addition, the insertion may occur in a partial or complete part of the two or more deleted parts.

The gRNA-CRISPR enzyme complex may impart various artificial manipulations or modifications to the AT gene and/or the TFPI gene according to the types of gRNA and CRISPR enzyme.

In one example, when the CRISPR enzyme is a SpCas9 protein, in the artificially manipulated or modified AT gene and/or the TFPI gene, one or more modifications may be included in a contiguous nucleotide sequence region of 1 to 50 bp, 1 to 40 bp or 1 to 30 bp, and preferably, 1 to 25 bp, located near the 5′ end and/or 3′ end of the PAM sequence of 5′-NGG-3′ (N is A, T, G or C) present in a target region of each gene. The modifications are as follows:
i) deletion of one or more nucleotides,
ii) substitution of one or more nucleotides with nucleotide(s) different from a wild-type gene,
iii) insertion of one or more nucleotides, or
iv) a combination of two or more selected from i) to iii).

In another example, when the CRISPR enzyme is a CjCas9 protein, in the artificially manipulated or modified AT gene and/or the TFPI gene, one or more modifications may be included in a contiguous nucleotide sequence region of 1 to 50 bp, 1 to 40 bp or 1 to 30 bp, and preferably, 1 to 25 bp, located near the 5′ end and/or 3′ end of the PAM sequence of 5′-NNNNRYAC-3′ (N is each independently A, T, C or G, R is A or G, and Y is C or T) present in a target region of each gene. The modifications are as follows:
i) deletion of one or more nucleotides,
ii) substitution of one or more nucleotides with nucleotide(s) different from a wild-type gene,
iii) insertion of one or more nucleotides, or
iv) a combination of two or more selected from i) to iii).

In still another example, when the CRISPR enzyme is a StCas9 protein, in the artificially manipulated or modified AT gene and/or the TFPI gene, one or more modifications may be included in a contiguous nucleotide sequence region of 1 to 50 bp, 1 to 40 bp or 1 to 30 bp, and preferably, 1 to 25 bp, located near the 5′ end and/or 3′ end of the PAM sequence of 5′-NNAGAAW-3′(N is each independently A, T, C or G, and W is A or T) present in a target region of each gene. The modifications are as follows:
i) deletion of one or more nucleotides,
ii) substitution of one or more nucleotides with nucleotide(s) different from a wild-type gene,
iii) insertion of one or more nucleotides, or
iv) a combination of two or more selected from i) to iii).

In one example, when the CRISPR enzyme is a NmCas9 protein, in the artificially manipulated or modified AT gene and/or the TFPI gene, one or more modifications may be included in a contiguous nucleotide sequence region of 1 to 50 bp, 1 to 40 bp or 1 to 30 bp, and preferably, 1 to 25 bp, located near the 5′ end and/or 3′ end of the PAM sequence of 5′-NNNNGATT-3′ (N is each independently A, T, C or G) present in a target region of each gene. The modifications are as follows:
i) deletion of one or more nucleotides,
ii) substitution of one or more nucleotides with nucleotide(s) different from a wild-type gene,
iii) insertion of one or more nucleotides, or
iv) a combination of two or more selected from i) to iii).

In yet another example, when the CRISPR enzyme is a SaCas9 protein, in the artificially manipulated or modified AT gene and/or the TFPI gene, one or more modifications may be included in a contiguous nucleotide sequence region of 1 to 50 bp, 1 to 40 bp or 1 to 30 bp, and preferably, 1 to 25 bp, located near the 5′ end and/or 3′ end of the PAM sequence of 5′-NNGRR(T)-3′ (N is each independently A, T, C or G, R is A or G, and (T) means any insertable sequence) present in a target region of each gene. The modifications are as follows:
i) deletion of one or more nucleotides,
ii) substitution of one or more nucleotides with nucleotide(s) different from a wild-type gene,
iii) insertion of one or more nucleotides, or
iv) a combination of two or more selected from i) to iii).

In yet another example, when the CRISPR enzyme is a Cpf1 protein, in the artificially manipulated or modified AT gene and/or the TFPI gene, one or more modifications may be included in a contiguous nucleotide sequence region of 1 to 50 bp, 1 to 40 bp or 1 to 30 bp, and preferably, 1 to 25 bp, located near the 5′ end and/or 3′ end of the PAM sequence of 5′-TTN-3′ (N is A, T, C or G) present in a target region of each gene. The modifications are as follows:
i) deletion of one or more nucleotides,
ii) substitution of one or more nucleotides with nucleotide(s) different from a wild-type gene,
iii) insertion of one or more nucleotides, or
iv) a combination of two or more selected from i) to iii).

The artificial manipulation effect on the AT gene and/or the TFPI gene, caused by the gRNA-CRISPR enzyme complex, may be knock-out.

The artificial manipulation effect on the AT gene and/or the TFPI gene, caused by the gRNA-CRISPR enzyme complex, may be to inhibit the expression of a protein encoded by each of the AT gene and/or the TFPI gene.

The artificial manipulation effect on the AT gene and/or the TFPI gene, caused by the gRNA-CRISPR enzyme complex, may be knock-down.

The artificial manipulation effect on the AT gene and/or the TFPI gene, caused by the gRNA-CRISPR enzyme complex, may be to reduce the expression of a protein encoded by each of the AT gene and/or the TFPI gene.

The artificial manipulation effect on the AT gene and/or the TFPI gene, caused by the gRNA-CRISPR enzyme complex, may be knock-in.

Here, the knock-in effect may be induced by the gRNA-CRISPR enzyme complex and a donor additionally including a foreign nucleotide sequence or gene.

The artificial manipulation effect on the AT gene and/or the TFPI gene, caused by the gRNA-CRISPR enzyme complex and a donor, may be to express a peptide or protein encoded by a foreign nucleotide sequence or gene.

One aspect disclosed in the present specification relates to a method of treating a coagulopathy using a composition for gene manipulation for treating a coagulopathy.

One embodiment disclosed in the present specification provides a use for treating a coagulopathy using a method including administering a composition for gene manipulation for artificially manipulating a blood coagulation inhibitory gene into a treatment subject.

Here, the treatment subject may be a mammal including primates such as a human, a monkey, etc. and rodents such as a rat, a mouse, etc.

The term “coagulopathy (i.e., a bleeding disorder)” refers to a condition in which the formation of blood clots is inhibited or suppressed by an abnormal blood coagulation system. This coagulopathy includes conditions in which blood coagulation, that is, thrombogenesis is inhibited or suppressed, and thus, bleeding does not stop or hemostasis is delayed. It refers to a pathological condition including all types of diseases caused thereby.

In this case, the coagulopathy includes all types of diseases induced by the abnormal blood coagulation system and the blood coagulation inhibitory factors.

The term “blood coagulation inhibitory factor-induced disease” refers to all the conditions that include all types of diseases caused due to the abnormal forms (for example, mutations, and the like) or abnormal expression of the blood coagulation inhibitory factor.

Coagulopathy may be hemophilia that is a disease caused by the abnormal blood coagulation system in vivo.

In one embodiment, coagulopathy may be hemophilia A, hemophilia B, or hemophilia C.

Hemophilia (or haemophilia) is a congenital bleeding disease that is inherently caused by the deficiency of blood coagulation factors. There are 12 blood coagulation factors known so far. Among the symptoms of congenital blood coagulation deficiency, hemophilia A is caused by the deficiency of factor VIII, hemophilia B is caused by the deficiency of factor IX (Christmas disease), and hemophilia C is caused by the deficiency of factor XI. Hemophilia A and B account for 95% or more of hemophilia, and may not be easily clinically distinguished from each other because both of the two diseases have coagulation factors associated with an intrinsic coagulation mechanism. Hemophilia A and B are inherited as an X-linked recessive trait, and females are silent carriers, and only the male babies remain as patients.

Hemophilia A is symptomatic of factor VIII deficiency. 20 to 30% of hemophilia A is caused by mutations without any family history. Its incidence is approximately one in every 4,000 to 10,000 male babies, and hemophilia A has an incidence approximately 5- to 8-fold higher than hemophilia B.

Hemophilia B is the second most common type of hereditary coagulopathic disorder, and the bleeding symptom of hemophilia B is observed to be more pronounced in children and teenagers than in adults. Female carriers have slight symptoms, but 10% of the female carriers have a risk of bleeding. Its incidence is approximately one in every 20,000 to 25,000 male babies.

Hemophilia C accounts for approximately 2 to 3% of all coagulation factor deficiencies. Hemophilia C cases have not been reported in Korea, which is inherited as autosomally recessive.

The clinical symptom of hemophilia A is uncontrolled bleeding after traumatic injury, tooth extraction, and surgery. Severe hemophilia A is likely to be diagnosed within a year after birth, and spontaneous bleeding symptoms appear at 2 to 5 months when it is not treated.

Also, moderately severe hemophilia A tends to develop spontaneous bleeding. However, due to a delay in the appearance of symptoms, it is diagnosed before the age of 5 to 6 years by uncontrolled bleeding after minor trauma. Spontaneous bleeding is not observed in the case of mild hemophilia A. Mild hemophilia A has a symptom of uncontrolled bleeding after surgery, tooth extraction, and severe injuries, and in many case, may not be diagnosed during a lifetime. The bleeding symptom of hemophilia A is observed to be more pronounced in children and teenagers than in adults. Female carriers have mild symptoms, but 10% of the female carriers have a risk of bleeding.

Due to the deficiency of factor IX, hemophilia B has a symptom of uncontrolled bleeding after initial bleeding after traumatic injury, tooth extraction, or surgery has stopped. Hemarthrosis is frequently observed in severe hemophilia B. Severe hemophilia B is likely to be diagnosed within a year after birth, and spontaneous bleeding symptoms appear at 2 to 5 months when it is not treated. Also, moderately severe hemophilia B tends to develop spontaneous bleeding, but its appearance is delayed compared to severe hemophilia B. Moderately severe hemophilia B has a symptom of uncontrolled bleeding after traumatic injury, and is diagnosed before the age of 5 to 6 years. Mild hemophilia B has no spontaneous bleeding, and has a symptom of uncontrolled bleeding after surgery, tooth extraction, and severe injuries. Mild hemophilia B may not be diagnosed at all during a lifetime because it has no symptoms.

General treatment of hemophilia is achieved by supplementing insufficient blood coagulation factors. In this case, factor VIII and factor IX preparations are currently used, and a bypass factor is administered when antibodies are produced during treatment (Kemton CL et al., Blood. 2009; 113(1): 11-17).

One embodiment of the disclosure of the present specification provides a pharmaceutical composition including a composition for gene manipulation, which may artificially manipulate a blood coagulation inhibitory gene.

The description related to the composition for gene manipulation is as described above.

In one embodiment, the composition for gene manipulation may include the following components:
(a) a guide nucleic acid capable of targeting a target sequence of a blood coagulation inhibitory gene or a nucleic acid sequence encoding the same; and
(b) an editor protein including one or more proteins selected from the group consisting of a Streptococcus pyogenes-derived Cas9 protein, a Campylobacter jejuni-derived Cas9 protein, a Streptococcus thermophilus-derived Cas9 protein, a Staphylococcus aureus-derived Cas9 protein, a Neisseria meningitidis-derived Cas9 protein and a Cpf1 protein, or nucleic acid sequence(s) encoding the same.

Here, the blood coagulation inhibitory gene may be a AT gene and/or a TFPI gene.

Here, the composition for gene manipulation may include a vector including nucleic acid sequences encoding a guide nucleic acid and/or an editor protein, respectively.

Here, the guide nucleic acid or a nucleic acid sequence encoding the same; and a nucleic acid sequence encoding the editor protein may be present in the form of one or more vectors. They may be present in form of a homologous or heterologous vector.

In another embodiment, the composition for gene manipulation may include the following components:
(a) a guide nucleic acid capable of targeting a target sequence of a blood coagulation inhibitory gene or a nucleic acid sequence encoding the same;
(b) an editor protein including one or more proteins selected from the group consisting of a Streptococcus pyogenes-derived Cas9 protein, a Campylobacter jejuni-derived Cas9 protein, a Streptococcus thermophilus-derived Cas9 protein, a Staphylococcus aureus-derived Cas9 protein, a Neisseria meningitidis-derived Cas9 protein and a Cpf1 protein, or nucleic acid sequence(s) encoding the same; and
(c) a donor including a nucleic acid sequence to be inserted or a nucleic acid sequence encoding the same.

Here, the nucleic acid sequence to be inserted may be a partial sequence of the blood coagulation inhibitory gene.

Alternatively, the nucleic acid sequence to be inserted may be a complete or partial sequence of a gene encoding a blood coagulation associated protein.

Here, the blood coagulation associated protein may be factor XII, factor Xlla, factor XI, factor Xla, factor IX, factor IXa, factor X, factor Xa, factor VIII, factor Villa, factor VII, factor Vlla, factor V, factor Va, prothrombin, thrombin, factor XIII, factor Xllla, fibrinogen, fibrin or Tissue factor.

Here, the guide nucleic acid or a nucleic acid sequence encoding the same; and a nucleic acid sequence encoding the editor protein; and a donor including a nucleic acid sequence to be inserted or a nucleic acid sequence encoding the same may be present in the form of one or more vectors. They may be present in the form of a homologous or heterologous vector.

The pharmaceutical composition may further include an additional component.

The additional component may include a suitable carrier for delivery into the body of a subject.

In another embodiment, the composition for gene manipulation may include the following components:
(a) a guide nucleic acid including guide sequence capable of forming a complementary bond with a target sequence of a blood coagulation inhibitory gene or a nucleic acid sequence encoding the same; and
(b) an editor protein including one or more proteins selected from the group consisting of a Streptococcus pyogenes-derived Cas9 protein, a Campylobacter jejuni-derived Cas9 protein, a Streptococcus thermophilus-derived Cas9 protein, a Staphylococcus aureus-derived Cas9 protein, a Neisseria meningitidis-derived Cas9 protein and a Cpf1 protein, or nucleic acid sequence(s) encoding the same.

Here, the blood coagulation inhibitory gene may be an AT gene and/or a TFPI gene.

Here, the composition for gene manipulation may include a vector including nucleic acid sequences encoding a guide nucleic acid and/or an editor protein, respectively.

Here, the guide nucleic acid or a nucleic acid sequence encoding the same; and a nucleic acid sequence encoding the editor protein may be present in the form of one or more vectors. They may be present in the form of a homologous or heterologous vector.

In another embodiment, the composition for gene manipulation may include the following components:
(a) a guide nucleic acid including guide sequence capable of forming a complementary bond with a target sequence of a blood coagulation inhibitory gene or a nucleic acid sequence encoding the same;
(b) an editor protein including one or more proteins selected from the group consisting of a Streptococcus pyogenes-derived Cas9 protein, a Campylobacter jejuni-derived Cas9 protein, a Streptococcus thermophilus-derived Cas9 protein, a Staphylococcus aureus-derived Cas9 protein, a Neisseria meningitidis-derived Cas9 protein and a Cpf1 protein, or nucleic acid sequence(s) encoding the same; and
(c) a donor including a nucleic acid sequence to be inserted or a nucleic acid sequence encoding the same.

Here, the nucleic acid sequence to be inserted may be a partial sequence of the blood coagulation inhibitory gene.

Alternatively, the nucleic acid sequence to be inserted may be a complete or partial sequence of a gene encoding a blood coagulation associated protein.

Here, the blood coagulation associated protein may be factor XII, factor Xlla, factor XI, factor Xla, factor IX, factor IXa, factor X, factor Xa, factor VIII, factor Villa, factor VII, factor Vlla, factor V, factor Va, prothrombin, thrombin, factor XIII, factor Xllla, fibrinogen, fibrin or Tissue factor.

Here, the guide nucleic acid or a nucleic acid sequence encoding the same; and a nucleic acid sequence encoding the editor protein; and a donor including a nucleic acid sequence to be inserted or a nucleic acid sequence encoding the same may be present in the form of one or more vectors. They may be present in the form of a homologous or heterologous vector.

The pharmaceutical composition may further include an additional component.

The additional component may include a suitable carrier for delivery into the body of a subject.

One embodiment of the disclosure of the present specification provides a method of treating a coagulopathy, which include administering a composition for gene manipulation to an organism with a coagulopathy to treat the coagulopathy.

The treatment method may be a treatment method of regulating a blood coagulation system by manipulating a gene of an organism. Such a treatment method may be achieved by directly injecting a composition for gene manipulation into a body to manipulate a gene of an organism.

The description related to the composition for gene manipulation is as described above.

In one embodiment, the composition for gene manipulation may include the following components:
(a) a guide nucleic acid capable of targeting a target sequence of a blood coagulation inhibitory gene or a nucleic acid sequence encoding the same; and
(b) an editor protein including one or more proteins selected from the group consisting of a Streptococcus pyogenes-derived Cas9 protein, a Campylobacter jejuni-derived Cas9 protein, a Streptococcus thermophilus-derived Cas9 protein, a Staphylococcus aureus-derived Cas9 protein, a Neisseria meningitidis-derived Cas9 protein and a Cpf1 protein, or nucleic acid sequence(s) encoding the same.

Alternatively, in another embodiment, the composition for gene manipulation may include the following components:
(a) a guide nucleic acid including guide sequence capable of forming a complementary bond with a target sequence of a blood coagulation inhibitory gene or a nucleic acid sequence encoding the same; and
(b) an editor protein including one or more proteins selected from the group consisting of a Streptococcus pyogenes-derived Cas9 protein, a Campylobacter jejuni-derived Cas9 protein, a Streptococcus thermophilus-derived Cas9 protein, a Staphylococcus aureus-derived Cas9 protein, a Neisseria meningitidis-derived Cas9 protein and a Cpf1 protein, or nucleic acid sequence(s) encoding the same.

Here, the blood coagulation inhibitory gene may be an AT gene and/or a TFPI gene.

The guide nucleic acid and the editor protein may each be present in one or more vectors in the form of a nucleic acid sequence, or present by forming a complex by combining the guide nucleic acid and the editor protein.

Optionally, the composition for gene manipulation may further include a donor including a nucleic acid sequence to be inserted or a nucleic acid sequence encoding the same.

Each of the guide nucleic acid and the editor protein, and/or a donor may be present in one or more vectors in the form of a nucleic acid sequence.

Here, the vector may be a plasmid or a viral vector.

Here, the viral vector may be one or more selected from the group consisting of a retrovirus, a lentivirus, an adenovirus, an adeno-associated virus (AAV), a vaccinia virus, a poxvirus and a herpes simplex virus.

The description related to the coagulopathy is as described above.

The coagulopathy may be hemophilia A, hemophilia B or hemophilia C.

The composition for gene manipulation may be administered into a treatment subject with a coagulopathy.

The treatment subject may be a mammal including primates such as a human, a monkey, etc. and rodents such as a rat, a mouse, etc.

The composition for gene manipulation may be administered to a treatment subject.

The administration may be performed by injection, transfusion, implantation or transplantation.

The administration may be performed via an administration route selected from intrahepatic, subcutaneous, intradermal, intraocular, intravitreal, intratumoral, intranodal, intramedullary, intramuscular, intravenous, intralymphatic or intraperitoneal routes.

A dose (a pharmaceutically effective amount for obtaining a predetermined, desired effect) of the composition for gene manipulation is approximately 104 to 109 cells/kg (body weight of an administration subject), for example, approximately 105 to 106 cells/kg (body weight), which may be selected from all integers in the numerical range, but the present invention is not limited thereto. The composition may be suitably prescribed in consideration of an age, health condition and body weight of an administration subject, the type of concurrent treatment, and if possible, the frequency of treatment and a desired effect.

When the blood coagulation inhibitory gene is artificially manipulated using the method and the composition according to some embodiment disclosed in the present specification, it is possible to normalize the blood coagulation system, thereby achieving an effect of inhibiting or improving an abnormal bleeding symptom, and the like.

One embodiment disclosed in the present specification relates to a method of modifying a blood coagulation inhibitory gene in eukaryotic cells, which may be performed in vivo, ex vivo or in vitro.

In some embodiments, the method includes sampling a cell or a cell population from a human or non-human animal, and modifying the cell or cells. Culturing may occur at any stage ex vivo. The cell or cells may even be re-introduced into a non-human animal or plant.

The method may be a method of artificially manipulating eukaryotic cells, which includes introducing a composition for gene manipulation into eukaryotic cells.

The description related to the composition for gene manipulation is as described above.

In one embodiment, the composition for gene manipulation may include the following components:
(a) a guide nucleic acid capable of targeting a target sequence of a blood coagulation inhibitory gene or a nucleic acid sequence encoding the same; and
(b) an editor protein including one or more proteins selected from the group consisting of a Streptococcus pyogenes-derived Cas9 protein, a Campylobacter jejuni-derived Cas9 protein, a Streptococcus thermophilus-derived Cas9 protein, a Staphylococcus aureus-derived Cas9 protein, a Neisseria meningitidis-derived Cas9 protein and a Cpf1 protein, or nucleic acid sequence(s) encoding the same.

Alternatively, in another embodiment, the composition for gene manipulation may include the following components:
(a) a guide nucleic acid including guide sequence capable of forming a complementary bond with a target sequence of a blood coagulation inhibitory gene or a nucleic acid sequence encoding the same; and
(b) an editor protein including one or more proteins selected from the group consisting of a Streptococcus pyogenes-derived Cas9 protein, a Campylobacter jejuni-derived Cas9 protein, a Streptococcus thermophilus-derived Cas9 protein, a Staphylococcus aureus-derived Cas9 protein, a Neisseria meningitidis-derived Cas9 protein and a Cpf1 protein, or nucleic acid sequence(s) encoding the same.

Here, the blood coagulation inhibitory gene may be a AT gene and/or a TFPI gene.

The guide nucleic acid and the editor protein may each be present in one or more vectors in the form of a nucleic acid sequence, or present by forming a complex by combining the guide nucleic acid and the editor protein.

The introduction step may be performed in vivo or ex vivo.

For example, the introduction step may be performed by one or more methods selected from electroporation, liposomes, plasmids, viral vectors, nanoparticles and a protein translocation domain (PTD) fusion protein method.

For example, the viral vector may be one or more selected from the group consisting of a retrovirus, a lentivirus, an adenovirus, an adeno-associated virus (AAV), a vaccinia virus, a poxvirus and a herpes simplex virus.

### EXAMPLES

Hereinafter, the present invention will be described in further detail with reference to examples.

These examples are merely provided to illustrate the present invention, and it will be obvious to those of ordinary skill in the art that the scope of the present invention is not limited by the following examples.

### Experimental Method

### 1. Design of sgRNA

Targets for CRISPR/SpCas9 or CRISPR/SaCas9 or CRISPR/CjCas9 were extracted from human SERPINC1 and TFPI genes, in consideration of each PAM, using a Cas-Designer program of CRISPR RGEN Tools (Institute for Basic Science, Korea). Also, the targets whose 1- and 2-base mismatched off-target sites are not expected in the human genome were screened using the Cas-Offinder program. The sgRNA used in this experiment was designed to include at least one of the guide sequences listed in Tables3 ~ 4 and Table 11.

### 2. Verification of Guide RNA Activity and Off-Target Analysis

HEK293 and Jurkat human cell lines (cell counts: 2 x 10⁵ cells) were transfected with 250 ng of an sgRNA expression vector cloned with each guide RNA sequence together with 750 ng of a Cas9 expression vector using Lipofectamine 2000. Alternatively, 1 µg of in vitro transcribed sgRNA and 4 µg of Cas9 were mixed in the form of an RNP complex, and transfected through electroporation. After approximately 2 to 3 days, genomic DNA was extracted, and at an on-target site was amplified by PCR, and the resulting PCR product was subjected to additional PCR to ligate an adaptor specific to sequencing primers for Next-Generation Sequencing and TruSeq HT Dual Index primers to the PCR product. Thereafter, reads obtained through the paired sequencing were analyzed to evaluate the activities of guide RNAs through confirmation of insertions or deletions (Indels) at on-target genomic sites.

For the off-target analysis of the screened guide RNAs, first, 3-base mismatched off-target lists were screened by an in-silico method using Cas-Offinder of the CRISPR RGEN Tools, and verified by a method through targeted-deep sequencing of a certain region of the genome corresponding to each off-target site. As a second method, the human whole genomic DNA, which had been treated with the guide RNA and a Cas9 protein overnight at 37° C., was subjected to whole genome sequencing, and potential lists were secured through Digenome-seq analysis. Then, it was verified whether the indels were added at off-target sites by a method through targeted-deep sequencing of a certain region of the genome of each of off-target candidates.

### 3. AAV Construction

An SpCas9 gene editing tool was delivered using a dual AAV system. That is, each of a vector (pAAV-SpCas9) including mammalian expression promoters (EFS, TBG, and ICAM2) between inverted tandem repeats (ITRs) of AAV2, human codon- optimized Cas9 having NLS- HA-tags at the C- or N-termini thereof, and BGHA and a vector (pAAV-U6-sgRNA) including a U6 promoter sgRNA sequence was constructed by synthesis.

Also, another dual AAV system used Split SpCas9. In one AAV, a vector including a promoter (TBG, ICAM2)-SpCas9-Nterm-lntein between the ITRs was constructed (pAAV-SpCas9-split-Nterm), and, in the other AAV, a vector including a promoter (TBG, ICAM2)-lntein-SpCas9-Cterm-U6-SgRNA between the ITRs was constructed (pAAV-SpCas9-split-C-term-U6-SgRNA).

A CjCas9 gene editing tool was delivered using a single AAV system. That is, a vector (pAAV-CjCas9-U6-sgRNA) including promoters (EFS, TBG, and ICAM2) between AAV2 ITRs, human codon-optimized Cas9 having NLS- HA-tags at the C— or N- termini thereof, BGHA, U6 promoter, and an sgRNA sequence was constructed by synthesis. For AAV production, HEK293 cells were transfected with a vector for a pseudotype of an AAV capsid, the constructed pAAV vector (pAAV-EFS-SpCas9, or pAAV-U6-sgRNA, or pAAV-EFS-CjCas9-U6-sgRNA), and a pHelper vector at a 1:1:1 molar concentration at the same time. After 72 hours, the cells were lysed, and the resulting virus particles were than separated and purified by a step-gradient ultracentrifuge using iodixanol (Sigma-Aldrich), and quantitative determination of AAV was performed by a titration method using qPCR.

### 4. Animals and Injection

F8—KO mice and F9—KO rats were used as hemophilia A and B model animals, respectively. Each CRISPR/Cas9 was delivered to an animal model using a method of intravenously or intraperitoneally injecting the constructed AAV and LNP.

### 5. In Vivo Editing Test

Approximately 4 to 6 weeks after AAV and LNP injection, the liver was removed, and genomic DNA was extracted to evaluate the indels at On-target and Off- target sites through PCR and targeted-deep-sequencing.

### 6. Hemophilia Indicator Test

After the AAV and LNP injection, blood was collected at various time points (1 W-4 W-8 W-16 W-32 W, and the like) to evaluate an amount of Serpinc1 and TFPI proteins in blood by ELISA or to evaluate a hemophilia indicator by PT, an aPTT assay, immunohistochemistry, a tail cutting assay (blood loss quantification), and the like.

### Experimental Results

1. Verification of activity of Guide RNA

The activity of gRNA targeting each of the SERPINC1 gene (AT gene) and the TFPI gene was confirmed through the indels (%) to screen the gRNA.

1) SERPINC1 gene (AT gene)
① SpCas9

TABLE 5

gRNA selection to target SERPINC1 gene(AT gene) for SpCas9

| Target name | Target(w/o PAM) (SEQ ID NO) | PAM | GC content | Indels (%) |
|---|---|---|---|---|
| hMyd88-Sp-Ctrl | GTCCATCTCCTCCGCCAGCG(SEQ ID NO: 847) | CGG | | 75.0 |
| hSerpinc1-Sp-3 | GCCATGTATTCCAATGTGAT(SEQ ID NO: 21) | AGG | 40 | 49.9 |
| hSerpinc1-Sp-4 | GTGATAGGAACTGTAACCTC(SEQ ID NO: 22) | TGG | 45 | 42.2 |
| hSerpinc1-Sp-12 | GGGACTGCGTGACCTGTCAC(SEQ ID NO: 30) | GGG | 65 | 61.2 |
| hSerpincl -Sp-13 | GTCCACAGGGCTCCCGTGAC(SEQ ID NO: 31) | AGG | 70 | 29.4 |
| hSerpinc1-Sp-19 | CATGGGAATGTCCCGCGGCT(SEQ ID NO: 37) | TGG | 65 | 56.1 |
| hSerpinc1-Sp-20 | GGATTCATGGGAATGTCCCG(SEQ ID NO: 38) | CGG | 55 | 1.3 |
| hSerpinc1-Sp-23 | GGGGAGCGGTAAATGCACAT(SEQ ID NO: 41) | GGG | 55 | 49.3 |
| hSerpinc1-Sp-24 | CGGGGAGCGGTAAATGCACA(SEQ ID NO: 42) | TGG | 60 | 4.2 |
| hSerpinc1-Sp-25 | CATGTGCATTTACCGCTCCC(SEQ ID NO: 43) | CGG | 55 | 58.3 |
| hSerpinc1-Sp-30 | TTACCGCTCCCCGGAGAAGA(SEQ ID NO: 48) | AGG | 60 | 49.5 |
| hSerpinc1-Sp-34 | GGGCTCAGAACAGAAGATCC(SEQ ID NO: 52) | CGG | 55 | 45.7 |
| hSerpinc1-Sp-36 | CACGCCGGTTGGTGGCCTCC(SEQ ID NO: 54) | GGG | 75 | 14.9 |
| hSerpinc1-Sp-37 | ACACGCCGGTTGGTGGCCTC(SEQ ID NO: 55) | CGG | 70 | 6.2 |
| hSerpinc1-Sp-39 | TTCCCAGACACGCCGGTTGG(SEQ ID NO: 57) | TGG | 65 | 22.5 |
| hSerpinc1-Sp-40 | CAGTTCCCAGACACGCCGGT(SEQ ID NO: 58) | TGG | 65 | 22.1 |
| hSerpinc1-Sp-42 | AGGCCACCAACCGGCGTGTC(SEQ ID NO: 60) | TGG | 70 | 0.2 |
| hSerpinc1-Sp-43 | GGCCACCAACCGGCGTGTCT(SEQ ID NO: 61) | GGG | 70 | 8.2 |
| hSerpinc1-Sp-45 | AGCAAAGCGGGAATTGGCCT(SEQ ID NO: 63) | TGG | 55 | 0.0 |
| hSerpinc1-Sp-49 | TGCCAGGTGCTGATAGAAAG(SEQ ID NO: 67) | TGG | 50 | 34.4 |
| hSerpinc1-Sp-50 | TACCACTTTCTATCAGCACC(SEQ ID NO: 68) | TGG | 45 | 27.3 |
| hSerpinc1-Sp-51 (Serpinc1_Ex2 #19) | TGTCATTCTTGGAATCTGCC(SEQ ID NO: 69) | AGG | 45 | 2.3% |
| hSerpinc1-Sp-53 (Serpinc1_Ex2 | TGGAGATACTCAGGGGTGAC(SEQ ID NO: 71) | AGG | 55 | 18.3% |
| hSerpinc1-Sp-54 (Serpinc1_Ex2 #21) | AAAGCCGTGGAGATACTCAG(SEQ ID NO: 72) | GGG | 50 | 67.7% |
| hSerpinc1-Sp-58 (Serpinc1_Ex2 #22) | CTTGGTCATAGCAAAAGCCG(SEQ ID NO: 76) | TGG | 50 | 33.5% |
| hSerpinc1-Sp-59 (Serpinc1_Ex2 #23) | GGCTTTTGCTATGACCAAGC(SEQ ID NO: 77) | TGG | 50 | 2.0% |
| hSerpinc1-Sp-60 (Serpinc1_Ex2 #24) | GCTTTTGCTATGACCAAGCT(SEQ ID NO: 78) | GGG | 45 | 14.3% |
| hSerpinc1-Sp-62 (Serpinc1_Ex2 #25) | TTGCTGGAGGGTGTCATTAC(SEQ ID NO: 80) | AGG | 50 | 22.9% |
| hSerpinc1-Sp-66 (Serpinc1_Ex2 #26) | TGACACCCTCCAGCAACTGA(SEQ ID NO: 84) | TGG | 55 | 19.8% |
| hSerpinc1-Sp-64 (Serpinc1_Ex2 #27) | GTACCTCCATCAGTTGCTGG(SEQ ID NO: 82) | AGG | 55 | 12.0% |
| hSerpinc1-Sp-70 (Serpinc1_Ex3 #1) | ATAGAGTCGGCAGTTCAGTT(SEQ ID NO: 88) | TGG | 45 | 56.0% |
| hSerpinc1-Sp-71 (Serpinc1_Ex3 #2) | TGTTGGCTTTTCGATAGAGT(SEQ ID NO: 89) | CGG | 40 | 15.2% |
| hSerpinc1-Sp-73 (Serpinc1_Ex3 #3) | ATTGGCTGATACTAACTTGG(SEQ ID NO: 91) | AGG | 40 | 17.3% |
| hSerpinc1-Sp-74 (Serpinc1_Ex3 #4) | GCGATTGGCTGATACTAACT(SEQ ID NO: 92) | TGG | 45 | 38.9% |
| hSerpinc1-Sp-76 (Serpinc1_Ex3 #5) | GTATCAGCCAATCGCCTTTT(SEQ ID NO: 94) | TGG | 45 | 4.4% |
| hSerpinc1-Sp-75 (Serpinc1_Ex3 #6) | TTTGTCTCCAAAAAGGCGAT(SEQ ID NO: 93) | TGG | 40 | 13.4% |
| hSerpinc1-Sp-79 (Serpinc1_Ex3 #7) | GGTAGGTCTCATTGAAGGTA(SEQ ID NO: 97) | AGG | 45 | 5.2% |
| hSerpinc1-Sp-80 (Serpinc1_Ex3 #8) | GTCCTGGTAGGTCTCATTGA(SEQ ID NO: 98) | AGG | 50 | 59.9% |
| hSerpinc1-Sp-83 | ATACCAACTCACTGATGTCC(SEQ | TGG | 45 | 73.4% |
| hSerpinc1-Sp-98 (Serpinc1_Ex4 #1) | CAGAGCGGCCATCAACAAAT(SEQ ID NO: 116) | GGG | 50 | 35.9% |
| hSerpinc1-Sp-99 (Serpinc1_Ex4 #2) | GATTCGGCCTTCGGTCTTAT(SEQ ID NO: 117) | TGG | 50 | 11.2% |
| hSerpinc1-Sp-101 (Serpinc1_Ex4 #3) | GACATCGGTGATTCGGCCTT(SEQ ID NO: 119) | CGG | 55 | 0.3% |
| hSerpinc1-Sp-104 (Serpinc1_Ex4 #4) | AATCACCGATGTCATTCCCT(SEQ ID NO: 122) | CGG | 45 | 19.4% |
| hSerpinc1-Sp-106 (Serpinc1_Ex4 #5) | GAGCTCATTGATGGCTTCCG(SEQ ID NO: 124) | AGG | 55 | 4.9% |
| hSerpinc1-Sp-114 (Serpinc1_Ex5 #1) | GGCTGAACTTTGACTTCCAC(SEQ ID NO: 132) | AGG | 50 | 8.1% |
| hSerpinc1-Sp-120 (Serpinc1_Ex5 #2) | GAACTGTTCTACAAGGCTGA(SEQ ID NO: 138) | TGG | 45 | 23.0% |
| hSerpinc1-Sp-123 (Serpinc1_Ex5 #3) | GATAACGGAACTTGCCTTCC(SEQ ID NO: 141) | TGG | 50 | 1.2% |
| hSerpinc1-Sp-124 (Serpinc1_Ex5 #4) | AGGAAGGCAAGTTCCGTTAT(SEQ ID NO: 142) | CGG | 45 | 28.4% |
| hSerpinc1-Sp-126 (Serpinc1_Ex5 #5) | CAAGTTCCGTTATCGGCGCG(SEQ ID NO: 144) | TGG | 60 | 6.1% |
| hSerpinc1-Sp-125 (Serpinc1_Ex5 #6) | CTTCAGCCACGCGCCGATAA(SEQ ID NO: 143) | CGG | 60 | 0.9% |
| hSerpinc1-Sp-127 (Serpinc1_Ex5 #7) | CGTTATCGGCGCGTGGCTGA(SEQ ID NO: 145) | AGG | 65 | 9.8% |
| hSerpinc1-Sp-142 (Serpinc1_Ex5 #8) | GTTCCTTCTCTACCTTGGCC(SEQ ID NO: 160) | AGG | 55 | 5.4% |
| hSerpinc1-Sp-156 (Serpinc1_Ex5 #9) | CCGTCCTCAATGCGGAAGCG(SEQ ID NO: 174) | GGG | 65 | 12.8% |
| hSerpinc1-Sp-157 (Serpinc1_Ex5 #10) | GCCGTCCTCAATGCGGAAGC(SEQ ID NO: 175) | GGG | 65 | 10.7% |
| hSerpinc1-Sp-158 (Serpinc1_Ex5 #11) | AGCCGTCCTCAATGCGGAAG(SEQ ID NO: 176) | CGG | 60 | 16.7% |
| hSerpinc1-Sp-160 (Serpinc1_Ex5 #12) | AACTGAAGCCGTCCTCAATG(SEQ ID NO: 178) | CGG | 50 | 59.5% |
| hSerpinc1-Sp-162 | TGAGGACGGCTTCAGTTTGA(SE | AGG | 50 | 6.6% |
| hSerpinc1-Sp-165 (Serpinc1_Ex5 #14) | CAGGGCTGAACAGATCGACA(SEQ ID NO: 183) | AGG | 55 | 60.2% |
| hSerpinc1-Sp-167 (Serpinc1_Ex5 #15) | CCTGGGAGTTTGGACTTTTC(SEQ ID NO: 185) | AGG | 50 | 26.4% |
| hSerpinc1-Sp-170 (Serpinc1_Ex6 #1) | CGGCCTTCTGCAACAATACC(SEQ ID NO: 188) | TGG | 55 | 28.8% |
| hSerpinc1-Sp-172 (Serpinc1_Ex6 #2) | CTGAGACATAGAGGTCATCT(SEQ ID NO: 190) | CGG | 45 | 48.5% |
| hSerpinc1-Sp-178 (Serpinc1_Ex7 #1) | ACGGCCAGCAATCACAACAG(SEQ ID NO: 196) | CGG | 55 | 81.6% |
| hSerpinc1-Sp-181 (Serpinc1_Ex7 #2) | GCCGTTCGCTAAACCCCAAC(SEQ ID NO: 199) | AGG | 60 | 47.7% |
| hSerpinc1-Sp-182 (Serpinc1_Ex7 #3) | CCGTTCGCTAAACCCCAACA(SEQ ID NO: 200) | GGG | 55 | 89.0% |
| hSerpinc1-Sp-180 (Serpinc1_Ex7 #4) | CCCTGTTGGGGTTTAGCGAA(SEQ ID NO: 198) | CGG | 55 | 47.3% |

Here, the description of “Ex-No” means the exon number of the gene.

Specific guide sequences for the targets described above are as follows:

TABLE 11

| | | |
|---|---|---|
| | Target(w/o PAM) | Guide sequence |
| hSerpinc1-Sp-3 | GCCATGTATTCCAATGTGAT(SEQ ID NO: 21) | GCCAUGUAUUCCAAUGUGAU(SEQ ID NO: 849) |
| hSerpinc1-Sp-4 | GTGATAGGAACTGTAACCTC(SEQ ID NO: 22) | GUGAUAGGAACUGUAACCUC(SEQ ID NO: 850) |
| hSerpinc1-Sp-12 | GGGACTGCGTGACCTGTCAC(SEQ ID NO: 30) | GGGACUGCGUGACCUGUCAC(SEQ ID NO: 851) |
| hSerpinc1-Sp-13 | GTCCACAGGGCTCCCGTGAC(SEQ ID NO: 31) | GUCCACAGGGCUCCCGUGAC(SEQ ID NO: 852) |
| hSerpinc1-Sp-19 | CATGGGAATGTCCCGCGGCT(SEQ ID NO: 37) | CAUGGGAAUGUCCCGCGGCU(SEQ ID NO: 853) |
| hSerpinc1-Sp-20 | GGATTCATGGGAATGTCCCG(SEQ ID NO: 38) | GGAUUCAUGGGAAUGUCCCG(SEQ ID NO: 854) |
| hSerpinc1-Sp-23 | GGGGAGCGGTAAATGCACAT(SEQ ID NO: 41) | GGGGAGCGGUAAAUGCACAU(SEQ ID NO: 855) |
| hSerpinc1-Sp-24 | CGGGGAGCGGTAAATGCACA(SEQ ID NO: 42) | CGGGGAGCGGUAAAUGCACA(SEQ ID NO: 856) |
| hSerpinc1-Sp-25 | CATGTGCATTTACCGCTCCC(SEQ ID NO: 43) | CAUGUGCAUUUACCGCUCCC(SEQ ID NO: 857) |
| hSerpinc1-Sp-30 | TTACCGCTCCCCGGAGAAGA(SEQ ID NO: 48) | UUACCGCUCCCCGGAGAAGA(SEQ ID NO: 858) |
| hSerpinc1-Sp-34 | GGGCTCAGAACAGAAGATCC(SEQ ID NO: 52) | GGGCUCAGAACAGAAGAUCC(SEQ ID NO: 859) |
| hSerpinc1-Sp-36 | CACGCCGGTTGGTGGCCTCC(SEQ ID NO: 54) | CACGCCGGUUGGUGGCCUCC(SEQ ID NO: 860) |
| hSerpinc1-Sp-37 | ACACGCCGGTTGGTGGCCTC(SEQ ID NO: 55) | ACACGCCGGUUGGUGGCCUC(SEQ ID NO: 861) |
| hSerpinc1-Sp-39 | TTCCCAGACACGCCGGTTGG(SEQ ID NO: 57) | UUCCCAGACACGCCGGUUGG(SEQ ID NO: 862) |
| hSerpinc1-Sp-40 | CAGTTCCCAGACACGCCGGT(SEQ ID NO: 58) | CAG U UCCCAGACACGCCGGU(SEQ ID NO: 863) |
| hSerpinc1-Sp-42 | AGGCCACCAACCGGCGTGTC(SEQ ID NO: 60) | AGGCCACCAACCGGCGUGUC(SEQ ID NO: 864) |
| hSerpinc1-Sp-43 | GGCCACCAACCGGCGTGTCT(SEQ ID NO: 61) | GGCCACCAACCGGCGUGUCU(SEQ ID NO: 865) |
| hSerpinc1-Sp-45 | AGCAAAGCGGGAATTGGCCT(SEQ ID NO: 63) | AGCAAAGCGGGAAUUGGCCU(SEQ ID NO: 866) |
| hSerpinc1-Sp-49 | TGCCAGGTGCTGATAGAAAG(SEQ ID NO: 67) | UGCCAGGUGCUGAUAGAAAG(SEQ ID NO: 867) |
| hSerpinc1-Sp-50 | TACCACTTTCTATCAGCACC(SEQ ID NO: 68) | UACCACUUUCUAUCAGCACC(SEQ ID NO: 868) |
| hSerpinc1-Sp-51 (Serpinc1_Ex2 #19) | TGTCATTCTTGGAATCTGCC(SEQ ID NO: 69) | UGUCAUUCUUGGAAUCUGCC(SEQ ID NO: 869) |
| hSerpinc1-Sp-53 (Serpinc1_Ex2 #20) | TGGAGATACTCAGGGGTGAC(SEQ ID NO: 71) | UGGAGAUACUCAGGGGUGAC(SEQ ID NO: 870) |
| hSerpinc1-Sp-54 (Serpinc1_Ex2 #21) | AAAGCCGTGGAGATACTCAG(SEQ ID NO: 72) | AAAGCCGUGGAGAUACUCAG(SEQ ID NO: 871) |
| hSerpinc1-Sp-58 (Serpinc1_Ex2 | CTTGGTCATAGCAAAAGCCG(SEQ ID NO: 76) | CUUGGUCAUAGCAAAAGCCG(SEQ ID NO: 872) |
| hSerpinc1-Sp-59 (Serpinc1_Ex2 #23) | GGCTTTTGCTATGACCAAGC(SEQ ID NO: 77) | GGCUUUUGCUAUGACCAAGC(SEQ ID NO: 873) |
| hSerpinc1-Sp-60 (Serpinc1_Ex2 #24) | GCTTTTGCTATGACCAAGCT(SEQ ID NO: 78) | GCUUUUGCUAUGACCAAGCU(SEQ ID NO: 874) |
| hSerpinc1-Sp-62 (Serpinc1_Ex2 #25) | TTGCTGGAGGGTGTCATTAC(SEQ ID NO: 80) | UUGCUGGAGGGUGUCAUUAC(SEQ ID NO: 875) |
| hSerpinc1-Sp-66 (Serpinc1_Ex2 #26) | TGACACCCTCCAGCAACTGA(SEQ ID NO: 84) | UGACACCCUCCAGCAACUGA(SEQ ID NO: 876) |
| hSerpinc1-Sp-64 (Serpinc1_Ex2 #27) | GTACCTCCATCAGTTGCTGG(SEQ ID NO: 82) | GUACCUCCAUCAGUUGCUGG(SEQ ID NO: 877) |
| hSerpinc1-Sp-70 (Serpinc1_Ex3 #1) | ATAGAGTCGGCAGTTCAGTT(SEQ ID NO: 88) | AUAGAGUCGGCAGUUCAGUU(SEQ ID NO: 878) |
| hSerpinc1-Sp-71 (Serpinc1_Ex3 #2) | TGTTGGCTTTTCGATAGAGT(SEQ ID NO: 89) | UGUUGGCUUUUCGAUAGAGU(SEQ ID NO: 879) |
| hSerpinc1-Sp-73 (Serpinc1_Ex3 #3) | ATTGGCTGATACTAACTTGG(SEQ ID NO: 91) | AUUGGCUGAUACUAACUUGG(SEQ ID NO: 880) |
| hSerpinc1-Sp-74 (Serpinc1_Ex3 #4) | GCGATTGGCTGATACTAACT(SEQ ID NO: 92) | GCGAUUGGCUGAUACUAACU(SEQ ID NO: 881) |
| hSerpinc1-Sp-76 (Serpinc1_Ex3 #5) | GTATCAGCCAATCGCCTTTT(SEQ ID NO: 94) | GUAUCAGCCAAUCGCCUUUU(SEQ ID NO: 882) |
| hSerpinc1-Sp-75 (Serpinc1_Ex3 #6) | TTTGTCTCCAAAAAGGCGAT(SEQ ID NO: 93) | UUUGUCUCCAAAAAGGCGAU(SEQ ID NO: 883) |
| hSerpinc1-Sp-79 (Serpinc1_Ex3 #7) | GGTAGGTCTCATTGAAGGTA(SEQ ID NO: 97) | GGUAGGUCUCAUUGAAGGUA(SEQ ID NO: 884) |
| hSerpinc1-Sp-80 (Serpinc1_Ex3 #8) | GTCCTGGTAGGTCTCATTGA(SEQ ID NO: 98) | GUCCUGGUAGGUCUCAUUGA(SEQ ID NO: 885) |
| hSerpinc1-Sp-83 (Serpinc1_Ex3 #9) | ATACCAACTCACTGATGTCC(SEQ ID NO: 101) | AUACCAACUCACUGAUGUCC(SEQ ID NO: 886) |
| hSerpinc1-Sp-98 (Serpinc1_Ex4 #1) | CAGAGCGGCCATCAACAAAT(SEQ ID NO: 116) | CAGAGCGGCCAUCAACAAAU(SEQ ID NO: 887) |
| hSerpinc1-Sp-99 (Serpinc1_Ex4 #2) | GATTCGGCCTTCGGTCTTAT(SEQ ID NO: 117) | GAUUCGGCCUUCGGUCUUAU(SEQ ID NO: 888) |
| hSerpinc1-Sp-101 | GACATCGGTGATTCGGCCT | GACAUCGGUGAUUCGGC |
| hSerpinc1-Sp-104 (Serpinc1_Ex4 #4) | AATCACCGATGTCATTCCCT(SEQ ID NO: 122) | AAUCACCGAUGUCAUUCCCU(SEQ ID NO: 890) |
| hSerpinc1-Sp-106 (Serpinc1_Ex4 #5) | GAGCTCATTGATGGCTTCCG(SEQ ID NO: 124) | GAGCUCAUUGAUGGCUUCCG(SEQ ID NO: 891) |
| hSerpinc1-Sp-114 (Serpinc1_Ex5 #1) | GGCTGAACTTTGACTTCCAC(SEQ ID NO: 132) | GGCUGAACUUUGACUUCCAC(SEQ ID NO: 892) |
| hSerpinc1-Sp-120 (Serpinc1_Ex5 #2) | GAACTGTTCTACAAGGCTGA(SEQ ID NO: 138) | GAACUGUUCUACAAGGCUGA(SEQ ID NO: 893) |
| hSerpinc1-Sp-123 (Serpinc1_Ex5 #3) | GATAACGGAACTTGCCTTCC(SEQ ID NO: 141) | GAUAACGGAACUUGCCUUCC(SEQ ID NO: 894) |
| hSerpinc1-Sp-124 (Serpinc1_Ex5 #4) | AGGAAGGCAAGTTCCGTTAT(SEQ ID NO: 142) | AGGAAGGCAAGUUCCGUUAU(SEQ ID NO: 895) |
| hSerpinc1-Sp-126 (Serpinc1_Ex5 #5) | CAAGTTCCGTTATCGGCGCG(SEQ ID NO: 144) | CAAGUUCCGUUAUCGGCGCG(SEQ ID NO: 896) |
| hSerpinc1-Sp-125 (Serpinc1_Ex5 #6) | CTTCAGCCACGCGCCGATAA(SEQ ID NO: 143) | CUUCAGCCACGCGCCGAUAA(SEQ ID NO: 897) |
| hSerpinc1-Sp-127 (Serpinc1_Ex5 #7) | CGTTATCGGCGCGTGGCTGA(SEQ ID NO: 145) | CGUUAUCGGCGCGUGGCUGA(SEQ ID NO: 898) |
| hSerpinc1-Sp-142 (Serpinc1_Ex5 #8) | GTTCCTTCTCTACCTTGGCC(SEQ ID NO: 160) | GUUCCUUCUCUACCUUGGCC(SEQ ID NO: 899) |
| hSerpinc1-Sp-156 (Serpinc1_Ex5 #9) | CCGTCCTCAATGCGGAAGCG(SEQ ID NO: 174) | CCGUCCUCAAUGCGGAAGCG(SEQ ID NO: 900) |
| hSerpinc1-Sp-157 (Serpinc1_Ex5 #10) | GCCGTCCTCAATGCGGAAGC(SEQ ID NO: 175) | GCCGUCCUCAAUGCGGAAGC(SEQ ID NO: 901) |
| hSerpinc1-Sp-158 (Serpinc1_Ex5 #11) | AGCCGTCCTCAATGCGGAAG(SEQ ID NO: 176) | AGCCGUCCUCAAUGCGGAAG(SEQ ID NO: 902) |
| hSerpinc1-Sp-160 (Serpinc1_Ex5 #12) | AACTGAAGCCGTCCTCAATG(SEQ ID NO: 178) | AACUGAAGCCGUCCUCAAUG(SEQ ID NO: 903) |
| hSerpinc1-Sp-162 (Serpinc1_Ex5 #13) | TGAGGACGGCTTCAGTTTGA(SEQ ID NO: 180) | UGAGGACGGCUUCAGUUUGA(SEQ ID NO: 903) |
| hSerpinc1-Sp-165 (Serpinc1_Ex5 #14) | CAGGGCTGAACAGATCGACA(SEQ ID NO: 183) | CAGGGCUGAACAGAUCGACA(SEQ ID NO: 904) |
| hSerpinc1-Sp-167 | CCTGGGAGTTTGGACTTTT | CCUGGGAGUUUGGACUU |
| hSerpinc1-Sp-170 (Serpinc1_Ex6 #1) | CGGCCTTCTGCAACAATACC(SEQ ID NO: 188) | CGGCCUUCUGCAACAAUACC(SEQ ID NO: 906) |
| hSerpinc1-Sp-172 (Serpinc1_Ex6 #2) | CTGAGACATAGAGGTCATCT(SEQ ID NO: 190) | CUGAGACAUAGAGGUCAUCU(SEQ ID NO: 907) |
| hSerpinc1-Sp-178 (Serpinc1_Ex7 #1) | ACGGCCAGCAATCACAACAG(SEQ ID NO: 196) | ACGGCCAGCAAUCACAACAG(SEQ ID NO: 908) |
| hSerpinc1-Sp-181 (Serpinc1_Ex7 #2) | GCCGTTCGCTAAACCCCAAC(SEQ ID NO: 199) | GCCGUUCGCUAAACCCCAAC(SEQ ID NO: 909) |
| hSerpinc1-Sp-182 (Serpinc1_Ex7 #3) | CCGTTCGCTAAACCCCAACA(SEQ ID NO: 200) | CCGUUCGCUAAACCCCAACA(SEQ ID NO: 910) |
| hSerpinc1-Sp-180 (Serpinc1_Ex7 #4) | CCCTGTTGGGGTTTAGCGAA(SEQ ID NO: 198) | CCCUGUUGGGGUUUAGCGAA(SEQ ID NO: 911) |

② SaCas9

TABLE 6

gRNA selection to target SERPINC1 gene(AT gene) for SaCas9

| Target name | Target(w/o PAM) (SEQ ID NO) | PAM | GC content | Indels (%) |
|---|---|---|---|---|
| hAPOC3-Cj-Ctrl | GAGAGGGCCAGAAATCACCCAA(SEQ ID NO: 848) | AGACACAC | | 64.1 |
| hSerpinc1-Sa-1 | GGTTACAGTTCCTATCACAT(SEQ ID NO: 216) | TGGAAT | 40 | 51.6 |
| hSerpinc1-Sa-2 | CCAAGCCGCGGGACATTCCC(SEQ ID NO: 217) | ATGAAT | 70 | 65.2 |
| hSerpinc1-Sa-3 | TAAATGCACATGGGATTCAT(SEQ ID NO: 218) | GGGAAT | 35 | 49.2 |
| hSerpinc1-Sa-4 | CGGGGAGCGGTAAATGCACA(SEQ ID NO: 219) | TGGGAT | 60 | 34.5 |
| hSerpinc1-Sa-5 | CCCCGGAGAAGAAGGCAACT(SEQ ID NO: 220) | GAGGAT | 60 | 44.7 |
| hSerpinc1-Sa-6 | ACACGCCGGTTGGTGGCCTC(SEQ ID NO: 221) | CGGGAT | 70 | 0.8 |
| hSerpinc1-Sa-7 | ATAGAAAGTGGTAGCAAAGC(SEQ ID NO: 222) | GGGAAT | 40 | 68.3 |
| hSerpinc1-Sa-9 | AATGTTATCATTGTCATTCT(SEQ ID NO: 224) | TGGAAT | 25 | 12.9 |
| hSerpinc1-Sa-11 | CAAAAGCCGTGGAGATACTC(SEQ ID NO: 226) | AGGGGT | 50 | 58.9 |
| hSerpinc1-Sa-13 | CGTACCTCCATCAGTTGCTG(SEQ ID NO: 228) | GAGGGT | 55 | 75.5 |
| hSerpinc1-Sa-14 | TTCAGTTTGGCAAAGAAGAA(SEQ ID NO: 229) | GTGGAT | 35 | 26.5 |
| hSerpinc1-Sa-17 | GGTAGGTCTCATTGAAGGTA(SEQ ID NO: 232) | AGGGAT | 45 | 61.8 |
| hSerpinc1-Sa-18 | TGAGACCTACCAGGACATCA(SEQ ID NO: 233) | GTGAGT | 50 | 70.2 |
| hSerpinc1-Sa-20 | CCCATTTGTTGATGGCCGCT(SEQ ID NO: 235) | CTGGAT | 55 | 3.4 |
| hSerpinc1-Sa-21 | TCCAGAGCGGCCATCAACAA(SEQ ID NO: 236) | ATGGGT | 55 | 68.0 |

③ CjCas9

TABLE 7

gNA selection to target SERPINC1 gene(AT gene) for CjCas9

| Target name | Target(w/o PAM) | PAM | GC content | Indels (%) |
|---|---|---|---|---|
| hAPOC3-Cj-Ctrl | GAGAGGGCCAGAAATCACCCAA(SEQ ID NO: 848) | AGACACAC | | 36.8 |
| hSerpinc1-Cj-1 | GAGGTTACAGTTCCTATCACAT(SEQ ID NO: 253) | TGGAATAC | 41 | 24.6 |
| hSerpinc1-Cj-2 | CTGTCACGGGAGCCCTGTGGAC(SEQ ID NO: 254) | ATCTGCAC | 68 | 0.3 |
| hSerpinc1-Cj-3 | GCCTTCTTCTCCGGGGAGCGGT(SEQ ID NO: 255) | AAATGCAC | 68 | 1.3 |
| hSerpinc1-Cj-4 | GGGAATTGGCCTTGGACAGTTC(SEQ ID NO: 256) | CCAGACAC | 55 | 3.9 |
| hSerpinc1-Cj-5 | TCCCGCTTTGCTACCACTTTCT(SEQ ID NO: 257) | ATCAGCAC | 50 | 0 |
| hSerpinc1-Cj-6 | GCTTGGTCATAGCAAAAGCCGT(SEQ ID NO: 258) | GGAGATAC | 50 | 3.1 |
| hSerpinc1-Cj-7 | TATGACCAAGCTGGGTGCCTGT(SEQ ID NO: 259) | AATGACAC | 55 | 17.5 |
| hSerpinc1-Cj-8 | TCAGTTGCTGGAGGGTGTCATT(SEQ ID NO: 260) | ACAGGCAC | 50 | 0.9 |
| hSerpinc1-Cj-9 | ATGACACCCTCCAGCAACTGAT(SEQ ID NO: 261 | GGAGGTAC | 50 | 3.2 |
| hSerpinc1-Cj-10 | TTGACTTCTATAGGTATTTAAG(SEQ ID NO: 262) | TTTGACAC | 27 | 0.3 |
| hSerpinc1-Cj-11 | TTTCTCAGATATGGTGTCAAAC(SEQ ID NO: 263) | TTAAATAC | 36 | 0 |
| hSerpinc1-Cj-12 | TTTGTCTCCAAAAAGGCGATTG(SEQ ID NO: 264) | GCTGATAC | 41 | 0.2 |
| hSerpinc1-Cj-13 | GTCCAGGGGCTGGAGCTTGGCT(SEQ ID NO: 265) | CCATATAC | 68 | 0 |
| hSerpinc1-Cj-14 | GGTGATTCGGCCTTCGGTCTTA(SEQ ID NO: 266) | TTGGACAC | 55 | 0.1 |
| hSerpinc1-Cj-15 | TGAGCTCACTGTTCTGGTGCTG(SEQ ID NO: 267) | GTTAACAC | 55 | 7.7 |
| hSerpinc1-Cj-16 | TACCTTGAAGTAAATGGTGTTA(SEQ ID NO: 268) | ACCAGCAC | 32 | 0.1 |
| hSerpinc1-Cj-17 | TGCTGGTTAACACCATTTACTT(SEQ ID NO: 269) | CAAGGTAC | 36 | 0.1 |
| hSerpinc1-Cj-18 | GTGGAAGTCAAAGTTCAGCCCT(SEQ ID NO: 270) | GAGAACAC | 50 | 20.4 |
| hSerpinc1-Cj-19 | GGAGAGTCGTGTTCAGCATCTA(SEQ ID NO: 271) | TGATGTAC | 50 | 0 |
| hSerpinc1-Cj-20 | CCTTCCTGGTACATCATAGATG(SEQ ID NO: 272) | CTGAACAC | 45 | 10.1 |
| hSerpinc1-Cj-21 | CGCCGATAACGGAACTTGCCTT(SEQ ID NO: 273) | CCTGGTAC | 55 | 0.1 |
| hSerpinc1-Cj-22 | GTTCCGTTATCGGCGCGTGGCT(SEQ ID NO: 274) | GAAGGCAC | 64 | 0.8 |
| hSerpinc1-Cj-23 | GTCATCACCTTTGAAGGGCAAC(SEQ ID NO: 275) | TCAAGCAC | 50 | 0.1 |
| hSerpinc1-Cj-24 | CTCCAATTCATCCAGCCACTCT(SEQ ID NO: 276) | TGCAGCAC | 50 | 0 |
| hSerpinc1-Cj-25 | AGAGGTCATCTCGGCCTTCTGC(SEQ ID NO: 277) | AACAATAC | 59 | 0.3 |
| hSerpinc1-Cj-26 | ATTCCATAAGGCATTTCTTGAG(SEQ ID NO: 278) | GTGAGTAC | 36 | 2.7 |
| hSerpinc1-Cj-28 | GCGAACGGCCAGCAATCACAAC(SEQ ID NO: 280) | AGCGGTAC | 59 | 0.4 |
| hSerpinc1-Cj-29 | GGTTTTTATAAGAGAAGTTCCT(SEQ ID NO: 281) | CTGAACAC | 32 | 1.3 |
| hSerpinc1-Cj-30 | TGCAAAGAATAAGAACATTTTA(SEQ ID NO: 282) | CTTAACAC | 23 | 0.1 |

2) TFPI gene
① SpCas9

TABLE 8

gRNA selection to target TFPI gene for SpCas9

| Target name | Target(w/o PAM) (SEQ ID NO) | PAM | GC content | Indels (%) |
|---|---|---|---|---|
| hMyd88-Sp-Ctrl | GTCCATCTCCTCCGCCAGCG(SEQ ID NO: 847) | CGG | | 75.2 |
| hTfpi-Sp-4 | ATCAGCATTAAGAGGGGCAG(SEQ ID NO: 286) | GGG | 50 | 54.9 |
| hTfpi-Sp-6 | GAATCAGCATTAAGAGGGGC(SEQ ID NO: 288) | AGG | 50 | 26.4 |
| hTfpi-Sp-17 | TGTGCATTCAAGGCGGATGA(SEQ ID NO: 299) | TGG | 50 | 45.7 |
| hTfpi-Sp-21 | AGTGCGAAGAATTTATATAT(SEQID NO: 303) | GGG | 25 | 62.1 |
| hTfpi-Sp-22 | GTGCGAAGAATTTATATATG(SEQID NO: 304) | GGG | 30 | 31.1 |
| hTfpi-Sp-33 | ATATAACCTCGACATATTCC(SEQID NO: 315) | AGG | 35 | 26.7 |
| hTfpi-Sp-38 | TGTGAACGTTTCAAGTATGG(SEQID NO: 320) | TGG | 40 | 57.0 |
| hTfpi-Sp-43 | TGCAAGAACATTTGTGAAGA(SEQID NO: 325) | TGG | 35 | 1.0 |
| hTfpi-Sp-45 | TCCACCTGGAAACCATTCGC(SEQ ID NO: 327) | TGG | 55 | 25.5 |
| hTfpi-Sp-47 | TCCAGCGAATGGTTTCCAGG(SEQ ID NO: 329) | TGG | 55 | 26.0 |
| hTfpi-Sp-52 | CTTGGTTGATTGCGGAGTCA(SEQ ID NO: 334) | GGG | 50 | 33.6 |
| hTfpi-Sp-53 | CCTTGGTTGATTGCGGAGTC(SEQ ID NO: 335) | AGG | 55 | 3.2 |
| hTfpi-Sp-54 | CTGGGAACCTTGGTTGATTG(SEQ ID NO: 336) | CGG | 50 | 37.0 |
| hTfpi-Sp-60 | CCACAAGATTCTTACCAAAA(SEQID NO: 342) | AGG | 35 | 13.2 |

② SaCas9

TABLE 9

gRNA selection to target TFPI gene for SaCas9

| Target name | Target(w/o PAM) (SEQ ID NO) | PAM | GC content | Indels (%) |
|---|---|---|---|---|
| hAPOC3-Cj-Ctrl | GAGAGGGCCAGAAATCACCCAA(SEQ ID NO: 848) | AGACACAC | | 57.3 |
| hTfpi-Sa-3 | ATCCGCCTTGAATGCACAAA(SEQ ID NO: 375) | ATGAAT | 45 | 3.0 |
| hTfpi-Sa-5 | TACATGGGCCATCATCCGCC(SEQ ID NO: 377) | TTGAAT | 60 | 68.2 |
| hTfpi-Sxla-8 | GTGCGAAGAATTTATATATG(SEQ ID NO: 380) | GGGGAT | 30 | 34.6 |
| hTfpi-Sa-10 | GAATCGATTTGAAAGTCTGG(SEQ ID NO: 382) | AAGAGT | 40 | 72.2 |
| hTfpi-Sa-13 | AATATAACCTCGACATATTC(SEQ ID NO: 385) | CAGGAT | 30 | 15.1 |
| hTfpi-Sa-14 | GTGTGAACGTTTCAAGTATG(SEQ ID NO: 386) | GTGGAT | 40 | 38.8 |
| hTfpi-Sa-16 | TTCCAGCGAATGGTTTCCAG(SEQ ID NO: 388) | GTGGAT | 50 | 58.6 |
| hTfpi-Sa-17 | GAGTTATTCACAGCATTGAG(SEQ ID NO: 389) | CTGGGT | 40 | 62.6 |
| hTfpi-Sa-18 | ATGGAACCCAGCTCAATGCT(SEQ ID NO: 390) | GTGAAT | 50 | 38.9 |
| hTfpi-Sa-19 | CTTGGTTGATTGCGGAGTCA(SEQ ID NO: 391) | GGGAGT | 50 | 67.1 |
| hTfpi-Sa-20 | CTGGGAACCTTGGTTGATTG(SEQ ID NO: 392) | CGGAGT | 50 | 73.6 |
| hTfpi-Sa-22 | CGACACAATCCTCTGTCTGC(SEQ ID NO: 394) | TGGAGT | 55 | 47.0 |
| hTfpi-Sa-24 | TCCCAATGACTGAATTGTAG(SEQ ID NO: 396) | TAGAAT | 40 | 49.5 |
| hTfpi-Sa-25 | TGGGCGGCATTTCCCAATGA(SEQ ID NO: 397) | CTGAAT | 55 | 57.1 |
| hTfpi-Sa-26 | ATGCCGCCCATTTAAGTACA(SEQ ID NO: 398) | GTGGAT | 45 | 39.0 |

③ CjCas9

TABLE 10

gRNA selection to target TFPI gene for CjCas9

| Target name | Target(w/o PAM)(SEQ ID NO) | PAM | GC content | Indels (%) |
|---|---|---|---|---|
| hAPOC3-Cj-Ctrl | GAGAGGGCCAGAAATCACCCAA(SEQ ID NO: 848) | AGACACAC | | 36.8 |
| hTfpi-Cj-1 | TGGGTTCTGTATTTCAGAGATG(SEQ ID NO: 403) | ATTTACAC | 41 | 0 |
| hTfpi-Cj-2 | TCTTCATTGTGTAAATCATCTC(SEQ ID NO: 404) | TGAAATAC | 32 | 1.2 |
| hTfpi-Cj-3 | CAGAGATGATTTACACAATGAA(SEQ ID NO: 405) | GAAAGTAC | 32 | 3.9 |
| hTfpi-Cj-5 | CAGGCATACAGAAGCCCAAAGT(SEQ ID NO: 407) | GCATGTAC | 50 | 0.8 |
| hTfpi-Cj-6 | GGCAGGGGCAAGATTAAGCAGC(SEQ ID NO: 408) | AGGCATAC | 59 | 19.3 |
| hTfpi-Cj-7 | AATGCTGATTCTGAGGAAGATG(SEQ ID NO: 409) | AAGAACAC | 41 | 22.1 |
| hTfpi-Cj-8 | TGCTGATTCTGAGGAAGATGAA(SEQ ID NO: 410) | GAACACAC | 41 | 17.5 |
| hTfpi-Cj-9 | TACATGGGCCATCATCCGCCTT(SEQ ID NO: 411) | GAATGCAC | 55 | 0 |
| hTfpi-Cj-10 | TCACATCCCCCATATATAAATT(SEQ ID NO: 412) | CTTCGCAC | 32 | 0 |
| hTfpi-Cj-11 | AAGTCTGGAAGAGTGCAAAAAA(SEQ ID NO: 413) | ATGTGTAC | 36 | 0.3 |
| hTfpi-Cj-12 | AACCTACCTCTTGTACACATTT(SEQ ID NO: 414) | TTTTGCAC | 36 | 0 |
| hTfpi-Cj-13 | AGGGTTCCCAGAAACCTACCTC(SEQ ID NO: 415) | TTGTACAC | 55 | 1.6 |
| hTfpi-Cj-14 | CACTGTTTTGTCTGATTGTTAT(SEQ ID NO: 416) | AAAAATAC | 32 | 0.1 |
| hTfpi-Cj-15 | AGGCATCCACCATACTTGAAAC(SEQ ID NO: 417) | GTTCACAC | 45 | 1 |
| hTfpi-Cj-16 | TTGTTCATATTGCCCAGGCATC(SEQ ID NO: 418) | CACCATAC | 45 | 0.3 |
| hTfpi-Cj-17 | GCCTGGGCAATATGAACAATTT(SEQ ID NO: 419) | TGAGACAC | 41 | 0.1 |
| hTfpi-Cj-18 | CACAATCCTCTGTCTGCTGGAG(SEQ ID NO: 420) | TGAGACAC | 55 | 16.5 |
| hTfpi-Cj-19 | TAGTAGAATCTGTTCTCATTGG(SEQ ID NO: 421) | CACGACAC | 36 | 8.7 |
| hTfpi-Cj-20 | AATTGTAGTAGAATCTGTTCTC(SEQID NO: 422) | ATTGGCAC | 32 | 0.1 |
| hTfpi-Cj-21 | GTCATTGGGAAATGCCGCCCAT(SEQ ID NO: 423) | TTAAGTAC | 55 | 1.5 |
| hTfpi-Cj-22 | TTGTTTTCATTTCCCCCACATC(SEQ ID NO: 424) | CACTGTAC | 41 | 0 |

Through the experiments of items 4 to 6 of “Experimental Methods”, a thrombin peak and total thrombin formation ability were confirmed, and it was found that the thrombin formation ability was improved in the mice. In addition, it was confirmed that an enhanced thrombosis reduced the frequency of red blood cells in the liver tissue, thereby reducing spontaneous bleeding and further reducing secondary hemophilia complications.

Therefore, it is expected that the composition of the present application, which includes gRNA and Cas9 targeting the AT gene and the TFPI gene presented in this study, may be efficiently used to treat hemophilia.

### Industrial Applicability

According to the present invention, a blood coagulation system can be regulated using a composition for gene manipulation, which includes a guide nucleic acid targeting a blood coagulation inhibitory gene, and an editor protein, by artificially manipulating and/or modifying the blood coagulation inhibitory gene to regulate the function and/or expression of the blood coagulation inhibitory gene, and thus coagulopathy can be treated or improved using the composition for gene manipulation.

### Sequence List Text

Target sequences of AT gene and TFPI gene and guide sequences capable of targeting the target sequences.

This application contains references to amino acid sequences and/or nucleic acid sequences which have been submitted concurrently herewith as the sequence listing

XML file entitled “000027uscpb_SequenceListing.XML”, file size 907,824 bytes, created on 20 Jan. 2023. The aforementioned sequence listing is hereby incorporated by reference in its entirety pursuant to 37 C.F.R. §1.52(e)(5).

What is claimed is:

1. A composition for AT (antithrombin) gene manipulation, comprising:
i) a CRISPR enzyme, or a nucleic acid sequence encoding the CRISPR enzyme; and
ii) a guide RNA, or a nucleic acid sequence encoding the guide RNA;
wherein the guide RNA includes: a guide domain; and one or more domains selected from a first complementary domain, a second complementary domain, a linker domain, a proximal domain and a tail domain,
wherein the guide domain includes a guide sequence capable of forming a complementary bond with a target sequence located in the AT gene,
wherein the target sequence is a guide nucleic acid binding sequence,
wherein the guide sequence is at least one guide sequence selected from a SEQ ID NO: 425 ~ 688 and SEQ ID NO: 849 ~911.

2. The composition of claim 1, wherein the guide sequence is at least one guide sequence selected from the group consisting of SEQ ID NOs: 427, 428, 436, 437, 443, 447, 449, 454, 458, 463, 464, 473, 474, 478, 482, 486, 494, 498, 504, 507, 522, 544, 548, 584, 589, 591, 594, 596, 602, 604, 605, 606, 622, 623, 624, 625, 626, 628, 632, 634, 635, 638, 639, 642, 659 and 676.

3. The composition of claims 1, wherein the CRISPR enzyme is at least one Cas9 protein selected from the group consisting of a Streptococcus pyogenes-derived Cas9 protein, a Staphylococcus aureus-derived Cas9 protein and a Campylobacter jejuni- derived Cas9 protein.

4. The composition of claim 1, wherein the composition includes a form of guide RNA-CRISPR enzyme complex combined guide RNA and CRISPR enzyme.

5. The composition of claim 1, wherein the guide RNA and the CRISPR enzyme are present in one vector or are present in separated vectors in a form of a nucleic acid sequence, respectively.

6. The composition of claim 5, wherein the vector is a plasmid or viral vector.

7. The composition of claim 6, wherein the viral vector is a one or more viral vectors selected from the group consisting of a retrovirus, a lentivirus, an adenovirus, an adeno- associated virus (AAV), a vaccinia virus, a poxvirus and a herpes simplex virus.

8. The composition of claim 7, wherein the viral vector is an adeno-associated virus (AAV).

9. The composition of claim 1, wherein the composition includes an organic nanoparticle comprising the nucleic acid sequence encoding the CRISPR enzyme and nucleic acid sequence encoding the guide RNA.

10. The composition of claim 9, wherein the organic nanoparticle is LNP.

11. The composition of claim 9, wherein the LNP comprises a polyethylene glycol (PEG)-coated lipid.

12. A method for artificially manipulating a AT (antithrombin) gene to treat a hemophilia, the method comprising
introducing a composition to a subject,
wherein the composition comprises i) a CRISPR enzyme, or a nucleic acid sequence encoding the CRISPR enzyme; and ii) a guide RNA, or a nucleic acid sequence encoding the guide RNA,
wherein the guide RNA comprises a guide sequence selected from SEQ ID NO: 425 ~ 688 and SEQ ID NO: 849 ~911,
wherein the guide sequence is capable of forming a complementary bond with a target sequence located in the AT gene, and
wherein the target sequence is a guide nucleic acid binding sequence.

13. The method of claim 12, wherein the guide sequence is at least one guide sequence selected from the group consisting of SEQ ID NOs: 427, 428, 436, 437, 443, 447, 449, 454, 458, 463, 464, 473, 474, 478, 482, 486, 494, 498, 504, 507, 522, 544, 548, 584, 589, 591, 594, 596, 602, 604, 605, 606, 622, 623, 624, 625, 626, 628, 632, 634, 635, 638, 639, 642, 659 and 676.

14. The method of claim 12, wherein the CRISPR enzyme is at least one Cas9 protein selected from the group consisting of a Streptococcus pyogenes-derived Cas9 protein, a Staphylococcus aureus-derived Cas9 protein and a Campylobacter jejuni-derived Cas9 protein.

15. The method of claim 12, wherein the composition comprises an adeno-associated virus (AAV).

16. The method of claim 12, wherein the composition comprises an organic nanoparticle comprising the nucleic acid sequence encoding the CRISPR enzyme and nucleic acid sequence encoding the guide RNA.

17. The composition of claim 16, wherein the organic nanoparticle is LNP.

18. The method of claim 12, wherein the hemophilia is a hemophilia A or hemophilia B.

19. The method of claim 12, wherein the target sequence located in exon 1, exon 2, exon 3, exon 4, exon 5, exon 6 or exon 7 of AT gene.
